SEQUENCE LISTING (654 sequences)

SEQ ID NO: 1 - DNA, 20 bp, Artificial sequence
  Single strand DNA oligonucleotide
gtgstgcayggyygtcgtca

SEQ ID NO: 2 - DNA, 20 bp, Artificial sequence
  Single strand DNA oligonucleotide
  misc_feature (12)..(12): n is a, c, g, or t
acgtcrtccmcnccttcctc

SEQ ID NO: 3 - DNA, 20 bp, Artificial sequence
  Single strand DNA oligonucleotide
ggattagatacccsggtagt

SEQ ID NO: 4 - DNA, 21 bp, Artificial sequence
  Single strand DNA oligonucleotide
gttgartccaattaaaccgca

SEQ ID NO: 5 - DNA, 20 bp, Artificial sequence
  Single strand DNA oligonucleotide
gctttcgwtggtagtgtatt

SEQ ID NO: 6 - DNA, 19 bp, Artificial sequence
  Single strand DNA oligonucleotide
cttgccctcyaatcgtwct

SEQ ID NO: 7 - DNA, 251 bp, Artificial sequence
  16S rRNA  amino acid sequence
tacggaggatgcgagcgttatccggatttattgggtttaaagggtgcgtaggtggtttgttaagtttgtggtgaaagcgtgcggctcaaccgtaccaagccatgaaaactggcgaacttgagtgcaaacgaggtaggcggaatgtgatgtgtagcggtgaaatgcttagatatgtcacagaaccccgattgcgaaggcagcttaccagcatgcaactgacactgaggcacgaaagcgtgggtatcaaacag

SEQ ID NO: 8 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagagatttggaagaataccggaggcgaaggcggctctctggaccgatactgacactgaagagcgaaagcgtggggagcaaacag

SEQ ID NO: 9 - DNA, 252 bp, Artificial sequence
  16S rRNA  amino acid sequence
tacggaggatccgagcgttatccggatttattgggtttaaagggagcgtaggcggaggatcaagtcagttgtgaaatgtaggcgcttaacgtctgcactgcagttgaaactggttcccttgagtgcgtaagaggcaggcggaattcgtcgtgtagcggtgaaatgcttagatatgacgaagaactccgatcgcgaaggcagcttgccgggccgcaactgacgctgaagctcgaaggtgcgggtatcgaacag

SEQ ID NO: 10 - DNA, 252 bp, Artificial sequence
  16S rRNA  amino acid sequence
tacggaggatgcaagcgttatccggatttattgggtttaaagggtgcgcaggcgggctgttaagtcagcggtgaaatgtcccggctcaaccggggcactgccgttgatactggcagcctagagtacagacggcgccggcggaatgtgtcatgtagcggtgaaatgcttagatatgccacagaacaccgatcgcgaaggcagctggcgaggctgggactgacgctcaggcacgaaagcgtggggatcaaacag

SEQ ID NO: 11 - DNA, 252 bp, Artificial sequence
  16S rRNA  amino acid sequence
tacgtaggtggcgagcgttgttcggatttattgggcgtaaagggtccgcaggtggttggataagtttgacgtgaaatcccagagctcaactctggaactgcgttgaaaactgtctgactggagtatcggagaggagattggaattccaggtgtagcggtggaatgcgtagatatctggaagaacaccgaaagcgaaggcagatctctggacgataactgacactcagggacgaaagtatggggatcaaacag

SEQ ID NO: 12 - DNA, 252 bp, Artificial sequence; identical to SEQ ID NO: 11
  16S rRNA  amino acid sequence

SEQ ID NO: 13 - DNA, 252 bp, Artificial sequence; identical to SEQ ID NO: 11
  16S rRNA  amino acid sequence

SEQ ID NO: 14 - DNA, 252 bp, Artificial sequence
  16S rRNA  amino acid sequence
tacggaaggtccgggcgttatccggatttattgggtttaaagggagcgtaggccggatattaagcgtgttgtgaaatgtagatgctcaacgtctgcactgcagcgcgaactggtgtccttgagtgcgcaggaagtcggcggaattcgttgtgtagcggtgaaatgcttagatatgacgaagaactccgattgcgaaggcagctgactgtagcgttactgacgctgaagctcgaaggtgcgggtatcgaacag

SEQ ID NO: 15 - DNA, 252 bp, Artificial sequence
  16S rRNA  amino acid sequence
tacagaggtcgcaagcgttgttcggatttactgggcgtaaagggcgcgtaggcggccgtgcgtgtcggatgtgaaatctccgggctcaacccggaaagtgcgtccgaaactgcatggctcgagttcgggagagggaatcggaatgcagggtgtagcggtgaaatgcgttgatatcctgcagaacaccggaggcgaaggcggattcctggaacgaaactgacgctcaggcgcgaaagtcgggggagcaaacag

SEQ ID NO: 16 - DNA, 251 bp, Artificial sequence; identical to SEQ ID NO: 7
  16S rRNA  amino acid sequence

SEQ ID NO: 17 - DNA, 252 bp, Artificial sequence; identical to SEQ ID NO: 9
  16S rRNA  amino acid sequence

SEQ ID NO: 18 - DNA, 252 bp, Artificial sequence; identical to SEQ ID NO: 9
  16S rRNA  amino acid sequence

SEQ ID NO: 19 - DNA, 252 bp, Artificial sequence; identical to SEQ ID NO: 9
  16S rRNA  amino acid sequence

SEQ ID NO: 20 - DNA, 252 bp, Artificial sequence; identical to SEQ ID NO: 14
  16S rRNA  amino acid sequence

SEQ ID NO: 21 - DNA, 252 bp, Artificial sequence; identical to SEQ ID NO: 14
  16S rRNA  amino acid sequence

SEQ ID NO: 22 - DNA, 252 bp, Artificial sequence
  16S rRNA  amino acid sequence
tacgaggggtgcaagcgttgttcggaattactgggcgtaaagggagcgtaggcggagattcaagcggattgtacaatcccggggcccaaccccggctctgcagtccgaactggatctcttggatagttcaggggcaggcggaattcctggtgtagcggtggaatgcgtagagatcaggaagaacaccgatggcgaaggcagcctgctggggactcatcgacgctgaggctcgaaagtgcgggtagcaaacag

SEQ ID NO: 23 - DNA, 252 bp, Artificial sequence; identical to SEQ ID NO: 15
  16S rRNA  amino acid sequence

SEQ ID NO: 24 - DNA, 252 bp, Artificial sequence
  16S rRNA  amino acid sequence
tacgtatggagcaagcgttatccggatttactgggtgtaaagggagcgtagactgcacggcaagtcagatgtgaaagcccggggctcaacctcggaactgcatttgaaactgtcgagctagagtacaggagaggtaagtggaattcctagtgtagcggtgaaatgcgtagatattaggaggaacaccggtggcgaaggcggcttactggactgtaactgacgttgaggctcgaaagcgtggggagcaaacag

SEQ ID NO: 25 - DNA, 252 bp, Artificial sequence; identical to SEQ ID NO: 8
  16S rRNA  amino acid sequence

SEQ ID NO: 26 - DNA, 252 bp, Artificial sequence; identical to SEQ ID NO: 10
  16S rRNA  amino acid sequence

SEQ ID NO: 27 - DNA, 252 bp, Artificial sequence; identical to SEQ ID NO: 11
  16S rRNA  amino acid sequence

SEQ ID NO: 28 - DNA, 252 bp, Artificial sequence
  16S rRNA  amino acid sequence
tacggaaggtccgggcgttatccggatttattgggtttaaagggagcgcaggcggagacttaagcgtgctgtgaaatgtaccggctcaaccggtgagttgcagcgcgaactgggtttcttgagtgagtgcgaggtatgcggaattcgtggtgtagcggtgaaatgcttagatatcacgaagaactccgattgcgaaggcagcgtaccagtccttaactgacgctaaagctcgaaagtgcgggtatcgaacag

SEQ ID NO: 29 - DNA, 251 bp, Artificial sequence; identical to SEQ ID NO: 7
  16S rRNA  amino acid sequence

SEQ ID NO: 30 - DNA, 252 bp, Artificial sequence; identical to SEQ ID NO: 8
  16S rRNA  amino acid sequence

SEQ ID NO: 31 - DNA, 252 bp, Artificial sequence; identical to SEQ ID NO: 9
  16S rRNA  amino acid sequence

SEQ ID NO: 32 - DNA, 252 bp, Artificial sequence; identical to SEQ ID NO: 10
  16S rRNA  amino acid sequence

SEQ ID NO: 33 - DNA, 252 bp, Artificial sequence; identical to SEQ ID NO: 11
  16S rRNA  amino acid sequence

SEQ ID NO: 34 - DNA, 252 bp, Artificial sequence; identical to SEQ ID NO: 11
  16S rRNA  amino acid sequence

SEQ ID NO: 35 - DNA, 252 bp, Artificial sequence; identical to SEQ ID NO: 11
  16S rRNA  amino acid sequence

SEQ ID NO: 36 - DNA, 252 bp, Artificial sequence; identical to SEQ ID NO: 14
  16S rRNA  amino acid sequence

SEQ ID NO: 37 - DNA, 252 bp, Artificial sequence; identical to SEQ ID NO: 15
  16S rRNA  amino acid sequence

SEQ ID NO: 38 - DNA, 251 bp, Artificial sequence; identical to SEQ ID NO: 7
  16S rRNA  amino acid sequence

SEQ ID NO: 39 - DNA, 252 bp, Artificial sequence; identical to SEQ ID NO: 9
  16S rRNA  amino acid sequence

SEQ ID NO: 40 - DNA, 252 bp, Artificial sequence; identical to SEQ ID NO: 9
  16S rRNA  amino acid sequence

SEQ ID NO: 41 - DNA, 252 bp, Artificial sequence; identical to SEQ ID NO: 9
  16S rRNA  amino acid sequence

SEQ ID NO: 42 - DNA, 252 bp, Artificial sequence; identical to SEQ ID NO: 14
  16S rRNA  amino acid sequence

SEQ ID NO: 43 - DNA, 252 bp, Artificial sequence; identical to SEQ ID NO: 14
  16S rRNA  amino acid sequence

SEQ ID NO: 44 - DNA, 252 bp, Artificial sequence; identical to SEQ ID NO: 22
  16S rRNA  amino acid sequence

SEQ ID NO: 45 - DNA, 252 bp, Artificial sequence; identical to SEQ ID NO: 15
  16S rRNA  amino acid sequence

SEQ ID NO: 46 - DNA, 252 bp, Artificial sequence; identical to SEQ ID NO: 24
  16S rRNA  amino acid sequence

SEQ ID NO: 47 - DNA, 252 bp, Artificial sequence; identical to SEQ ID NO: 8
  16S rRNA  amino acid sequence

SEQ ID NO: 48 - DNA, 252 bp, Artificial sequence; identical to SEQ ID NO: 10
  16S rRNA  amino acid sequence

SEQ ID NO: 49 - DNA, 252 bp, Artificial sequence; identical to SEQ ID NO: 11
  16S rRNA  amino acid sequence

SEQ ID NO: 50 - DNA, 252 bp, Artificial sequence; identical to SEQ ID NO: 28
  16S rRNA  amino acid sequence

SEQ ID NO: 51 - DNA, 252 bp, Artificial sequence
  16S rRNA  amino acid sequence
cacgtaagccccgagcgttgttcggaattattgggcgtaaagggcatgtaggcggctttgcaagtctggtgtgaaaggcaggagcttaactcctggactgcgctggaaactgcatggcttgagtcactgaggggcatccggaattcctggtgtaggggtgaaatctgtagatatcaggaagaacaccgatggcgaaggcagggtgccagcggatgactgacgctgaggtgcgaaggtgcggggagcaaacag

SEQ ID NO: 52 - DNA, 252 bp, Artificial sequence
  16S rRNA  amino acid sequence
tacgtagggagcgagcgttgtccggaattactgggtgtaaagggagcgtaggcgggattgcaagtcaggtgtgaaatgcggaggctcaacctccgagctgcacttgaaactgtagttcttgagtgaagtagaggtaagcggaattcctggtgtagcggtgaaatgcgtagatatcaggaggaacatcggtggcgaaggcggcttactgggcttttactgacgctgaggctcgaaagcgtggggagcaaacag

SEQ ID NO: 53 - DNA, 252 bp, Artificial sequence
  16S rRNA  amino acid sequence
tacggaggatgcgagcgttatccggatttattgggtttaaagggtgcgtaggcgggttgttaagtcagtggtgaaaagccatcgctcaacgatggtcgtgccattgatactggcagtctggagttccgttgccgtgggaggaatgagtagtgtagcggtgaaatgcatagatattactcagaacaccgattgtgaagacatctcacgaagcggcgactgacgctgaggcacgaaagcgtggggatcgaacag

SEQ ID NO: 54 - DNA, 252 bp, Artificial sequence
  16S rRNA  amino acid sequence
tacggaaggtccgggcgttatccggatttattgggtttaaagggagcgcaggccgattattaagcgtgacgtgaaatgtagatgctcaacatctgaactgcgtcgcgaactggaggccttgagtgcattcgacgtcggcggaattcgtggtgtagcggtgaaatgcttagatatcacgaagaactccgattgcgcaggcagctgacgagactgtaactgacgctaaagctcgaaagtgcgggtatcgaacag

SEQ ID NO: 55 - DNA, 252 bp, Artificial sequence
  16S rRNA  amino acid sequence
tacggaggatgcgagcgttatccggatttattgggtttaaagggagcacaggccggctgtcaagcgtgttgtgaaatgtaggggctcaacctctgaactgcagcgcgaactgacggtcttgagtgcgcacaacgttggcggaattcgtcgtgtagcggtgaaatgcttagatatgacgaagaactccgattgcgaaggcagctgacgggagcgttactgacgcttaagctcgaaagtgcgggtatcgaacag

SEQ ID NO: 56 - DNA, 251 bp, Artificial sequence
  16S rRNA  amino acid sequence
tacggaaggtccgggcgttatccggatttattgggtttaaagggtgcgcaggcggcctgtcaagtcagcggtcaaatttcggcgctcaacgccgtaccgccgttgaaactggcagtctcgagtgcgtgggaggatggcggaatgcgcggtgtagcggtgaaatgcatagatatcgcgcagaaccccgattgcgaaggcagctgtccatcacgccactgacgctcatgcacgaaagcgtgggtatcgaacag

SEQ ID NO: 57 - DNA, 251 bp, Artificial sequence
  16S rRNA  amino acid sequence
tacggaagatgcgagcgttatccggatttattgggtttaaagggagcgtaggcggcccgttaagtcagcggttaaatgtcagtgcccaacattggcatgccgttgatactggtgggcttgaatacacacaaggatggtggaattcgtcgtgtagcggtgaaatgcttagatatgacgaagaactccgattgcgaaggcagccatctggggtgcgattgacgctgaggctcgaaagtgcgggaatcaaacag

SEQ ID NO: 58 - DNA, 252 bp, Artificial sequence
  16S rRNA  amino acid sequence
tacgaaggtggcgagcgttactcggaattactaggcgtaaagcgtatgtaggtggttggataagtctatggtgaaatttcctggctcaactgggaagggaacatagatactgtccggcttgagtgtgtgagggggagacggaattcctggtgtagcggtgaaatgcgtagatatcaggaggaacaccgatggcgaaagcagtctcctggcacaaaactgacactgaggtacgaaagctagggtagcaaacag

SEQ ID NO: 59 - DNA, 252 bp, Artificial sequence
  16S rRNA  amino acid sequence
tacggaggatgcgagcgttatccggatttattgggtttaaagggagcacaggccggctgtcaagcgtgttgtgaaatgtaggcgctcaacgcctgacttgcagcgcgaactgacggtcttgagtgcgcgcaacgttggcggaatttgtcgtgtagcggtgaaatgcttagatatgacgaagaactccgattgcgaaggcagctgacgggagcgccactgacgcttaagctcgaaagtgcgggtatcgaacag

SEQ ID NO: 60 - DNA, 251 bp, Artificial sequence
  16S rRNA  amino acid sequence
tacgtaggtggcaagcgttgtccggaattattgggcgtaaagcgcgcgcaggcggccctttaagtccatcttaaaagtgcggggcttaaccccgtgatgggatggaaactggagagctggagtatcggagaggaaagtggaattcctagtgtagcggtgaaatgcgtagagattgggaggaacaccagtggcgaaggcgacttactggactgtaactgacgctgaggcgcgaaagtgtggggagcaaacag

SEQ ID NO: 61 - DNA, 252 bp, Artificial sequence
  16S rRNA  amino acid sequence
tacgtagggggcaagcgttatccggatttactgggtgtaaagggtgcgcaggcggcctgccaagtcagcggtgaaagcccggggcccaaccccggaagtgccgttgatactggtgggctggaatgcggtcgaggcgggcggaacgtggcgtgtagcggtgaaatgcgtagatatgccacagaacgccgatagcggaggcagctcgccaggcctgcattgacgctcgggcacgaaagcgtggggatcgaacag

SEQ ID NO: 62 - DNA, 252 bp, Artificial sequence
  16S rRNA  amino acid sequence
tacagaggtccctagcgttgttcggatttactgggcgtaaagggcacgtaggtggctaggcgtgtcgcgtgtgaaatctccgggcccaacccggaaagtgcgcgcgaaactacctggctggagtacgggagagggagtcggaatgcagggtgtagcggtgaaatgcgttgatatcctgcagaacaccggaggcgaaggcggactcctggaacgttactgacgctaaggtgcgaaagtcgggggagcaaacag

SEQ ID NO: 63 - DNA, 252 bp, Artificial sequence
  16S rRNA  amino acid sequence
tacggaaggtccaggcgttatccggatttattgggtttaaagggagcgtaggccggagattaagtgtgttgtgaaatttaggcgctcaacgtctgcactgcagcgcgaactggctgacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 64 - DNA, 252 bp, Artificial sequence
  16S rRNA  amino acid sequence
tacgtatggagcaagcgttatccggatttactgggtgtaaagggagcgtaggcggtcttgcaagtctgaagtgaaaaccctgggctcaaccctgggattgctttggaaactgtaggactggagtgtcggaggggcatgtggaattcctggtgtagcggtgaaatgcgtagatatcaggaggaacaccagtggcgaaggcggcatgctggacgataactgacgctgatgctcgaaagcgtggggagcaaacag

SEQ ID NO: 65 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagagatagggaggaacaccagtggcgaaggcggcctgctgggctttaactgacgctgaggcacgaaagcgtgggtagcaaacag

SEQ ID NO: 66 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcacaacgcgggcggaatttgtcgtgtagcggtgaaatgcttagatatgacgaagaaccccgattgcgaaggcagctgacgagtccttaactgacgctaaagctcgaaagtgcgggtatcgaacag

SEQ ID NO: 67 - DNA, 252 bp, Artificial sequence; identical to SEQ ID NO: 66
  16S rRNA  amino acid sequence

SEQ ID NO: 68 - DNA, 252 bp, Artificial sequence
  16S rRNA  amino acid sequence
tacggagggtgcaagcgttaatcggaataactgggcgtaaagggcatgcaggcggttgtataagtagggtgtgaaatccccgggctcaacctgggaactgcattctaaactgtacaactagagtattgcagggggagacggaattccaggtgtagcggtggaatgcgtagatatctggaagaacaccaaaggcgaaggcagtctcctgggcaaatactgacgctcatatgcgaaagcgtgggtagcaaacag

SEQ ID NO: 69 - DNA, 251 bp, Artificial sequence
  16S rRNA  amino acid sequence
tacggaggatgcgagcgttatccggatttattgggtttaaagggtgcgcaggcggcgtgtcaagtcagcggtcaaactgcggggctcaaccccgtaccgccgttgaaactggcgcgctcgagtgtgtgcgaggatggcggaatgcgcggtgtagcggtgaaatgcatagatatcgcgcagaactccgattgcgaaggcagctgtccagcacgccactgacgctcatgcacgaaagcgtgggtatcgaacag

SEQ ID NO: 70 - DNA, 252 bp, Artificial sequence
  16S rRNA  amino acid sequence
tacggaaggtccgggcgttatccggatttattgggtttaaagggagcgcaggctgattgttaagcgtgacgtgaaatgtagccgctcaacggctgaactgcgtcgcgaactggcagtcttgagtgagtacgacgtcagcggaattcgtggtgtagcggtgaaatgcttagatatcacgaagaaccccgattgcgaaggcagctgacgagtcctttactgacgctaaagctcgaaggtgcgggtatcgaacag

SEQ ID NO: 71 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgcccaacgcctgcactgcagcgcgaactggctgacttgagtacgcacgacgttggcggaatttgccgtgtagcggtgaaatgcttagatatggcgaagaactccgattgcgaaggcagcttacgggagcgcaactgacgctgaagctcgaaagtgcgggtatcgaacag

SEQ ID NO: 72 - DNA, 252 bp, Artificial sequence
  16S rRNA  amino acid sequence
tacggagggtgcgagcgttaatcggaataactgggcgtaaagagctcgtaggcggttagtcaagtcagatgtgaaagccccgggcttaacctgggattggctcgtgaaactgtttaactagagtacggtagaggggagtagaattccaagtgtagcggtgaaatgcgtagagatttggaagaataccggaggcgaaggcggctctctggaccgatactgacactgaagagcgaaagcgtggggagcaaacag

SEQ ID NO: 73 - DNA, 252 bp, Artificial sequence
  16S rRNA  amino acid sequence
tacggaaggtccgggcgttatccggatttattgggtttaaagggagcgtaggcgggatgtcaagcgtgctgtgaaaagctgcggctcaaccgtagtcgtgcagcgcgaactggcttccttgagtgcgcgggaggcacgcggaattcgtggtgtagcggtgaaatgcttagatatcacgaagaaccccgattgcgaaggcagcgtgccgtagcgttactgacgctgaagctcgaaggtgcgggtatcgaacag

SEQ ID NO: 74 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatcacgaagaactccaattgcgaaggcagctcgctgtagtgttactgacgctaaagctcgaaagtgcgggtatcgaacag

SEQ ID NO: 75 - DNA, 252 bp, Artificial sequence
  16S rRNA  amino acid sequence
tacggaaggtccgggcgttatccggatttattgggtttaaagggagcgcaggccgccgattaagcgtgttgtgaaatgtaggggctcaacctctgaattgcagcgcgaactggtcggcttgagatagcgcgaggtaggcggaattcgtggtgtagcggtgaaatgcttagatatcacgaagaaccccgattgcgaaggcagcttaccagagctattctgacgctaaagctcgaaggtgcgggtatcgaacag

SEQ ID NO: 76 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgacttgcagcgcgaactggctgacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 77 - DNA, 252 bp, Artificial sequence
  16S rRNA  amino acid sequence
tacgtagggggcaagcgttatccggatttactgggtgtaaagggagtgtaggtggtcatgcaagtccgaagtgaaatcccggggctcaaccccgtgactgctttggaaactgtgtgactggagtgcaggagaggtaagtggaattcctagtgtagcggtgaaatgcgtagatattaggaagaacaccggtggcgaaggcggcttactggactgtaactgacactgaggctcgaaagcgtggggagcaaacag

SEQ ID NO: 78 - DNA, 252 bp, Artificial sequence
  16S rRNA  amino acid sequence
tacggaaggtccgggcgttatccggatttattgggtttaaagggagcgcagacggaagagcaagtctgaagtgaaaacctgcggcttaactgcaggattgctttggaaactgtttttcttgagtactggagaggtaagcggaattcctggtgtagtagtgaaatgcgtagatatcaggaagaacaccggaggcgaaggcggcttactggacagcaactgacgttgaggctcgaaagcgtggggagcaaacag

SEQ ID NO: 79 - DNA, 252 bp, Artificial sequence
  16S rRNA  amino acid sequence
tacggaaggtccgggcgttatccggatttattgggtttaaagggagcgcaggctgatgattaagcgtgacgtgaaatgtagccgctcaacggctgaactgcgtcgcgaactggcagtcttgagtgagtacgacgtcagcggaattcgtggtgtagcggtgaaatgcttagatatcacgaagaactccgattgcgaaggcagcccacaaggcctttactgacgctaaagctcgaaggtgcgggtatcgaacag

SEQ ID NO: 80 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcgcaacgtttgcggaatttgtcgtgtagcggtgaaatgcttagatatgacgaagaactccgattgcgaaggcagcagacgggagcgcaactgacgctgaagctcgaaagtgcgggtatcgaacag

SEQ ID NO: 81 - DNA, 252 bp, Artificial sequence; identical to SEQ ID NO: 80
  16S rRNA  amino acid sequence

SEQ ID NO: 82 - DNA, 252 bp, Artificial sequence
  16S rRNA  amino acid sequence
tacggaaggtccgggcgttatccggatttattgggtttaaagggagcgcaggctgatgattaagcgtgacgtgaaatgtagccgctcaacggctgaactgcgtcgcgaactggttatcttgagtgagttcgatgttggcggaattcgtggtgtagcggtgaaatgcttagatatggcgaagaactccgattgcgaaggcagctgacgggagcgcaactgacgctcatgctcgaaagtgcgggtatcgaacag

SEQ ID NO: 83 - DNA, 252 bp, Artificial sequence
  16S rRNA  amino acid sequence
tacggaggatgcgagcgttatccggatttattgggtttaaagggagcgcaggctgattattaagcgtgacgtgaaatgtagccgctcaacggctgaactgcgtcgcgaactggtagtcttgagtgagttcgatgtaggcggaattcgtggtgtagcggtgaaatgcttagatatcacgaagaactccgattgcgaaggcagcctacaaggcctttactgacgctaaagctcgaaggtgcgggtatcgaacag

SEQ ID NO: 84 - DNA, 252 bp, Artificial sequence
  16S rRNA  amino acid sequence
tacggaaggtccgggcgttatccggatttattgggtttaaagggagcgcaggctgaaggttaagcgtgacgtgaaatgtaccggctcaaccggtgaattgcgtcgcgaactggtcttcttgagtgagtgcgacgtcagcggaattcgtggtgtagcggtgaaatgcttagatatcacgaagaactccgattgcgaaggcagctgacgagtccttaactgacgctaaagctcgaaagtgcgggtatcgaacag

SEQ ID NO: 85 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcacaacgcgggcggaatttgtcgtgtagcggtgaaatgcttagatatcacgaagaactccgattgcgaaggcagccaacaaggcctttactgacgctaaagctcgaaggtgcgggtatcgaacag

SEQ ID NO: 86 - DNA, 252 bp, Artificial sequence; identical to SEQ ID NO: 85
  16S rRNA  amino acid sequence

SEQ ID NO: 87 - DNA, 252 bp, Artificial sequence; identical to SEQ ID NO: 85
  16S rRNA  amino acid sequence

SEQ ID NO: 88 - DNA, 252 bp, Artificial sequence
  16S rRNA  amino acid sequence
tacggaaggtccgggcgttatccggatttattgggtttaaagggagcgtaggcgggctgttaagcgtgtcgtgaaaagcatcggctcaaccggtgtcgtgcggcgcggactggcggtcttgagtgggtagaaagccggcggaattcgtggtgtagcggtgaaatgcttagatatcacgaagaactccgattgcgaaggcagctggctgtatccttactgacgctaaagctcgaaagcgcgggtatcgaacag

SEQ ID NO: 89 - DNA, 252 bp, Artificial sequence
  16S rRNA  amino acid sequence
tacggaaggtccgggcgttatccggattcattgggtttaaagggagcgtaggccgccccttaagcgtgttgtgaaatgccgcggctcaaccgtggcactgcagcgcgaactggggggcttgagtgcgcgcagcgaaggcggaattcgtggtgtagcggtgaaatgcttagatatcacgaagaactccgatcgcgaaggcagccttcgggagcgcaactgacgctgaagctcgaaagtgcgggtatcgaacag

SEQ ID NO: 90 - DNA, 252 bp, Artificial sequence; identical to SEQ ID NO: 89
  16S rRNA  amino acid sequence

SEQ ID NO: 91 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatgacgaagaactccgattgcgaaggcagcctgctgtagcgccactgacgctgatgctcgaaagcgtgggtatcgaacag

SEQ ID NO: 92 - DNA, 252 bp, Artificial sequence
  16S rRNA  amino acid sequence
tacggaaggtccgggcgttatccggatttattgggtttaaagggagcgcaggcggccttttaagcgtgacgtgaaatgcagacgcccaacgtctgaactgcgtcgcgaactggagggcttgagtgggtacgacgccggcggaattcgtggtgtagcggtgaaatgcttagatatcacgaagaaccccgattgcgaaggcagccggcgagtcctcaactgacgctaaagctcgaaagtgcgggtatcgaacag

SEQ ID NO: 93 - DNA, 252 bp, Artificial sequence
  16S rRNA  amino acid sequence
tacggaaggtccgggcgttatccggatttattgggtttaaagggagcgcaggctgatgattaagcgtgacgtgaaatgtagccgctcaacggctgaactgcgtcgcgaactggttatcttgagtgagttcgatgttggcggaattcgtggtgtagcggtgaaatgcttagatatgacgaagaaccccgattgcgaaggcagctcgcgggagcgcaactgacgctgaagctcgaaagtgcgggtatcgaacag

SEQ ID NO: 94 - DNA, 252 bp, Artificial sequence
  16S rRNA  amino acid sequence
tacgtagggggcaagcgttatccggatttactgggtgtaaagggagcgcagacggaactgcaagtctgatgtgaaagcccggggctcaaccgcgggactgcatcggatactgtagatctggagtgccggaggggcaagcggaattcctggtgtagcggtgaaatgcgtagatatcaggaggaacaccggcggcgaaggcggcttgctggacggcaactgacgttgaggctcgaaagcgtggggagcaaacag

SEQ ID NO: 95 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgacttgcagcgcgaactgacggtcttgagtacgcacaacgttggcggaatttgtcgtgtagcggtgaaatgcttagatatcacgaagaactccgattgcgaaggcagctttccagtccttaactgacgctaaagctcgaaagtgcgggtatcgaacag

SEQ ID NO: 96 - DNA, 252 bp, Artificial sequence
  16S rRNA  amino acid sequence
tacgtagggggcaagcgttatccggatttactgggtgtaaagggagcgcagacggcagtgcaagtctgatgtgaaagcccggggctcaaccccgggactgcatttgaaactgtgaagctggagtgtcggagaggtaagtggaattcctagtgtagcggtgaaatgcgtagatattaggaagaacaccagtggcgaaggcggcttactggacgattactgacgctgaggctcgaaagcgtggggagcaaacag

SEQ ID NO: 97 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtttccttgagtacacgcaacgttggcggaattcgtcgtgtagcggtgaaatgcttagatatgacgaagaactccgattgcgaaggcagctgacgggagtgcaactgacgctgaagctcgaaagtgcgggtatcgaacag

SEQ ID NO: 98 - DNA, 252 bp, Artificial sequence
  16S rRNA  amino acid sequence
tacgtagggagcaagcgttatccggatttattgggtgtaaagggtgcgtagacggataaacaagttagttgtgaaatacctcggcttaactgaggaactgcaactaaaactatatatcttgagtactggaggggaaagcggaattcctagtgtagcggtgaaatgcgtagatattaggaagaacaccggtggcgaaggcggctttctggacagcaactgacgttgaggcacgaaagtgtggggagcaaacag

SEQ ID NO: 99 - DNA, 251 bp, Artificial sequence
  16S rRNA  amino acid sequence
tacggaagatgcgagcgttatccggatttattgggtttaaagggagcgtaggcggaagcttaagtcagtggtcaaatcgcgtggctcaaccatgcgccgccgttgatactgggcttcttgagtgcacataaggatggtggaattcgtcgtgtagcggtgaaatgcttagatatgacgaagaactccgattgcgaaggcagctgtccggggtgtaactgacgctaatgctcgaaagtgcgggtatcaaacag

SEQ ID NO: 100 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatgacgaagaactccgattgcgaaggcagctgactgtagcgttactgacgctgaagctcgaaggtgcgggtatcgaacag

SEQ ID NO: 101 - DNA, 252 bp, Artificial sequence
  16S rRNA  amino acid sequence
tacggaaggtccgggcgttatccggatttattgggtttaaagggagcgcaggcggaggcttaagcgtgacgtgaaatgtagccgctcaacggctgagttgcgtcgcgaactgggtttcttgagtgagtgcgacgtcagcggaattcgtggtgtagcggtgaaatgcttagatatcacgaagaactccgattgcgaaggcagctgacgagtccttaactgacgctaaagctcgaaagtgcgggtatcgaacag

SEQ ID NO: 102 - DNA, 252 bp, Artificial sequence
  16S rRNA  amino acid sequence
tacgaggggtgcaagcgttgttcggaatcactgggcgtaaagggagcgtaggcggagatgcaagcagtctgtacaataccggggcccaaccccggacctgcagactggactgtgtctcttggatagttcaggggcaggcggaattcctggtgtagcggtggaatgcgtagagatcaggaagaacaccgatggcgaaggcagcctgctggggacttatcgacgctgaggctcgaaagtgcgggtagcaaacag

SEQ ID NO: 103 - DNA, 252 bp, Artificial sequence
  16S rRNA  amino acid sequence
tacgtagggggcgagcgttgtccggaatgattgggcgtaaagggcgcgtaggcggcccggtaagtctggagtgaaagtcctgcttttaaggtgggaattgctttggatactgtcgggcttgagtgcaggagaggtaagtggaattcccagtgtagcggtgaaatgcgtagagatttggaagaataccggaggcgaaggcggctctctggaccgatactgacactgaagagcgaaagcgtggggagcaaacag

SEQ ID NO: 104 - DNA, 252 bp, Artificial sequence
  16S rRNA  amino acid sequence
tacgtatggagcaagcgttatccggatttactgggtgtaaagggagtgtaggcggctttgcaagtcagatgtgaaagcccggggctcaaccccgggactgcatttgaaactgtgaagctggagtgtcggagaggtaagtggaattcctagtgtagcggtgaaatgcgtagatattaggaagaacaccagtggcgaaggcggcttactggacgattactgacgctgaggctcgaaagcgtggggagcaaacag

SEQ ID NO: 105 - DNA, 252 bp, Artificial sequence; identical to SEQ ID NO: 104
  16S rRNA  amino acid sequence

SEQ ID NO: 106 - DNA, 252 bp, Artificial sequence
  16S rRNA  amino acid sequence
tacgtagggagcaagcgttatccggatttattgggtgtaaagggtgcgtagacggtttagtaagttggttgtgaaaacccggggcttaaccccggaattgcaaccaaaactaccaatcttgagtactggaggggaatgcggaattcctagtgtagcggtgaaatgcgtagatattaggaagaacaccggtggcgaaggcggcattctggacagcaactgacgttgaggcacgaaagtgtggggagcaaacag

SEQ ID NO: 107 - DNA, 251 bp, Artificial sequence
  16S rRNA  amino acid sequence
tacgtaggtggcaagcgttgtccggaattattgggcgtaaagcgcgcgcaggcggcctcctaagtccatcttaaaagtgcggggcttaaccccgtgatgggatggaaactgggaagctggagtatcggagaggaaagtggaattcctagtgtagcggtgaaatgcgtagatattaggaagaacaccagtggcgaaggcggcttactggacgattactgacgctgaggctcgaaagcgtggggagcaaacag

SEQ ID NO: 108 - DNA, 252 bp, Artificial sequence
  16S rRNA  amino acid sequence
tacggaaggtccgggcgttatccggatttattgggtttaaagggagcgtaggccggatggcaagcgtgttgtgaaatgtcgtggcccaaccagggcactgcagcgcgaactgccgttcttgagtgcgcgcaacgttggcggaatttgtcgtgtagcggtgaaatgcttagatatgacgaagaactccgattgcgaaggcagctgacgggagcgcaactgacgctgaagctcgaaagtgcgggtatcgaacag

SEQ ID NO: 109 - DNA, 252 bp, Artificial sequence; identical to SEQ ID NO: 108
  16S rRNA  amino acid sequence

SEQ ID NO: 110 - DNA, 252 bp, Artificial sequence
  16S rRNA  amino acid sequence
tacgtatggagcaagcgttatccggatttactgggtgtaaagggagcgtagacggtaaggcaagtctgatgtgaaaggtgtgggctcaacccgcaaactgcattggaaactgtcgtgctagagtgtcggagaggtaagtggaattcctagtgtagcggtgaaatgcgtagatattaggaggaacaccagcggcgaaggcggcttactggacgataactgacgttgaggctcgaaagcgtggggagcaaacag

SEQ ID NO: 111 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagagattaggaagaacaccggtggcgaaggcgactttctggacgacaactgacgctgaggcgcgaaagcgtggggagcaaacag

SEQ ID NO: 112 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagatattaggaggaacaccagtggcgaaggcggctttctggactgttactgacactgaggcacgaaagcgtggggagcaaacag

SEQ ID NO: 113 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagagatgtggaagaataccagaggcgaaggcggcctcctggacagatactgacgctgaagctcgaaagtgcgggtatcgaacag

SEQ ID NO: 114 - DNA, 252 bp, Artificial sequence
  16S rRNA  amino acid sequence
tacggaaggtccgggcgttatccggatttattgggtttaaagggagcgcaggccgatgcttaagcgtgacgtgaaatgtagccgctcaacggctgaactgcgtcgcgaactgggtatcttgagtgagtacgacgtggacggaattcgtggtgtagcggtgaaatgcttagatatcacgaagaactccgattgcgaaggcagcccacaaggcctttactgacgctaaagctcgaaggtgcgggtatcgaacag

SEQ ID NO: 115 - DNA, 251 bp, Artificial sequence
  16S rRNA  amino acid sequence
tacggaggatgcgagcgttatccggatttattgggtttaaagggtgcgcaggcggcacgtcaagtcagcggtcaaactgcggggctcaaccccgtaccgccgttgaaactggcgcgctggagtatgtgcgatggtggcggaatgcgcagtgtagcggtgaaatgcatagatattgcgcagaactccgattgcgaaggcagctgccaagcacatgactgacgctcatgcacgaaagcgtgggtatcgaacag

SEQ ID NO: 116 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcgaactgacggtcttgagtacgcacaacgttggcggaatttgtcgtgtagcggtgaaatgcttagatatggcgaagaactccgattgcgaaggcagctgacgggagcgcaactgacgctcatgctcgaaagtgcgggtatcgaacag

SEQ ID NO: 117 - DNA, 252 bp, Artificial sequence
  16S rRNA  amino acid sequence
tacagaggtcgcaagcgttgttcggatttactgggcgtaaagggcgcgtaggcggcagggcgtgtcaagtgtgaaatctccgggctcaacccggaaagtgcgcttgaaactaccttgctcgagtatgggagagggaatcggaatatagggtgtagcggtgaaatgcgttgatatcctatagaacaccggaggcgaaggcggattccttgaacatcactgacgctgaggcgcgaaagtcgggggagcaaacag

SEQ ID NO: 118 - DNA, 251 bp, Artificial sequence
  16S rRNA  amino acid sequence
tacggaaggtccgggcgttatccggatttattgggtttaaagggagcgcaggcggagggtcaagtcagccgtaaaatcgtcctgctcaacaggatcccgcggttgaaactggtccccttgggtgcacaagaggcacgcggaactcgtggtgtagcggtgaaatgcatagatatcacgaagaacgccgattgcgaaggcagcgtgctggggtgtaaccgacgctcatgctcgaaggcgcgggtatcaaacag

SEQ ID NO: 119 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagagatttggaagaataccggaggcgaaggcggcttactggactgtaactgacgttgaggctcgaaagcgtggggagcaaacag

SEQ ID NO: 120 - DNA, 252 bp, Artificial sequence
  16S rRNA  amino acid sequence
tacgtagggggcgagcgttgtccggaatgattgggcgtaaagggcgcgtaggcggcctgttaagtctggagtgaaagtcctgttttcaagatgggaattgctttggatactgatgggcttgagtgcaggagaggttatcggaattcccggtgtagcggtgaaatgcgtagagatcgggaggaacaccagtggcgaaggcgggtaactggactgcaactgacgctgaggcgcgaaagtgtggggagcaaacag

SEQ ID NO: 121 - DNA, 251 bp, Artificial sequence
  16S rRNA  amino acid sequence
tacggaggacccgagcgttatccggattcattgggtttaaagggtgcgcaggcggcttcttaagtcagcggtaaaatcgcggggctcaaccccgtcccgccgttgatactggggagcttgattacggtcgaggcaggcggaattcgtcgtgtagcggtgaaatgcatagatatgacgaagaacaccgattgcgtaggcagcctaccaggccgttaatgacgctcatgcacgaaagtgcggggatcgaacag

SEQ ID NO: 122 - DNA, 253 bp, Artificial sequence
  16S rRNA  amino acid sequence
tacggaaggtccgggcgttatccggatttattgggtttaaagggagcgcaggcggacagttaagcgtgacgtgaaatgcagcggctcaaccgtatgaagtgcgtcgcgaactggcagtcttgagtgagtacgatgtaggcggaatttgtggtgtagcggtgaaatgcttagatatgacgaagaaccccgattgcgaaggcagctcgcgggagcgcaactgacgctgaagctcgaaagtgcgggtatcgaacag

SEQ ID NO: 123 - DNA, 252 bp, Artificial sequence
  16S rRNA  amino acid sequence
tacggaaggtccgggcgttatccggatttattgggtttaaagggagcgcaggctggattttaagcgtgctgtgaaatgtaccggctcaaccggtgacgtgcagcgcgaactggagtccttgagtgagtacgaggtatgcggaattcgtggtgtagcggtgaaatgcttagatatcacgaagaactccgattgcgaaggcagcgtaccagtccttaactgacgctaaagctcgaaagtgcgggtatcgaacag

SEQ ID NO: 124 - DNA, 252 bp, Artificial sequence
  16S rRNA  amino acid sequence
tacagaggtccctagcgttgttcggatttactgggcgtaaagggcgcgtaggcggcgaggcaagtcgagtgtgaaatctccgggctcaacccggaaagtgcgctcgagactgccccgctggaggacggaagagggaaggggaatgcagggtgtagcggtgaaatgcgttgatatcctgcagaacaccggaggcgaaggcgccttccttgtacgtacctgacgctgaggcgcgaaagtcgggggagcaaacag

SEQ ID NO: 125 - DNA, 252 bp, Artificial sequence
  16S rRNA  amino acid sequence
tacgtatggtgcaagcgttatccggatttactgggtgtaaagggtgcgtaggtggaatggtaagtcagatgtgaaaactatgggctcaacccatagactgcatttgaaactgccgatctagagtgcaggagaggaaagtggaattcctagtgtagcggtgaaatgcgtagatattaggaggaacaccagtggcgaaggcggctttctggactgttactgacactgaggcacgaaagcgtggggagcaaacag

SEQ ID NO: 126 - DNA, 252 bp, Artificial sequence
  16S rRNA  amino acid sequence
tacatagggggcaagcgttatccggatttattgggtgtaaagggtgcgtagacggaggagcaagttggttgtgaaatccctcggcttaactgaggaactgcaactgaaactacttcccttgagtgtcggaggggaaagtggaattcctagtgtagcggtgaaatgcgtagatattaggaggaacaccggtggcgaaggcgactttctggacgataactgacgttgaggcacgaaagtgtggggagcaaacag

SEQ ID NO: 127 - DNA, 252 bp, Artificial sequence
  16S rRNA  amino acid sequence
tacggaaggtccgggcgttatccggatttattgggtttaaagggagcgtaggccgcagattaagtgtgttgtgaaatgtaggggctcaacctctgacttgcagcgcgaactgacggtcttgagtacgcacaacgttggcggaatttgtcgtgtagcggtgaaatgcttagatatgacgaagaactccgattgcgaaggcagctgacgggagcgccactgacgcttaagctcgaaagtgcgggtatcgaacag

SEQ ID NO: 128 - DNA, 251 bp, Artificial sequence
  16S rRNA  amino acid sequence
tacggaagatgcgagcgttatccggatttattgggtttaaagggagcgtaggcgggcctttaagccagcggtcaaatgtcatggcccaaccttggcatgccgttggaactggtggccttgaatacacacaaggaaggtggaattcgtcgtgtagcggtgaaatgcttagatatgacgaagaactccgattgcgaaggcagccttctggggtgcgattgacgctgaggctcgaaagtgcgggtatcgaacag

SEQ ID NO: 129 - DNA, 251 bp, Artificial sequence
  16S rRNA  amino acid sequence
tacggaagatgcgagcgttatccggatttattgggtttaaagggagcgtaggcggatgcttaagtctgcggtaaaatgtcggcgctcaacgccggcccgccgtggaaactgggtatcttgaatgtgcacaaggtggatggaattcgtggtgtagcggtgaaatgcttagatatcacgaagaactccgattgcgaaggcagtccactggggcatgattgacgctgaggctcgaaagtgcgggaatcaaacag

SEQ ID NO: 130 - DNA, 252 bp, Artificial sequence
  16S rRNA  amino acid sequence
tacagaggtctcgagcgttgttcggatttactgggcgtaaagggcgcgtaggcggcgcggcgtgtcgagtgtgaaatctccgggctcaacccggaaagtgcgtccgaaactaccgtgctggagtatgggagggggaatcggaatatagggtgtagcggtgaaatgcgttgatatcctatagaacaccggaggcgaaggcggattccttgaacatcactgacgctgaggcgcgaaagtcgggggagcaaacag

SEQ ID NO: 131 - DNA, 252 bp, Artificial sequence
  16S rRNA  amino acid sequence
tacggaggatgcaagcgttatccggatttattgggtttaaagggtgcgtaggcggcttgttaagcgtgaggtgaaatgccggagctcaaccccggaactgcctcgcgaactggcgagcttgaattctcctgccgtgggaggaatgtgtggtgtagcggtgaaatgcatagagatcacacagaacaccgattgcgaaggcatctcacgaaggggcgattgacgctgaggcacgaaagcgtggggatcaaacag

SEQ ID NO: 132 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagatataaggaagaacaccagtggcgaaggcggtctactggacagtaactgacgctgaggcgcgaaagcgtggggagcaaacag

SEQ ID NO: 133 - DNA, 251 bp, Artificial sequence
  16S rRNA  amino acid sequence
tacggaaggtccgggcgttatccggatttattgggtttaaagggagcgcaggcggactgctaagcgtgccgtgaaagcgcggggctcaaccccgtacagcggcgcgaactggcggtcttggttgcgggaaggggaggcggaactcgtggtgtagcggtgaaatgcatagatatcacgaagaactccgatcgcgaaggcagcctcccggcccgttaacgacgctcatgctcgaaggcgcgggtatcaaacag

SEQ ID NO: 134 - DNA, 251 bp, Artificial sequence
  16S rRNA  amino acid sequence
tacgtaggtggcgagcgttgtccggaattattgggcgtaaagggagcgcaggcgggaaggtaagtctatcttaaaagtgcggggctcaaccccgtgaggggatagaaactatttttcttgagtgcaggagaggaaagcggaattcctagtgtagcggtgaaatgcgtagatattaggaggaacaccagtggcgaaggcggctttctggactgtaactgacgctgaggctcgaaagccaggggagcgaacgg

SEQ ID NO: 135 - DNA, 252 bp, Artificial sequence
  16S rRNA  amino acid sequence
tacggagggtgcaagcgttaatcggaattactgggcgtaaagcgcgcgtaggttgttttgtaagtcagaggtgtaatcccacggcttaaccgtggaactgcctttgatactgcataacttggatccgggagaggacagcggaattccaggtgtaggagtgaaatccgtagatatctggaagaacatcagtggcgaaggcggctgtctggaccggtattgacgctgaggcgcgaaagcgtgggtagcaaacag

SEQ ID NO: 136 - DNA, 252 bp, Artificial sequence
  16S rRNA  amino acid sequence
tacgtagggggcaagcgttatccggatttactgggtgtaaagggcgtgtaggcgggactgcaagtcagatgtgaaagcacgaggctcaacttcgtgtctgcatttgaaactgtagttcttgagtactggagaggtaagcggaattcctagtgtagcggtgaaatgcgtagatattaggaagaacaccagtggcgaaggcggcttactggacagcaactgacgctgaggcgcgaaagcgtggggagcaaacag

SEQ ID NO: 137 - DNA, 252 bp, Artificial sequence
  16S rRNA  amino acid sequence
tacggaaggtccgggcgttatccggatttattgggtttaaagggagcgcaggctggattttaagcgtgttgtgaaatgtaccggctcaaccggtgatgtgcagcgcgaactggagtccttgagtgagtacgacgtcagcggaattcgtggtgtagcggtgaaatgcttagatatcacgaagaaccccgattgcgaaggcagctgacgagtccttaactgacgctaaagctcgaaagtgcgggtatcgaacag

SEQ ID NO: 138 - DNA, 252 bp, Artificial sequence
  16S rRNA  amino acid sequence
tacagaggcccgtagcgttgttcggatttactgggcgtaaagggcgcgtaggcggccaggcgtgtcggatgtgaaatctccgggctcaacccggaaagtgcgtccgaaactgcccggctcgagtgcgggatggggaaggggaatgcagggtgtagcggtgaaatgcgttgatatcctgcagaacaccggaggcgaaggcgccttcctggaacgcaactgacgctgaggcgcgaaagtcgggggagcaaacag

SEQ ID NO: 139 - DNA, 251 bp, Artificial sequence
  16S rRNA  amino acid sequence
tacgtagggggcgagcgttatccggattcattgggcgtaaagcgcgcgtaggcggcctgttaggtcgggagtcaaatccgggggctcaacccccgcccgctcccgataccggcgggcttgagtctggtaggggaaggtggaactccaagtgtagcggtggaatgcgcagatatttggaagaacaccggtggcgaaggcggccttctgggccacgactgacgctgaggcgcgaaagctaggggagcgaacag

SEQ ID NO: 140 - DNA, 251 bp, Artificial sequence
  16S rRNA  amino acid sequence
tacgtagggggcaagcgttatccggattcattgggcgtaaagcgcgcgtaggcggcctgctaggtcgggactcaaatccgggggctcaacccccgcccggtcccgataccgacaggcttgagtctggtaggggaaggcggaattccaagtgtagcggtggaatgcgcagatatttggaagaacaccggtggcgaaggcggccttctgggccacgactgacgctgaggcgcgaaagctaggggagcgaacag

SEQ ID NO: 141 - DNA, 252 bp, Artificial sequence
  16S rRNA  amino acid sequence
tacggaaggtccgggcgttatccggatttattgggtttaaagggagcgcaggcgggtttttaagcgtgttgtgaaatgtaccggctcaaccggtgacttgcagcgcgaactggagaccttgagtgagtacgaggaaggcggaattcgtggtgtagcggtgaaatgcttagatatcacgaagaactccgatcgcgaaggcagccttcgggagcgcaactgacgctgaagctcgaaagtgcgggtatcgaacag

SEQ ID NO: 142 - DNA, 251 bp, Artificial sequence
  16S rRNA  amino acid sequence
tacggaggatgcgagcgttatccggatttattgggtttaaagggtgcgtaggtggtttgttaagtttgtggtgaaagcgtgcggctcaaccgtaccaagccatgaaaactggcgaacttgagtgcaaacgaggtaggcggaatgtgatgtgtagcggtgaaatgcttagatatgtcacagaaccccgattgcgaaggcagctgacgagtccttaactgacgctaaagctcgaaagtgcgggtatcgaacag

SEQ ID NO: 143 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtctgcttgagtacgcacaacgtaggcggaatttgtcgtgtagcggtgaaatgcttagatatgacgaagaactccgattgcgaaggcagcttacgggagcgcaactgacgctgaagctcgaaagtgcgggtatcgaacag

SEQ ID NO: 144 - DNA, 252 bp, Artificial sequence
  16S rRNA  amino acid sequence
tacgtaggtggcaagcgttgtccggatttactgggtgtaaagggagcgtaggcgggagaacaagtcagaagtgaaatccatcggcttaaccggtgaactgcttttgaaactgtttttcttgagtgaagtagaggtaggcggaattcccggtgtagcggtgaaatgcgtagatatcgggaggaacaccagtggcgaaggcggcctactgggcttttactgacgctgaggctcgaaagcatgggtagcaaacag

SEQ ID NO: 145 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtctgcttgagtacgcacaacgtaggcggaatttgtcgtgtagcggtgaaatgcttagatatgacgaagaactccgattgcgaaggcagcctgctgtagcgccactgacgctgatgctcgaaagcgtgggtatcgaacag

SEQ ID NO: 146 - DNA, 251 bp, Artificial sequence
  16S rRNA  amino acid sequence
tacgtaggtggcaagcgttgtccggaattattgggcgtaaagcgcgcgcaggcggccctttaagtccatcttaaaagtgcggggcttaaccccgtgatgggatggaaactggagagctggagtatcggagaggaaagtggaattcctagtgtagcggtgaaatgcgtagagatgtggaagaataccagaggcgaaggcggcctcctggacagatactgacactgaggtgcgaaagcgtgggtagcaaacag

SEQ ID NO: 147 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactgtcctgcttgagtgcgcgcaacgtttgcggaatttgtcgtgtagcggtgaaatgcttagatatgacgaagaaccccgattgcgaaggcagctcgcgggagcgcaactgacgctgaagctcgaaagtgcgggtatcgaacag

SEQ ID NO: 148 - DNA, 252 bp, Artificial sequence
  16S rRNA  amino acid sequence
tacggaaggtccgggcgttatccggatttattgggtttaaagggagcgcaggccgactggtaagcgtgacgtgaaatgtagccgctcaacggctgaactgcgtcgcgaactgtcagtcttgagtgagttcgatgttggcggaattcgtggtgtagcggtgaaatgcttagatatcacgaagaactccgattgcgaaggcagccaacaaggcctttactgacgctaaagctcgaaggtgcgggtatcgaacag

SEQ ID NO: 149 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagagatttggaagaataccggaggcgaaggcggattccttgaacatcactgacgctgaggcgcgaaagtcgggggagcaaacag

SEQ ID NO: 150 - DNA, 252 bp, Artificial sequence
  16S rRNA  amino acid sequence
tacgtaggtggcaagcgttgtccggatttactgggtgtaaagggcgtgtaggcggagatgcaagtcaggagtgaaatctaggggctcaacccctaaactgcttttgaaactgtatcccttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacag

SEQ ID NO: 151 - DNA, 252 bp, Artificial sequence
  16S rRNA  amino acid sequence
tacggaggatgcgagcgttatccggatttattgggtttaaagggtgcgcaggcggccgtgcaagtcagcggtgaaagcccggggctcaaccccggaagtgccgttgatactgcatggctggaatgcggtcgaggcgggcggaacgtggcgtgtagcggtgaaatgcgtagatatgccacagaacgccgatagcggaggcagctcgccaggcctgcattgacgctcgggcacgaaagcgtggggatcgaacag

SEQ ID NO: 152 - DNA, 252 bp, Artificial sequence
  16S rRNA  amino acid sequence
tacggaaggtccaggcgttatccggatttattgggtttaaagggagcgcaggccggctgttaagcgtgacgtgaaatgtagatgctcaacatctgcactgcgtcgcgaactgacagtcttgagtgagttcgatgtaggcggaattcgtggtgtagcggtgaaatgcttagatatcacgaagaactccgattgcgaaggcagcctacaaggcctttactgacgctaaagctcgaaggtgcgggtatcgaacag

SEQ ID NO: 153 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagagattgggaggaacaccagtggcgaaggcgacttactggactgtaactgacgctgaggcgcgaaagtgtggggagcaaacag

SEQ ID NO: 154 - DNA, 252 bp, Artificial sequence
  16S rRNA  amino acid sequence
tacggaaggtccaggcgttatccggatttattgggtttaaagggagcgcaggccggaggtcaagcgtgacgtgaaatgtagccgctcaacggctgcactgcgtcgcgaactggctatcttgagtgagttcgatgttggcggaattcgtggtgtagcggtgaaatgcttagatatcacgaagaactccgattgcgaaggcagccaacaaggcctttactgacgctaaagctcgaaggtgcgggtatcgaacag

SEQ ID NO: 155 - DNA, 252 bp, Artificial sequence
  16S rRNA  amino acid sequence
tacgtagggggcgagcgttgtccggaattactgggcgtaaagggtgcgtaggtggcactttaagttggatgtgaaatccccgtgcttaacatgggagctgcatccaatactggagagctagagtgcaggagaggtaagcggaattcctagtgtagcggtgaaatgcgtagatattaggaggaacaccagtggcgaaggcggcttactggactgtaactgacactgaggcacgaaagcgtggggagcaaacag

SEQ ID NO: 156 - DNA, 252 bp, Artificial sequence
  16S rRNA  amino acid sequence
tacgtagggggcaagcgttatccggatttactgggtgtaaagggagcgcagacggaagagcaagtctgaagtgaaaacctgcggcttaactgcaggattgctttggaaactgtttttcttgagtactggagaggtaagcggaattcctggtgtagtagtgaaatgcgtagatatcaggaagaacaccggaggcgaaggcggcttactggacagcaactgacgttgaggctcgaaagcgtggggagcaaacag

SEQ ID NO: 157 - DNA, 252 bp, Artificial sequence
  16S rRNA  amino acid sequence
cacgtgggtggcgagcgttattcggattcactgggcgtaaagggtccgcagggggcccggcgcgcccgccgtgaaacgccaaggcccaaccttggccctgcggcgggtacggccgggcttgagcgcgcgatgggcagacggaactccgggtgtagcggtggaatgcgtggatatccggaagaacaccgacggcgaaggcggtctgctggcgcgcgactgaccctcagggacgagagcgtgggtagcgaacag

SEQ ID NO: 158 - DNA, 252 bp, Artificial sequence
  16S rRNA  amino acid sequence
tacggaaggtccgggcgttatccggatttattgggtttaaagggagcacaggccggctgtcaagcgtgttgtgaaatgtagaggctcaacctctgaattgcagcgcatactggtctgcttgagtacgcacaacgtaggcggaatttgtcgtgtagcggtgaaatgcttagatatgacgaagaactccgattgcgaaggcagcttacgggagcgcaactgacgctgaagctcgaaagtgcgggtatcgaacag

SEQ ID NO: 159 - DNA, 252 bp, Artificial sequence; identical to SEQ ID NO: 158
  16S rRNA  amino acid sequence

SEQ ID NO: 160 - DNA, 251 bp, Artificial sequence
  16S rRNA  amino acid sequence
tacggaaggtccgggcgttatccggatttattgggtttaaagggagcgcaggcggagagtgaagcgtgccgtgaaagcgcggggctcaaccccgtatagcggcgcgaactcaccctcttggttgcgggagaggaaggcggaactcgtggtgtagcggtgaaatgcatagatatcacgaggaaccccgatcgcgaaggcagccttccggcccgttaacgacgctcatgctcgaaggcgcgggtatcaaacag

SEQ ID NO: 161 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagagatttggaagaacaccaaaggcgaaggcagtctcctgggcaaatactgacgctcatatgcgaaagcgtgggtagcaaacag

SEQ ID NO: 162 - DNA, 252 bp, Artificial sequence
  16S rRNA  amino acid sequence
cacgtaaggtgcgagcgttgttcggaattattgggcgtaaagggcatgtaggcggcccaggaagcccggtgtgaaatacgggggctcaacccccgggctgcaccgggaactcccgggctggaatggccgaggcggagccggaattccaggtgtaggggtgaaatctgtagatatctggaagaacaccgatggcgaaggcaggctccgagcggacgattgacgctgaggtgcgaaggtgcggggagcgaacag

SEQ ID NO: 163 - DNA, 252 bp, Artificial sequence
  16S rRNA  amino acid sequence
tacggagggtgcgagcgttaatcggaataactgggcgtaaagagctcgtaggcggttagtcaagtcagatgtgaaagccccgggcttaacctgggaactgcatttgaaactgacgaactagagtactgtagagggaggtagaattccaggtgtagcggtgaaatgcgtagatatctggaggaataccggtggcgaaggcggcctcctggacagagactgacgctgaggagcgaaagcgtggggagcaaacag

SEQ ID NO: 164 - DNA, 252 bp, Artificial sequence
  16S rRNA  amino acid sequence
tacggaggatgcaagcgttatccggatttattgggtttaaagggtgcgcaggcggcgcgttaagtcagcggtgaaatctcccggctcaaccggggaactgccattgatactggcgtgctagagtacggacggcgccggcggaatgtgtcatgtagcggtgaaatgcttagatatgccacagaacaccgatcgcgaaggcagctggcgaggccggtactgacgctcaggcacgaaagcgtggggatcaaacag

SEQ ID NO: 165 - DNA, 252 bp, Artificial sequence
  16S rRNA  amino acid sequence
tacggaggacgcgagcgttatccggattcattgggtttaaagggtgcgcaggcggctcggcaagtcagcggtgaaagcccggggcccaaccccggaagtgccgttgatactgtcgggctggaatgcggtcgaggcgggcggaacgtggcgtgtagcggtgaaatgcgtagatatgccacagaacgccgatagcggaggcagctcgccaggcctgcattgacgctcgggcacgaaagcgtggggatcgaacag

SEQ ID NO: 166 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgcccaacgcctgcactgcagcgcgaactggctgacttgagtacgcacgacgttggcggaatttgccgtgtagcggtgaaatgcttagatatggcgaagaactccgattgcgaaggcagcttacgggagcgcaactgacgctgaagctcgaaagtgcgggtatcgaacag

SEQ ID NO: 167 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggtgcccaacacctgcactgcagcgcgaactggctgacttgagtacgcacgacgttggcggaatttgccgtgtagcggtgaaatgcttagatatgacgaagaactccgattgcgaaggcagctgacgggagcgttactgacgcttaagctcgaaagcgcgggtatcgaacag

SEQ ID NO: 168 - DNA, 251 bp, Artificial sequence
  16S rRNA  amino acid sequence
tacggaggatgcgagcgttatccggatttattgggtttaaagggtgcgtaggtggtttgttaagtttgtggtgaaagcgtgcggctcaaccgtaccaagccatgaaaactggcgaacttgagtgcaaacgaggtaggcggaatgtgatgtgtagcggtgaaatgcgtagatatgccacagaacgccgatagcggaggcagctcgccaggcctgcattgacgctcgggcacgaaagcgtggggatcgaacag

SEQ ID NO: 169 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtctgcttgagtacgcacaacgtaggcggaatttgtcgtgtagcggtgaaatgcttagatatgacgaagaactccgattgcgaaggcagccaacaaggcctttactgacgctaaagctcgaaggtgcgggtatcgaacag

SEQ ID NO: 170 - DNA, 252 bp, Artificial sequence; identical to SEQ ID NO: 169
  16S rRNA  amino acid sequence

SEQ ID NO: 171 - DNA, 252 bp, Artificial sequence
  16S rRNA  amino acid sequence
tacggaaggtccgggcgttatccggatttattgggtttaaagggagcgtaggcgggatgttaagcgtgctgtgaaaagctgcggctcaaccgcagtcgtgcagcgcgaactggctttcttgagtgcgcggaaggcatgcggaattcgtggtgtagcggtgaaatgcttagatatcacgaggaactccgattgcgaaggcagcatgccgcagcgctactgacgctgaagctcgaaggtgcgggtatcgaacag

SEQ ID NO: 172 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatggcgaagaactccgattgcgaaggcagctttccagtccttaactgacgctaaagctcgaaagtgcgggtatcgaacag

SEQ ID NO: 173 - DNA, 251 bp, Artificial sequence
  16S rRNA  amino acid sequence
tacggaaggtccgggcgttatccggatttattgggtttaaagggagcgcagacgggctgttaagcgtgacgtgaaatccgggggctcaaccttcggctgcgtcgcgaactggcggccttgagtggacggtaggcaggcggaattcgtggtgtagcggtgaaatgcttagatatcacgaagaaccccgattgcgaaggcagctcgcgggagcgcaactgacgctgaagctcgaaagtgcgggtatcgaacag

SEQ ID NO: 174 - DNA, 252 bp, Artificial sequence
  16S rRNA  amino acid sequence
tacggaggatgcaagcgttatccggatttattgggtttaaagggtgcgcaggcggcttgttaagcgggaggtgaaatgccggcgctcaacgtcggaagtgcctcccgaactggtgagcttgaatgactctgccgtgggaggaatgtgtagtgtagcggtgaaatgcttagagattacacagaacaccgattgcgaaggcatctcacgaagggttgattgacgctgaggcacgaaagcgtggggatcaaacag

SEQ ID NO: 175 - DNA, 252 bp, Artificial sequence
  16S rRNA  amino acid sequence
tacggaggatgcaagcgttatccggatttattgggtttaaagggtgcgcaggcggatagttaagtcagcggtgaaatctcacggctcaaccgtggaactgccgttgatactggctgtctggagtacggacggcgccggcggaatgtgtcatgtagcggtgaaatgcttagatatgccacagaacaccgatcgcgaaggcagctggcgaggccggtactgacgctcaggcacgaaagcgtggggatcaaacag

SEQ ID NO: 176 - DNA, 252 bp, Artificial sequence
  16S rRNA  amino acid sequence
tacggaggatgcgagcgttatccggatttattgggtttaaagggtgcgtaggcggacgattaagtcagctgtgaaatgcccgcgcttaacgcgggaagtgcagttgatactggttgtctggagtgccgttgccgtggggggaatgtgtagtgtagcggtgaaatgcatagatattacacagaacgccgattgtgaagacacctcacgaagcggtaactgacgctgaggcacgaaagcgtggggatcgaacag

SEQ ID NO: 177 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtctgcttgagtacgcacaacgtaggcggaatttgtcgtgtagcggtgaaatgcttagatatcacgaagaactccaattgcgaaggcagctcgctgtagtgttactgacgctaaagctcgaaagtgcgggtatcgaacag

SEQ ID NO: 178 - DNA, 251 bp, Artificial sequence
  16S rRNA  amino acid sequence
tacggaggatgcgagcgttatccggatttattgggtttaaagggtgcgtaggcggaatgttaagtcagtggtgaaagcgtgcggctcaaccgtaccgagccattgaaactggcgttcttgagtgcaagcgaggtaagcggaatgtgacgtgtagcggtgaaatgcttagatatgtcccagaactccgattgcgaaggcagcttaccagtatgtaactgacgctgaggcacgaaagcgtgggtatcaaacag

SEQ ID NO: 179 - DNA, 252 bp, Artificial sequence
  16S rRNA  amino acid sequence
tacggagggtgcaagcgttaatcggaattactgggcgtaaagcgcacgtaggttgtcctgtaagtcaggggtgaaagcccgcggctcaactgcggaactgcccttgatactgcaggacttggatccgggagaggtcagcggaattccaggtgtaggagtgaaatccgtagatatctggaggaacatcagtggcgaaggcggctgactggaccggcattgacgctgaggtgcgaaagcgtgggtagcaaacag

SEQ ID NO: 180 - DNA, 251 bp, Artificial sequence
  16S rRNA  amino acid sequence
tacggaaggtccgggcgttatccggatttattgggtttaaagggagcgtaggcgggcctttaagtcagcggttaaatgtcagtgcccaacgttggcatgccgttgatactggaggtcttgaatacacacaaggatggtggaattcgtcgtgtagcggtgaaatgcttagatatgacgaagaactccgattgcgaaggcagccatctggggtgcgattgacgctgaggctcgaaagtgcgggtatcaaacag

SEQ ID NO: 181 - DNA, 252 bp, Artificial sequence
  16S rRNA  amino acid sequence
tacggaggatgcgagcgttatccggatttattgggtttaaagggtgcgtaggctgttcgtcaagtcagtggtgaaatgccggggctcaaccccggagctgccattgatactgtcgggcttgaatcctgctgccgtgggaggaatgagtagtgtagcggtgaaatgcatagatattactcagaacaccgattgcgaaggcatctcacgaaacaggcattgacgctgaggcacgaaagcgtggggatcaaacag

SEQ ID NO: 182 - DNA, 252 bp, Artificial sequence
  16S rRNA  amino acid sequence
tacggaaggtccgggcgttatccggatttattgggtttaaagggagcgcaggctgattattaagcgtgacgtgaaatgtagccgctcaacggctgaactgcgtcgcgaactggtagtcttgagtgagttcgatgtaggcggaattcgtggtgtagcggtgaaatgcttagatatcacgaagaactccgattgcgaaggcagctttccagtccttaactgacgctaaagctcgaaagtgcgggtatcgaacag

SEQ ID NO: 183 - DNA, 252 bp, Artificial sequence
  16S rRNA  amino acid sequence
tacgtagggggcaagcgttatccggatttactgggtgtaaagggagcgcagacggaagagcaagtctgaagtgaaaacctgcggcttaactgcaggattgctttggaaactgtttttcttgagtactggagaggtaagcggaattcctggtgtagtagtgaaatgcgtagatatcaggaagaacaccggaggcgaaggcggcctcctggacagatactgacactgaggtgcgaaagcgtgggtagcaaacag

SEQ ID NO: 184 - DNA, 251 bp, Artificial sequence
  16S rRNA  amino acid sequence
tacggaggatccgagcgttatccggattcattgggtttaaagggtgcgcaggcggtgccttaagtcagcggtaaaatcgtggggctcaaccccatcccgccgttgatactggggcacttgattacggtcgaggcaggcggaattcgtcgtgtagcggtgaaatgcatagatatgacgaagaacaccgattgcgtaggcagcctaccaggccgttaatgacgctcatgcacgaaagtgcggggatcgaacag

SEQ ID NO: 185 - DNA, 252 bp, Artificial sequence
  16S rRNA  amino acid sequence
tacggaggatgcaagcgttatccggatttattgggtttaaagggtgcgtaggctgttcgacaagtcaggggtgaaagcccggcgcctaacgccggaactgcctttgatactgccgagctggaatacggatgccgtgggaggaatgagtagtgtagcggtgaaatgcatagatattactcagaacaccgattgcgaaggcatctcacgaatccgtcattgacgctgaggcacgaaagcgtggggatcaaacag

SEQ ID NO: 186 - DNA, 252 bp, Artificial sequence
  16S rRNA  amino acid sequence
tacgtatggtgcaagcgttatccggatttactgggtgtaaagggtgcgtaggtggtatggcaagtcagatgtgaaagcacggggctcaaccccgtgactgcatttgaaactgtcaaactagagtgcaggagaggaaagtggaattcctagtgtagcggtgaaatgcgtagatattaggaagaacaccagtggcgaaggcggctttctggactgtaactgacactgaggcacgaaagcgtggggagcaaacag

SEQ ID NO: 187 - DNA, 251 bp, Artificial sequence
  16S rRNA  amino acid sequence
tacggaggatgcgagcgttatccggatttattgggtttaaagggtgcgcaggcggcgagtcaagtcagcggtcaaactgcggcgctcaacgccgtaccgccgttgaaactggcgcgctcgagtttgtgcgaggatggcggaatgcgcagtgtagcggtgaaatgcatagatattgcgcagaactccgattgcgaaggcagctgacgggagcgcaactgacgctcatgctcgaaagtgcgggtatcgaacag

SEQ ID NO: 188 - DNA, 252 bp, Artificial sequence
  16S rRNA  amino acid sequence
tacgtagggagcaagcgttgtccggatttactgggtgtaaagggtgcgcaggcggctctgcaagtcagctgtgaaatctatgggctcaacccataaattgcagttgaaactgtggagcttgagtgaagtagaggcaggcggaattccctgtgtagcggtgaaatgcgtagagatagggaggaacaccagtggcgaaggcggcctgctgggctttaactgacgctgaggcacgaaagcgtgggtagcaaacag

SEQ ID NO: 189 - DNA, 252 bp, Artificial sequence
  16S rRNA  amino acid sequence
tacggaaggtccgggcgttatccggatttattgggtttaaagggagcgtaggccggagattaagcgtgttgtgaaatgtagacgctcaacgtctgcactgcagcgcgaactggtttccttgagtacgcataaagtgggcggaattcgtggtgtagcggtgaaatgcttagatatcacgaagaactccgattgcgaaggcagctcactggggcgcaactgacgctgaagctcgaaagcgcgggtatcgaacag

SEQ ID NO: 190 - DNA, 252 bp, Artificial sequence; identical to SEQ ID NO: 189
  16S rRNA  amino acid sequence

SEQ ID NO: 191 - DNA, 252 bp, Artificial sequence
  16S rRNA  amino acid sequence
tacggaaggtccgggcgttatccggatttattgggtttaaagggagcgtaggccggggattaagcgtgttgtgaaatgtagacgctcaacgtctgaattgcagcgcgaactggttctcttgagtgcgcgggaagttggcggaattcgttgtgtagcggtgaaatgcttagatatgacgaagaactccgattgcgaaggcagctgactgtagcgtaactgacgctgaagctcgaaagtgtgggtatcgaacag

SEQ ID NO: 192 - DNA, 252 bp, Artificial sequence
  16S rRNA  amino acid sequence
tacgtagggggcaagcgttatccggatttactgggtgtaaagggagcgcagacggcgcagcaagtctgaagtgaaagcccggggcttaaccccgggactgctttggaaactgctgtgcttgagtatcggaggggcaggcggaattcccagtgtagcggtgaaatgcgtagatattgggaagaacaccagtggcgaaggcggcctgctggacgataactgacgttgaggctcgaaggcgtggggagcaaacag

SEQ ID NO: 193 - DNA, 252 bp, Artificial sequence
  16S rRNA  amino acid sequence
tacgtatggagcgagcgttatccggatttactgggtgtaaagggagcgtaggcggcgcaccaagtccgatgtgaaaccccggggctcaaccccgggcttgcattggaaactggcgtgctggagtgtcggaggggcgggcggaattcctggtgtagcggtgaaatgcgtagatatcaggaggaacaccggtggcgaaggcggcccgctggacgacaactgacgctgagactcgaaagcgtggggagcaaacag

SEQ ID NO: 194 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagatataaggaagaacaccagtggcgaaggcggtctactggacagtaactgacgctgaggcgcgaaagcgtggggagcaaacag

SEQ ID NO: 195 - DNA, 252 bp, Artificial sequence
  16S rRNA  amino acid sequence
cacgtaaggtgctagcgttgttcggaatcattgggcgtaaagggcatgtaggcggccgcgcaagtctggagtgaaatccccgggcccaacccgggaactgctttggaaactgcgtggcttgagtcgccgaggcggagccggaattccaggtgtaggggtgaaatctgtagatatctggaagaataccaatggcgaaggcaggctgccagcagatgactgacgctgaggtgcgaaggtgcggggagcaaacag

SEQ ID NO: 196 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcgcaacgcgggcggaatttgtcgtgtagcggtgaaatgcttagatatgacgaagaactccgattgcgaaggcagctgacgggagcgttactgacgcttaagctcgaaagcgcgggtatcgaacag

SEQ ID NO: 197 - DNA, 252 bp, Artificial sequence
  16S rRNA  amino acid sequence
tacggaggatgcaagcgttatccggatttattgggtttaaagggtgcgtaggcggaccgttaagtcagcggtgaaaggtcccggctcaaccggggagctgccgttgatactggcggacttgagtccaaacggcgccggcggaatatgtaatgtagcggtgaaatgcatagatattacatagaacaccgatcgcgaaggcagctggcgaggatggcactgacgctgaggcacgaaggcgtggggatcgaacag

SEQ ID NO: 198 - DNA, 252 bp, Artificial sequence
  16S rRNA  amino acid sequence
tacagaggtcccgagcgttgttcggatttactgggcgtaaagggcgcgtaggcggcggaacgtgtcggatgtgaaatctccgggctcaacccggaaagtgcgtccgaaactaccgtgctggagtatgggagggggaatcggaatacagggtgtagcggtgaaatgcgttgatatcctgtagaacaccggaggcgaaggcggattccttgaacatcactgacgctgaggcgcgaaagtcgggggagcaaacag

SEQ ID NO: 199 - DNA, 252 bp, Artificial sequence
  16S rRNA  amino acid sequence
tacggaaggtccgggcgttatccggatttattgggtttaaagggagcgcaggccgtgggttaagcgtgacgtgaaaagcagcggctcaaccgttgtcgtgcgtcgcgaactggcccgcttgagtgggatcgacgtaggcggaattcgtggtgtagcggtgaaatgcttagatatcacgaagaaccccgattgcgaaggcagcctacgaggcctctactgacgctaaagctcgaaggtgcgggtatcgaacag

SEQ ID NO: 200 - DNA, 252 bp, Artificial sequence
  16S rRNA  amino acid sequence
tacggaaggtccgggcgttatccggatttattgggtttaaagggagcgcaggcggaggcttaagcgtgacgtgaaatgtagccgctcaacggctgagttgcgtcgcgaactgggtttcttgagtgagtgcgacgtcagcggaattcgtggtgtagcggtgaaatgcttagatatcacgaagaaccccgattgcgaaggcagctgacgagccctttactgacgctaaagctcgaaggtgcgggtatcgaacag

SEQ ID NO: 201 - DNA, 252 bp, Artificial sequence
  16S rRNA  amino acid sequence
tacggaaggtccgggcgttatccggatttattgggtttaaagggagcgcaggccggaggtcaagcgtgacgtgaaatgtagccgctcaacggctgagttgcgtcgcgaactggtttccttgagtgagtacgacgtcagcggaattcgtggtgtagcggtgaaatgcttagatatcacgaagaaccccgattgcgaaggcagctgacgagtcctttactgacgctaaagctcgaaggtgcgggtatcgaacag

SEQ ID NO: 202 - DNA, 252 bp, Artificial sequence
  16S rRNA  amino acid sequence
tacggaaggtccgggcgttatccggatttattgggtttaaagggagcgcaggcggaggcttaagcgtgacgtgaaatgtagccgctcaacggctgagttgcgtcgcgaactgggtttcttgagtgagttcgatgtgggcggaattcgtggtgtagcggtgaaatgcttagatatcacgaagaactccgattgcgaaggcagcccacaaggcctttactgacgctaaagctcgaaggtgcgggtatcgaacag

SEQ ID NO: 203 - DNA, 252 bp, Artificial sequence
  16S rRNA  amino acid sequence
tacggaggatacaagcgttatccggatttattgggtttaaagggtgcgcaggcggaccgctaagtcaggggtgaaaagttcgtgcttaacatgaatcgtgcctttgatactggcggactagagtgcgataggcgtggccggaatgtggtgtgtagcggtgaaatgcttagatatgccacagaacaccgatcgagaagtcaggtcacgagttcgcaactgacgctcatgcacgaaagcgtggggatcaaacag

SEQ ID NO: 204 - DNA, 252 bp, Artificial sequence
  16S rRNA  amino acid sequence
cacgtaaggcgcgagcgttgttcggaatcattgggcgtaaagggcatgtaggcggccgtgcaagtctggagtgaaaggcgggggcccaacccccggacagctctggaaactgcgcggctggagtcgccgaggcggggccggaattccaggtgtaggggtgaaatctgtagatatctggaagaacaccggtggcgaaggcaggccccgagcggacgactgacgctgaggtgcgaaggtgcggggagcgaacag

SEQ ID NO: 205 - DNA, 252 bp, Artificial sequence
  16S rRNA  amino acid sequence
tacgtagggggcgagcgttgtccggaatgattgggcgtaaagggcgcgtaggcggcccggtaagtctggagtgaaagtcctgcttttaaggtgggaattgctttggatactgtcgggcttgagtgcaggagaggtaagtggaattcccagtgtagcggtgaaatgcgtagagattgggaggaacaccagtggcgaaggcgacttactggactgtaactgacgctgaggcgcgaaagtgtggggagcaaacag

SEQ ID NO: 206 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagagatgtggaagaataccagaggcgaaggcggcctcctggacagtaactgacgttgaggcacgaaagtgtggggagcaaacag

SEQ ID NO: 207 - DNA, 252 bp, Artificial sequence
  16S rRNA  amino acid sequence
tacagaggtccctagcgttgttcggatttactgggcgtaaagggcacgtaggcggcatggcgtgtcgagtgtgaaatctccgggcccaacccggaaagtgcgctcgaaactaccatgctggagtacgggagagggaatcggaatgcagggtgtagcggtgaaatgcgttgatatcctgcagaacaccggaggcgaaggcggattcctggaacgttactgacgctaaggtgcgaaagtcgggggagcaaacag

SEQ ID NO: 208 - DNA, 251 bp, Artificial sequence
  16S rRNA  amino acid sequence
tacggaggatgcgagcgttatccggatttattgggtttaaagggtgcgcaggcggcatgtcaagtcagcggtcaaaatgcggcgctcaacgccgtaccgccgttgaaactggcgcgcttgagttcgtgggaggatcgcggaatgcgcggtgtagcggtgaaatgcatagatatcgcgcagaaccccgattgcgaaggcagcggtccatcacgcgactgacgctcatgcacgaaagcgtgggtatcgaacag

SEQ ID NO: 209 - DNA, 252 bp, Artificial sequence
  16S rRNA  amino acid sequence
tacagaggtctcgagcgttgttcggatttactgggcgtaaagggcgcgtaggcggcgcggcgtgtcgagtgtgaaatctccgggctcaacccggaaagtgcgctcgaaactgccgtgctcgagtgcgggagagggaatcggaatacagggtgtagcggtgaaatgcgttgatatcctgtagaacaccggatgcgaaggcggattcctggaacgcaactgacgctgaggcgcgaaagtcgggggagcaaacag

SEQ ID NO: 210 - DNA, 252 bp, Artificial sequence
  16S rRNA  amino acid sequence
cacgtaagttgcgagcgttgttcggaattattgggcgtaaagggcatgtaggcggtcttgcaagtctggtgtgaaatgccggggctcaaccccggaactgcgttggaaactgtaagactggagtcgctgaggggcaggcagaattcctggtgtaggggtgaaatctgtagatatcaggaagaataccaatggcgaaggcagcctgccagcagatgactgacgctgaggtgcgaaggtgcggggagcaaacag

SEQ ID NO: 211 - DNA, 252 bp, Artificial sequence
  16S rRNA  amino acid sequence
tacggagggcgcaagcgttaatcggaataactgggcgtaaagcgcgcgcaggcggtcatgcaagtcagatgtgaaatcccggtgcttaacgtcgggcggtcatttgaaactgcatgactagagtactatatgggaggctggaatttcaggtgtagcggtgaaatgcgcagatatctgaaagaacaccggcggcgaaggcgggcctctggatagtaactgacgctcaggcgcgaaagcgtggggagcaaacag

SEQ ID NO: 212 - DNA, 252 bp, Artificial sequence
  16S rRNA  amino acid sequence
tacggaaggtccgggcgttatccggatttattgggtttaaagggagcgcaggcggccttttaagcgtgacgtgaaatgcaggtgctcaacatctgagctgcgtcgcgaactggagggcttgagtgagttcgatgtcagcggaattcgtggtgtagcggtgaaatgcttagatatcacgaagaaccccgattgcgaaggcagctggcaaggcctttactgacgctaaagctcgaaggtgcgggtatcgaacag

SEQ ID NO: 213 - DNA, 252 bp, Artificial sequence
  16S rRNA  amino acid sequence
tacggaaggtccgggcgttatccggatttattgggtttaaagggagcgcaggccgatggttaagcgtgacgtgaaatgtagccgctcaacggctgcactgcgtcgcgaactggctgacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 214 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcacaacgcgggcggaatttgtcgtgtagcggtgaaatgcttagatatcacgaagaactccaattgcgaaggcagctcgctgtagtgttactgacgctaaagctcgaaagtgcgggtatcgaacag

SEQ ID NO: 215 - DNA, 252 bp, Artificial sequence
  16S rRNA  amino acid sequence
tacggaaggtccgggcgttatccggatttattgggtttaaagggagcgcaggcggccttttaagcgtgacgtgaaatgccggggctcaaccttggaattgcgtcgcgaactggcgggcttgagtacgctcgaggcaggcggaattcgtggtgtagcggtgaaatgcttagatatcacgaggaaccccgattgcgaaggcagcctgccggggtgttactgacgctcatgctcgaaggtgcgggtatcgaacag

SEQ ID NO: 216 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagagatgtggaagaataccagaggcgaaggcggcctcctggacagatactgacactgaggtgcgaaagcgtgggtagcaaacag

SEQ ID NO: 217 - DNA, 252 bp, Artificial sequence; identical to SEQ ID NO: 216
  16S rRNA  amino acid sequence

SEQ ID NO: 218 - DNA, 252 bp, Artificial sequence; identical to SEQ ID NO: 216
  16S rRNA  amino acid sequence

SEQ ID NO: 219 - DNA, 252 bp, Artificial sequence; identical to SEQ ID NO: 216
  16S rRNA  amino acid sequence

SEQ ID NO: 220 - DNA, 252 bp, Artificial sequence; identical to SEQ ID NO: 216
  16S rRNA  amino acid sequence

SEQ ID NO: 221 - DNA, 252 bp, Artificial sequence; identical to SEQ ID NO: 216
  16S rRNA  amino acid sequence

SEQ ID NO: 222 - DNA, 252 bp, Artificial sequence
  16S rRNA  amino acid sequence
tacggaggatacaagcgttatccggatttattgggtttaaagggtgcgcaggcggaccgttaagtcaggggtgaaaagttggggctcaactccaatcgtgcccttgatactggcggactagagtgcgataggcgtggccggaatgtgatgtgtagcggtgaaatgcttagatatgtcacagaacaccgatcgagaagtcaggtcacgagttcgcaactgacgctcatgcacgaaagcgtggggatcaaacag

SEQ ID NO: 223 - DNA, 252 bp, Artificial sequence
  16S rRNA  amino acid sequence
tacggagggtgcaagcgttaatcggaataactgggcgtaaagggcatgcaggcggttagacaagtaggatgtgaaatccccgggctcaacctgggagctgcatactaaactgtctgactagagtattgcagggggagacggaattccaggtgtagcggtggaatgcgtagatatctggaagaacaccaaaggcgaaggcagtctcctgggcaaatactgacgctcatatgcgaaagcgtgggtagcaaacag

SEQ ID NO: 224 - DNA, 252 bp, Artificial sequence; identical to SEQ ID NO: 223
  16S rRNA  amino acid sequence

SEQ ID NO: 225 - DNA, 252 bp, Artificial sequence
  16S rRNA  amino acid sequence
tacagaggccaagagcgttgttcggatttactgggcgtaaagggcgcgtaggcggcgaggcgtgtcggatgtgaaatctccgggctcaacccggaaagtgcgtccgaaactaccttgctggagtccgggagggggaatcggaatacagggtgtagcggtgaaatgcgttgatatcctgtagaacaccggaggcgaaggcggattccttgaacggtactgacgctgaggcgcgaaagtcgggggagcaaacag

SEQ ID NO: 226 - DNA, 252 bp, Artificial sequence
  16S rRNA  amino acid sequence
tacggagggtgcgagcgttaatcggaataactgggcgtaaagagctcgtaggcggtttgtcaagtcagatgtgaaagccccgggcttaacctgggaaccgcatttgaaactgacagactagagtactgtagagggaggtagaattccaggtgtagcggtgaaatgcgtagatatctggaggaataccggtggcgaaggcggcctcctggacagatactgacactgaggtgcgaaagcgtgggtagcaaacag

SEQ ID NO: 227 - DNA, 251 bp, Artificial sequence
  16S rRNA  amino acid sequence
tacgtaggtggcaagcgttgtccggaattattgggcgtaaagcgcgcgcaggcggccctttaagtccatcttaaaagtgcggggcttaaccccgtgatgggatggaaactggagagctggagtatcggagaggaaagtggaattcctagtgtagcggtgaaatgcgtagagattaggaagaacaccggtggcgaaggcgactttctggacgacaactgacgctgaggcgcgaaagcgtggggagcaaacag

SEQ ID NO: 228 - DNA, 251 bp, Artificial sequence; identical to SEQ ID NO: 227
  16S rRNA  amino acid sequence

SEQ ID NO: 229 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtctgcttgagtacgcacaacgtaggcggaatttgtcgtgtagcggtgaaatgcttagatatcacgaagaactccgattgcgaaggcagctttccagtccttaactgacgctaaagctcgaaagtgcgggtatcgaacag

SEQ ID NO: 230 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagagatgtggaagaataccagaggcgaaggcggcttactggacgattactgacgctgaggctcgaaagcgtggggagcaaacag

SEQ ID NO: 231 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgctcaacgcctgacttgcagcgcgaactgacggtcttgagtgcgcgcaacgttggcggaatttgtcgtgtagcggtgaaatgcttagatatcacgaagaaccccgattgcgaaggcagctgacgagtcctttactgacgctaaagctcgaaggtgcgggtatcgaacag

SEQ ID NO: 232 - DNA, 252 bp, Artificial sequence; identical to SEQ ID NO: 231
  16S rRNA  amino acid sequence

SEQ ID NO: 233 - DNA, 252 bp, Artificial sequence
  16S rRNA  amino acid sequence
tacgtagggggcaagcgttatccggatttactgggtgtaaagggcacgcaggtggcgagataagttggatgtgaaattcccgtgcttaacatggggactgcatccaaaactgttgaactggagtactgtagagggaggtagaattccacgtgtagcggtgaaatgcgtagagatgtggaagaataccagaggcgaaggcggcctcctggacagatactgacactgaggtgcgaaagcgtgggtagcaaacag

SEQ ID NO: 234 - DNA, 252 bp, Artificial sequence
  16S rRNA  amino acid sequence
tacgtagggggcgagcgttatccggatttactgggtgtaaagggagcgcagacggagaagcaagtctgatgtgaaagcccgcggctcaaccgcgggactgcattggaaactgtttatctggagtactggaagggcaagcggaattcctggtgtagcggtgaaatgcgtagatatcaggaggaacaccggtggcgaaggcggcttgctggacagtaactgacgttgaggctcgaaagcgtggggagcaaacag

SEQ ID NO: 235 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagatatcaggaagaacaccggaggcgaaggcggcttactggacagcaactgacgttgaggctcgaaagcgtggggagcaaacag

SEQ ID NO: 236 - DNA, 252 bp, Artificial sequence
  16S rRNA  amino acid sequence
tacggaaggtccgggcgttatccggatttattgggtttaaagggagcgtaggccgcaggttaagtgtgttgtgaaatgtaggcgcccaacgtctgaattgcagcgcatactgtcctgcttgagtgcgcgcaacgtttgcggaatttgtcgtgtagcggtgaaatgcttagatatgacgaagaactccgattgcgaaggcagcagacgggagcgcaactgacgctgaagctcgaaagtgcgggtatcgaacag

SEQ ID NO: 237 - DNA, 252 bp, Artificial sequence; identical to SEQ ID NO: 236
  16S rRNA  amino acid sequence

SEQ ID NO: 238 - DNA, 252 bp, Artificial sequence
  16S rRNA  amino acid sequence
tacgtaggtggcaagcgttgtccggatttactgggtgtaaagggcgcgtaggcgggcaggcaagtcagatgtgaaatctccgggctcaacccggaaattgcatttgaaactgcaggtcttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacag

SEQ ID NO: 239 - DNA, 252 bp, Artificial sequence
  16S rRNA  amino acid sequence
tacggaaggtccgggcgttatccggatttattgggtttaaagggagcgtaggcggtatattaagcgtgttgtgaaatgtaggtgctcaacatctgacttgcagcgcgaactggtttacttgagtacgcacaacgtaggcggaattcgtcgtgtagcggtgaaatgcatagatatgacgaagaactccgattgcgaaggcagcttacgggagcgcaactgacgctgaagctcgaaagtgcgggtatcgaacag

SEQ ID NO: 240 - DNA, 253 bp, Artificial sequence
  16S rRNA  amino acid sequence
tacagaggccaagagcgttgttcggatttactgggcgtaaagggcgcgtaggcggcaaggcgtgtcaagtgtgaaatctccgggcccaacccggaaagtgcgcttgaaactaccatgctttgagtccgggagggggaatcggaatacagggtgtagcggtgaaatgcgttgatatcctgtagaacaccggaggcgaaggcggattccttgaacggtactgacgctgaggcgcgaaagtcgggggagcaaacag

SEQ ID NO: 241 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgcctgacttgcagcgcgaactgacggtcttgagtgcgcgcaacgttggcggaatttgtcgtgtagcggtgaaatgcttagatatgacgaagaactccgattgcgaaggcagctgacgggagcgttactgacgcttaagctcgaaagcgcgggtatcgaacag

SEQ ID NO: 242 - DNA, 252 bp, Artificial sequence; identical to SEQ ID NO: 241
  16S rRNA  amino acid sequence

SEQ ID NO: 243 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcacaacgcgggcggaatttgtcgtgtagcggtgaaatgcttagatatgacgaagaaccccgattgcgaaggcagctcgcgggagcgcaactgacgctgaagctcgaaagtgcgggtatcgaacag

SEQ ID NO: 244 - DNA, 252 bp, Artificial sequence; identical to SEQ ID NO: 243
  16S rRNA  amino acid sequence

SEQ ID NO: 245 - DNA, 252 bp, Artificial sequence
  16S rRNA  amino acid sequence
tacagaggtccctagcgttgttcggatttactgggcgtaaagggcgcgtaggctgcatggcgtgtcaagtgtgaaatctccgggctcaacccggaaagtgcgcttgaaactaccatgctggaggccgggagagggaatcggaacatagggtgtagcggtgaaatgcgttgatatcctatagaacaccggaggcgaaggcggattcctggaacggcactgacgctgaggcgcgaaagtcgggggagcaaacag

SEQ ID NO: 246 - DNA, 252 bp, Artificial sequence
  16S rRNA  amino acid sequence
tacggaaggtccgggcgttatccggatttattgggtttaaagggagcgtaggccgcagattaagtgtgttgtgaaatgtaggcgctcaacgtctgacttgcagcgcatactggtccacttgagtgcgcgcaacgcgggcggaatttgtcgtgtagcggtgaaatgcttagatatgacgaagaactccgattgcgaaggcagctgacgggagcgccactgacgcttaagctcgaaagtgcgggtatcgaacag

SEQ ID NO: 247 - DNA, 252 bp, Artificial sequence; identical to SEQ ID NO: 246
  16S rRNA  amino acid sequence

SEQ ID NO: 248 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 249 - DNA, 252 bp, Artificial sequence
  16S rRNA  amino acid sequence
cacgtaaggtgctagcgttgttcggaatcattgggcgtaaagggcatgtaggcggccgcgcaagtctggagtgaaatccccgggcccaacccgggaactgctttggaaactgcgtggcttgagtcgccgaggcggagccggaattccaggtgtaggggtgaaatctgtagatatctggaagaacaccggtggcgaaggcaggcttcgagcggacgactgacgctgaggtgcgaaggtgcggggagcgaacag

SEQ ID NO: 250 - DNA, 252 bp, Artificial sequence
  16S rRNA  amino acid sequence
tacggagggcgcaagcgttaatcggaataactgggcgtaaagcgcacgcaggcggttatgcaagtcagatgtgaaatcccagcgcttaacgttgggcggtcatttgaaactgcaagactagagtactataaggggagctggaattccaggtgtagcggtgaaatgcgtagatatctggaagaacaccggtggcgaaggcgagcttctggatagtaactgacgctcaggtgcgaaagcgtggggagcaaacag

SEQ ID NO: 251 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggtgcccaacacctgcactgcagcgcgaactggctgacttgagtacgcacgacgttggcggaatttgccgtgtagcggtgaaatgcttagatatgacgaagaaccccgattgcgaaggcagctcgcgggagcgcaactgacgctgaagctcgaaagtgcgggtatcgaacag

SEQ ID NO: 252 - DNA, 252 bp, Artificial sequence; identical to SEQ ID NO: 251
  16S rRNA  amino acid sequence

SEQ ID NO: 253 - DNA, 252 bp, Artificial sequence; identical to SEQ ID NO: 251
  16S rRNA  amino acid sequence

SEQ ID NO: 254 - DNA, 251 bp, Artificial sequence
  16S rRNA  amino acid sequence
tacggaagatgcgagcgttatccggaatcattgggtttaaagggagcgtaggcggccttttgagtcagcggttaaatgtcagcgcccaacgttggcgtgccgttgatactggagggcttgagttcacacaaggaaggtggaattcgtcgtgtagcggtgaaatgcttagatatgacgaggaaccccgattgcgaaggcatccttctggggtgttactgacgctgaggctcgaaagcgcgggtatcaaacag

SEQ ID NO: 255 - DNA, 252 bp, Artificial sequence
  16S rRNA  amino acid sequence
tacggaaggtccgggcgttatccggatttattgggtttaaagggtgcgtaggcggtttgctaagcgggaggtgaaatgccggcgctcaacgtcggaagtgcctcccgaactggtggactggaatgactctgccgtgggaggaatgtgtagtgtagcggtgaaatgcttagagattacacagaacaccgattgcgaaggcatctcacgaagggttgattgacgctgaggcacgaaagcgtggggatcaaacag

SEQ ID NO: 256 - DNA, 252 bp, Artificial sequence
  16S rRNA  amino acid sequence
tacagaggtcccgagcgttgttcggatttactgggcgtaaagggcgcgtaggcggcggaacgtgtcggatgtgaaatctccgggctcaacccggaaagtgcgtccgaaactgttccgctggagtccgggagaggtggccggaatgcagggtgtagcggtgaaatgcgttgatatcctgcagaacaccggaggcgaaggcgggccactggaacggtactgacgctgaggcgcgaaagtcgggggagcaaacag

SEQ ID NO: 257 - DNA, 252 bp, Artificial sequence
  16S rRNA  amino acid sequence
tacggaaggtccgggcgttatccggatttattgggtttaaagggagcgcaggctgatgattaagcgtgacgtgaaatgtaggcgctcaacgtctgacttgcagcgcatactggtccacttgagtgcgcgcaacgcgggcggaatttgtcgtgtagcggtgaaatgcttagatatgacgaagaaccccgattgcgaaggcagctcgcgggagcgcaactgacgctgaagctcgaaagtgcgggtatcgaacag

SEQ ID NO: 258 - DNA, 252 bp, Artificial sequence
  16S rRNA  amino acid sequence
tacgtatggtgcaagcgttatccggatttactgggtgtaaagggagtgcaggcggtcttgcaagtcagatgtgaaaacccaaggctcaaccatgggactgcatttgaaactgtgagactagagtatcggagaggtaagtggaattcctagtgtagcggtgaaatgcgtagatattaggaagaacaccagtggcgaaggcggcttactggacgattactgacgctgaggctcgaaagcgtggggagcaaacag

SEQ ID NO: 259 - DNA, 252 bp, Artificial sequence
  16S rRNA  amino acid sequence
tacggaggatgcaagcgttatccggatttattgggtttaaagggtgcgtaggctgtttgtcaagtcaggggtgaaatacctgagcctaactcaggaactgcctttgatactggcaggctggaatacggatgccgtgggaggaatgagtagtgtagcggtgaaatgcatagatattactcagaacaccgattgcgaaggcatctcacgaatccgtcattgacgctgaggcacgaaagcgtggggatcaaacag

SEQ ID NO: 260 - DNA, 251 bp, Artificial sequence
  16S rRNA  amino acid sequence
tacgtaggtggcaagcgttgtccggaattattgggcgtaaagcgcgcgcaggcggcctcctaagtccatcttaaaagtgcggggcttaaccccgtgatgggatggaaactgggaagctggagtatcggagaggaaagtggaattcctagtgtagcggtgaaatgcgtagagattaggaagaacaccggtggcgaaggcgactttctggacagtaactgacgttgaggcacgaaagtgtggggagcaaacag

SEQ ID NO: 261 - DNA, 251 bp, Artificial sequence
  16S rRNA  amino acid sequence
tacggaagatgcgagcgttatccggatttattgggtttaaagggagcgtaggcggacctttaagtcagcggtaaaatgtcaatgcccaacattggcccgccgttgatactgttggtcttgaatacacacaaggaaggtggaattcgttgtgtagcggtgaaatgcttagatatgacgaagaactccgattgcgaaggcagccttctggggtgcgattgacgctgaggctcgaaagtgcgggtatcaaacag

SEQ ID NO: 262 - DNA, 252 bp, Artificial sequence
  16S rRNA  amino acid sequence
cacgtaaggtgcgagcgttgttcggaatcattgggcgtaaagggcgcgtaggcggcctgagaagcggggtgtgaaaggcgggggcccaacccccggactgcgccccgaactctctggctggagtggctgtggcgccggcggaattccaggtgtaggggtgaaatctgtagatatctggaagaacaccggtggcgaaggcggccggcgagcagacgactgacgctgaggcgcgaaggtgcggggagcgaacag

SEQ ID NO: 263 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatcacgaagaactccgattgcgaaggcagccaacaaggcctttactgacgctaaagctcgaaggtgcgggtatcgaacag

SEQ ID NO: 264 - DNA, 252 bp, Artificial sequence; identical to SEQ ID NO: 263
  16S rRNA  amino acid sequence

SEQ ID NO: 265 - DNA, 252 bp, Artificial sequence
  16S rRNA  amino acid sequence
tacgtagggggcaagcgttatccggatttactgggtgtaaagggagcgcagacggaagagcaagtctgaagtgaaaacctgcggcttaactgcaggattgctttggaaactgtttttcttgagtactggagaggtaagcggaattcctggtgtagtagtgaaatgcgtagatatcaggaggaacaccggtggcgaaggcggcttactgggcttttactgacgctgaggctcgaaagcgtggggagcaaacag

SEQ ID NO: 266 - DNA, 252 bp, Artificial sequence
  16S rRNA  amino acid sequence
tacggaaggtccgggcgttatccggatttattgggtttaaagggagcgcaggcgggtttttaagcgtgttgtgaaatgtaccggctcaaccggtgacttgcagcgcgaactggagaccttgagtgagtacgaggaaggcggaattcgtggtgtagcggtgaaatgcttagatatggcgaagaactccgattgcgaaggcagctgacgggagcgcaactgacgctcatgctcgaaagtgcgggtatcgaacag

SEQ ID NO: 267 - DNA, 252 bp, Artificial sequence
  16S rRNA  amino acid sequence
tacggaggatgcgagcgttatccggatttattgggtttaaagggtgcgtaggcggagcgttaagtcagcggtgaaatgtcccggctcaaccggggagctgccgttgatactggcgcactagggtccgagcggcgccggcggaatgtgcaatgtagcggtgaaatgcatagatattgcacagaacaccgatcgcgaaggcagctggcgaggacgggaccgacgctgaggcacgaaggcgtggggatcgaacag

SEQ ID NO: 268 - DNA, 251 bp, Artificial sequence
  16S rRNA  amino acid sequence
tacggaggatgcgagcgttatccggatttattgggtttaaagggtgcgcaggcggcgcgtcaagtcagcggtcaaactgcggggctcaaccccgtatcgccgttgaaactggcgagctggagttcgtgcgacggaggcggaatgcgcggtgtagcggtgaaatgcatagatatcgcgcagaaccccgattgcgaaggcagcttccgagcacgcgactgacgctcatgcacgaaagcgtgggtatcgaacag

SEQ ID NO: 269 - DNA, 252 bp, Artificial sequence
  16S rRNA  amino acid sequence
tacgtagggggcaagcgttatccggatttactgggtgtaaagggagcgtagacggcatggcaagtctgatgtgaaaggtgcgggctcaacccgtaaactgcattggaaactgtcacgctggagtgcaggagaggtaagtggaattcctagtgtagcggtgaaatgctcagatattaggaggaacaccagcggcgaaggcggcttactggactgtaactgacgttgaggctcgaaagcgtgggtagcaaacag

SEQ ID NO: 270 - DNA, 252 bp, Artificial sequence
  16S rRNA  amino acid sequence
tacgtaggtggcaagcgttatccggatttactgggtgtaaagggcgcgtaggcggggatgcaagtcagatgtgaaatctatgggcttaactcataaactgcatttgaaactgtatctcttgagtgctggagaggtagacggaattccttgtgtagcggtgaaatgcgtagatataaggaagaacaccagtggcgaaggcggtctactggacagtaactgacgctgaggcgcgaaagcgtggggagcaaacag

SEQ ID NO: 271 - DNA, 251 bp, Artificial sequence
  16S rRNA  amino acid sequence
tacggaagatgcgagcgttatccggatttattgggtttaaagggagcgtaggcgggcctttaagccagcggtcaaatgtcatggcccaaccttggcatgccgttggaactggtggccttgaatacacacaaggaaggtggaattcgtcgtgtagcggtgaaatgcttagatatgacgaagaactccgattgcgaaggcagcttacgggagcgcaactgacgctgaagctcgaaagtgcgggtatcgaacag

SEQ ID NO: 272 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcacaacgcgggcggaatttgtcgtgtagcggtgaaatgcttagatatgacgaagaactccgattgcgaaggcagcctgctgtagcgccactgacgctgatgctcgaaagcgtgggtatcgaacag

SEQ ID NO: 273 - DNA, 252 bp, Artificial sequence
  16S rRNA  amino acid sequence
tacggaaggtccgggcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatcacgaagaactccgattgcgaaggcagccggcgagtcctcaactgacgctaatgctcgaaagtgcgggtatcgaacag

SEQ ID NO: 274 - DNA, 252 bp, Artificial sequence
  16S rRNA  amino acid sequence
tacggaaggtccgggcgttatccggatttattgggtttaaagggagcgcaggccgtttggtaagcgtgttgtgaaatgtccgggctcaacctgggcactgcagcgcgaactgtcagacttgagtgcacaggaagcgggcggaattcgtggtgtagcggtgaaatgcttagatatgacgaagaaccccgattgcgaaggcagctcgcgggagcgcaactgacgctgaagctcgaaagtgcgggtatcgaacag

SEQ ID NO: 275 - DNA, 252 bp, Artificial sequence
  16S rRNA  amino acid sequence
cacgtaaggcgcgagcgttgttcggaatcattgggcgtaaagggcgtgtaggcggccgcgcaagtctggagtgaaatccccgggcccaacccgggggccgctctggaaactgcgcggctggagtcgccgaggcgcggccggaattccaggtgtaggggtgaaatctgtagatatctggaagaacaccggtggcgaaggcaggccgcgagcggacgactgacgctgaggcgcgaaggcgcggggagcgaacag

SEQ ID NO: 276 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgacttgcagcgcgaactgacggtcttgagtacgcacaacgttggcggaatttgtcgtgtagcggtgaaatgcttagatatgacgaagaactccgattgcgaaggcagcctgctgtagcgccactgacgctgatgctcgaaagcgtgggtatcgaacag

SEQ ID NO: 277 - DNA, 252 bp, Artificial sequence
  16S rRNA  amino acid sequence
tacgtatggagcaagcgttatccggatttactgggtgtaaagggagtgtaggcggtggatcaagtctgatgtgaaacccccgggctcaacctggggattgcattggaaactggttaactggagtgtcggagaggcaagtggaattcctagtgtagcggtgaaatgcgtagatattaggaggaacaccagtggcgaaggcggcttgctggacgataactgacgctgaggctcgaaagcgtggggagcaaacag

SEQ ID NO: 278 - DNA, 252 bp, Artificial sequence
  16S rRNA  amino acid sequence
tacggaaggtccgggcgttatccggatttattgggtttaaagggagcgcaggcggggagctaagcgtgacgtgaaatgtagccgctcaacggctgaactgcgtcgcgaactggctttcttgagtgagtacgacgtcagcggaattcgtggtgtagcggtgaaatgcttagatatcacgaagaaccccgattgcgaaggcagctgacgagtccttaactgacgctaaagctcgaaagtgcgggtatcgaacag

SEQ ID NO: 279 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgacttgcagcgcgaactgacggtcttgagtacgcacaacgttggcggaatttgtcgtgtagcggtgaaatgcttagatatgacgaagaactccgattgcgaaggcagctgacgggagcgccactgacgcttaagctcgaaagtgcgggtatcgaacag

SEQ ID NO: 280 - DNA, 252 bp, Artificial sequence; identical to SEQ ID NO: 279
  16S rRNA  amino acid sequence

SEQ ID NO: 281 - DNA, 252 bp, Artificial sequence
  16S rRNA  amino acid sequence
tacggagggtgcgagcgttaatcggaataactgggcgtaaagagctcgtaggcggttagtcaagtcagatgtgaaagccccgggcttaacctgggaactgcatttgaaactgacgaactagagtactgtagagggaggtagaattccaggtgtagcggtgaaatgcgtagagatttggaagaataccggaggcgaaggcggctctctggaccgatactgacactgaagagcgaaagcgtggggagcaaacag

SEQ ID NO: 282 - DNA, 252 bp, Artificial sequence
  16S rRNA  amino acid sequence
tacgtagggggcaagcgttatccggatttactgggtgtaaagggagcgcagacggtcaagcaagtctgaagtgaaaccccacggctcaaccgtgggcttgctttggaaactgtttgactagagtactggagaggtaagcggaattcctagtgtagcggtgaaatgcgtagatattaggaggaacatcggtggcgaaggcggcttactggacagcaactgacgttgaggctcgaaggcgtggggagcaaacag

SEQ ID NO: 283 - DNA, 252 bp, Artificial sequence
  16S rRNA  amino acid sequence
tacagaggtccctagcgttgttcggatttactgggcgtaaagggcgcgtaggcggccgagcaagtcaagtgtgaaatctccgggctcaacccggaacgtgcgcttgagactgcctggctcgaggccgggagggggaatcggaatacagggtgtagcggtgaaatgcgttgatatcctgtagaacaccggaggcgaaggcggattccttggacggcactgacgctgaggcgcgaaagtcgggggagcaaacag

SEQ ID NO: 284 - DNA, 252 bp, Artificial sequence
  16S rRNA  amino acid sequence
tacggaggatgcgagcgttatccggatttattgggtttaaagggtgcgtaggcggcttgacaagtcagcggtgaaatgcaagggcctaacccttgacgtgccgttgaaactgccgggctagaatgcggatgctgtgggaggaatgtgtagtgtagcggtgaaatgcatagatattacacagaacaccgattgcgaaggcatctcacaaatccgctattgacgctgaggcacgaaagtgcggggatcaaacag

SEQ ID NO: 285 - DNA, 252 bp, Artificial sequence
  16S rRNA  amino acid sequence
tacggaaggtccgggcgttatccggatttattgggtttaaagggagcgtaggccgtcgtttaagcgtgttgtgaaatgtaggcgcccaacgtctgcactgcagcgcgaactggatgacttgagtacgcgggaagcaggcggaattcgtcgtgtagcggtgaaatgcttagatatgacgaagaaccccgattgcgaaggcagctcgcgggagcgcaactgacgctgaagctcgaaagtgcgggtatcgaacag

SEQ ID NO: 286 - DNA, 252 bp, Artificial sequence
  16S rRNA  amino acid sequence
tacggaaggtccgggcgttatccggatttattgggtttaaagggagcgtaggccggatggcaagcgtgttgtgaaatgtcgtggcccaaccagggcactgcagcgcgaactgccgttcttgagtgcgcgcaacgttggcggaatttgtcgtgtagcggtgaaatgcttagatatgacgaagaactccgattgcgaaggcagccaacaaggcctttactgacgctaaagctcgaaggtgcgggtatcgaacag

SEQ ID NO: 287 - DNA, 253 bp, Artificial sequence
  16S rRNA  amino acid sequence
tacgtatggagcaagcgttatccggatttactgggtgtaaagggagtgtaggcggtgcatcaagtctgatgtgaaaacccggggctcaactccgcggattgcattggaaactggtgtactggagtgtcggagaggcaagcggaattcctagtgtagcggtgaaatgcgtagatattaggaggaacaccagtggcgaaggcggcttgctggacgataactgacgctgaggctcgaaagcgtggggagcaaacag

SEQ ID NO: 288 - DNA, 253 bp, Artificial sequence; identical to SEQ ID NO: 287
  16S rRNA  amino acid sequence

SEQ ID NO: 289 - DNA, 253 bp, Artificial sequence; identical to SEQ ID NO: 287
  16S rRNA  amino acid sequence

SEQ ID NO: 290 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgacttgcagcgcgaactgacggtcttgagtacgcacaacgttggcggaatttgtcgtgtagcggtgaaatgcttagatatgacgaagaactccgattgcgaaggcagccaacaaggcctttactgacgctaaagctcgaaggtgcgggtatcgaacag

SEQ ID NO: 291 - DNA, 252 bp, Artificial sequence; identical to SEQ ID NO: 290
  16S rRNA  amino acid sequence

SEQ ID NO: 292 - DNA, 252 bp, Artificial sequence; identical to SEQ ID NO: 290
  16S rRNA  amino acid sequence

SEQ ID NO: 293 - DNA, 252 bp, Artificial sequence
  16S rRNA  amino acid sequence
tacgtaggtggcgagcgttatccggaattactgggtgtaaagggtgtgtaggcgggattgcaagtcaggcgtgaaatcataaggctcaaccttgtgactgcgcttgaaactgtaattcttgagagtgggagaggtaaacggaattcctggtgtagtagtgaaatgcgtagatatcaggaggaacaccggtggcgaaggcggtttactggaccacaactgacgctgagacacgaaagcgtggggagcaaacag

SEQ ID NO: 294 - DNA, 252 bp, Artificial sequence
  16S rRNA  amino acid sequence
tacggaaggtccgggcgttatccggatttattgggtttaaagggagcgcaggcgggttattaagcgtgttgtgaaatgcggcggctcaaccgtcgaattgcagcgcgaactggttaccttgagtgagtacgaggaaggcggaattcgtggtgtagcggtgaaatgcttagatatcacgaagaactccgattgcgaaggcagctttccagtccttaactgacgctaaagctcgaaagtgcgggtatcgaacag

SEQ ID NO: 295 - DNA, 252 bp, Artificial sequence
  16S rRNA  amino acid sequence
tacggaggatgcgagcgttatccggatttattgggtttaaagggtgcgtaggcggtttgtcaagtcagcggtgaaacgcaagggcttaacccttgacctgccgttgaaactgatggactagaatgcggatgctgtgggaggaatgtgtggtgtagcggtgaaatgcatagatatcacacagaacaccgattgcgaaggcatctcacaaatccgtgattgacgctgaggcacgaaagtgtggggatcaaacag

SEQ ID NO: 296 - DNA, 252 bp, Artificial sequence
  16S rRNA  amino acid sequence
tacgtagggagcgagcgttgtccggatttactgggtgtaaagggtgcgtaggcggctatgcaagtcagatgtgaaatgtatgggctcaacccatgaactgcatttgaaactgcgtagcttgagtgaagtagaggcaggcggaattccctgtgtagcggtgaaatgcgtagagatagggaggaacaccagtggcgaaggcggcctgctgggctttaactgacgctcaggcacgaaagcgtgggtagcaaacag

SEQ ID NO: 297 - DNA, 252 bp, Artificial sequence
  16S rRNA  amino acid sequence
tacggagggtacgagcgttaatcggaatgactgggcgtaaagggcacgtaggtggatagataagtcacaagtgaaatacccgggcttaacctgggattggctcgtgaaactgtttaactagagtacggtagaggggagtagaattccaagtgtagcggtgaaatgcgtagatatctggaggaataccggtggcgaaggcggcctcctggacagagactgacgctgaggagcgaaagcgtggggagcaaacag

SEQ ID NO: 298 - DNA, 252 bp, Artificial sequence
  16S rRNA  amino acid sequence
tacggaggatgcgagcgttatccggattcattgggtttaaagggtgcgcaggcggcgcggtaagtcagcggtgaaagtcaggggcccaaccccggaagtgccgttgatactgccgtgcttgaatgcggtcgaggcgggcggaatgtggtgtgtagcggtgaaatgcatagatatgccacagaacgccgatagcggaggcagctcgccaggcctgcattgacgctcgggcacgaaagcgtggggatcgaacag

SEQ ID NO: 299 - DNA, 251 bp, Artificial sequence
  16S rRNA  amino acid sequence
tacggaaggtccgagcgttatccggatttattgggtttaaagggagcgtaggcggaatggtaagtcagccgttaaatagcggggctcaaccccgtagtgcggttgaaactgccgttcttgagtgcacgagaggtaggtggaattcgtggtgtagcggtgaaatgcttagatatcacgaagaactccgattgcgaaggcagcttactggtgtgttactgacgctgatgctcgaaggtgcgggtatcaaacag

SEQ ID NO: 300 - DNA, 252 bp, Artificial sequence
  16S rRNA  amino acid sequence
tacgtagggagcaagcgttgtccggatttactgggtgtaaagggtgcgcaggcggctttgcaagtcagctgtgaaatctatgggctcaacccataaattgcagttgaaactgtagagcttgagtgaagtagaggtaggcggaattcccggtgtagcggtgaaatgcgtagagatcgggaggaacaccagtggcgaaggcggcctactgggctttaactgacgctcaggcacgaaagcatgggtagcaaacag

SEQ ID NO: 301 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgctcaacgtctgacttgcagcgcatactggtccacttgagtgcgcacaacgcgggcggaatttgtcgtgtagcggtgaaatgcttagatatgacgaagaaccccgattgcgaaggcagctcgcgggagcgcaactgacgctgaagctcgaaagtgcgggtatcgaacag

SEQ ID NO: 302 - DNA, 252 bp, Artificial sequence; identical to SEQ ID NO: 301
  16S rRNA  amino acid sequence

SEQ ID NO: 303 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgctcaacgcctgacttgcagcgcgaactgacggtcttgagtgcgcgcaacgttggcggaatttgtcgtgtagcggtgaaatgcttagatatgacgaagaactccgattgcgaaggcagctgacgggagcgttactgacgcttaagctcgaaagcgcgggtatcgaacag

SEQ ID NO: 304 - DNA, 252 bp, Artificial sequence
  16S rRNA  amino acid sequence
tacagaggccaagagcgttgttcggatttactgggcgtaaagggcgcgtaggcggcatggcgtgtcggatgtgaaatctccgggctcaacccggaaagtgcgtccgaaactaccgtgctggagtatgggagggggaatcggaatgcagggtgtagcggtgaaatgcgttgatatcctgcagaacaccggaggcgaaggcggattccttgaacatcactgacgctgaggcgcgaaagtcgggggagcaaacag

SEQ ID NO: 305 - DNA, 252 bp, Artificial sequence
  16S rRNA  amino acid sequence
tacagaggcccgtagcgttgttcggatttactgggcgtaaagggcgcgtaggcggccatgcgtgtcggatgtgaaatctccgggctcaacccggaaagtgcgtccgaaactaccgtgctggagtatgggagggggaatcggaatatagggtgtagcggtgaaatgcgttgatatcctatagaacaccggaggcgaaggcggattccttgaacatcactgacgctgaggcgcgaaagtcgggggagcaaacag

SEQ ID NO: 306 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgctcaacgcctgacttgcagcgcgaactgacggtcttgagtgcgcgcaacgcgggcggaatttgtcgtgtagcggtgaaatgcttagatatgacgaagaaccccgattgcgaaggcagctcgcgggagcgcaactgacgctgaagctcgaaagtgcgggtatcgaacag

SEQ ID NO: 307 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgaactgcagcgcgaactgacggtcttgagtgcgcacaacgttggcggaattcgtcgtgtagcggtgaaatgcttagatatgacgaagaaccccgattgcgaaggcagctcgcgggagcgcaactgacgctgaagctcgaaagtgcgggtatcgaacag

SEQ ID NO: 308 - DNA, 252 bp, Artificial sequence
  16S rRNA  amino acid sequence
tacgtaggtggcaagcgttgtccggatttactgggtgtaaagggcgcgtaggcgggtcgtcaagtcagatgtgaaatctatgggctcaactcataaactgcatttgaaactgacggtcttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacag

SEQ ID NO: 309 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 310 - DNA, 252 bp, Artificial sequence; identical to SEQ ID NO: 309
  16S rRNA  amino acid sequence

SEQ ID NO: 311 - DNA, 252 bp, Artificial sequence
  16S rRNA  amino acid sequence
tacggaaggtccgggcgttatccggatttattgggtttaaagggagcgcaggccgttggttaagcgtgccgtgaaatgtaccggctcaaccggtgagttgcggcgcgaactgtccgacttgagtgcgtacgagggtggcggaattcgtggtgtagcggtgaaatgcttagatatcacgaagacctccgattgcgaaggcagctgcccagtccgtaactgacgctaatgctcgaaggtgcgggtatcgaacag

SEQ ID NO: 312 - DNA, 195 bp, Artificial sequence
  16S rRNA  amino acid sequence
gtttgtaataattttttttgattttaaaaaaaaattatttatggttttgtctatccctaaaattggtttgttgtgaaatgaatcaaatttagggaataggctttcataaataagttttttttaaagtcttaaagaccttttttaaaattaaacttttgtattcatttgtctaaattttttttaaataatttaaaa

SEQ ID NO: 313 - DNA, 225 bp, Artificial sequence
  16S rRNA  amino acid sequence
taccatggctctcacgggtaacagggaattagggttcgattctggagaaggagcctgagaaacggctactacatctacggaaggcagcaggcgcgtaaattacccaatcctgactcagggaggtggtgacaagatataacaacgagactataaatctcgattgtagtgagggtattctaaatagaacctatagtacgattagagggcaagtctggtgccagagcc

SEQ ID NO: 314 - DNA, 252 bp, Artificial sequence
  16S rRNA  amino acid sequence
tacggaaggtccgggcgttatccggatttattgggtttaaagggagcacaggccggctgtcaagcgtgttgtgaaatgtagccgctcaacggctgaactgcgtcgcgaactggttatcttgagtgagttcgatgttggcggaattcgtggtgtagcggtgaaatgcttagatatcacgaagaactccgattgcgaaggcagccaacaaggcctttactgacgctaaagctcgaaggtgcgggtatcgaacag

SEQ ID NO: 315 - DNA, 252 bp, Artificial sequence; identical to SEQ ID NO: 52
  16S rRNA  amino acid sequence

SEQ ID NO: 316 - DNA, 252 bp, Artificial sequence
  16S rRNA  amino acid sequence
tacagaggtcgcaagcgttgttcggatttactgggcgtaaagggcgcgtaggcggcaaggcgtgtcggatgtgaaatctccgggctcaacccggaaagtgcgtccgaaactaccttgctcgagtatgggagggggaatcggaatatagggtgtagcggtgaaatgcgttgatatcctatagaacaccggaggcgaaggcggattccttgaacatcactgacgctgaggcgcgaaagtcgggggagcaaacag

SEQ ID NO: 317 - DNA, 252 bp, Artificial sequence
  16S rRNA  amino acid sequence
tacggagggtgcaggcgttatccggatttattaggtttaaagggtgcgcaggcgggacattaagtcggcggtgaaataccgtggcccaactacggggctgccgtcgatactgatgaccttgagttatggtgaggtaaccggaatgtggagtgtagcggtgaaatgcatagatattccacagaacaccgattgcgaaggcaggttaccaactgtacactgacgctcatgcacgaaagcgcgggtagcgaacag

SEQ ID NO: 318 - DNA, 252 bp, Artificial sequence; identical to SEQ ID NO: 317
  16S rRNA  amino acid sequence

SEQ ID NO: 319 - DNA, 252 bp, Artificial sequence; identical to SEQ ID NO: 55
  16S rRNA  amino acid sequence

SEQ ID NO: 320 - DNA, 251 bp, Artificial sequence; identical to SEQ ID NO: 57
  16S rRNA  amino acid sequence

SEQ ID NO: 321 - DNA, 252 bp, Artificial sequence; identical to SEQ ID NO: 58
  16S rRNA  amino acid sequence

SEQ ID NO: 322 - DNA, 252 bp, Artificial sequence; identical to SEQ ID NO: 59
  16S rRNA  amino acid sequence

SEQ ID NO: 323 - DNA, 252 bp, Artificial sequence
  16S rRNA  amino acid sequence
tacggaaggtccaggcgttatccggatttattgggtttaaagggagcacaggccggctgtcaagcgtgttgtgaaatgtaggcgctcaacgcctgacttgcagcgcgaactgacggtcttgagtgcgcgcaacgttggcggaatttgtcgtgtagcggtgaaatgcttagatatgacgaagaactccgattgcgaaggcagctgacgggagcgcaactgacgctcatgctcgaaagtgcgggtatcgaacag

SEQ ID NO: 324 - DNA, 252 bp, Artificial sequence
  16S rRNA  amino acid sequence
tacggaaggtccgggcgttatccggatttattgggtttaaagggagcgcaggccgatggttaagcgtgacgtgaaatgtaggggctcaacctttgaattgcgtcgcgaactggctgtcttgagtgagtacgacgtcagcggaattcgtggtgtagcggtgaaatgcttagatatcacgaagaaccccgattgcgaaggcagctgacgagtcctttactgacgctaaagctcgaaggtgcgggtatcgaacag

SEQ ID NO: 325 - DNA, 252 bp, Artificial sequence
  16S rRNA  amino acid sequence
tacggaaggtccgggcgttatccggatttattgggtttaaagggtgcgtaggcggagctttaagcgtgaggtgaaatgcccgggctcaacctggggactgcctcgcgaactggagttctggaatgcccctgccgtgggaggaatgtgtggtgtagcggtgaaatgcatagagatcacacagaacaccgattgcgaaggcatctcacgaaggggtgattgacgctgaggcacgaaagcgtggggatcaaacag

SEQ ID NO: 326 - DNA, 252 bp, Artificial sequence; identical to SEQ ID NO: 61
  16S rRNA  amino acid sequence

SEQ ID NO: 327 - DNA, 252 bp, Artificial sequence; identical to SEQ ID NO: 62
  16S rRNA  amino acid sequence

SEQ ID NO: 328 - DNA, 252 bp, Artificial sequence
  16S rRNA  amino acid sequence
tacgaggggtgcaagcgttgttcggaatcactgggcgtaaagggagcgtaggcggaatcgcaagcggattgtacaataccggggcccaaccccggacctgcagtccgaactgcggttcttggatagttcaggggcaggcggaattcctggtgtagcggtggaatgcgtagagatcaggaagaacaccgatggcgaaggcagcctgctggggacttatcgacgctgaggctcgaaagcgcgggtagcaaacag

SEQ ID NO: 329 - DNA, 252 bp, Artificial sequence
  16S rRNA  amino acid sequence
tacggaaggtccgggcgttatccggatttattgggtttaaagggagcgcaggccggagattaagcgtgacgtgaaatgtaggggctcaacctttgaattgcgtcgcgaactggttttcttgagtgagtacgacggaggcggaattcgtggtgtagcggtgaaatgcttagatatcacgaagaaccccgattgcgaaggcagcctccgagtccttaactgacgctaatgctcgaaagtgcgggtatcgaacag

SEQ ID NO: 330 - DNA, 251 bp, Artificial sequence
  16S rRNA  amino acid sequence
tacggaaggtccgagcgttatccggatttattgggtttaaagggtgcgcaggcggaatgtcaagtcagccgtgaaatcgcgtggctcaaccacgctgagcggttgaaactggtattcttgagtgcggtgaaggaaggcggaattcgtggtgtagcggtgaaatgcatagatatcacgaagaactccgattgcgtaggcagctttccgtaccgttactgacgctcatgcacgaaggtgcgggtatcaaacag

SEQ ID NO: 331 - DNA, 252 bp, Artificial sequence; identical to SEQ ID NO: 65
  16S rRNA  amino acid sequence

SEQ ID NO: 332 - DNA, 252 bp, Artificial sequence; identical to SEQ ID NO: 70
  16S rRNA  amino acid sequence

SEQ ID NO: 333 - DNA, 252 bp, Artificial sequence
  16S rRNA  amino acid sequence
cacgtaagccccgagcgttgttcggaattattgggcgtaaagggcatgtaggcggttttgcaagtctggtgtgaaaggcaggagctcaactcctggactgcgctggaaactgcatgacttgagtttctgaggggcactcggaattcctggtgtaggggtgaaatctgtagatatcaggaagaacaccggtggcgaaggcggggtgccagcagaagactgacgctgaggtgcgaaggtgcggggagcaaacag

SEQ ID NO: 334 - DNA, 252 bp, Artificial sequence; identical to SEQ ID NO: 71
  16S rRNA  amino acid sequence

SEQ ID NO: 335 - DNA, 252 bp, Artificial sequence
  16S rRNA  amino acid sequence
tacggaaggtccgggcgttatccggatttattgggtttaaagggagcgcaggccgtttggtaagcgtgttgtgaaatgtccgggctcaacctgggcactgcagcgcgaactgccagacttgagtgcacaggaagcgggcggaattcgtggtgtagcggtgaaatgcttagatatggcgaagaactccgattgcgaaggcagctgacgggagcgcaactgacgctcatgctcgaaagtgcgggtatcgaacag

SEQ ID NO: 336 - DNA, 252 bp, Artificial sequence; identical to SEQ ID NO: 72
  16S rRNA  amino acid sequence

SEQ ID NO: 337 - DNA, 252 bp, Artificial sequence; identical to SEQ ID NO: 74
  16S rRNA  amino acid sequence

SEQ ID NO: 338 - DNA, 252 bp, Artificial sequence
  16S rRNA  amino acid sequence
tacggaggatgcgagcgttatccggatttattgggtttaaagggtgcgtaggcggaccgttaagtcagcggtgaaatatcccggcccaaccggggggctgccgttgatactggcggactagggtccaaacggcgccggcggaatatgtaatgtagcggtgaaatgcatagatattacatagaacaccgatcgcgaaggcagctggcgaggatggcactgacgctgaggcacgaaggcgtggggatcaaacag

SEQ ID NO: 339 - DNA, 252 bp, Artificial sequence
  16S rRNA  amino acid sequence
tacgtagggagcgagcgttatccggatttattgggtgtaaagcgtgcgtagacgggcaattaagttagttgtgaaatccctcggcttaactgaggaactgcagctaaaactggttgtcttgagtactggaggggaaggcggaattcctagtgtagcggtgaaatgcgtagatattaggaagaacaccggtggcgaaggcggccttctggacagaaactgacgttgaggcacgaaagtgtggggagcaaacag

SEQ ID NO: 340 - DNA, 252 bp, Artificial sequence; identical to SEQ ID NO: 75
  16S rRNA  amino acid sequence

SEQ ID NO: 341 - DNA, 252 bp, Artificial sequence
  16S rRNA  amino acid sequence
tacggaaggtccgggcgttatccggatttattaggtctaaagggtgcgcaggcggctctctaagtcggcggtgaaaacccgtggcccaaccacggacgtgccatcgatactggggggcttgagtgcggatgaggtatgcggaatgagtggtgtagcggtgaaatgcttagatattactcagaacaccgattgcgaaggcagcgtgccaatccgttactgacgctcatgcacgaaagcgcgggtagcgaacag

SEQ ID NO: 342 - DNA, 252 bp, Artificial sequence; identical to SEQ ID NO: 76
  16S rRNA  amino acid sequence

SEQ ID NO: 343 - DNA, 252 bp, Artificial sequence
  16S rRNA  amino acid sequence
tacgtagggggcaagcgttatccggatttactgggtgtaaagggagcgtagacggagaagcaagtcagatgtgaaaggccggggctcaaccccgggattgcatttgaaactgtctctcttgagtaccggaagggcagacggaattcctggtgtagcggtgaaatgcgtagagatcaggaggaacaccggtggcgaaagcggtctgctggacggtaactgacgttgaggctcgaaggcgtggggagcaaacag

SEQ ID NO: 344 - DNA, 252 bp, Artificial sequence; identical to SEQ ID NO: 78
  16S rRNA  amino acid sequence

SEQ ID NO: 345 - DNA, 252 bp, Artificial sequence
  16S rRNA  amino acid sequence
tacggaggatgcaagcgttatccggatttattgggtttaaagggtgcgtaggcgggattttaagcgtgaggtgaaatgcccgggctcaacctggggactgcctcgcgaactggagttctggaatgcccctgccgtgggaggaatgtgtggtgtagcggtgaaatgcatagagatcacacagaacaccgattgcgaaggcatctcacgaaggggtgattgacgctgaggcacgaaagcgtggggatcaaacag

SEQ ID NO: 346 - DNA, 252 bp, Artificial sequence
  16S rRNA  amino acid sequence
tacggaaggtccgggcgttatccggatttattgggtttaaagggagcgtaggcgggctgttaagcgtgtcgtgaaaagcatcggctcaaccggtgtcgtgcggcgcggactggcggtcttgagtgggtagaaagccggcggaattcgtggtgtagcggtgaaatgcttagatatcacgaagaactccgattgcgaaggcagccaacaaggcctttactgacgctaaagctcgaaggtgcgggtatcgaacag

SEQ ID NO: 347 - DNA, 252 bp, Artificial sequence; identical to SEQ ID NO: 346
  16S rRNA  amino acid sequence

SEQ ID NO: 348 - DNA, 252 bp, Artificial sequence
  16S rRNA  amino acid sequence
tacggaaggtccgggcgttatccggatttattgggtttaaagggagcgcaggcggagacttaagcgtgctgtgaaatgctgcggctcaaccgtggaagtgcagcgcgaactgggtttcttgagtgagtacgaggtatgcggaattcgtggtgtagcggtgaaatgcttagatatcacgaagaactccgattgcgaaggcagccaacaaggcctttactgacgctaaagctcgaaggtgcgggtatcgaacag

SEQ ID NO: 349 - DNA, 252 bp, Artificial sequence; identical to SEQ ID NO: 88
  16S rRNA  amino acid sequence

SEQ ID NO: 350 - DNA, 252 bp, Artificial sequence; identical to SEQ ID NO: 88
  16S rRNA  amino acid sequence

SEQ ID NO: 351 - DNA, 252 bp, Artificial sequence
  16S rRNA  amino acid sequence
tacggaaggtccgggcgttatccggatttattgggtttaaagggagcgcaggccgatggttaagcgtgacgtgaaatgtagccgctcaacggctgaactgcgtcgcgaactggctgtcttgagtgagttcgatgtgggcggaattcgtggtgtagcggtgaaatgcttagatatcacgaagaaccccgattgcgaaggcagctgacgagccctttactgacgctaaagctcgaaggtgcgggtatcgaacag

SEQ ID NO: 352 - DNA, 252 bp, Artificial sequence; identical to SEQ ID NO: 351
  16S rRNA  amino acid sequence

SEQ ID NO: 353 - DNA, 252 bp, Artificial sequence
  16S rRNA  amino acid sequence
tacgtagggtgcaagcgttatccggatttactgggtgtaaagggcgagtagacggcgttgcaagtcagatgtgaaatcttggggctcaacttcaaaattgcatctgaaactgtaatgctagagtgcaggagaggtaaacggaattcctagtgtagcggtgaaatgcgtagatattaggaagaacatcagtggcgaaggcggtttactggactgtaactgacgttgaggcgcgaaagcgtggggagcaaacag

SEQ ID NO: 354 - DNA, 252 bp, Artificial sequence; identical to SEQ ID NO: 353
  16S rRNA  amino acid sequence

SEQ ID NO: 355 - DNA, 252 bp, Artificial sequence; identical to SEQ ID NO: 353
  16S rRNA  amino acid sequence

SEQ ID NO: 356 - DNA, 252 bp, Artificial sequence; identical to SEQ ID NO: 353
  16S rRNA  amino acid sequence

SEQ ID NO: 357 - DNA, 252 bp, Artificial sequence
  16S rRNA  amino acid sequence
tacgtagggagcaagcgttatccggatttattgggtgtaaagggtgcgtagacgggaagtcaagttagttgtgaaatccctcggcttaactgaggaattgcaactaaaactggttttcttgagtgttggagaggaaagtggaattcctagtgtagcggtgaaatgcgtagatattaggaggaacaccggtggcgaaggcgactttctggacaataactgacgttgaggcacgaaagtgtggggagcaaacag

SEQ ID NO: 358 - DNA, 252 bp, Artificial sequence
  16S rRNA  amino acid sequence
tacggaaggtccgggcgttatccggatttattgggtttaaagggagcgtaggccgcagattaagtgtgttgtgaaatgtaggcgctcaacgtctgacttgcagcgcatactggtccacttgagtgcgcgcaacgcgggcggaatttgtcgtgtagcggtgaaatgcttagatatggcgaagaactccgattgcgaaggcagctgacgggagcgcaactgacgctcatgctcgaaagtgcgggtatcgaacag

SEQ ID NO: 359 - DNA, 252 bp, Artificial sequence
  16S rRNA  amino acid sequence
tacgaggggtgcaagcgttgttcggaatcactgggcgtaaagggagcgtaggcggaccgccaagcgttctgtgcaatcccggggcccaaccccggacccgcagagcgaactgacggtcttgagtagttcagaggcaggcggaattcctggtgtagcggtggaatgcgtagatatcaggaagaacaccgatggcgaaggcagcctgctggggactaactgacgctgaggctcgaaagcgtgggaagcaaacag

SEQ ID NO: 360 - DNA, 252 bp, Artificial sequence; identical to SEQ ID NO: 93
  16S rRNA  amino acid sequence

SEQ ID NO: 361 - DNA, 252 bp, Artificial sequence; identical to SEQ ID NO: 95
  16S rRNA  amino acid sequence

SEQ ID NO: 362 - DNA, 252 bp, Artificial sequence; identical to SEQ ID NO: 96
  16S rRNA  amino acid sequence

SEQ ID NO: 363 - DNA, 252 bp, Artificial sequence; identical to SEQ ID NO: 97
  16S rRNA  amino acid sequence

SEQ ID NO: 364 - DNA, 252 bp, Artificial sequence; identical to SEQ ID NO: 98
  16S rRNA  amino acid sequence

SEQ ID NO: 365 - DNA, 251 bp, Artificial sequence
  16S rRNA  amino acid sequence
tacggaaggtccgggcgttatccggatttattgggtttaaagggagcgtaggcgggtttataagcgtgtcgtgaaattatgtggctcaaccatgtcgtgcggcgcgaactgtagaccttgagtgcgctgaaagcgggcggaattcgtggtgtagcggtgaaatgcttagatatcacgaagaactccgattgcgaaggcagctcgctgtagcgttactgacgctgaagctcgaaggcgcgggtatcgaacag

SEQ ID NO: 366 - DNA, 252 bp, Artificial sequence; identical to SEQ ID NO: 100
  16S rRNA  amino acid sequence

SEQ ID NO: 367 - DNA, 252 bp, Artificial sequence
  16S rRNA  amino acid sequence
tacggaaggtccgggcgttatccggatttattgggtttaaagggagcgcaggctgatgattaagcgtgacgtgaaatgtagccgctcaacggctgaactgcgtcgcgaactggctgacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 368 - DNA, 252 bp, Artificial sequence; identical to SEQ ID NO: 103
  16S rRNA  amino acid sequence

SEQ ID NO: 369 - DNA, 252 bp, Artificial sequence
  16S rRNA  amino acid sequence
tacgtagggagcaagcgttgtccggatttactgggtgtaaagggtgcgtaggcggctttttaagtcagatgtgaaatctatgggctcaacccataaactgcatttgaaactagagagcttgagtgaagtagaggcaggcggaattccctgtgtagcggtgaaatgcgtagagatagggaggaacaccagtggcgaaggcggcctgctgggctttaactgacgctgaggcacgaaagcgtgggtagcaaacag

SEQ ID NO: 370 - DNA, 252 bp, Artificial sequence
  16S rRNA  amino acid sequence
tacgtaggtggcaagcgttgtccggatttactgggtgtaaagggagcgtagactgcatggcaagtcagatgtgaaagcccggggcttaacctcggaactgcatttgaaactgccaagctagagtacaggagaggtaagtggaattcctagtgtagcggtgaaatgcgtagatattaggaggaacaccggtggcgaaggcggcttactggactgtaactgacgttgaggctcgaaagcgtggggagcaaacag

SEQ ID NO: 371 - DNA, 252 bp, Artificial sequence
  16S rRNA  amino acid sequence
tacggaaggtccaggcgttatccggatttattgggtttaaagggagcgtaggccggagattaagtgtgttgtgaaatttaggcgctcaacgtctgcactgcagcgcatactggtttccttgagtacacgcaacgttggcggaattcgtcgtgtagcggtgaaatgcttagatatcacgaagaactccgattgcgaaggcagccaacaaggcctttactgacgctaaagctcgaaggtgcgggtatcgaacag

SEQ ID NO: 372 - DNA, 252 bp, Artificial sequence
  16S rRNA  amino acid sequence
tacggaaggtccgggcgttatccggatttattgggtttaaagggagcgtaggcggtattttaagcgtgcagtgaaaaccaggtgcttaacatctgacgtgctgcgcgaactggagtacttgagtgagcggaacggaggcggaatttgtggtgtagcggtgaaatgcttagatatcacgaagaactccgattgcgaaggcagccaacaaggcctttactgacgctaaagctcgaaggtgcgggtatcgaacag

SEQ ID NO: 373 - DNA, 251 bp, Artificial sequence; identical to SEQ ID NO: 107
  16S rRNA  amino acid sequence

SEQ ID NO: 374 - DNA, 252 bp, Artificial sequence; identical to SEQ ID NO: 111
  16S rRNA  amino acid sequence

SEQ ID NO: 375 - DNA, 252 bp, Artificial sequence; identical to SEQ ID NO: 112
  16S rRNA  amino acid sequence

SEQ ID NO: 376 - DNA, 252 bp, Artificial sequence
  16S rRNA  amino acid sequence
tacggaaggtccgggcgttatccggatttattgggtttaaagggagcgtaggccgcagattaagtgtgttgtgaaatgtagaggctcaacctctgaattgcagcgcatactggtctgcttgagtacgcacaacgtaggcggaatttgtcgtgtagcggtgaaatgcttagatatgacgaagaactccgattgcgaaggcagccttctggggtgcgattgacgctgaggctcgaaagtgcgggtatcgaacag

SEQ ID NO: 377 - DNA, 252 bp, Artificial sequence
  16S rRNA  amino acid sequence
tacgtagggggcaagcgttatccggatttactgggtgtaaagggagcgcaggcggtcgggcaagtcagaagtggaagcacggggctcaaccccgtgactgcttttgaaactggtcgacttgagtacgggagaggcaggcagaattcctggtgtagcggtgaaatgcatagatatcaggaagaataccggtggcgaaggcggcctgctggaccgagactgacgctgaggctcgaaagcgtgggtagcaaacag

SEQ ID NO: 378 - DNA, 252 bp, Artificial sequence; identical to SEQ ID NO: 117
  16S rRNA  amino acid sequence

SEQ ID NO: 379 - DNA, 252 bp, Artificial sequence; identical to SEQ ID NO: 119
  16S rRNA  amino acid sequence

SEQ ID NO: 380 - DNA, 251 bp, Artificial sequence; identical to SEQ ID NO: 121
  16S rRNA  amino acid sequence

SEQ ID NO: 381 - DNA, 253 bp, Artificial sequence; identical to SEQ ID NO: 122
  16S rRNA  amino acid sequence

SEQ ID NO: 382 - DNA, 252 bp, Artificial sequence
  16S rRNA  amino acid sequence
tacggaaggtccgggcgttatccggatttattgggtttaaagggagcgcaggcggggaggtaagcgtgacgtgaaatgtagccgctcaacggctgaactgcgtcgcgaactgtctctcttgagtgagtacgacgtcagcggaatttgtggtgtagcggtgaaatgcttagatatcacgaagaaccccgattgcgaaggcagctgacgagtcctttactgacgctaaagctcgaaggtgcgggtatcgaacag

SEQ ID NO: 383 - DNA, 252 bp, Artificial sequence; identical to SEQ ID NO: 124
  16S rRNA  amino acid sequence

SEQ ID NO: 384 - DNA, 252 bp, Artificial sequence
  16S rRNA  amino acid sequence
tacggaggatgcaagcgttatccggatttattgggtttaaagggtgcgtaggcggacttttaagcgtgaggtgaaatgcgcgggctcaacctgtgaactgcctcgcgaactgggagtctggaatgtccgtgccgtgggaggaatgtgtggtgtagcggtgaaatgcatagagatcacacagaacaccgattgcgaaggcatctcacgaacggatgattgacgctgaggcacgaaagcgtggggatcaaacag

SEQ ID NO: 385 - DNA, 252 bp, Artificial sequence; identical to SEQ ID NO: 125
  16S rRNA  amino acid sequence

SEQ ID NO: 386 - DNA, 252 bp, Artificial sequence; identical to SEQ ID NO: 125
  16S rRNA  amino acid sequence

SEQ ID NO: 387 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgcctgacttgcagcgcgaactgacggtcttgagtgcgcgcaacgttggcggaatttgtcgtgtagcggtgaaatgcttagatatgacgaagaaccccgattgcgaaggcagctcgcgggagcgcaactgacgctgaagctcgaaagtgcgggtatcgaacag

SEQ ID NO: 388 - DNA, 252 bp, Artificial sequence; identical to SEQ ID NO: 127
  16S rRNA  amino acid sequence

SEQ ID NO: 389 - DNA, 251 bp, Artificial sequence; identical to SEQ ID NO: 128
  16S rRNA  amino acid sequence

SEQ ID NO: 390 - DNA, 252 bp, Artificial sequence; identical to SEQ ID NO: 130
  16S rRNA  amino acid sequence

SEQ ID NO: 391 - DNA, 252 bp, Artificial sequence
  16S rRNA  amino acid sequence
tacggaggatgcgggcgttatccggatttattaggtctaaagggtgcgcaggcggctctctaagtcggcggtgaaaacccgtggcccaaccacggacgtgccatcgatactggggagcttgagtctggatgaggtatgcggaatgagtggtgtagcggtgaaatgcttagatattactcagaacaccgattgcgaaggcagcgtgccaatcctcgactgacgctcatgcacgaaagcgcgggtagcgaacag

SEQ ID NO: 392 - DNA, 252 bp, Artificial sequence; identical to SEQ ID NO: 391
  16S rRNA  amino acid sequence

SEQ ID NO: 393 - DNA, 252 bp, Artificial sequence; identical to SEQ ID NO: 131
  16S rRNA  amino acid sequence

SEQ ID NO: 394 - DNA, 252 bp, Artificial sequence
  16S rRNA  amino acid sequence
tacgtagggagcaagcgttgtccggatttactgggtgtaaagggtgcgtaggcggattggcaagtcagtcgtgaaatctgtgggctcaactcacaaattgcgattgaaactgtcagtcttgagtgaagtagaggcaggcggaattcccggtgtagcggtgaaatgcgtagagatcgggaggaacaccagtggcgaaggcggcctgctgggctttaactgacgctgaggcacgaaagcatgggtagcaaacag

SEQ ID NO: 395 - DNA, 252 bp, Artificial sequence; identical to SEQ ID NO: 394
  16S rRNA  amino acid sequence

SEQ ID NO: 396 - DNA, 252 bp, Artificial sequence
  16S rRNA  amino acid sequence
tacggaggatgcgagcgttatccggatttattgggtttaaagggtgcgtaggcgggttgccaagtcaggggtgaaatcccggagcctaactccggaactgcctttgatactggcagtcttgaattcggatgccgtgggaggaatgagtagtgtagcggtgaaatgcatagatattactcagaacaccgattgcgaaggcatctcacgaatccgacattgacgctgaggcacgaaagcgtggggatcaaacag

SEQ ID NO: 397 - DNA, 252 bp, Artificial sequence; identical to SEQ ID NO: 132
  16S rRNA  amino acid sequence

SEQ ID NO: 398 - DNA, 251 bp, Artificial sequence; identical to SEQ ID NO: 134
  16S rRNA  amino acid sequence

SEQ ID NO: 399 - DNA, 252 bp, Artificial sequence; identical to SEQ ID NO: 135
  16S rRNA  amino acid sequence

SEQ ID NO: 400 - DNA, 252 bp, Artificial sequence; identical to SEQ ID NO: 136
  16S rRNA  amino acid sequence

SEQ ID NO: 401 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggggctcaacctctgaactgcagcgcgaactgacggtcttgagtgcgcgcaacgttggcggaattcgtcgtgtagcggtgaaatgcttagatatgacgaagaactccgattgcgaaggcagcccacaaggcctttactgacgctaaagctcgaaggtgcgggtatcgaacag

SEQ ID NO: 402 - DNA, 252 bp, Artificial sequence
  16S rRNA  amino acid sequence
tacggaaggtccgggcgttatccggatttattgggtttaaagggagcgcaggccggctggtaagcgtgacgtgaaatgtagttgctcaacatctgcactgcgtcgcgaactgtcagccttgagtgagtacgatgttggcggaattcgtggtgtagcggtgaaatgcttagatatcacgaagaactccgattgcgaaggcagccaacaaggcctttactgacgctaaagctcgaaggtgcgggtatcgaacag

SEQ ID NO: 403 - DNA, 252 bp, Artificial sequence; identical to SEQ ID NO: 137
  16S rRNA  amino acid sequence

SEQ ID NO: 404 - DNA, 252 bp, Artificial sequence; identical to SEQ ID NO: 137
  16S rRNA  amino acid sequence

SEQ ID NO: 405 - DNA, 252 bp, Artificial sequence; identical to SEQ ID NO: 138
  16S rRNA  amino acid sequence

SEQ ID NO: 406 - DNA, 253 bp, Artificial sequence
  16S rRNA  amino acid sequence
tacggaaggtccgggcgttatccggatttattgggtttaaagggagcgcaggcggacagttaagcgtgacgtgaaatgcagcggctcaaccgtatgaagtgcgtcgcgaactggcagtcttgagtgagtacgatgtaggcggaattcgtggtgtagcggtgaaatgcttagatatcacgaagaactccgattgcgaaggcagcctacaagtcctttactgacgctaaagctcgaaggtgcgggtatcgaacag

SEQ ID NO: 407 - DNA, 251 bp, Artificial sequence; identical to SEQ ID NO: 140
  16S rRNA  amino acid sequence

SEQ ID NO: 408 - DNA, 252 bp, Artificial sequence; identical to SEQ ID NO: 141
  16S rRNA  amino acid sequence

SEQ ID NO: 409 - DNA, 252 bp, Artificial sequence
  16S rRNA  amino acid sequence
tacggaggatgcaagcgttatccggatttattgggtttaaagggtgcgtaggcggaccgttaagtcagcggtgaaatgtcccggctcaaccggggagctgccgttgatactggcggactagggtccaaacggcgccggcggaatgtgtcatgtagcggtgaaatgcttagatatgccacagaacaccgatcgcgaaggcagctggcgaggccggtactgacgctcaggcacgaaagcgtggggatcaaacag

SEQ ID NO: 410 - DNA, 252 bp, Artificial sequence
  16S rRNA  amino acid sequence
tacggaggatgcaagcgttatccggatttattgggtttaaagggtgcgtaggctgtggggaaagtcaggggtgaaagcccggcgcttaacgccggaactgcctttgatacttctccgctggaatacggatgccgtgggaggaatgagtagtgtagcggtgaaatgcatagatattactcagaacaccgattgcgaaggcatctcacgaatccgtcattgacgctgaggcacgaaagcgtggggatcaaacag

SEQ ID NO: 411 - DNA, 252 bp, Artificial sequence; identical to SEQ ID NO: 145
  16S rRNA  amino acid sequence

SEQ ID NO: 412 - DNA, 252 bp, Artificial sequence
  16S rRNA  amino acid sequence
tacggaaggtccgggcgttatccggatttattgggtttaaagggagcgcaggcggggagctaagcgtgacgtgaaatgtagccgctcaacggctgaactgcgtcgcgaactggctttcttgagtgagtacgacgtcagcggaattcgtggtgtagcggtgaaatgcttagatatcacgaagaactccgattgcgaaggcagccaacaaggcctttactgacgctaaagctcgaaggtgcgggtatcgaacag

SEQ ID NO: 413 - DNA, 252 bp, Artificial sequence; identical to SEQ ID NO: 412
  16S rRNA  amino acid sequence

SEQ ID NO: 414 - DNA, 251 bp, Artificial sequence; identical to SEQ ID NO: 146
  16S rRNA  amino acid sequence

SEQ ID NO: 415 - DNA, 252 bp, Artificial sequence; identical to SEQ ID NO: 147
  16S rRNA  amino acid sequence

SEQ ID NO: 416 - DNA, 252 bp, Artificial sequence; identical to SEQ ID NO: 148
  16S rRNA  amino acid sequence

SEQ ID NO: 417 - DNA, 252 bp, Artificial sequence; identical to SEQ ID NO: 148
  16S rRNA  amino acid sequence

SEQ ID NO: 418 - DNA, 252 bp, Artificial sequence
  16S rRNA  amino acid sequence
tacgtagggagcaagcgttgtccggatttactgggtgtaaagggtgcgtaggcggccgagtaagtcagatgtgaaatctatgggctcaacccataaactgcatttgaaactacccggcttgagtgaagtagaggcaggcggaattccctgtgtagcggtgaaatgcgtagagatagggaggaacaccagtggcgaaggcggcctgctgggctttaactgacgctgaggcacgaaagcgtgggtagcaaacag

SEQ ID NO: 419 - DNA, 251 bp, Artificial sequence
  16S rRNA  amino acid sequence
tacgtagggggcgagcgttatccggattcattgggcgtaaagcgcgcgtaggccgcccgcttggtcgggagtaaaatccgggggctcaacccccgcccgctcccgataccggcgggcttgagtctggcaggggaaggtggaattccgagtgtagcggtggaatgcgcagatattcggaagaacaccggtggcgaaggcggccttctgggccacgactgacgctgaggcgcgaaagctaggggagcgaacag

SEQ ID NO: 420 - DNA, 252 bp, Artificial sequence; identical to SEQ ID NO: 155
  16S rRNA  amino acid sequence

SEQ ID NO: 421 - DNA, 252 bp, Artificial sequence
  16S rRNA  amino acid sequence
tacggaaggtccgggcgttatccggatttattgggtttaaagggagcgcaggccggaggtcaagcgtgacgtgaaatgtagccgctcaacggctgaactgcgtcgcgaactggctttcttgagtgagttcgatgtgggcggaattcgtggtgtagcggtgaaatgcttagatatcacgaagaactccgattgcgaaggcagcccacaaggcctttactgacgctaaagctcgaaggtgcgggtatcgaacag

SEQ ID NO: 422 - DNA, 252 bp, Artificial sequence; identical to SEQ ID NO: 157
  16S rRNA  amino acid sequence

SEQ ID NO: 423 - DNA, 252 bp, Artificial sequence
  16S rRNA  amino acid sequence
tacggaaggtccgggcgttatccggatttattgggtttaaagggagcgcaggcgggttattaagcgtgttgtgaaatgcggcggctcaaccgtcgaattgcagcgcgaactggttaccttgagtgagtacgacgtcagcggaattcgtggtgtagcggtgaaatgcttagatatcacgaagaactccgattgcgaaggcagccaacaaggcctttactgacgctaaagctcgaaggtgcgggtatcgaacag

SEQ ID NO: 424 - DNA, 252 bp, Artificial sequence
  16S rRNA  amino acid sequence
tacggaaggtccgggcgttatccggatttattgggtttaaagggagcgcaggctgaagattaagcgtgacgtgaaatgtcccggctcaaccggggagttgcgtcgcgaactggttttcttgagtgagtacgacgtttgcggaattcgtggtgtagcggtgaaatgcttagatatcacgaagaactccgattgcgaaggcagcagacgagtcctttactgacgctaatgctcgaaagtgcgggtatcgaacag

SEQ ID NO: 425 - DNA, 251 bp, Artificial sequence; identical to SEQ ID NO: 160
  16S rRNA  amino acid sequence

SEQ ID NO: 426 - DNA, 252 bp, Artificial sequence; identical to SEQ ID NO: 161
  16S rRNA  amino acid sequence

SEQ ID NO: 427 - DNA, 252 bp, Artificial sequence
  16S rRNA  amino acid sequence
tacggaaggtccgggcgttatccggatttattgggtttaaagggagcgtaggccgtggattaagtgtgttgtgaaatgtaggcgctcaacgtctgacttgcagcgcatactggtccacttgagtgcgcacaacgcgggcggaatttgtcgtgtagcggtgaaatgcttagatatgacgaagaactccgattgcgaaggcagccttctggggtgcgattgacgctgaggctcgaaagtgcgggtatcgaacag

SEQ ID NO: 428 - DNA, 252 bp, Artificial sequence; identical to SEQ ID NO: 163
  16S rRNA  amino acid sequence

SEQ ID NO: 429 - DNA, 252 bp, Artificial sequence; identical to SEQ ID NO: 164
  16S rRNA  amino acid sequence

SEQ ID NO: 430 - DNA, 252 bp, Artificial sequence; identical to SEQ ID NO: 164
  16S rRNA  amino acid sequence

SEQ ID NO: 431 - DNA, 252 bp, Artificial sequence
  16S rRNA  amino acid sequence
tacggaaggtccgggcgttatccggatttattgggtttaaagggagcgcaggcgggaccttaagcgtgttgtgaaatgtggcggctcaaccgccgaactgcagcgcgaactggggttcttgagtgagtacgaggaaggcggaattcgtggtgtagcggtgaaatgcttagatatcacgaagaactccgattgcgaaggcagctttccagtcctttactgacgctaatgctcgaaggtgcgggtatcgaacag

SEQ ID NO: 432 - DNA, 252 bp, Artificial sequence; identical to SEQ ID NO: 166
  16S rRNA  amino acid sequence

SEQ ID NO: 433 - DNA, 252 bp, Artificial sequence; identical to SEQ ID NO: 167
  16S rRNA  amino acid sequence

SEQ ID NO: 434 - DNA, 252 bp, Artificial sequence
  16S rRNA  amino acid sequence
tacggaaggtccaggcgttatccggatttattgggtttaaagggagcgtaggccggagattaagtgtgttgtgaaatttaggcgctcaacgtctgcactgcagcgcatactggtctgcttgagtacgcacaacgtaggcggaatttgtcgtgtagcggtgaaatgcttagatatgacgaagaactccgattgcgaaggcagcttacgggagcgcaactgacgctgaagctcgaaagtgcgggtatcgaacag

SEQ ID NO: 435 - DNA, 251 bp, Artificial sequence
  16S rRNA  amino acid sequence
tacgtagggagcgagcgttatccggaattattgggcgtaaagggtgcgtagatggcaatataagttttatgtaaaattgttgggctcaacccaatagggcataagatactgtatagctagagtattaaagaggcaagtggaactccatgtgtagcggtaaaatgcgtaaatatatggaagaacaccagtggcgaaggcggcttgctagttaaatactgacattgaggcacgaaagcgtggggagcaaacag

SEQ ID NO: 436 - DNA, 252 bp, Artificial sequence
  16S rRNA  amino acid sequence
tacgtatggtgcaagcgttatccggatttactgggtgtaaagggtgcgtaggtggcatggtaagtcagatgtgaaaactatgggctcaacccatagcctgcatttgaaactgtcgagctagagtacaggagaggaaagtggaattcctagtgtagcggtgaaatgcgtagatattaggaggaacaccagtggcgaaggcggctttctggactgttactgacactgaggtgcgaaagcgtgggtagcaaacag

SEQ ID NO: 437 - DNA, 251 bp, Artificial sequence; identical to SEQ ID NO: 173
  16S rRNA  amino acid sequence

SEQ ID NO: 438 - DNA, 252 bp, Artificial sequence; identical to SEQ ID NO: 176
  16S rRNA  amino acid sequence

SEQ ID NO: 439 - DNA, 252 bp, Artificial sequence; identical to SEQ ID NO: 177
  16S rRNA  amino acid sequence

SEQ ID NO: 440 - DNA, 252 bp, Artificial sequence
  16S rRNA  amino acid sequence
tacggaaggtccgggcgttatccggatttattgggtttaaagggagcgcaggccgtttggtaagcgtgttgtgaaatgtccgggctcaacctgggcactgcagcgcgaactggctgacttgagtacgcacgacgttggcggaatttgccgtgtagcggtgaaatgcttagatatggcgaagaactccgattgcgaaggcagctgacgggagcgcaactgacgctcatgctcgaaagtgcgggtatcgaacag

SEQ ID NO: 441 - DNA, 252 bp, Artificial sequence
  16S rRNA  amino acid sequence
tacggaaggtccgggcgttatccggatttattgggtttaaagggagcgcaggcggccttttaagcgtgacgtgaaatgtaggcgctcaacgtctgaattgcgtcgcgaactgatgggcttgagtctacggaatgccggcggaattcgtggtgtagcggtgaaatgcttagatatcacgaagaactccgattgcgaaggcagctggcagcagtcagactgacgctcatgctcgaaagtgcgggtatcgaacag

SEQ ID NO: 442 - DNA, 252 bp, Artificial sequence
  16S rRNA  amino acid sequence
tacggaaggtccgggcgttatccggatttattgggtttaaagggagcgcaggccgggttttaagcgtgacgtgaaatgtggcggctcaaccgccgaactgcgtcgcgaactggagtccttgagtgagtacgacggaggcggaattcgtggtgtagcggtgaaatgcttagatatcacgaagaaccccgattgcgaaggcagcctccgagtccttaactgacgctaaagctcgaaagtgcgggtatcgaacag

SEQ ID NO: 443 - DNA, 252 bp, Artificial sequence
  16S rRNA  amino acid sequence
tacgtagggagcgagcgttgtccggaattactgggtgtaaagggagcgtaggcggtatcataagtcagatgtgaaatgtatgggctcaacccataaattgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacag

SEQ ID NO: 444 - DNA, 252 bp, Artificial sequence; identical to SEQ ID NO: 179
  16S rRNA  amino acid sequence

SEQ ID NO: 445 - DNA, 252 bp, Artificial sequence; identical to SEQ ID NO: 183
  16S rRNA  amino acid sequence

SEQ ID NO: 446 - DNA, 251 bp, Artificial sequence; identical to SEQ ID NO: 184
  16S rRNA  amino acid sequence

SEQ ID NO: 447 - DNA, 252 bp, Artificial sequence
  16S rRNA  amino acid sequence
tacggaaggtccgggcgttatccggatttattgggtttaaagggagcgcaggcggagacttaagcgtgctgtgaaatgctgcggctcaaccgtggaagtgcagcgcgaactgggtttcttgagtgagtgcgaggtatgcggaattcgtggtgtagcggtgaaatgcttagatatcacgaagaaccccgattgcgaaggcagcgtaccagtccttaactgacgctaaagctcgaaagtgcgggtatcgaacag

SEQ ID NO: 448 - DNA, 252 bp, Artificial sequence; identical to SEQ ID NO: 185
  16S rRNA  amino acid sequence

SEQ ID NO: 449 - DNA, 252 bp, Artificial sequence; identical to SEQ ID NO: 186
  16S rRNA  amino acid sequence

SEQ ID NO: 450 - DNA, 252 bp, Artificial sequence
  16S rRNA  amino acid sequence
tacagagggtgcaagcgttgttcggaattactgggcgtaaagggcgtgtaggcggttagataagtcagatgttaaagacccgggcttaacctgggggctgcgtttgatactgtttaactagagagcaggaggggagaatggaatttccggtgtagaggtaaaattcgtagatatcggaaggaacaccagtggcgaaggcgattctctggactgcaactgacgctgaggcgcgaaagcgtggggagcaaacag

SEQ ID NO: 451 - DNA, 252 bp, Artificial sequence
  16S rRNA  amino acid sequence
tacggaaggtccgggcgttatccggatttattgggtttaaagggagcgtaggcggtattttaagcgtgcagtgaaaaccaggtgcttaacatctgacgtgctgcgcgaactggagtacttgagtgagcggaacggaggcggaatttgtggtgtagcggtgaaatgcttagatatcacgaagaactccgattgcgaaggcagctttcggtagctttactgacgctgatgctcgaaggtgcgggtatcgaacag

SEQ ID NO: 452 - DNA, 252 bp, Artificial sequence
  16S rRNA  amino acid sequence
tacgtagggagcgagcgttgtccggaattactgggtgtaaagggagcgtaggcgggatggcaagtcagatgtgaaaactatgggctcaacccatagactgcatttgaaactgttgttcttgagtgaagtagaggtaagcggaattcctggtgtagcggtgaaatgcgtagatatcaggaggaacatcggtggcgaaggcggcttactgggcttttactgacgctgaggctcgaaagcgtggggagcaaacag

SEQ ID NO: 453 - DNA, 252 bp, Artificial sequence; identical to SEQ ID NO: 192
  16S rRNA  amino acid sequence

SEQ ID NO: 454 - DNA, 252 bp, Artificial sequence; identical to SEQ ID NO: 193
  16S rRNA  amino acid sequence

SEQ ID NO: 455 - DNA, 252 bp, Artificial sequence; identical to SEQ ID NO: 194
  16S rRNA  amino acid sequence

SEQ ID NO: 456 - DNA, 252 bp, Artificial sequence; identical to SEQ ID NO: 195
  16S rRNA  amino acid sequence

SEQ ID NO: 457 - DNA, 252 bp, Artificial sequence; identical to SEQ ID NO: 196
  16S rRNA  amino acid sequence

SEQ ID NO: 458 - DNA, 252 bp, Artificial sequence; identical to SEQ ID NO: 197
  16S rRNA  amino acid sequence

SEQ ID NO: 459 - DNA, 252 bp, Artificial sequence; identical to SEQ ID NO: 197
  16S rRNA  amino acid sequence

SEQ ID NO: 460 - DNA, 252 bp, Artificial sequence; identical to SEQ ID NO: 199
  16S rRNA  amino acid sequence

SEQ ID NO: 461 - DNA, 252 bp, Artificial sequence
  16S rRNA  amino acid sequence
tacggaaggtccgggcgttatccggatttattgggtttaaagggagcgcaggctggagattaagcgtgacgtgaaatgccgcggctcaaccgtggcactgcagcgcgaactggggggcttgagtgcgcgcagcgaaggcggaattcgtggtgtagcggtgaaatgcttagatatcacgaagaactccgatcgcgaaggcagccttcgggagcgcaactgacgctgaagctcgaaagtgcgggtatcgaacag

SEQ ID NO: 462 - DNA, 252 bp, Artificial sequence; identical to SEQ ID NO: 201
  16S rRNA  amino acid sequence

SEQ ID NO: 463 - DNA, 251 bp, Artificial sequence
  16S rRNA  amino acid sequence
tacggaggatgcgagcgttatccggatttattgggtttaaagggtgcgtaggtggtttgttaagtttgtggtgaaagcgtgcggctcaaccgtaccaagccatgaaaactggcgaacttgagtgcaaacgaggtaggcggaatgtgatgtgtagcggtgaaatgcttagatatggcgaagaactccgattgcgaaggcagctgacgggagcgcaactgacgctcatgctcgaaagtgcgggtatcgaacag

SEQ ID NO: 464 - DNA, 252 bp, Artificial sequence
  16S rRNA  amino acid sequence
tacggaaggtccgggcgttatccggatttattgggtttaaagggagcgcaggccgatggttaagcgtgacgtgaaatgtagccgctcaacggctgaactgcgtcgcgaactggctttcttgagtgagtacgacgtcagcggaattcgtggtgtagcggtgaaatgcttagatatcacgaagaaccccgattgcgaaggcagctgacgagtccttaactgacgctaaagctcgaaggtgcgggtatcgaacag

SEQ ID NO: 465 - DNA, 252 bp, Artificial sequence; identical to SEQ ID NO: 202
  16S rRNA  amino acid sequence

SEQ ID NO: 466 - DNA, 251 bp, Artificial sequence
  16S rRNA  amino acid sequence
tacgtaggtggcgagcgttgtccggaattattgggcgtaaagggagcgcaggcgggaaggcaagtcagtcttaaaagtgcggggctcaaccccgtgatgggattgaaactgtctttcttgagtgcaggagaggaaagcggaattcctagtgtagcggtgaaatgcgtagatattaggaggaacaccagtggcgaaggcggctttctggactgtaactgacgctgaggctcgaaagcgtggggagcgaacag

SEQ ID NO: 467 - DNA, 252 bp, Artificial sequence
  16S rRNA  amino acid sequence
tacggaaggtccgggcgttatccggattcattgggtttaaagggagcgtaggccgccccttaagcgtgttgtgaaatgccgcggctcaaccgtggcactgcagcgcgaactggggggcttgagtgcgcgcagcgaaggcggaattcgtggtgtagcggtgaaatgcttagatatcacgaagaactccgattgcgaaggcagccaacaaggcctttactgacgctaaagctcgaaggtgcgggtatcgaacag

SEQ ID NO: 468 - DNA, 252 bp, Artificial sequence
  16S rRNA  amino acid sequence
tacgtatggtgcaagcgttatccggatttactgggtgtaaagggagcgtaggcggttttacaagtctgatgtgaaagcccggggctcaactccggtattgcattggaaactgtagaactagagtgtcggaggggtaagtggaattcctagtgtagcggtgaaatgcgtagatattaggaggaacaccagtggcgaaggcggcttactggacgattactgacgctgaggctcgaaagcgtggggagcaaacag

SEQ ID NO: 469 - DNA, 252 bp, Artificial sequence
  16S rRNA  amino acid sequence
tacggaggatgcaagcgttatccggatttattgggtttaaagggtgcgtaggcggagctttaagcgtgaggtgaaatgccacggctcaaccgtggaactgcctcgcgaactggagttctggaatgcccctgccgtgggaggaatgtgtggtgtagcggtgaaatgcatagagatcacacagaacaccgattgcgaaggcatctcacgaaggggtgattgacgctgaggcacgaaagcgtggggatcaaacag

SEQ ID NO: 470 - DNA, 252 bp, Artificial sequence; identical to SEQ ID NO: 203
  16S rRNA  amino acid sequence

SEQ ID NO: 471 - DNA, 252 bp, Artificial sequence; identical to SEQ ID NO: 204
  16S rRNA  amino acid sequence

SEQ ID NO: 472 - DNA, 251 bp, Artificial sequence
  16S rRNA  amino acid sequence
tacggaaggtccgggcgttatccggatttattgggtttaaagggagcgtaggcggcccgttaagtcagcggtcaaatgtcagtgcccaacattggcaagccgttgatactggtgggcttgaatacacacaaggaaggtggaattcgtcgtgtagcggtgaaatgcttagatatgacgaagaactccgattgcgaaggcagccttctggggtgcgattgacgctgaggctcgaaagtgcgggtatcgaacag

SEQ ID NO: 473 - DNA, 252 bp, Artificial sequence; identical to SEQ ID NO: 209
  16S rRNA  amino acid sequence

SEQ ID NO: 474 - DNA, 252 bp, Artificial sequence; identical to SEQ ID NO: 209
  16S rRNA  amino acid sequence

SEQ ID NO: 475 - DNA, 252 bp, Artificial sequence; identical to SEQ ID NO: 213
  16S rRNA  amino acid sequence

SEQ ID NO: 476 - DNA, 252 bp, Artificial sequence; identical to SEQ ID NO: 214
  16S rRNA  amino acid sequence

SEQ ID NO: 477 - DNA, 252 bp, Artificial sequence; identical to SEQ ID NO: 216
  16S rRNA  amino acid sequence

SEQ ID NO: 478 - DNA, 252 bp, Artificial sequence; identical to SEQ ID NO: 216
  16S rRNA  amino acid sequence

SEQ ID NO: 479 - DNA, 252 bp, Artificial sequence
  16S rRNA  amino acid sequence
cacgtaagttgcgagcgttgttcggaattattgggcgtaaagggcatgtaggcggaaccgcaagtctggtgtgaaaggcaggggctcaacccctggactgcgttggaaactgcggtactagagtcgctgaggggcaggcagaattccaggtgtaggggtgaaatctgtagatatctggaagaataccgatggcgaaggcagcctgccagcagatgactgacgctgaggtgcgaaggtgcggggagcaaacag

SEQ ID NO: 480 - DNA, 252 bp, Artificial sequence; identical to SEQ ID NO: 223
  16S rRNA  amino acid sequence

SEQ ID NO: 481 - DNA, 252 bp, Artificial sequence
  16S rRNA  amino acid sequence
tacgtagggggcaagcgttatccggatttactgggtgtaaagggagcgtagacggatttgcaagtcagatgtgaaaggcgggggcttaacccctggattgcatttgaaactgcagatcttgagtaccggaagggcagatggaattcctggtgtagcggtgaaatgcgtagagatcaggaggaacaccggtggcgaaagcggtctgctggacggtaactgacgttgaggctcgaaggcgtggggagcaaacag

SEQ ID NO: 482 - DNA, 252 bp, Artificial sequence
  16S rRNA  amino acid sequence
tacggaaggtccgggcgttatccggatttattgggtttaaagggagcgcaggcgggttattaagcgtgttgtgaaatgcggcggctcaaccgtcgaattgcagcgcgaactggttaccttgagtgagtacgacgtcagcggaattcgtggtgtagcggtgaaatgcttagatatcacgaagaaccccgattgcgaaggcagctgacgagtccttaactgacgctaatgctcgaaagtgcgggtatcgaacag

SEQ ID NO: 483 - DNA, 252 bp, Artificial sequence
  16S rRNA  amino acid sequence
tacggaggatgcgagcgttatccggatttattgggtttaaagggagcgtagatgggagcttaagccagttgtgaaaagcgcgggcccaacccgcgtcgtgcaattggaactgtgttccttgagtgggttcggggcaggtggaattcgtggtgtagcggtgaaatgcttagatatcacgaagaactccgacagcgtaggcatcctgccaggcccttactgacattgaagctcgaaggtgcgggtatcaaacag

SEQ ID NO: 484 - DNA, 251 bp, Artificial sequence; identical to SEQ ID NO: 227
  16S rRNA  amino acid sequence

SEQ ID NO: 485 - DNA, 252 bp, Artificial sequence; identical to SEQ ID NO: 230
  16S rRNA  amino acid sequence

SEQ ID NO: 486 - DNA, 252 bp, Artificial sequence; identical to SEQ ID NO: 231
  16S rRNA  amino acid sequence

SEQ ID NO: 487 - DNA, 252 bp, Artificial sequence; identical to SEQ ID NO: 233
  16S rRNA  amino acid sequence

SEQ ID NO: 488 - DNA, 252 bp, Artificial sequence; identical to SEQ ID NO: 234
  16S rRNA  amino acid sequence

SEQ ID NO: 489 - DNA, 252 bp, Artificial sequence; identical to SEQ ID NO: 235
  16S rRNA  amino acid sequence

SEQ ID NO: 490 - DNA, 252 bp, Artificial sequence; identical to SEQ ID NO: 236
  16S rRNA  amino acid sequence

SEQ ID NO: 491 - DNA, 252 bp, Artificial sequence; identical to SEQ ID NO: 238
  16S rRNA  amino acid sequence

SEQ ID NO: 492 - DNA, 253 bp, Artificial sequence; identical to SEQ ID NO: 240
  16S rRNA  amino acid sequence

SEQ ID NO: 493 - DNA, 252 bp, Artificial sequence; identical to SEQ ID NO: 243
  16S rRNA  amino acid sequence

SEQ ID NO: 494 - DNA, 252 bp, Artificial sequence; identical to SEQ ID NO: 243
  16S rRNA  amino acid sequence

SEQ ID NO: 495 - DNA, 252 bp, Artificial sequence; identical to SEQ ID NO: 245
  16S rRNA  amino acid sequence

SEQ ID NO: 496 - DNA, 252 bp, Artificial sequence
  16S rRNA  amino acid sequence
tacggaaggtccgggcgttatccggatttattgggtttaaagggagcgcaggccggaggtcaagcgtgacgtgaaatgtagccgctcaacggctgaactgcgtcgcgaactggctttcttgagtgagttcgatgtgggcggaattcgtggtgtagcggtgaaatgcttagatatcacgaagaaccccgattgcgaaggcagctgacgagtccttaactgacgctaaagctcgaaagtgcgggtatcgaacag

SEQ ID NO: 497 - DNA, 252 bp, Artificial sequence; identical to SEQ ID NO: 246
  16S rRNA  amino acid sequence

SEQ ID NO: 498 - DNA, 252 bp, Artificial sequence; identical to SEQ ID NO: 248
  16S rRNA  amino acid sequence

SEQ ID NO: 499 - DNA, 252 bp, Artificial sequence; identical to SEQ ID NO: 250
  16S rRNA  amino acid sequence

SEQ ID NO: 500 - DNA, 252 bp, Artificial sequence
  16S rRNA  amino acid sequence
tacggagggtgcgagcgttaatcggaatgactgggcgtaaagggcacgcaggtggcgagataagttggatgtgaaattcccgtgcttaacatggggactgcatccaaaactgttgaactggagtactgtagagggaggtagaattccacgtgtagcggtgaaatgcgtagagattgggaggaacaccagtggcgaaggcgacttactggactgtaactgacgctgaggcgcgaaagtgtggggagcaaacag

SEQ ID NO: 501 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgctcaacgcctgacttgcagcgcgaactgacggtcttgagtgcgcgcaacgttggcggaatttgtcgtgtagcggtgaaatgcttagatatcacgaagaactccgatcgcgaaggcagccttcgggagcgcaactgacgctgaagctcgaaagtgcgggtatcgaacag

SEQ ID NO: 502 - DNA, 252 bp, Artificial sequence; identical to SEQ ID NO: 251
  16S rRNA  amino acid sequence

SEQ ID NO: 503 - DNA, 252 bp, Artificial sequence
  16S rRNA  amino acid sequence
tacgtagggggcaagcgttatccggatttactgggtgtaaagggcacgtaggtggatagataagtcacaagtgaaatacccgggcttaacctgggattggctcgtgaaactgtttaactagagtacggtagaggggagtagaattccaagtgtagcggtgaaatgcgtagagatttggaagaataccggaggcgaaggcggctctctggaccgatactgacactgaagagcgaaagcgtggggagcaaacag

SEQ ID NO: 504 - DNA, 251 bp, Artificial sequence; identical to SEQ ID NO: 254
  16S rRNA  amino acid sequence

SEQ ID NO: 505 - DNA, 252 bp, Artificial sequence; identical to SEQ ID NO: 255
  16S rRNA  amino acid sequence

SEQ ID NO: 506 - DNA, 252 bp, Artificial sequence; identical to SEQ ID NO: 256
  16S rRNA  amino acid sequence

SEQ ID NO: 507 - DNA, 252 bp, Artificial sequence; identical to SEQ ID NO: 257
  16S rRNA  amino acid sequence

SEQ ID NO: 508 - DNA, 251 bp, Artificial sequence; identical to SEQ ID NO: 260
  16S rRNA  amino acid sequence

SEQ ID NO: 509 - DNA, 251 bp, Artificial sequence; identical to SEQ ID NO: 261
  16S rRNA  amino acid sequence

SEQ ID NO: 510 - DNA, 251 bp, Artificial sequence; identical to SEQ ID NO: 261
  16S rRNA  amino acid sequence

SEQ ID NO: 511 - DNA, 252 bp, Artificial sequence
  16S rRNA  amino acid sequence
tacggaaggtccgggcgttatccggatttattgggtttaaagggagcgcaggcgggttgttaagcgtgacgtgaaatgtagccgctcaacggctgcactgcgtcgcgaactggcagtcttgagtgagtacgacgcggacggaattcgtggtgtagcggtgaaatgcttagatatcacgaagaaccccgattgcgaaggcagtccgcgagtcctttactgacgctaaagctcgaaggtgcgggtatcgaacag

SEQ ID NO: 512 - DNA, 252 bp, Artificial sequence; identical to SEQ ID NO: 266
  16S rRNA  amino acid sequence

SEQ ID NO: 513 - DNA, 251 bp, Artificial sequence; identical to SEQ ID NO: 268
  16S rRNA  amino acid sequence

SEQ ID NO: 514 - DNA, 252 bp, Artificial sequence; identical to SEQ ID NO: 269
  16S rRNA  amino acid sequence

SEQ ID NO: 515 - DNA, 252 bp, Artificial sequence; identical to SEQ ID NO: 270
  16S rRNA  amino acid sequence

SEQ ID NO: 516 - DNA, 251 bp, Artificial sequence; identical to SEQ ID NO: 271
  16S rRNA  amino acid sequence

SEQ ID NO: 517 - DNA, 252 bp, Artificial sequence
  16S rRNA  amino acid sequence
tacggaaggtccgggcgttatccggatttattgggtttaaagggagcacaggccggctgtcaagcgtgttgtgaaatgtaggcgctcaacgcctgacttgcagcgcgaactgacggtcttgagtgcgcgcaacgttggcggaatttgtcgtgtagcggtgaaatgcttagatatcacgaagaactccgattgcgaaggcagctgacgagtccttaactgacgctaaagctcgaaagtgcgggtatcgaacag

SEQ ID NO: 518 - DNA, 251 bp, Artificial sequence
  16S rRNA  amino acid sequence
tacggaggatgcgagcgttatccggatttattgggtttaaagggtgcgtaggcgggatgttaagtcagtggtgaaagcgtgtagctcaactataccgagccattgaaactggcgttcttgagtgcaaacgaggtaggcggaatgtgatgtgtagcggtgaaatgcttagatatgtcacagaaccccgattgcgaaggcagcttaccagcatgcaactgacgctgaggcacgaaagcgtgggtatcaaacag

SEQ ID NO: 519 - DNA, 252 bp, Artificial sequence
  16S rRNA  amino acid sequence
tacggaggatgcaagcgttatccggatttattgggtttaaagggtgcgcaggcggcttgttaagcgggaggtgaaatgccggcgctcaacgtcggaagtgcctcccgaactggtgagcttgaatgactctgccgtgggaggaatgtgtagtgtagcggtgaaatgcttagatatgtcacagaaccccgattgcgaaggcagcttaccagcatgcaactgacactgaggcacgaaagcgtgggtatcaaacag

SEQ ID NO: 520 - DNA, 252 bp, Artificial sequence; identical to SEQ ID NO: 273
  16S rRNA  amino acid sequence

SEQ ID NO: 521 - DNA, 252 bp, Artificial sequence; identical to SEQ ID NO: 274
  16S rRNA  amino acid sequence

SEQ ID NO: 522 - DNA, 252 bp, Artificial sequence
  16S rRNA  amino acid sequence
tacgtagggagcaagcgttgtccggatttactgggtgtaaagggtgcgtaggcggcttttcaagtcagctgtgaaatctgtgggctcaacccacaaattgcagttgaaactgtaaagcttgagtgaagtagaggtaggcggaattcccagtgtagcggtgaaatgcgtagagattgggaggaacaccagtggcgaaggcggcctactgggctttaactgacgctgaggcacgaaagcatgggtagcaaacag

SEQ ID NO: 523 - DNA, 252 bp, Artificial sequence
  16S rRNA  amino acid sequence
tacggaaggtccgggcgttatccggatttattgggtttaaagggagcgcaggccggaggctaagcgtgacgtgaaatgtaggcgctcaacgtctgaattgcgtcgcgaactggtttccttgagtgagtgcgacgtgcacggaattcgtggtgtagcggtgaaatgcttagatatcacgaagaaccccgattgcgaaggcagtgcacgagtcctttactgacgctaaagctcgaaagtgcgggtatcgaacag

SEQ ID NO: 524 - DNA, 252 bp, Artificial sequence; identical to SEQ ID NO: 276
  16S rRNA  amino acid sequence

SEQ ID NO: 525 - DNA, 252 bp, Artificial sequence; identical to SEQ ID NO: 277
  16S rRNA  amino acid sequence

SEQ ID NO: 526 - DNA, 252 bp, Artificial sequence
  16S rRNA  amino acid sequence
tacggaaggtccgggcgttatccggatttattgggtttaaagggagcgtaggccgtttcttaagcgtgttgtgaaatgtaggcgcccaacgtctgcactgcagcgcgaactggggaacttgagtacgcgggaagtaggcggaattcgtcgtgtagcggtgaaatgcttagatatgacgaagaactccgattgggaagccagcctgctgtagcgccactgacgctgatgctcgaaagcgtgggtatcgaacag

SEQ ID NO: 527 - DNA, 252 bp, Artificial sequence
  16S rRNA  amino acid sequence
cacgtaggtggcgagcgttgttcggatttattgggcgtaaagggcgcgtaggcggaggctcaagcttggtgtaaaataccccggctcaactggggagatgcgctgagaactgagccactagagtccaggagggggagccggaattccaggtgtaggggtgaaatctgtagatatctggaagaacaccggtggcgaaggcgggctcctggccatggactgacgctgaggcgcgaaagtgtggggagcgaacag

SEQ ID NO: 528 - DNA, 252 bp, Artificial sequence; identical to SEQ ID NO: 281
  16S rRNA  amino acid sequence

SEQ ID NO: 529 - DNA, 252 bp, Artificial sequence; identical to SEQ ID NO: 284
  16S rRNA  amino acid sequence

SEQ ID NO: 530 - DNA, 252 bp, Artificial sequence; identical to SEQ ID NO: 286
  16S rRNA  amino acid sequence

SEQ ID NO: 531 - DNA, 252 bp, Artificial sequence; identical to SEQ ID NO: 294
  16S rRNA  amino acid sequence

SEQ ID NO: 532 - DNA, 252 bp, Artificial sequence; identical to SEQ ID NO: 296
  16S rRNA  amino acid sequence

SEQ ID NO: 533 - DNA, 252 bp, Artificial sequence; identical to SEQ ID NO: 297
  16S rRNA  amino acid sequence

SEQ ID NO: 534 - DNA, 252 bp, Artificial sequence; identical to SEQ ID NO: 298
  16S rRNA  amino acid sequence

SEQ ID NO: 535 - DNA, 251 bp, Artificial sequence; identical to SEQ ID NO: 299
  16S rRNA  amino acid sequence

SEQ ID NO: 536 - DNA, 252 bp, Artificial sequence; identical to SEQ ID NO: 300
  16S rRNA  amino acid sequence

SEQ ID NO: 537 - DNA, 252 bp, Artificial sequence; identical to SEQ ID NO: 304
  16S rRNA  amino acid sequence

SEQ ID NO: 538 - DNA, 252 bp, Artificial sequence; identical to SEQ ID NO: 304
  16S rRNA  amino acid sequence

SEQ ID NO: 539 - DNA, 252 bp, Artificial sequence
  16S rRNA  amino acid sequence
tacggaagatgcgagcgttatccggatttattgggtttaaagggagcgcaggccgatggttaagcgtgacgtgaaatgtagccgctcaacggctgaactgcgtcgcgaactggccgtcttgagtgagttcgatgtgggcggaattcgtggtgtagcggtgaaatgcttagatatcacgaagaactccgattgcgaaggcagcccacaaggcctttactgacgctaaagctcgaaggtgcgggtatcgaacag

SEQ ID NO: 540 - DNA, 252 bp, Artificial sequence; identical to SEQ ID NO: 306
  16S rRNA  amino acid sequence

SEQ ID NO: 541 - DNA, 252 bp, Artificial sequence; identical to SEQ ID NO: 307
  16S rRNA  amino acid sequence

SEQ ID NO: 542 - DNA, 252 bp, Artificial sequence; identical to SEQ ID NO: 309
  16S rRNA  amino acid sequence

SEQ ID NO: 543 - DNA, 252 bp, Artificial sequence; identical to SEQ ID NO: 311
  16S rRNA  amino acid sequence

SEQ ID NO: 544 - DNA, 171 bp, Artificial sequence
  16S rRNA  amino acid sequence
gtttgaaataatttttttccaaaaaaattattgaatgtctttccctgtttggtggagcttgtaaaagtgaagccatcaggggaatggcaaacaataattattttgaaaaagaccttttttaaaattaaacttttgtattcatttgtctaaaattatttttataatataaaa

SEQ ID NO: 545 - DNA, 171 bp, Artificial sequence
  16S rRNA  amino acid sequence
tgtttgaaataatttttttattaaaaaattattgaatgtctttccctgtttggtggagcttgtaaaagtgaagccatcaggggaatggcaaacaataattattttgaaaaagaccttttttaaaattaaactttgtattcatttgtctaaaattatttttataatataaaa

SEQ ID NO: 546 - DNA, 196 bp, Artificial sequence
  16S rRNA  amino acid sequence
gtttgtaataattttttttgattttaaaaaaaattatttatggttttgtctatccctaaaattggtttgttgtgaaatgaatcaaatttagggaataggctttcataaataagttttttttaaagtcttaaagacctttttttaaaattaaacttttgtattcatttgtctaaaattttttttaaataatttaaaa

SEQ ID NO: 547 - DNA, 225 bp, Artificial sequence
  16S rRNA  amino acid sequence
taccatggctctcacgggtaacagggaattagggttcgattctggagaaggagcctgagaaacggctactacatctacggaaggcagcaggcgcgtaaattacccaatcctaaatcagggaggtggtgacaagatataacgacgcggttattatcgcgattgtagtgagggtattctaaacagaacctatagtacgattagagggcaagtctggtgccagcagcc

SEQ ID NO: 548 - DNA, 226 bp, Artificial sequence
  16S rRNA  amino acid sequence
taccatggctttcacgggtaacagggaattagggttcgattctggagaaggagcctgagaaacggctactacatctacggaaggcagcaggcgcgtaaattacccaatcctgactcagggaggtggtgacaagatataatgacgtgatttttaatctcgattgtagtgagggttttctaaatagaacctatagtacgattagagggcaagtctggtgccagcagcc

SEQ ID NO: 549 - DNA, 252 bp, Artificial sequence; identical to SEQ ID NO: 53
  16S rRNA  amino acid sequence

SEQ ID NO: 550 - DNA, 252 bp, Artificial sequence
  16S rRNA  amino acid sequence
tacggaaggtccgggcgttatccggatttattgggtttaaagggagcgcaggccgatgattaagcgtgacgtgaaatgctgaggctcaaccttaggattgcgtcgcgaactggttgtcttgagtgtgtgcgatgttggcggaattcgtggtgtagcggtgaaatgcttagatatcacgaagaactccgattgcgaaggcagctgacaagcacattactgacgctaaagctcgaaagcgcgggtatcgaacag

SEQ ID NO: 551 - DNA, 252 bp, Artificial sequence; identical to SEQ ID NO: 63
  16S rRNA  amino acid sequence

SEQ ID NO: 552 - DNA, 252 bp, Artificial sequence; identical to SEQ ID NO: 64
  16S rRNA  amino acid sequence

SEQ ID NO: 553 - DNA, 252 bp, Artificial sequence; identical to SEQ ID NO: 66
  16S rRNA  amino acid sequence

SEQ ID NO: 554 - DNA, 252 bp, Artificial sequence; identical to SEQ ID NO: 68
  16S rRNA  amino acid sequence

SEQ ID NO: 555 - DNA, 252 bp, Artificial sequence; identical to SEQ ID NO: 80
  16S rRNA  amino acid sequence

SEQ ID NO: 556 - DNA, 252 bp, Artificial sequence; identical to SEQ ID NO: 82
  16S rRNA  amino acid sequence

SEQ ID NO: 557 - DNA, 252 bp, Artificial sequence; identical to SEQ ID NO: 85
  16S rRNA  amino acid sequence

SEQ ID NO: 558 - DNA, 252 bp, Artificial sequence; identical to SEQ ID NO: 89
  16S rRNA  amino acid sequence

SEQ ID NO: 559 - DNA, 252 bp, Artificial sequence; identical to SEQ ID NO: 91
  16S rRNA  amino acid sequence

SEQ ID NO: 560 - DNA, 252 bp, Artificial sequence; identical to SEQ ID NO: 101
  16S rRNA  amino acid sequence

SEQ ID NO: 561 - DNA, 252 bp, Artificial sequence; identical to SEQ ID NO: 104
  16S rRNA  amino acid sequence

SEQ ID NO: 562 - DNA, 252 bp, Artificial sequence; identical to SEQ ID NO: 108
  16S rRNA  amino acid sequence

SEQ ID NO: 563 - DNA, 252 bp, Artificial sequence; identical to SEQ ID NO: 113
  16S rRNA  amino acid sequence

SEQ ID NO: 564 - DNA, 251 bp, Artificial sequence; identical to SEQ ID NO: 133
  16S rRNA  amino acid sequence

SEQ ID NO: 565 - DNA, 252 bp, Artificial sequence; identical to SEQ ID NO: 143
  16S rRNA  amino acid sequence

SEQ ID NO: 566 - DNA, 252 bp, Artificial sequence; identical to SEQ ID NO: 144
  16S rRNA  amino acid sequence

SEQ ID NO: 567 - DNA, 252 bp, Artificial sequence
  16S rRNA  amino acid sequence
tacggaggatgcgagcgttatccggatttattgggtttaaagggagcgcaggctgtcggttaagcgtgttgtgaaatgtaggcgcccaacgcctgcactgcagcgcgaactggctgacttgagtacgcacgacgttggcggaatttgccgtgtagcggtgaaatgcttagatatgacgaagaactccgattgcgaaggcagcttacgggagcgcaactgacgctgaagctcgaaagtgcgggtatcgaacag

SEQ ID NO: 568 - DNA, 252 bp, Artificial sequence; identical to SEQ ID NO: 151
  16S rRNA  amino acid sequence

SEQ ID NO: 569 - DNA, 252 bp, Artificial sequence; identical to SEQ ID NO: 153
  16S rRNA  amino acid sequence

SEQ ID NO: 570 - DNA, 252 bp, Artificial sequence; identical to SEQ ID NO: 156
  16S rRNA  amino acid sequence

SEQ ID NO: 571 - DNA, 252 bp, Artificial sequence; identical to SEQ ID NO: 158
  16S rRNA  amino acid sequence

SEQ ID NO: 572 - DNA, 252 bp, Artificial sequence; identical to SEQ ID NO: 423
  16S rRNA  amino acid sequence

SEQ ID NO: 573 - DNA, 252 bp, Artificial sequence; identical to SEQ ID NO: 163
  16S rRNA  amino acid sequence

SEQ ID NO: 574 - DNA, 252 bp, Artificial sequence; identical to SEQ ID NO: 165
  16S rRNA  amino acid sequence

SEQ ID NO: 575 - DNA, 252 bp, Artificial sequence; identical to SEQ ID NO: 169
  16S rRNA  amino acid sequence

SEQ ID NO: 576 - DNA, 252 bp, Artificial sequence; identical to SEQ ID NO: 172
  16S rRNA  amino acid sequence

SEQ ID NO: 577 - DNA, 252 bp, Artificial sequence; identical to SEQ ID NO: 174
  16S rRNA  amino acid sequence

SEQ ID NO: 578 - DNA, 252 bp, Artificial sequence; identical to SEQ ID NO: 175
  16S rRNA  amino acid sequence

SEQ ID NO: 579 - DNA, 251 bp, Artificial sequence; identical to SEQ ID NO: 178
  16S rRNA  amino acid sequence

SEQ ID NO: 580 - DNA, 251 bp, Artificial sequence
  16S rRNA  amino acid sequence
tacggaggatgcgagcgttatccggatttattgggtttaaagggtgcgcaggcggcgtgtcaagtcagcggtcaaactgcggggctcaaccccgtaccgccgttgaaactggcatgctcgagttcgtgcgatggtggcggaatgcgcggtgtagcggtgaaatgcatagatatcgcgcagaactccgattgcgaaggcagctgccaagcactggactgacgctcatgcacgaaagcgtgggtatcgaacag

SEQ ID NO: 581 - DNA, 252 bp, Artificial sequence
  16S rRNA  amino acid sequence
tacggaaggtccaggcgttatccggatttattgggtttaaagggagcgtaggccggagattaagtgtgttgtgaaatttaggcgctcaacgtctgcactgcagcgcatactggtttccttgagtacacgcaacgttggcggaattcgtcgtgtagcggtgaaatgcttagatatgacgaagaactccgattgcgaaggcagctgacgggagtgcaactgacgctgaagctcgaaagtgcgggtatcgaacag

SEQ ID NO: 582 - DNA, 251 bp, Artificial sequence; identical to SEQ ID NO: 184
  16S rRNA  amino acid sequence

SEQ ID NO: 583 - DNA, 252 bp, Artificial sequence
  16S rRNA  amino acid sequence
tacgtagggagcgagcgttgtccggaattactgggtgtaaagggagcgtaggcgggattgcaagtcaggtgtgaaatgcaggggctcaacccctgaactgcacttgaaactgtagttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacag

SEQ ID NO: 584 - DNA, 252 bp, Artificial sequence; identical to SEQ ID NO: 189
  16S rRNA  amino acid sequence

SEQ ID NO: 585 - DNA, 252 bp, Artificial sequence; identical to SEQ ID NO: 197
  16S rRNA  amino acid sequence

SEQ ID NO: 586 - DNA, 252 bp, Artificial sequence; identical to SEQ ID NO: 200
  16S rRNA  amino acid sequence

SEQ ID NO: 587 - DNA, 252 bp, Artificial sequence
  16S rRNA  amino acid sequence
tacggaaggtccgggcgttatccggatttattgggtttaaagggagcgcaggccgacgtgtaagcgtgttgtgaaatgtaccggctcaaccggtgagttgcagcgcgaactgcatgtcttgagtgcacccgaggagggcggaattcgtggtgtagcggtgaaatgcttagatatcacgaagaactccgattgcgaaggcagccctccagcgtgtaactgacgctaatgctcgaaagtgcgggtatcgaacag

SEQ ID NO: 588 - DNA, 252 bp, Artificial sequence; identical to SEQ ID NO: 205
  16S rRNA  amino acid sequence

SEQ ID NO: 589 - DNA, 252 bp, Artificial sequence; identical to SEQ ID NO: 206
  16S rRNA  amino acid sequence

SEQ ID NO: 590 - DNA, 252 bp, Artificial sequence; identical to SEQ ID NO: 211
  16S rRNA  amino acid sequence

SEQ ID NO: 591 - DNA, 252 bp, Artificial sequence; identical to SEQ ID NO: 216
  16S rRNA  amino acid sequence

SEQ ID NO: 592 - DNA, 252 bp, Artificial sequence; identical to SEQ ID NO: 225
  16S rRNA  amino acid sequence

SEQ ID NO: 593 - DNA, 252 bp, Artificial sequence; identical to SEQ ID NO: 226
  16S rRNA  amino acid sequence

SEQ ID NO: 594 - DNA, 252 bp, Artificial sequence; identical to SEQ ID NO: 229
  16S rRNA  amino acid sequence

SEQ ID NO: 595 - DNA, 252 bp, Artificial sequence; identical to SEQ ID NO: 241
  16S rRNA  amino acid sequence

SEQ ID NO: 596 - DNA, 252 bp, Artificial sequence; identical to SEQ ID NO: 243
  16S rRNA  amino acid sequence

SEQ ID NO: 597 - DNA, 252 bp, Artificial sequence; identical to SEQ ID NO: 248
  16S rRNA  amino acid sequence

SEQ ID NO: 598 - DNA, 252 bp, Artificial sequence; identical to SEQ ID NO: 251
  16S rRNA  amino acid sequence

SEQ ID NO: 599 - DNA, 252 bp, Artificial sequence; identical to SEQ ID NO: 258
  16S rRNA  amino acid sequence

SEQ ID NO: 600 - DNA, 252 bp, Artificial sequence; identical to SEQ ID NO: 259
  16S rRNA  amino acid sequence

SEQ ID NO: 601 - DNA, 252 bp, Artificial sequence; identical to SEQ ID NO: 262
  16S rRNA  amino acid sequence

SEQ ID NO: 602 - DNA, 252 bp, Artificial sequence; identical to SEQ ID NO: 263
  16S rRNA  amino acid sequence

SEQ ID NO: 603 - DNA, 252 bp, Artificial sequence; identical to SEQ ID NO: 267
  16S rRNA  amino acid sequence

SEQ ID NO: 604 - DNA, 252 bp, Artificial sequence; identical to SEQ ID NO: 272
  16S rRNA  amino acid sequence

SEQ ID NO: 605 - DNA, 252 bp, Artificial sequence; identical to SEQ ID NO: 279
  16S rRNA  amino acid sequence

SEQ ID NO: 606 - DNA, 252 bp, Artificial sequence; identical to SEQ ID NO: 285
  16S rRNA  amino acid sequence

SEQ ID NO: 607 - DNA, 253 bp, Artificial sequence; identical to SEQ ID NO: 287
  16S rRNA  amino acid sequence

SEQ ID NO: 608 - DNA, 252 bp, Artificial sequence; identical to SEQ ID NO: 290
  16S rRNA  amino acid sequence

SEQ ID NO: 609 - DNA, 252 bp, Artificial sequence; identical to SEQ ID NO: 301
  16S rRNA  amino acid sequence

SEQ ID NO: 610 - DNA, 252 bp, Artificial sequence; identical to SEQ ID NO: 303
  16S rRNA  amino acid sequence

SEQ ID NO: 611 - DNA, 252 bp, Artificial sequence; identical to SEQ ID NO: 309
  16S rRNA  amino acid sequence

SEQ ID NO: 612 - DNA, 252 bp, Artificial sequence
  16S rRNA  amino acid sequence
tacggaaggtccgggcgttatccggatttattgggtttaaagggagcgcaggccgattattaagcgtgacgtgaaatgtagatgctcaacatctgaactgcgtcgcgaactggtagtcttgagtgagttcgatgtgggcggaattcgtggtgtagcggtgaaatgcttagatatcacgaagaactccgattgcgaaggcagcccacaaggcctttactgacgctaaagctcgaaggtgcgggtatcgaacag

SEQ ID NO: 613 - DNA, 252 bp, Artificial sequence; identical to SEQ ID NO: 222
  16S rRNA  amino acid sequence

SEQ ID NO: 614 - DNA, 252 bp, Artificial sequence; identical to SEQ ID NO: 482
  16S rRNA  amino acid sequence

SEQ ID NO: 615 - DNA, 252 bp, Artificial sequence; identical to SEQ ID NO: 239
  16S rRNA  amino acid sequence

SEQ ID NO: 616 - DNA, 217 bp, Artificial sequence
  16S rRNA  amino acid sequence
gtttgaaataatttttttttgacgtggaataaaaaaaaaattatttatggttttgtctatccctaaattggtttgtttgttgtaaaagacaagtgaatcatttagggaataggctttcctaaataattgattttttttccaatgtcttaaaagacctttttttaaaattaaacttttgtattcatttgtctaaaataatttttaaaataatttaaaa

SEQ ID NO: 617 - DNA, 215 bp, Artificial sequence
  16S rRNA  amino acid sequence
gtttgtaataattttttttgacgtggaataaaaaaaaattatttatggttttgtctatccctaaattggtttgtttgttgtaaaagacaagtgaatcatttagggaataggctttcctaataattgatttttttttccaatgtcttaaaagacctttttttaaaattaaacttttgtattcatttgtctaaaataatttttaaaataatttaaaa

SEQ ID NO: 618 - DNA, 252 bp, Artificial sequence
  16S rRNA  amino acid sequence
tacggaaggtccgggcgttatccggatttattgggtttaaagggagcgcaggctggattttaagcgtgttgtgaaatgcggcggctcaaccgtcgatgtgcagcgcgaactggagtccttgagtgagtacgaggaaggcggaattcgtggtgtagcggtgaaatgcttagatatcacgaagaactccgattgcgaaggcagctttccagtccttaactgacgctaaagctcgaaagtgcgggtatcgaacag

SEQ ID NO: 619 - DNA, 252 bp, Artificial sequence
  16S rRNA  amino acid sequence
tacgtagggagcgagcgttgtccggaattactgggtgtaaagggagcgtaggcgggagtgcaagtcagatgtgaaatacatgggctcaacccatgggctgcatttgaaactgcatttcttgagtgaagtagaggtaagcggaattcctggtgtagcggtgaaatgcgtagatatcaggaggaacaccggtggcgaaggcggcttactgggcttttactgacgctgaggctcgaaagcgtggggagcaaacag

SEQ ID NO: 620 - DNA, 251 bp, Artificial sequence
  16S rRNA  amino acid sequence
tacggaaggtccgggcgttatccggatttattgggtttaaagggagcgcagacgggctgttaagcgtgacgtgaaatccgggggctcaaccttcggctgcgtcgcgaactggcggccttgagtggacggtaggcaggcggaattcgtggtgtagcggtgaaatgcttagatatcacgaagaaccccgattgcgaaggcagcttgccgcagtcttactgacgttcatgctcgaaggtgcgggtatcgaacag

SEQ ID NO: 621 - DNA, 251 bp, Artificial sequence
  16S rRNA  amino acid sequence
tacggaggatgcgagcgttatccggatttattgggtttaaagggtgcgcaggcggcaggtcaagtcagcggtaaaatctcggcgctcaacgccgttcggccgttgaaactgtccggctcgagtgcgtgggaggatggcggaatgcgcggtgtagcggtgaaatgcatagatatcgcgcagaaccccgattgcgaaggcagctgtccaccacgccactgacgctcatgcacgaaagcgtgggtatcgaacag

SEQ ID NO: 622 - DNA, 251 bp, Artificial sequence; identical to SEQ ID NO: 7
  16S rRNA  amino acid sequence

SEQ ID NO: 623 - DNA, 252 bp, Artificial sequence
  16S rRNA  amino acid sequence
tacggaaggtccgggcgttatccggatttattgggtttaaagggagcgcaggcggaaggttaagcgtgacgtgaaatgtaccggctcaaccggtgatgtgcgtcgcgaactggctttcttgagtgagtacgacgctggcggaattcgtggtgtagcggtgaaatgcttagatatcacgaagaactccgattgcgaaggcagccggcgagtcctcaactgacgctaatgctcgaaagtgcgggtatcgaacag

SEQ ID NO: 624 - DNA, 252 bp, Artificial sequence
  16S rRNA  amino acid sequence
tacggaaggtccgggcgttatccggatttattgggtttaaagggagcgcaggccgatggttaagcgtgacgtgaaatgtagccgctcaacggctgaactgcgtcgcgaactggctatcttgagtgagttcgatgtgggcggaattcgtggtgtagcggtgaaatgcttagatatcacgaagaactccgattgcgaaggcagcatgccaggcctttactgacgctaaagctcgaaggtgcgggtatcgaacag

SEQ ID NO: 625 - DNA, 252 bp, Artificial sequence
  16S rRNA  amino acid sequence
tacggaggatgcgagcgttatccggatttattgggtttaaagggtgcgcaggcggcgcgttaagtcagcggtgaaatgtcccggctcaaccggggagctgccgttgatactggcgtgctggagagcggacggcgccggcggaatgagtggtgtagcggtgaaatgcatagatatcactcagaacaccgatcgcgaaggcagctggcgaggccgcgactgacgctgaggcacgaaagcgtggggatcaaacag

SEQ ID NO: 626 - DNA, 252 bp, Artificial sequence
  16S rRNA  amino acid sequence
tacggaaggtccgggcgttatccggatttattgggtttaaagggagcgcaggcgggagtctaagcgtgccgtgaaaagctgtggctcaaccacagtcgtgcggcgcgaactggtcttcttgagttcacggtaagcgggcggaattcgtggtgtagcggtgaaatgcttagatatcacgaggaaccccgattgcgaaggcagcccgctgcagtgttactgacgctcatgctcgaaggcgcgggtatcgaacag

SEQ ID NO: 627 - DNA, 252 bp, Artificial sequence
  16S rRNA  amino acid sequence
tacgtagggagcaagcgttatccggatttattgggtgtaaagggtgcgtagacgggaaattaagttagttgtgaaatccctcggctcaactgaggaactgcaactaaaactggttttcttgagtgcaggagaggaaagtggaattcctagtgtagcggtgaaatgcgtagatattaggaggaacaccagtggcgaaggcgactttctggactgtaactgacgttgaggcacgaaagtgtggggagcaaacag

SEQ ID NO: 628 - DNA, 252 bp, Artificial sequence
  16S rRNA  amino acid sequence
tacggaaggtccgggcgttatccggatttattgggtttaaagggagcgcaggctggcttttaagcgtgttgtgaaatgtaccggctcaaccggtgatgtgcgtcgcgaactggctttcttgagtgggtacgacgctggcggaattcgtggtgtagcggtgaaatgcttagatatcacgaagaactccgattgcgaaggcagccggcgagtcctcaactgacgctaatgctcgaaagtgcgggtatcgaacag

SEQ ID NO: 629 - DNA, 252 bp, Artificial sequence
  16S rRNA  amino acid sequence
tacggaaggtccgggcgttatccggatttattgggtttaaagggtgcgtaggcgggattttaagcgtgaggtgaaatgccacggctcaaccgtggaactgcctcgcgaactgggattcttgaatgcccctgccgtgggaggaatgtgtggtgtagcggtgaaatgcatagagatcacacagaacaccgattgcgaaggcatctcacgaaggggtgattgacgctgaggcacgaaagcgtggggatcaaacag

SEQ ID NO: 630 - DNA, 252 bp, Artificial sequence; identical to SEQ ID NO: 8
  16S rRNA  amino acid sequence

SEQ ID NO: 631 - DNA, 252 bp, Artificial sequence; identical to SEQ ID NO: 9
  16S rRNA  amino acid sequence

SEQ ID NO: 632 - DNA, 252 bp, Artificial sequence; identical to SEQ ID NO: 10
  16S rRNA  amino acid sequence

SEQ ID NO: 633 - DNA, 252 bp, Artificial sequence
  16S rRNA  amino acid sequence
tacgtagggggcaagcgttatccggatttactgggtgtaaagggagcgtagacggtgaggcaagtccgatgtgaaaacccggggctcaaccccgggattgcattggaaactgtttgactcgagtactggaggggtaagcggaattcctagtgtagcggtgaaatgcgtagatattaggaggaacaccggtggcgaaggcggcttactggacagtaactgacgttgaggctcgaaagcgtggggagcaaacag

SEQ ID NO: 634 - DNA, 252 bp, Artificial sequence
  16S rRNA  amino acid sequence
tacggaaggtccgggcgttatccggatttattgggtttaaagggagcgcaggcggccttttaagcgtgacgtgaaatgtaggcgctcaacgtctgaattgcgtcgcgaactgttaggcttgagtagacggaatgtcggcggaattcgtggtgtagcggtgaaatgcttagatatcacgaagaactccgattgcgaaggcagctgacagcagttttactgacgctcatgctcgaaagtgcgggtatcgaacag

SEQ ID NO: 635 - DNA, 252 bp, Artificial sequence; identical to SEQ ID NO: 11
  16S rRNA  amino acid sequence

SEQ ID NO: 636 - DNA, 252 bp, Artificial sequence
  16S rRNA  amino acid sequence
tacgtagggagcgagcgttgtccggaattactgggtgtaaagggtgcgtaggcgggtttgcaagtcagatgtgaaatgccggggctcaaccccggagctgcatttgaaactgtagatcttgagtgaggtagaggtaagcggaattcctagtgtagcggtgaaatgcgtagatattaggaggaacatcggtggcgaaggcggcttactgggcctttactgacgctgaggcacgaaagcgtggggagcaaacag

SEQ ID NO: 637 - DNA, 251 bp, Artificial sequence
  16S rRNA  amino acid sequence
tacggaggatgcgagcgttatccggatttattgggtttaaagggtgcgcaggcggcgagtcaagtcagcggtcaaactgcggcgctcaacgccgtaccgccgttgaaactggcgcgctcgagtttgtgcgaggatggcggaatgcgcagtgtagcggtgaaatgcatagatattgcgcagaactccgattgcgaaggcagctgtccagcacactactgacgctcatgcacgaaagcgtgggtatcgaacag

SEQ ID NO: 638 - DNA, 252 bp, Artificial sequence
  16S rRNA  amino acid sequence
tacgtagggggcaagcgttatccggatttactgggtgtaaagggagcgtagacggcaaggcaagtctgatgtgaaaggtgtgggctcaactcacaaactgcattggaaactgttctgctagagtgcaggagaggtaagcggaattcctagtgtagcggtgaaatgcatagatattaggaggaacaccggcggcgaaggcggcttactggactgtaactgacgttgaggctcgaaagcgtgggtagcaaacag

SEQ ID NO: 639 - DNA, 252 bp, Artificial sequence; identical to SEQ ID NO: 14
  16S rRNA  amino acid sequence

SEQ ID NO: 640 - DNA, 252 bp, Artificial sequence; identical to SEQ ID NO: 22
  16S rRNA  amino acid sequence

SEQ ID NO: 641 - DNA, 252 bp, Artificial sequence
  16S rRNA  amino acid sequence
tacggaaggtccgggcgttatccggatttattgggtttaaagggagcgcaggccgatggttaagcgtgacgtgaaatgtagccgctcaacggctgcactgcgtcgcgaactggccgtcttgagtgagttcgatgtaggcggaattcgtggtgtagcggtgaaatgcttagatatcacgaagaactccgattgcgaaggcagcctacaaggcctttactgacgctaaagctcgaaggtgcgggtatcgaacag

SEQ ID NO: 642 - DNA, 251 bp, Artificial sequence
  16S rRNA  amino acid sequence
tacgtagggagcgagcgttatccggaattattgggcgtaaagggtgcgtagatggcaatataagtcttatgtaaaaatgctgggctcaacccagtagggcataagatactgtatagctcgagtattggagaggcaagtggaactccatgtgtagcggtaaaatgcgtaaatatatggaagaacaccagtggcgaaggcggcttgctagccagatactgacattgaggcacgaaagcgtggggagcaaacag

SEQ ID NO: 643 - DNA, 252 bp, Artificial sequence
  16S rRNA  amino acid sequence
tacggaggatgcaagcgttatccggatttattgggtttaaagggtgcgtaggcggatgattaagcgtgaggtgaaatgccactgctcaacagtggaactgcctcgcgaactggtcgtcttgaatgctcctgccgtgggaggaatgtgtggtgtagcggtgaaatgcatagagatcacacagaacaccgattgcgaaggcatctcacgaaggggtgattgacgctgaggcacgaaagcgtggggatcaaacag

SEQ ID NO: 644 - DNA, 252 bp, Artificial sequence; identical to SEQ ID NO: 28
  16S rRNA  amino acid sequence

SEQ ID NO: 645 - DNA, 252 bp, Artificial sequence; identical to SEQ ID NO: 15
  16S rRNA  amino acid sequence

SEQ ID NO: 646 - DNA, 251 bp, Artificial sequence
  16S rRNA  amino acid sequence
tacggaggatgcgagcgttatccggatttattgggtttaaagggtgcgcaggcggcgtgtcaagtcagcggtcaaaatgcggcgctcaacgccgtaccgccgttgaaactggcatgctcgagttcgtgcgaggatggcggaatgcgcggtgtagcggtgaaatgcatagatatcgcgcagaactccgattgcgaaggcagctgtccagcacacgactgacgctcatgcacgaaagcgtgggtatcgaacag

SEQ ID NO: 647 - DNA, 251 bp, Artificial sequence
  16S rRNA  amino acid sequence
tacggaaggtccgggcgttatccggatttattgggtttaaagggagcgcaggccggtgtgtaagcgtgacgtcaaatacagccgctcaacggttgtccgcgtcgcgaactgcatgccttgagtacgctgcaagtgggcggaattcgtggtgtagcggtgaaatgcttagatatcacgaagaactccgattgcgaaggcagcccgctgttgcgtcactgacgctgaggctcgaaggtgcgggtatcgaacag

SEQ ID NO: 648 - DNA, 252 bp, Artificial sequence
  16S rRNA  amino acid sequence
tacgtagggagcgagcgttgtccggaattactgggtgtaaagggagtgcaggcgggactgcaagtcagatgtgaaatgtaggggcttaacccctgaactgcatttgaaactgtagttcttgagtgaagtagaggtaagcggaattcctagtgtagcggtgaaatgcgtagatattaggaggaacatcagtggcgaaggcggcttactgggcttttactgacgctgaggctcgaaagcgtggggagcaaacag

SEQ ID NO: 649 - DNA, 252 bp, Artificial sequence
  16S rRNA  amino acid sequence
tacggaaggtccgggcgttatccggatttattgggtttaaagggagcgcaggccggtgcttaagcgtgacgtgaaatgtagccgctcaacggctgacgtgcgtcgcgaactgggtgccttgagtgagtacgacgtgtacggaattcgtggtgtagcggtgaaatgcttagatatcacgaagaaccccgattgcgaaggcagtgcacgagtccttaactgacgctaatgctcgaaagtgcgggtatcgaacag

SEQ ID NO: 650 - DNA, 252 bp, Artificial sequence; identical to SEQ ID NO: 24
  16S rRNA  amino acid sequence

SEQ ID NO: 651 - DNA, 252 bp, Artificial sequence
  16S rRNA  amino acid sequence
tacgtagggggcaagcgttatccggatttactgggtgtaaagggagcgcagacggcagtgcaagtctgatgtgaaagcccggggctcaaccccggaactgcattggaaactgtacagcttgagtgtcggagaggtaagcggaattcctagtgtagcggtgaaatgcgtagatattaggaggaacaccggtggcgaaggcggcttactggacgattactgacgttgaggctcgaaagcgtggggagcaaacag

SEQ ID NO: 652 - DNA, 251 bp, Artificial sequence
  16S rRNA  amino acid sequence
tacggaagatgcgagcgttatccggatttattgggtttaaagggagcgtaggcggacgcttaagcctgcggtcaaatgtcagcgcccaacgctggcctgccgtgggaactgggtgtcttgaatgtgcacaaggaaggtggaattcgtggtgtagcggtgaaatgcttagatatcacgaagaactccgattgcgaaggcagccttctggggcatgattgacgctgaggctcgaaagcgcgggaatcaaacag

SEQ ID NO: 653 - DNA, 252 bp, Artificial sequence
  16S rRNA  amino acid sequence
tacgaggggtgcaagcgttgttcggaattactgggcgtaaagggagcgtaggcggagattcaagcggattgtacaatcccggggcccaaccccggttctgcagtccgaactgggtttcttggatagttcaggggcaagcggaattcctggtgtagcggtggaatgcgtagagatcaggaagaacaccgatggcgaaggcagcttgctggggacttatcgacgctcatgctcgaaagtgcgggtagcaaacag

SEQ ID NO: 654 - DNA, 252 bp, Artificial sequence
  16S rRNA  amino acid sequence
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacgataactgacgctgaggctcgaaagcgtggggagcaaacag

(19) United States
(12) Patent Application Publication
(10) Pub. No.: US 2023/0063495 A1
(43) Pub. Date: Mar. 2, 2023

(54) BACTERIAL POPULATIONS FOR DESIRABLE TRAITS IN RUMINATING ANIMALS

(71) Applicant: The National Institute for Biotechnology in the Negev Ltd., Beer-Sheva (IL)

(72) Inventors: Itzhak MIZRAHI, LeHavim (IL); Goor SASSON, Petach Tikva (IL)

(73) Assignee: The National Institute for Biotechnology in the Negev Ltd., Beer-Sheva (IL)

(21) Appl. No.: 17/567,238

(22) Filed: Jan. 3, 2022

Related U.S. Application Data
(63) Continuation of application No. PCT/IL2020/050742, filed on Jul. 2, 2020.
(60) Provisional application No. 62/869,616, filed on Jul. 2, 2019.

(51) Int. Cl. A01K 67/02; C12Q 1/04; C12Q 1/682

(52) U.S. Cl. CPC A01K 67/02; C12Q 1/682; C12Q 1/04

(57) ABSTRACT

A method of selecting a ruminating animal having a desirable, hereditable trait is disclosed. The method comprises analyzing in the microbiome of the animal for an amount of a hereditable microorganism which is associated with the hereditable trait, wherein the amount of the hereditable microorganism is indicative as to whether the animal has a desirable hereditable trait.

### RELATED APPLICATIONS

This application is a Continuation of PCT Patent Application No. PCT/IL2020/050742 having International filing date of Jul. 2, 2020, which claims the benefit of priority under 35 USC § 119(e) of U.S. Provisional Patent Application No. 62/869,616 filed on Jul. 2, 2019. The contents of the above applications are all incorporated by reference as if fully set forth herein in their entirety.

### SEQUENCE LISTING STATEMENT

The ASCII file, entitled 90821SequenceListing.txt, created on Jan. 3, 2022, comprising 335,609 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to a method of selecting a ruminating animal for a desired hereditable trait based on the presence of particular bacteria in the microbiome thereof.

The bovine rumen microbiome essentially enables the hosting ruminant animal to digest its feed by degrading and fermenting it. In this sense this relationship is unique and different from the host-microbiome interactions that have evolved between in humans and non-herbivorous animals, where such dependence does not exist. This strict obligatory host-microbiome relationship, which was established approximately 50 million years ago, is thought to play a major role in host physiology. Despite its great importance, the impact of natural genetic variation in the host—brought about through sexual reproduction and meiotic recombination—on the complex relationship of rumen microbiome components and host physiological traits is poorly understood. It is known that associations between specific components of the rumen microbiome to animals physiology exist, mainly exemplified by the ability of the animal to harvest energy from its feed [Kruger Ben Shabat S, et al., 2016. ISME J 10:2958-2972].

These recent findings position the bovine rumen microbiome as the new frontier in the effort to increase the feed efficiency of milking cows. As human population is continually increasing this could have important implications for food security issues as an effort towards replenishing food sources available for human consumption while lowering environmental impact in global scale. Despite its great importance, the complex relationship of rumen microbiome components and host genetics and physiology is poorly understood.

Background art includes WO2019/030752, WO2017/187433 and WO2014/141274, Guan L L, et al., 2008. FEMS Microbiology Letters 288:85-9; Roehe R, et al., 2016. PLoS Genet 12:e1005846; Li Z, et al., 2016. Microbiology Reports 8:1016-102.

### SUMMARY OF THE INVENTION

According to an aspect of some embodiments of the present invention there is provided a method of selecting a ruminating animal having a desirable, hereditable trait comprising analyzing in the microbiome of the animal for an amount of at least one hereditable bacteria which is associated with the hereditable trait, wherein the amount of the hereditable bacteria is indicative as to whether the animal has a desirable hereditable trait, wherein the hereditable bacteria is of any one of the operational taxonomic units (OTUs) set forth in Table 1, wherein the trait is the corresponding trait to the at least one hereditable bacteria as set forth in Table 1, thereby selecting the ruminating animal having a desirable hereditable trait.

According to an aspect of some embodiments of the present invention there is provided a method of managing a herd of ruminating animals comprising:

(a) analyzing in the microbiome of a ruminating animal of the herd for an amount of at least one hereditable bacteria which is associated with the hereditable trait, wherein the amount of the hereditable bacteria is indicative that the animal has a non-desirable hereditable trait, wherein the hereditable bacteria is of any one of the operational taxonomic units (OTUs) set forth in Table 1, wherein the trait is the corresponding trait to the at least one hereditable bacteria as set forth in Table 1; and

(b) removing the animal with the non-desirable trait from the herd.

According to an aspect of some embodiments of the present invention there is provided a method for breeding a ruminating animal comprising breeding a ruminating animal that has been selected according to the methods described herein, thereby breeding the ruminating animal.

According to an aspect of some embodiments of the present invention there is provided a method of increasing the number of ruminating animals having a desirable microbiome comprising breeding a male and female of the ruminating animals, wherein the rumen microbiome of either of the male and/or the female ruminating animals comprises a hereditable microorganism having an OTU as set forth in Table 3 above a predetermined level, thereby increasing the number of ruminating animals having a desirable microbiome.

According to an aspect of some embodiments of the present invention there is provided a method of altering a trait of a ruminating animal comprising providing a microbial composition to the ruminating animal which comprises at least one microbe having an operational taxonomic unit (OTU) set forth in Table 2 and having a 16S rRNA sequence as set forth in SEQ ID NOs: 38-50 and 314-615, thereby altering the trait of the ruminating animal, wherein the microbial composition does not comprise a microbiome of the ruminating animal, wherein the trait is the corresponding trait to the at least one microbe as set forth in Table 2.

According to an aspect of some embodiments of the present invention there is provided a method of altering a trait of a ruminating animal comprising providing an agent which specifically downregulates an OTU set forth in Table 2 to the ruminating animal, thereby altering the trait of the ruminating animal, wherein the trait is the corresponding trait to the at least one microbe as set forth in Table 2.

According to an aspect of some embodiments of the present invention there is provided a microbial composition comprising at least one microbe having an OTU set forth in Table 2, the microbial composition not being a microbiome.

According to embodiments of the present invention, the hereditable bacteria is of the family lachnospiraceae or of the genus Prevotella.

According to embodiments of the present invention, the ruminating animal is a cow.

According to embodiments of the present invention, the method further comprises using the selected animal or a progeny thereof for breeding.

According to embodiments of the present invention, the analyzing an amount is effected by analyzing the expression of at least one gene of the genome of the at least one bacteria.

According to embodiments of the present invention, the analyzing an amount is effected by sequencing the DNA derived from a sample of the microbiome.

According to embodiments of the present invention, the microbiome comprises a rumen microbiome or a fecal microbiome.

According to embodiments of the present invention, the ruminating animal that has been selected is a female ruminating animal, the method comprises artificially inseminating the female ruminating animal with semen from a male ruminating animal.

According to embodiments of the present invention, the male ruminating animal has been selected according to the methods described herein.

According to embodiments of the present invention, when the ruminating animal that has been selected is a male ruminating animal, the method comprises inseminating a female ruminating animal with semen of the male ruminating animal.

According to embodiments of the present invention, the hereditable microorganism is associated with a hereditable trait.

According to embodiments of the present invention, the microbial composition comprises no more than 20 microbial species.

According to embodiments of the present invention, the microbial composition comprises no more than 50 microbial species.

According to embodiments of the present invention, the at least one microbe has an OTU set forth in Table 1.

According to embodiments of the present invention, the at least one microbe has a 16S rRNA sequence as set forth in SEQ ID NOs: 7-37 and 51-313.

According to embodiments of the present invention, the at least one microbe has an OTU set forth in Table 1.

According to embodiments of the present invention, the microbial composition comprises no more than 15 bacterial species.

According to embodiments of the present invention, the microbial composition comprises no more than 20 bacterial species.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-C. Host genetics explains core microbiome composition with heritable microbes serving as hubs within the microbial interaction networks. The core microbiome is associated with animal genetics as (A) the variance in the core microbiome (Y-axis) was significantly explained by host genetics. Canonical Correlation Analysis (CCA) was performed between the matrix of the first 30 microbial (OTU table) principal component scores and host genotype principal component scores based on common single nucleotide polymorphism (SNP). The analysis was accomplished for the largest Holstein farms in this study (X-axis). (B) Heritability analysis based on the genetic relatedness matrix (GRM) showed 39 microbes (X-axis) significantly correlating with the animal genotype. Heritability estimate—h² (Y-axis; barplots show mean estimate per microbe) and P-values were calculated using Genetics Complex Trait Analysis (GCTA) software, followed by a multiple testing correction with Benjamini-Hochberg method. Confidence intervals (95%) were estimated based on heritability estimates and the GRM with Fast Confidence IntErvals using Stochastic Approximation (FIESTA) software. (C) Heritable microbes are central to the microbial interaction network, as revealed by the higher mean connectivity (Y-axis) of these microbes compared to the non-heritable ones. The interaction network was built using Sparse InversE Covariance estimation for Ecological Association and Statistical Inference (SpiecEasi). Results are presented as mean number of microbial interactions with standard-error. Indicated P-values, P<0.05 with *, P<0.005 with **, P<0.0005 with ***.

FIGS. 2A-D. Core rumen microbiome composition is linked to host traits and could significantly predict them. (A) Association analysis between microbes and host traits revealed 339 microbes associated with at least one trait. In order for a microbe to be associated with a given trait it had to significantly and unidirectionally correlate with a trait within each of at least four farms (after Benjamini-Hochberg multiple testing correction) with no farm showing a significant correlation in the opposing direction. (B) The majority of the trait-associated microbes are associated with rumen propionate and acetate concentrations, while heritable microbes are enriched among Acetate co-abundant microbes and among Propionate anti-correlated microbes. (C) Enrichment analysis, using Fisher exact test, showed that the core microbes are much more present (enriched) within trait-associated microbes compared to the non-core microbiome (P<2.2E-16). Indicated P-values, P<0.05 with *, P<0.005 with **, P <0.0005 with ***. (D) Explained variation (r²) of different host traits as function of core microbiome composition. r² estimates were derived from a machine-learning approach where a trait-value was predicted for a given animal using the Ridge regression (Least Absolute Shrinkage and Selection Operator) that was constructed from all other animals in farm (leave-one-out regression). Thereafter, prediction r² value was calculated between the vectors of observed and predicted trait values. Indicated host traits were significantly explained (via prediction) by core microbe (OTU) abundance profiles. Dots stand for individual farms' prediction r² while bar heights represent mean of individual farms' r².

FIG. 3. Heritable microbes tend to explain experimental variables better in comparison to non-heritable core microbes. X-axis: experimental variable. Y-axis: Ridge regression R² value for explaining the phenotype. Point: R² when heritable microbes used as independent variables. Bar-lot and whiskers relate to mean and standard error of R² values obtained from 1.00 random samples of non-heritable core microbes that were used as independent variables. Wilcoxon paired rank-sums test was used to compare heritable microbes' R² values for explaining the different experimental variables to that of non-heritable core microbes (mean R²).

FIG. 4. Explained variation (r²) of different host traits as function of core microbiome composition. r² estimates were derived from a machine-learning approach where a trait-value was predicted for a given animal using a Random-Forest model that was constructed from all other animals in farm (leave-one-out regression). Thereafter, prediction r² value was calculated between the vectors of observed and predicted trait values. Indicated host traits were significantly explained (via prediction) by core microbe (OTU) abundance profiles. Dots stand for individual farms' prediction r² while bar heights represent mean of individual farms' r².

### DESCRIPTION OF SPECIFIC EMBODIMENTS OF THE INVENTION

The present invention, in some embodiments thereof, relates to a method of selecting a ruminating animal for a desired hereditable trait based on the presence of particular bacteria in the microbiome thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Ruminants sustain a long-lasting obligatory relationship with their rumen microbiome dating back 50 million years. In this unique host-microbiome relationship the host's ability to digest its feed is completely dependent on its coevolved microbiome. This extraordinary alliance raises questions regarding the dependence between ruminants' genetics and physiology and the rumen microbiome structure, composition and metabolism. To elucidate this relationship, the present inventors examined association of host genetics to phylogenetic and functional composition of the rumen microbiome. They accomplished this by studying a population of 1000 cows in four different European countries, using a combination of rumen microbiota data and other phenotypes from each animal with genotypic data from a subset of animals. This very large population size uncovered novel and unexpected bacteria that can be used to regulate desirable traits in these animals.

Thus, according to a first aspect of the present invention there is provided a method of selecting a ruminating animal having a desirable, hereditable trait comprising analyzing in the microbiome of the animal for an amount of at least one hereditable bacteria which is associated with the hereditable trait, wherein the amount of the hereditable bacteria is indicative as to whether the animal has a desirable hereditable trait, wherein the hereditable bacteria is of any one of the operational taxonomic units (OTUs) set forth in Table 1, wherein the trait is the corresponding trait to the at least one hereditable bacteria as set forth in Table 1, thereby selecting the ruminating animal having a desirable hereditable trait.

Ruminating animals contemplated by the present invention include for example cattle (e.g. cows), goats, sheep, giraffes, American Bison, European Bison, yaks, water buffalo, deer, camels, alpacas, llamas, wildebeest, antelope, pronghorn, and nilgai.

According to a particular embodiment, the ruminating animal is a bovine cow or bull—e.g. Bos taurus bovines or Holstein-Friesian bovines.

According to a particular embodiment, the animal which is selected is a newborn, typically not more than one day old. According to another embodiment, the animal which is selected is not more than two days old. According to another embodiment, the animal which is selected is not more than three days old. According to another embodiment, the animal which is selected is not more than 1 week old. According to another embodiment, the animal which is selected is not more than 2 weeks old. According to another embodiment, the animal which is selected is not more than 1 month old. According to another embodiment, the animal which is selected is not more than 3 months old. According to still another embodiment, the animal is an adult.

The phrase “hereditable trait” (also referred to as “heritable trait”) as used herein, refers to a trait of which the variation between the individuals in a given population is due in part (or in whole) to genetic variation. Due to these genetic variations, the relative or absolute abundance of particular microbial populations in the microbiome (which serve as markers) is similar from one generation to the next generation in a statistically significant manner.

A microorganism can be classified as being hereditable when changes in its abundance amongst a group of animals can be explained by the genetic variance amongst the animals.

Statistical methods which can be used in the context of the present invention include, but are not limited to Single component GRM approach, MAF-Stratified GREML (GREMLLMS), LDL and MAF-Stratified GREML (GREMLLLDMS), Single Component and MAF-Stratified LD-Adjusted Kinships (LDAK-SC and LDAK-MS), Extended Genealogy with Thresholded GRMs, Treelet Covariance Smoothing (TCS), LD-Score Regression and BOLT-REML.

According to a particular embodiment, the hereditable bacteria is set forth in Table 1, herein below. Thus, for example the hereditable bacteria may belong to the family lachnospiraceae or to the genus Prevotella.

In one embodiment, the trait is the corresponding trait to the bacteria as set forth in Table 1. Thus, the trait may be rumen propionate, rumen acetate, rumen butyrate, milk lactose, milk yield, milk fat, rumen pH and rumen Beta-Hydroxybutyric Acid (BHB).

Table 1, herein below also provides the correlation between the host trait and the amount of the particular bacteria in the rumen microbiome. Thus, for example, the first row of Table 1 relates to a bacteria (having a 16S rRNA sequence as set forth in SEQ ID NO: 7) whose abundance negatively correlates with rumen propionate. If the desired trait is low rumen propionate, the selected animal will have an amount of bacteria having a 16S rRNA sequence as set forth in SEQ ID NO: 7 above a predetermined level. If the desired trait is high rumen propionate, the selected animal will have an amount of bacteria having a 16S rRNA sequence as set forth in SEQ ID NO: 7 below a predetermined level. The other bacteria in Table 1 and their corresponding traits can be selected in the same way.

According to one embodiment, an animal can be classified as having a low trait (e.g. one that appears in Tables 1 or 2) when it has at least 0.05, 1, 2, 3, 4, 5 or even 6 standard deviations below the average amount of that trait of the herd (with a herd being at least 15 animals).

According to one embodiment, an animal can be classified as having a high trait (e.g. one that appears in Tables 1 or 2) when it has at least 0.05, 0.5, 1, 2, 3, 4, 5, or even 6 standard deviations above the average amount of that trait of the herd (with a herd being at least 15 animals).

The term “microbiome” as used herein, refers to the totality of microbes (bacteria, fungi, protists), their genetic elements (genomes) in a defined environment.

A microbiota sample comprises a sample of microbes and or components or products thereof from a microbiome.

According to a particular embodiment, the microbiome is a rumen microbiome. In still other embodiments, the microbiome is a fecal microbiome.

According to another embodiment, the microbiome is derived from a healthy animal (i.e. the microbiome is a non-pathogenic microbiome).

In order to analyze the microbes of a microbiome, a microbiota sample is collected from the animal. This is carried out by any means that allow recovery of microbes or components or products thereof of a microbiome and is appropriate to the relevant microbiome source e.g. rumen.

Rumen may be collected using methods known in the art and include for example use of a stomach tube with a rumen vacuum sampler. Typically rumen is collected after feeding.

In some embodiments, in lieu of analyzing a rumen sample, a fecal sample is used which mirrors the microbiome of the rumen. Thus, in this embodiment, a fecal microbiome is analyzed.

According to one embodiment of this aspect of the present invention, the abundance of particular bacterial taxa are analyzed in a microbiota sample.

Methods of quantifying levels of microbes (e.g. bacteria) of various taxa are described herein below.

In some embodiments, determining a level or set of levels of one or more types of microbes or components or products thereof comprises determining a level or set of levels of one or more DNA sequences. In some embodiments, one or more DNA sequences comprise any DNA sequence that can be used to differentiate between different microbial types. In certain embodiments, one or more DNA sequences comprise 16S rRNA gene sequences. In certain embodiments, one or more DNA sequences comprise 18S rRNA gene sequences. In some embodiments, 1, 2, 3, 4, 5, 10, 15, 20, 25, 50, 100, 1,000, 5,000 or more sequences are amplified.

Taxonomy assignment of species may be performed using a suitable computer program (e.g. BLAST) against the appropriate reference database (e.g. 16S rRNA reference database).

In determining whether a nucleic acid or protein is substantially homologous or shares a certain percentage of sequence identity with a sequence of the invention, sequence similarity may be defined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed.

According to one embodiment, in order to classify a microbe as belonging to a particular genus, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular genus. According to a particular embodiment, the sequence homology is at least 95%.

According to another embodiment, in order to classify a microbe as belonging to a particular species, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular species. According to a particular embodiment, the sequence homology is at least 97%.

In some embodiments, a microbiota sample is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QJAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes or components or products thereof comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, determining a level or set of levels of one or more types of microbes or components or products thereof comprises determining a level or set of levels of one or more microbial proteins. Methods of quantifying protein levels are well known in the art and include but are not limited to western analysis and mass spectrometry. These and all other basic protein detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York). In some embodiments, determining a level or set of levels of one or more types of microbes or components or products thereof comprises determining a level or set of levels of one or more microbial metabolites. In some embodiments, levels of metabolites are determined by mass spectrometry. In some embodiments, levels of metabolites are determined by nuclear magnetic resonance spectroscopy. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

In some embodiments, what is determined is the distribution of microbial families within the microbiome. However, characterization may be carried to more detailed levels, e.g. to the level of genus and/or species, and/or to the level of strain or variation (e.g. variants) within a species, if desired (including the presence or absence of various genetic elements such as genes, the presence or absence of plasmids, etc.). Alternatively, higher taxanomic designations can be used such as Phyla, Class, or Order. The objective is to identify which microbes (usually bacteria, but also optionally fungi (e.g. yeasts), protists, etc.) are present in the sample from the ruminating animal and the relative distributions of those microbes, e.g. expressed as a percentage of the total number of microbes that are present, thereby establishing a micro floral pattern or signature for the animal being tested.

In other embodiments of the invention, when many taxa are being considered, the overall pattern of microflora is assessed, i.e. not only are particular taxa identified, but the percentage of each constituent taxon is taken in account, in comparison to all taxa that are detected and, usually, or optionally, to each other. Those of skill in the art will recognize that many possible ways of expressing or compiling such data exist, all of which are encompassed by the present invention. For example, a “pie chart” format may be used to depict a microfloral signature; or the relationships may be expressed numerically or graphically as ratios or percentages of all taxa detected, etc. Further, the data may be manipulated so that only selected subsets of the taxa are considered (e.g. key indicators with strong positive correlations). Data may be expressed, e.g. as a percentage of the total number of microbes detected, or as a weight percentage, etc.

In order to identify microbial species where significant proportions of their variation in abundance profiles can be attributed to heritable genetic factors, the microbiota sample is analyzed so as to uncover taxa (e.g. species) of microbes showing similar abundance (either absolute or relative) in animals that share a similar genetic background.

Methods of analyzing the similarity of the genetic background of two ruminating animals may be carried out using genotyping assays known in the art.

As used herein, the term “genotyping’ refers to the process of determining genetic variations among individuals in a species. Single nucleotide polymorphisms (SNPs) are the most common type of genetic variation that are used for genotyping and by definition are single-base differences at a specific locus that is found in more than 1% of the population. SNPs are found in both coding and non-coding regions of the genome and can be associated with a phenotypic trait of interest such as a quantitative phenotypic trait of interest. Hence, SNPs can be used as markers for quantitative phenotypic traits of interest. Another common type of genetic variation that are used for genotyping are “InDels” or insertions and deletions of nucleotides of varying length. For both SNP and InDel genotyping, many methods exist to determine genotype among individuals. The chosen method generally depends on the throughput needed, which is a function of both the number of individuals being genotyped and the number of genotypes being tested for each individual. The chosen method also depends on the amount of sample material available from each individual or sample. For example, sequencing may be used for determining presence or absence of markers such as SNPs, e.g. such as Sanger sequencing and High Throughput Sequencing technologies (HTS). Sanger sequencing may involve sequencing via detection through (capillary) electrophoresis, in which up to 384 capillaries may be sequence analysed in one run. High throughput sequencing involves the parallel sequencing of thousands or millions or more sequences at once. HTS can be defined as Next Generation sequencing, i.e. techniques based on solid phase pyrosequencing or as Next-Next Generation sequencing based on single nucleotide real time sequencing (SMRT). HTS technologies are available such as offered by Roche, Illumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Each of these sequencing technologies have their own way of preparing samples prior to the actual sequencing step. These steps may be included in the high throughput sequencing method. In certain cases, steps that are particular for the sequencing step may be integrated in the sample preparation protocol prior to the actual sequencing step for reasons of efficiency or economy. For instance, adapters that are ligated to fragments may contain sections that can be used in subsequent sequencing steps (so-called sequencing adapters). Primers that are used to amplify a subset of fragments prior to sequencing may contain parts within their sequence that introduce sections that can later be used in the sequencing step, for instance by introducing through an amplification step a sequencing adapter or a capturing moiety in an amplicon that can be used in a subsequent sequencing step. Depending also on the sequencing technology used, amplification steps may be omitted.

Low density and high density chips are contemplated for use with the invention, including SNP arrays comprising from 3,000 to 800,000 SNPs. By way of example, a “50K” SNP chip measures approximately 50,000 SNPs and is commonly used in the livestock industry to establish genetic merit or genomic estimated breeding values (GEBVs). In certain embodiments of the invention, any of the following SNP chips may be used: BovineSNP50 v1 BeadChip (Illumina), Bovine SNP v2 BeadChip (Illumina), Bovine 3K BeadChip (Illumina), Bovine LD BeadChip (Illumina), Bovine HD BeadChip (Illumina), Geneseek® Genomic Profiler™ LD BeadChip, or Geneseek® Genomic Profiler™ HD BeadChip.

In one embodiment, in order to measure the genetic similarity between the animals the genetic relatedness between the animals based on the SNP data is calculated. To this end a matrix that estimates the genetic relatedness between each unique pair of animals can be produced. This matrix is based on the count of shared alleles, weighted by the allele's rareness:

$${A}_{jk} = \frac{1}{n} {\sum }_{i = l}^{n} ( \frac{( {x}_{ij} - 2 {p}_{i} ) ( {x}_{ik} - 2 {p}_{i} )}{2 {p}_{i} ( 1 - {p}_{i} )} )$$

where Ajk represents the genetic relationship estimate between animals j and k; xij and xik are the counts of the reference alleles in animals j and k, respectively; pi is the proportion of the reference allele in the population; and n is the total number of SNPs used for the relatedness estimation.

In one embodiment, microbes or OTUs that exhibits a significant heritable component are considered as such if their heritability estimate is of >0.01 and P value of <0.1. It will be appreciated that the confidence level may be increased or decreased according to the stringency of the test. Thus, for example in another embodiment, microbes that exhibits a significant heritable component are considered as such if their heritability estimate is of >0.01 and P value of <0.05. Other contemplated heritability estimates contemplated by the present inventors include >0.02 and P value of <0.1, >0.03 and P value of <0.1, >0.04 and P value of <0.1, >0.05 and P value of <0.1, >0.06 and P value of <0.1, >0.07 and P value of <0.1, >0.08 and P value of <0.1, >0.09 and P value of <0.1, >0.1 and P value of <0.1, >0.2 and P value of <0.1, >0.3 and P value of <0.1, >0.4 and P value of <0.1, >0.5 and P value of <0.1, >0.6 and P value of <0.1, >0.7 and P value of <0.1, >0.8 and P value of <0.1.

Other contemplated heritability estimates contemplated by the present inventors include >0.02 and P value of <0.05, >0.03 and P value of <0.05, >0.04 and P value of <0.05, >0.05 and P value of <0.05, >0.06 and P value of <0.05, >0.07 and P value of <0.05, >0.08 and P value of <0.05, >0.09 and P value of 0.05, >0.1 and P value of 0.05, >0.2 and P value of 0.05, >0.3 and P value of 0.05, >0.4 and P value of 0.05, >0.5 and P value of 0.05, >0.6 and P value of 0.05, >0.7 and P value of 0.05, >0.8 and P value of 0.05.

According to a particular embodiment, the heritability estimate is >0.7 and a P value of <0.05.

To increase the confidence of the analysis, the heritability analysis may be limited exclusively to bacterial taxa which are present in at least 20%, 25%, 30%, 40%, 50% or higher of the genotyped subset. In addition, heritability analyses for each bacterial taxa may be performed a number of times, e.g. on a number of different sampling days (e.g. 2, 3, 4, 5, or more days). Only bacterial taxa that exhibited a significant heritable component (e.g. heritability estimate of >0.7 and p-value <0.05) in all individual sampling days, could be considered as heritable.

The term “OTU” as used herein, refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. In 16S embodiments, OTUs that share greater than 97% average nucleotide identity across the entire 16S or some variable region of the 16S are considered the same OTU. See e.g., Claesson et al., 2010. Comparison of two next-generation sequencing technologies for resolving highly complex microbiota composition using tandem variable 16S rRNA gene regions. Nucleic Acids Res 38: e200. Konstantinidis et al., 2006. The bacterial species definition in the genomic era. Philos Trans R Soc Lond B Biol Sci 361: 1929-1940. In embodiments involving the complete genome, MLSTs, specific genes, other than 16S, or sets of genes OTUs that share, greater than 95% average nucleotide identity are considered the same OTU. See e.g., Achtman and Wagner. 2008. Microbial diversity and the genetic nature of microbial species. Nat. Rev. Microbiol. 6: 431-440; Konstantinidis et al., 2006, supra. The bacterial species definition in the genomic era. Philos Trans R Soc Lond B Biol Sci 361: 1929-1940. OTUs can be defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence. As used herein, a “type” of bacterium refers to an OTU that can be at the level of a strain, species, clade, or family.

The present invention further contemplates analysing a plurality of the above described OTUs. Thus, at least one OTU, at least two, at least three, at least four, at least five, at least six, at least seven, at least eight, at least nine, at least ten, at least 11, at least 12, at least 13, at least 14, at least 15 or all of the above described OTUs are analysed.

It will be appreciated that once the animal has been classified as having sufficient quantity of a heritable microorganism that correlates with a desirable phenotype, it may be selected (e.g. separated from the rest of the herd) and classified as having that phenotype. According to one embodiment, the animal branded such that it is clear that it comprises this phenotype.

As well as selecting the particular animal which has the desirable phenotype, the present inventors also contemplate removing (e.g. culling) animals from a herd that do not have the desirable phenotype. The animal may be branded as having the non-desirable phenotype. Thus, the present invention may be used to manage herds ensuring that the percentage of animals with a desirable phenotype in the herd is at its maximum and/or the percentage of animals with a non-desirable phenotype in the herd is at its minimum.

In one embodiment, the animal that has been deemed as having a desirable trait is selected as a candidate for breeding. Thus, the animal may be deemed suitable as a gamete donor for natural mating, artificial insemination or in vitro fertilization.

Thus, according to another aspect of the present invention there is provided a method for breeding a ruminating animal comprising: inseminating a female ruminating animal that has been selected according to the methods described herein with semen from a male ruminating animal, thereby breeding the ruminating animal.

In one embodiment, the male ruminating animal has also been selected as described herein.

According to another aspect of the present invention there is provided a method for breeding a ruminating animal comprising: inseminating a female ruminating animal with semen from a male ruminating animal that has been selected as described herein above, thereby breeding the ruminating animal.

The breeding of the one or more bovine bulls with the bovine cows is preferably by artificial insemination, but may alternatively be by natural insemination.

In one embodiment, the female ruminating animal has also been selected as described herein.

The present inventors have uncovered additional hereditable bacteria in the rumen microbiome. The hereditable bacteria are summarized in Table 3. By breeding animals that have rumen microbiomes containing one of these hereditable bacteria, it is possible to ensure that offspring of that animal will also contain that bacteria in their rumen microbiome. If the hereditable bacteria are associated with a particular trait (see Table 1), then by breeding animals that have rumen microbiomes containing one of these hereditable bacteria and the associated trait, it is possible to ensure that offspring of that animal will also contain that bacteria in their rumen microbiome, and therefore by virtue that trait.

Thus, according to another aspect of the present invention there is provided a method of increasing the number of ruminating animals having a desirable microbiome comprising breeding a male and female of said ruminating animals, wherein the rumen microbiome of either of said male and/or said female ruminating animals comprises a hereditable microorganism having an OTU as set forth in Table 3 above a predetermined level, thereby increasing the number of ruminating animals having a desirable microbiome.

As mentioned herein above, as well as selecting the particular animal which has the desirable microbiome, the present inventors also contemplate removing (e.g. culling) animals from a herd that do not have the desirable microbiome. Thus, the present invention may be used to manage herds ensuring that the percentage of animals with a desirable microbiome in the herd is at its maximum and/or the percentage of animals with a non-desirable microbiome in the herd is at its minimum.

The present inventors have also uncovered numerous bacteria that are associated with traits. Accordingly, the present inventors propose dictating the trait of a ruminating animal by altering its rumen microbiome.

According to this aspect of the present invention, the desirable microbiome is a microbiome which comprises a hereditable bacteria. Thus, the present inventors conceive that the hereditable bacteria itself may be considered as a hereditable trait.

Thus, according to another aspect of the present invention, there is provided a method of altering a trait of a ruminating animal comprising providing a microbial composition to the ruminating animal which comprises at least one microbe having an operational taxonomic unit (OTU) set forth in Table 2 and having a 16S rRNA sequence as set forth in SEQ ID NOs: 38-50 and 314-615, thereby altering the trait of the ruminating animal, wherein the microbial composition does not comprise a microbiome of the ruminating animal, wherein the trait is the corresponding trait to said at least one microbe as set forth in Table 2.

According to a particular embodiment, the bacteria is one that has a 16S rRNA sequence as set forth in SEQ ID NOs: 38-50 and 314-615.

In one embodiment, the microbial composition comprises at least one, at least two, at least three, at least four, at least five, at least six, at least seven, at least, eight, at least nine, at least ten, at least 11, at least 12, at least 13, at least 14, at least 15, at least 16, at least 17, at least 18, at least 19 at least 20 or more microbial species mentioned in Table 2.

Preferably, the microbial compositions of this aspect of the present invention comprise at least two microbial species. In one embodiment, the microbial compositions of this aspect of the present invention comprise less than 100 microbial species, less than 50 microbial species, less than 40 microbial species, less than 30 microbial species. Exemplary ranges of microbial species include 2-100, 2-50, 2-25, 2-20, 2-15. 2-10.

The microbial composition may be derived directly from a microbiota sample of the high energy efficient animal. Alternatively, the microbial composition may be artificially created by adding known amounts of different microbes. It will be appreciated that the microbial composition which is derived from the microbiota sample of an animal may be manipulated prior to administrating by increasing the amount of a particular species (e.g. increasing the amount of/or depleting the amount of a particular species). In another embodiment, the microbial compositions are not treated in any way which serves to alter the relative balance between the microbial species and taxa comprised therein. In some embodiments, the microbial composition is expanded ex vivo using known culturing methods prior to administration. In other embodiments, the microbial composition is not expanded ex vivo prior to administration.

According to one embodiment, the microbial composition is not derived from fecal material.

According to still another embodiment, the microbial composition is devoid (or comprises only trace quantities) of fecal material (e.g., fiber).

Prior to administration, the animal may be pretreated with an agent which reduces the number of naturally occurring rumen microbiome (e.g. by antibiotic treatment). According to a particular embodiment, the treatment significantly eliminates the naturally occurring rumen microflora by at least 20%, 30% 40%, 50%, 60%, 70%, 80% or even 90%.

As well as increasing the above mentioned bacterial populations in the rumen microbiome of the animals, the present inventors further contemplate decreasing any one of the bacterial species set forth in Table 2 herein below to alter a corresponding trait.

According to a particular embodiment, the bacteria has a 16S rRNA sequence as set forth in SEQ ID NOs: 1-37 and 51-313.

According to one embodiment, the agent which decreases the abundance of a bacteria is not an antibiotic agent.

According to another embodiment, the agent which decreases the abundance of the bacteria is an antimicrobial peptide.

According to still another embodiment, the agent which decreases the abundance of a bacteria is a bacteriophage.

According to still another embodiment, the agent which decreases the abundance of a bacteria is capable of downregulating an essential gene of at least one of the bacterial species described herein below.

Thus, for example, the present inventors contemplate the use of meganucleases, such as Zinc finger nucleases (ZFNs), transcription-activator like effector nucleases (TALENs) and CRISPR/Cas system to downregulate the essential gene.

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

The compositions described herein (e.g. microbial compositions) may be administered per se (e.g. using a catheter or syringe) or may be administered together in the feed (e.g. as a feed additive) of the animal or the drink of the animal.

These ruminants may be fed the feed additive composition of the present invention at any time and in any amount during their life. That is, the ruminant may be fed the feed additive composition of the present invention either by itself or as part of a diet which includes other feedstuffs. Moreover, the ruminant may be fed the feed additive composition of the present invention at any time during its lifetime. The ruminant may be fed the feed additive composition of the present invention continuously, at regular intervals, or intermittently. The ruminant may be fed the feed additive composition of the present invention in an amount such that it accounts for all, a majority, or a minority of the feed in the ruminant's diet for any portion of time in the animal's life. According to one embodiment, the ruminant is fed the feed additive composition of the present invention in an amount such that it accounts for a majority of the feed in the animal's diet for a significant portion of the animal's lifetime.

Examples of additional rumen active feed additives which may be provided together with the feed additive of the present invention include buffers, fermentation solubles, essential oils, surface active agents, monensin sodium, organic acids, and supplementary enzymes.

Also contemplated is encapsulation of the microbes in nanoparticles or microparticles using methods known in the art including those disclosed in EP085805, EP1742728 A1, WO2006100308 A2 and U.S. Pat. No. 8,449,916, the contents of which are incorporated by reference.

The compositions may be administered orally, rectally or any other way which is beneficial to the animal such that the microbes reach the rumen of the animal.

In another embodiment, the present invention provides novel processes for raising a ruminant by feeding the ruminant such a feed additive composition.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Experimental Design and Subject Details

The primary objective of this research was to relate the animal genome to the rumen microbiome, feed efficiency, and methane emissions in lactating dairy cows. The following research questions were specified at the outset: Does host genetics have a significant effect on the overall microbiome composition and to what extent? How consistent is the rumen microbiome across geographic locations, breeds and diets? On discovery of a heritable core rumen microbiome, the following additional research questions arose: Do heritable rumen microbes interact with the rest of the core rumen microbes? How do heritable microbes integrate in the overall microbe-host phenotype interaction network?

The objectives were addressed in an observational study involving collection of phenotypic data describing animal metabolism, digestion efficiency and emissions of methane and nitrogen. Samples of rumen digesta and blood were collected for molecular analysis and subsequent statistical analysis to identify correlations and genetic associations.

The final population sampled was 1016 cows to allow a small margin in case any individuals or samples had to be excluded.

Prospective inclusion criteria for animal selection were that cows must be between 10 and 40 weeks postpartum, have received the standard diet for at least 14 days, and had no health issue in the current lactation. Prospective data exclusion criteria were missing samples (e.g. milk, blood, rumen, feces), sample processing issues (e.g. inadequate DNA yield, assay problems, laboratory mishaps), and implausible outliers. Statistical outliers were defined as values greater than three standard deviations from the mean. All statistical outliers were investigated and calculations corrected or assays repeated where appropriate. Otherwise, outliers were retained for data analysis unless they were implausible. Data for any excluded sample were omitted, but the remaining data for the individual were retained.

Six milk samples were missing due to a faulty sampling device, and one blood sample was missing from a cow that could not be sampled. Two rumen fluid samples were lost during laboratory analysis. Two estimates of feed intake were considered implausible (200% of expected) due to abnormal fecal alkane values.

Animal work was conducted by four research teams in United Kingdom (UK), Italy (IT), Sweden (SE) and Finland (FI). In total, 1,016 cows on seven farms were sampled, and associated data collected. UK sampled 409 cows on two farms (UK1: N=243, and UK2: N=164); IT sampled 409 cows on three farms (IT1: N=185, IT2: N=176, and IT3: N=48); SE sampled 100 cows on one farm (SE1); and FI sampled 100 cows on one farm (FI1).

Recordings and collection of biological samples were performed over a 5-day period for each cow that had received the standard diet for at least 14 days. To reach 1,016 cows, sampling was conducted over a period of 26 months in 78 sessions with between 1 and 40 cows per session. At time of recording and sampling, all cows were in established lactation (between 10 and 40 weeks postpartum) when energy balance is close to zero and methane output is relatively stable (26).

Housing and Feeding Systems:

Cows on all farms were group-housed in loose housing barns, except in FI where cows were housed in individual standings during the sampling period. To minimize environmental variation, all cows were offered diets that were standardized within farms, i.e. all cows on a farm were fed on the same diet at any sampling period, and any changes to diet formulation when batches of forage changed were made at least 14 days before sampling commenced. Diets were based on maize silage, grass silage or grass hay, and concentrates in UK and IT, and were based on grass silage and concentrates in SE and FI. Diets were fed as ad libitum total-mixed rations (TMR) in IT, SE and FI, and as ad libitum partial-mixed rations (PMR) plus concentrates during robotic milking in UK. The PMR and TMR were delivered along feed fences in UK and IT, and TMR were delivered into individual feed bins in SE and FI.

Milk and Body Weight Recording:

Milk yield was recorded at every milking and daily mean calculated for each cow. Cows were milked twice daily in herringbone parlors in IT and SE, twice daily at their individual standings in FI, and in automatic milking stations (Lely Astronaut A3, Lely UK Ltd., St Neots, UK), on average 2.85 times per day, in UK.

Milk samples were collected from each cow at four milkings during the sampling period, preserved with broad spectrum microtabs II containing bronopol and natamycin (D & F Control Systems Inc, San Ramon Calif.) or Bronopol (Valio Ltd., Finland), and stored at 4° C. until analyzed. Milk samples were analyzed for fat, protein, lactose and urea concentrations using mid-infrared instruments (Foss Milkoscan, Foss, Denmark, or similar). Mean concentrations of milk components were calculated by weighting concentrations proportionally to respective milk yields from evening and morning milkings.

Body weight was recorded three (SE) or two (IT, FI) times during each sampling period, and automatically at each milking in UK. Mean body weight was calculated for each cow.

Feed Intake Measurement and Estimation

Feed intake was recorded individually on a daily basis throughout each sampling period using Roughage Intake Control (RIC) feeders (Insentec B. V., Marknesse, The Netherlands) in SE and manually in FI. Feed intake was estimated using indigestible markers (alkanes) in feed and feces (27) in UK and IT. Alkanes (C30 and C32) were administered via concentrates fed during milking in UK, and via a bolus gun whilst cows were restrained in locking head yokes during feeding in IT. Validation of the alkane method for estimating feed intake was provided by concurrent direct measurement of individual feed intake in 50 cows in UK via RIC feeders (Fullwood Ltd., Ellesmere, UK), and by applying the method to individually fed cows in a research herd in IT (28).

Collection of Rumen Samples

The method of sampling rumen fluid was standardized at all centers and involved using a ruminal probe specially designed for cattle (Ruminator; profs-products(dot)com). The probe comprises a perforated brass cylinder attached to a reinforced flexible pipe, a suction pump and a collection vessel. The brass cylinder was pushed gently to the back of a cow's mouth and gentle pressure applied until the device was swallowed as far as a ring on the pipe that indicates correct positioning in the rumen. The first liter of rumen fluid was discarded to avoid saliva contamination and the next 0.5 L was retained for sampling. The device was flushed thoroughly with tap water between cows.

Rumen fluid samples were collected on one day during the sampling period between 2 and 5 hours after feed was delivered to cows in the morning. For all samples, pH of rumen fluid was recorded immediately. After swirling, four aliquots of 1 ml each were pipetted into freeze resistant tubes (2 ml capacity), immediately frozen in liquid nitrogen or dry ice, stored at −80° C. and freeze dried within one month from the sampling date. Four additional aliquots of 2.5 mL were pipetted into centrifuge tubes with 0.5 mL of 25% metaphosphoric acid for VFA and ammonia-N analysis, centrifuged at 1000 g for 3 min, and supernatant was transferred to fresh tubes. Tubes were sealed and frozen at −20° C. until laboratory analysis.

Rumen Volatile Fatty Acids Measurement

Volatile fatty acid concentrations were determined by gas chromatography using the method of Playne (29). Ammonia-N concentration was determined by a photometric test with a Clinical Chemistry Autoanalyzer using an enzymatic ultraviolet method (e.g. Randox Laboratories Ltd, Crumlin, UK).

DNA Extraction

Total genomic DNA was isolated from 1 ml freeze dried rumen samples according to Yu and Morrison (30). This method combines bead beating with the column filtration steps of the QIAamp DNA Stool Mini Kit (Qiagen, Hilden, Germany).

Amplicon Sequencing

Primers for PCR amplification of bacterial and archaeal 16S rRNA genes, ciliate protozoal 18S rRNA genes and fungal ITS1 genes were designed in silico using ecoPrimers (31), the OBITools software suite (32) and a database created from sequences stored in GenBank. For each sample, PCR amplifications were performed in duplicate. An eight-nucleotide tag unique to each PCR duplicate was attached to the primer sequence, in order to enable the pooling of all PCR products for sequencing and the subsequent assignation of sequence reads to their respective samples. PCR amplicons were combined in equal volumes and purified using QIAquick PCR purification kit (Qiagen, Germany). After library preparation using a standard protocol with only five PCR cycles, amplicons were sequenced using the MiSeq technology from Illumina (Fasteris, S A, Geneva, Switzerland), which produced 250-base paired-end reads for all markers, except for the archaeal marker which was sequenced with the HiSeq technology from Illumina, generating 100-base pair-end reads.

Methane and CO₂ Emission Measurement

Methane was measured using breath sampling either during milking in UK (33) or when cows visited a bait station in IT and SE (GreenFeed) (34). Methane was measured in FI by housing cows in respiration chambers for 5 days (35). Carbon dioxide was measured simultaneously with methane in IT, SE and FI.

Blood Sampling and Analysis

Blood samples were collected at the same time as rumen sampling using jugular venipuncture and collection into evacuated tubes (Vacutainer). One tube containing Lithium heparin or Na-EDTA as anticoagulant was collected for metabolic parameters, and two tubes containing sodium citrate were collected for genotyping. Tubes were gently inverted 8-10 times following collection to ensure optimal additive activity and prevent clotting. Tubes were chilled at 2-8° C. immediately after collection by placing in chilled water in a fridge or in a mixture of ice and water. Tubes collected for metabolic parameters were centrifuged for 10-15 min (3500 g at 4° C.) and the plasma obtained was divided into four aliquots. Blood samples collected for genotyping were not centrifuged. All samples were stored at −20° C. until analyzed.

Plasma non-esterified fatty acids, beta-hydroxybutyrate, glucose, albumin, cholesterol, urea and creatinine were analyzed at each center using commercial kits (Instrumentation Laboratory, Bedford, Mass., USA; Wako Chemicals GmbH, Neuss, Germany; Randox Laboratories Ltd, Crumlin, UK). Blood samples from each center were sent to IT for haptoglobulin determination, according to the method of Skinner et al. (36).

Quantitative PCR of 16S and 18S rRNA Genes

DNA was diluted to 0.1 ng/μl in 5 μg/ml herring sperm DNA for amplification with universal bacterial primers UniF (GTGSTGCAYGGYYGTCGTCA—SEQ ID NO: 1) and UniR (ACGTCRTCCMCNCCTTCCTC—SEQ ID NO: 2) (37) and 1 ng/μl in 5 μg/ml herring sperm DNA for amplification of other groups (38). Quantitative PCR was carried out using a BioRad CFX96 as described by Ramirez-Farias et al. (39). Amplification of archaeal 16S RNA genes was carried out using the primers Met630f (GGATTAGATACCCSGGTAGT—SEQ ID NO: 3) and Met803r (GTTGARTCCAATTAAACCGCA—SEQ ID NO: 4) as described by Hook et al. (40) and calibrated using DNA extracted from Methanobrevibacter smithii PS, a gift from M. P. Bryant, University of Illinois. For total bacteria amplification efficiency was evaluated using template DNA from Roseburia hominis A2-183 (DSM 16839T). Amplification of protozoal 18S rRNA gene was carried out using primers 316f (GCTTTCGWTGGTAGTGTATT—SEQ ID NO: 5) and 539r (CTTGCCCTCYAATCGTWCT—SEQ ID NO: 6) (41) and calibrated using DNA amplified from bovine rumen digesta with primers 54f and 1747r (41). Bacterial abundance was calculated from quadruplicate Ct values using the universal bacterial calibration equation.

Bovine Genotyping

From blood samples, genomic DNA was extracted and quantified for SNP genotyping. All animals were genotyped on the Bovine GGP HD (GeneSeek Genomic Profilers). The 200 cows coming from Finland and Sweden were genotyped using the Bovine GGP HD chip v1 (80K) that included 76.883 SNPs, while the 800 samples from UK and Italy were genotyped using the Bovine GGP HD chip v2 (150K) that included 138.892 SNPs, as the v1 of the chip was no longer available from the manufacturer. The v2 of the chip includes all the SNPs that were present in the previous v1 of the chip, while at the same time providing more markers for the same final processing cost. Neogen company performed the DNA hybridisation, image scanning and data acquisition of the genotyping chips according to the manufacturer's protocols (Illumina Inc.) All individuals had a call rate higher than 0.90 (93.5% of individuals with call rate higher than 0.99). More than 99% of SNPs had a call rate higher than 0.99, (93.2% of SNPs with call rate higher than 0.99). Minor allele frequency (MAF) distribution evidences more than 90% of markers with a MAF>5% and nearly 4% of monomorphic SNPs.

Quantification and Statistical Analysis

Statistical methods and software used are detailed in subsequent sections, and in figure legends and results. Statistical significance was declared at P<0.05, P<0.01 and P<0.001, as appropriate.

Utilization of Primer Sets Derived Microbiome Data in the Statistical Analysis

Associations of microbial domain richness were based on amplicon sequencing data from the following primer sets: Bact (bacteria), Arch (archaea), Neoc (fungi), Cili (protozoa). Associations of individual microbes (as species-level OTUs) were based on amplicon sequencing data from the following primer sets: ProkA (bacteria and archaea), Neoc (fungi), Cili (protozoa).

Converting OBITools Intermediate Fasta Files to QIIME Ready Format

Amplicon sequences were initially processed with OBITools (32) which removed barcodes and split each sample from each of the two sequencing rounds into an individual FASTQ file. Within each domain's amplicon sequences, individual samples sequences from both rounds were then pooled together into a single FASTQ file in the format required for further processing in QIIME (42) for picking OTU. In detail, the header of each FASTQ entry was appended with a prefix following the format [round_id] [sample_id] [running_number] [space].

Clustering of Microbial Marker Gene Amplicon Sequences and Picking Representative Denovo Species OTU

The marker gene sequences coming from each domain's primer-set (Archaea, Bacteria, Prokaryote, Ciliate, protozoa, and Fungi) were clustered using 97% nucleotide sequence similarity threshold, using the UCLUST algorithm (43), following the QIIME command: pick_otus.py-m uclust-s 0.97). Representative OTUs for each OTU cluster were chosen with QIIME command: pick_rep_set.py-m most_abundant.

Assigning Taxonomy to OTU

The OTU within each domain were assigned taxonomy using the Ribosomal Database Project (RDP) classifier (44), following QIIME command: assign_taxonomy.py-m rdp. The OTUs from the amplicon domains of Prokaryotic, Archaea and Bacteria were assigned taxonomy according to GreenGenes database (45). The OTU from Ciliate protozoa were assigned taxonomy according to SILVA database; release 123 (46). Fungal OTU were assigned taxonomy according to a Neocallimastigomycota ITS ldatabase from Koetschan (47).

Creation of OTU Tables, Samples Subsetting and Subsampling

Amplicon domain OTU tables were created from the representative OTU set counts in each sample along with their assigned taxonomy, using QIIME command: make_otu_table.py. Each OTU table was then subsetted to include only the sample from each animal (out of the two samples sequenced in two different sequencing rounds) that gained the highest sequence depth. Further on, amplicon domain OTU tables were subsampled to 7,000 reads depth for all analyses, with the following exceptions: domain richness (8,000 reads) and microbe abundance to trait association (8,000 reads) and inter-domain microbial interaction analysis, where no subsampling was taking place.

Correlating Microbial Domains Cell Count

The quantitative PCR derived microbial counts in each domain were correlated to each other using Spearman r correlation using R (48) cor function. The P-values for all inter-domain correlations within each farm were corrected using Bonferroni-Hochberg (49) procedure (BH).

Correlating Microbial Domain Cell Counts to Experimental Variables

Within each farm, each experimental variable was correlated to each microbial domain's cell count (Spearman r). Next, the analysis proceeded only with experimental variable—domain count pairs whose correlation direction was identical in all farms. Subsequently, P-values for the correlation of the selected experimental variable—domain cell count pairs from within each farm were combined by meta-analysis using the weighted sum of z procedure (50,51), weighted by the farm size. Meta-analysis was carried by using R package metap (52). Finally, combined P-values were corrected using the BH procedure.

Correlating Microbial Domain Richness to Experimental Variables

Separately within farms, each experimental variable was correlated to each microbial domain's richness, as observed species count (Spearman r), using domain specific primers. Next, the analysis proceeded only with experimental variable—domain richness pairs whose correlation direction was identical in all farms. Subsequently, P-values for the correlation of the selected experimental variable—domain richness pairs from within each farm were combined by meta-analysis using the weighted sum of z procedure, weighted by number of cows on each farm.

Meta-analysis was carried by R package metap (52). Finally, combined P-values were corrected using the BH procedure.

Prediction of Phenotypes and Other Experimental Variables by Core Microbiome

The abundances of the core microbes within each farm were used as features fed into a Ridge regression (56) in order to predict each of the traits (separately). Our approach followed a k-fold cross-validation methodology (k=10) where each fold was omitted once from the entire set and the model built from all the other folds (training set) was used to predict the trait value of the excluded samples (animal). This was implemented using the function cv.glmnet (alpha=0, k=10) from the GLMNET R package (57). Then, the overall prediction r² was calculated using R code

1—model_fit$cvm[which(model_fit$glmnet.fit$lambda==model_fit$lambda.min)]/var(exp_covar). Cross-Validation Procedure was Repeated 1.00 Times and R² Measurements were Averaged.

Prediction of Phenotypes by Core Microbiome while Correcting for Diet

In order to estimate the phenotypic variability explained by core microbes with omission of diet components effect, we repeated the analysis above with one difference. That is, prior to the running the regression, both phenotypic values and microbial OUT counts were corrected for diet. In detail, a Ridge regression (19) was used based on diet components as independent variables and the phenotype or OUT as the dependent variable. Thereafter, the phenotype residuals (diet predicted phenotype—actual phenotype) and OUT residuals (diet predicted OTU count—actual OTU count) were used to feed the GLMNET function (20).

Prediction of Phenotypes by Diet Components

Diet components within each farm were used as features fed into a Ridge regression (19) in order to predict each of the phenotypes (separately). Our approach followed a k-fold cross-validation methodology (k=10) where each fold was omitted once from the entire set and the model built from all the other folds (training set) was used to predict the trait value of the excluded samples (animal). This was implemented using the function cv.glmnet (alpha=0, k=10) from the GLMNET R package (20). Then, the overall prediction r² was calculated using R code

1—model_fit$cvm[which(model_fit$glmnet.fit$lambda==model_fit$lambda.min)]/var(exp_covar). Cross-Validation Procedure was Repeated 1.00 Times and R² Measurements were Averaged.

Prediction of Phenotypes and Other Experimental Variables by Core Microbiome Using Random Forest

As an additional analysis in order to further verify our findings of core microbiome explainability (by prediction) of host phenotypes and experimental variables, we repeated that analysis using RandomForest (RF) regression. The abundances of the core microbes within each farm were used as features fed into a RF regression model (21,22) in order to predict each of the traits (separately). Our approach followed a Leave-one-out cross-validation methodology where in each iteration one sample (animal) was omitted from the entire set and the model built from all the other animals (training set) was used to predict the trait value of the excluded sample (animal). Thereafter, the prediction R² value between vector of actual and predicted values was calculated using R

CARET package function R².

Bovine genotypes quality control Genotypes of the two breed types were processed independently. Genotypes were first subjected to QC filtering including 5% minor frequency allele, 5% genotype missingness and 5% individual missingness, following PLINK (54) command: plink --noweb --cow --maf 0.05 --geno 0.05 --mind 0.05. The QC for the genotypes used for association/heritability analysis (Holstein excluding Farm UK2) resulted with 5377 SNPs failed missingness, 14119 SNPs failed frequency and 48 of 635 individuals removed for low genotyping, resulting with 587 individuals and 121066 remaining.

Testing association of the global rumen prokaryotic core with host genetics Within each farm, the first 30 principal components (PCs) for core OTU were extracted (R prcomp). In addition, first genotypes PCs were extracted using R snpgdsPCA (55). Then, canonical correlation analysis (CCA) (56) was performed between the matrices of OTUs PCs and genotypes PCS, and total fraction of OTUs variance accounted for genotypes variables, through all canonical variates were calculated. This actual value was than compared to that of 1,000 random permutations, where the order of phenotypes PCs was shuffled.

Creation of Genetic Relationship Matrix

A genetic relatedness matrix (GRM) was created including all Holstein animals except Farm UK2, (57), using the command: gcta64 --make-grm-bin --make-bed --autosome-num 29 --autosome.

Heritability Estimation

For estimating OTUs heritability, the core microbes counts were quantile-normalized and were then provided to GCTA to estimate phenotypic variance explained by all SNPs with GREML method (57,58), with farms as qualitative covariates and the first five GRM PCs and diet components as quantitative covariates, following the GCTA command: gcta64--rend-pheno [phenotype_file] -mpheno [phneotype_index]--grm --autosome-num 29 -covar [farms_covars_file]--qcovar [quant_covariates_file].

Heritability Confidence Intervals Estimation

Heritability confidence intervals at 95% were estimated based on the heritability estimates and the GRM using the GRM eigenvalues and farms as covariates with the program FIESTA (59). The command used: fiesta.py --kinship_eigenvalues [GRM_eigenvalues_file] --[heritability_estimates_file] --covariates [farms_covariate_file] --confidence 0.95 --iterations 100 --output_filename [otu_file].

Bovine Genome SNPs—Microbe Association Effort

Microbial species-level OTUs phenotypes within the Holstein subset (excluding UK2 cohort that showed a different genetic makeup by genotypes PCA and ADMIXTURE ancestral background analysis) relative abundance data were transformed using quantile-normalization. Moreover, the top five genotypes top principal components (PCs) and the farm identity were used as a continuous and categorical covariate, respectively. The analysis was performed with the mixed-linear-model option (mlma) where SNP under inspection was accounted as fixed effect along with the covariates, and GRM effect as random. No association p-value surpassed the Bonferroni corrected significance threshold (9.076876e-10) for the number of phenotypes (455) and the number of SNPs included in the association analysis (121,066).

Estimating Kinship Matrix

Farm wise animal genetic kinship matrices as estimated based on genomic relatedness inferred from common single nucleotide polymorphisms (SNPs) that were filtered-in after the above quality control procedure. The tool used for the estimation was EMMAX, with the following command line: emmax-kin-intel64 -v -M 10 farm_genotypes_typed_file -o farm.hBN.kinf

Genomic Prediction

Genomic prediction was performed based on the each farm's kinship matrix. The GAPIT tool was used to predict phenotypic values, with the function GAPIT (parameters PCA.total=3, SNP.test=FALSE). creareFolds command from R caret package (53) was used to create three folds, where in each one fold observations are omitted and are predicted by the model built from the remaining two folds. R² is estimated between the observed and predicted trait values were then correlated using caret R² function. The process was repeated 10 times for a given trait in a given farm and mean of all measurements was then calculated.

Associating Microbes' Abundance with Experimental Variables

Separately for each farm and domain, OTUs occupying more than 10% of the animals in that farm were pairwise correlated (Spearman) to each of the experimental variables. Following that, all P-values resulted from correlation tests within a given domain and farm were subjected to multiple correction using BH procedure. Finally, an OTU that showed a significant correlation (corrected P<0.05) to a certain experimental variable in most (>3) of the farms with same r coefficient sign and no significant correlation with opposite r sign in the remaining farms, was identified as associated with that variable.

Inference of Microbial Interaction Network within Domains

Within each domain and farm, an OTU-table with subset of samples (animals) that contain a depth of at least 5,000 reads was created, followed by removal of OTU present in <50% of animals. The raw counts in the OTU table were fed into the R SpiecEasi (60) framework and edges were identified using spiec.easi function (‘mb’ method). Edges were given weights using symBeta function as suggested by the package authors. Thereafter, the resulting network was filtered to include only edges whose absolute weight was greater than 0.2. Finally, all individual farms within a certain domain were merged and edges connecting nodes (microbes) with the same taxonomic annotation were removed.

Inference of Inter-Domain Microbial Network

Within each farm, OTU from different domains were correlated to each other using Spearman correlation, followed by BH correction for all the correlations examined the farm and filtering in correlations with corrected P<0.05. Then, significant correlations were aggregated from all farms. Finally, correlations with correlation coefficient r<0.5 were removed.

Comparing Phylogenetic Relatedness of Core Prokaryotic Microbes to Random Sampling

Multiple sequence alignment between all core prokaryotic microbes was calculated using MAFFT (61,62) with default parameters. A phylogenetic tree-based distance matrix was obtained from aligned sequences using Fasttree (63,64), following the command: fasttree -nt -makematrix. Thereafter, the median phylogenetic between core microbes was calculated. Next, random sets (n=100) of OTU sequences were subjected to the same procedure. The P-value was calculated as P=(I(mcsd>mrsd)+1)/101, where mcsd represents median core phylogenetic distance and mrsd represents a vector of median phylogenetic distances calculated for the randomly sampled set.

Examining Core and Trait-Related Microbiome for Taxonomic Enrichment

The odds-ratio (O.R.) of each prokaryotic order appearing in the examined group (either core microbiome or trait-related microbiome), between the examined group and the whole prokaryotic microbiome catalog, was calculated. Next, orders showing an O.R.>1 (higher in the examined group) were filtered in. Finally, the O.R. P-value was calculated (Fisher Exact, two-tailed) and corrected using the BH procedure.

Comparing Heritable Microbes to Other Core Miocrobes' Ability to Explain Experimental Variables

In order to compare the ability of heritable microbes vs. other core microbes to explain the experimental variables, we used Ridge regression fit the heritable microbes as independent variables and the experimental variable as the predictable variable. We then contrasted this R² value with other 1.00 R² values achieved from random samples of non-heritable core microbes of same size (39 random microbes). Ridge regression was performed by the R glmnet package. We then compared the R² of heritable microbes to the mean R² of non-heritable core microbes for all the experimental variables altogether, using a paired Wilcoxon rank-sums test.

Seasonality Test:

In each farm core microbes were corrected for diet. Thereafter, the samples in the farm were partitioned into two groups, winter (fall equinox to spring equinox) and summer (spring equinox to fall equinix). Following, each microbial OTU abundance were compared using Wilcoxon rank-sums test that was used to test for difference between the abundance of the given OTU between the two seasons, followed by a multiple comparison correction using the Bonferroni method. Core microbial OTU with corrected P<0.05 in at least one farm were considered as showing a seasonal association.

Results

The study cohort consisted of 1016 animals, with 816 Holstein dairy cows from two UK and three Italian farms. Additionally, two hundred Nordic Red dairy cows were sampled from Sweden and Finland. The Holsteins received a maize silage-based diet, while the Nordic Reds received a nutritionally equivalent diet based on grass silage as forage. Animals were genotyped using common single nucleotide polymorphisms (SNPs) and measured for milk output and composition; feed intake and digestibility; plasma components; methane and CO₂ emissions; and rumen microbiome based on ss rRNA gene analysis.

The abundance and richness of the bacterial, protozoal, fungal and archaeal communities were mutually dependent, and correlated to multiple host phenotypes in ways that have become widely understood, including rumen metabolites, milk production indices and plasma metabolites. In order to focus down on host-microbiome-phenotype relationships, the present inventors proceeded to investigate (i) how many and which species were common in our large animal cohorts, (ii) if a common, or core, group could be identified, (iii) if the core was influenced by the host genome, and (iv) how the core and non-core species determined phenotypic and production characteristics.

Taxonomic analysis revealed a core group of rumen microbes (512 species-level microbial operation taxonomic units (OTUs), 454 prokaryotes, 12 protozoa and 46 fungi) present in at least 50% of animals, within each of the seven farms studied. The group comprised eleven prokaryotic orders, one fungal and two protozoal orders that share some similarity with published core microbial communities (4,15). The core group was shared between Holstein and Nordic Red dairy breeds, and the results are particularly useful because they apply to the most popular and productive milking cow breed used in developed countries, the Holstein, and the smaller breed used in northern European latitudes, the Nordic Red. The results demonstrate once again, however, that this microbial community is representative of ruminants in general, especially with respect to bacterial and protozoal species. This core community is significantly enriched in Bacteroidales, Spirochetales and the WCHB1-4 order. The core microbiome consists of less than 0.25% of the overall microbial species pool (512 out of 250,000 OTUs), yet it is highly abundant, representing 30-60% of the overall microbiome. The core group is also tightly associated with the overall microbiome, as reflected by high correlation between the beta diversity metrics of the identified core microbiome and the overall microbiome across farms (R value between 0.45 and 0.7), this strengthens the notion of strong connectivity between microbes in such a metabolically complex ecosystem where multiple microbial interactions are potentially facilitated. These core microbes show highly conserved abundance rank structure across geography, breed and diet, where the species abundance order is kept almost identical across different individuals. Furthermore, core members are more closely related to each other than to non-core microbiome members, as indicated by differences in phylogenetic distances determined by ss rRNA gene tree. Thus, such relatedness between the members of the rumen core microbiome could indicate that they are sharing a set of functional traits, integral to this environment and potentially compatible with host requirements as suggested for species relatedness in other ecosystems (16). Although the rumen microbiome contains many hundreds of species, these core species generally belong to a rather narrow section of the whole bacterial phylome (17).

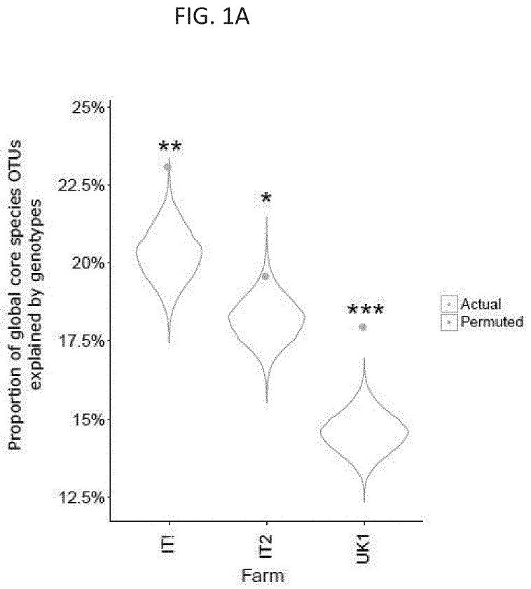

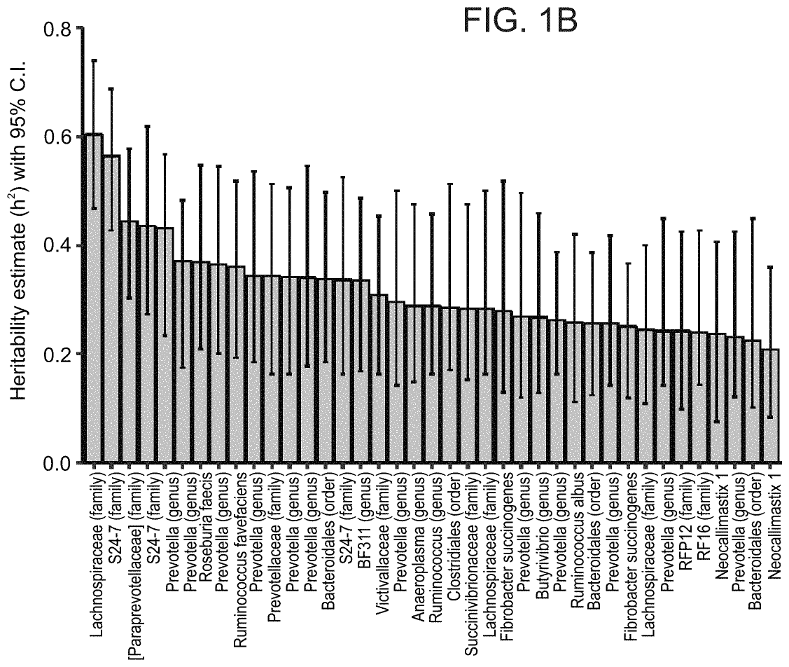

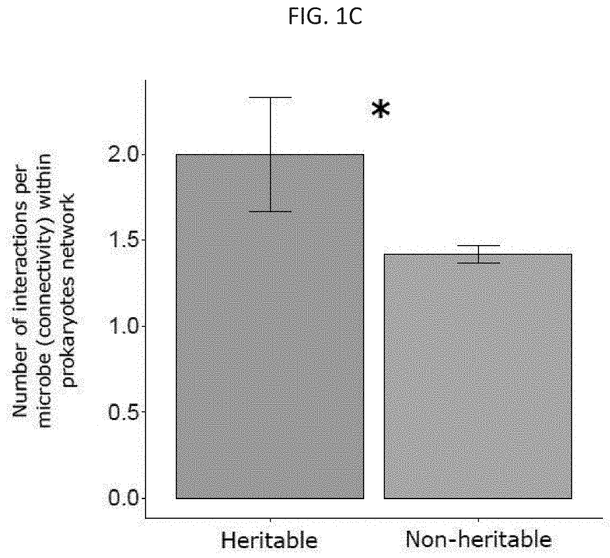

The core microbiome was found to be significantly correlated with host genetics as revealed by Canonical Correlation Analysis (CCA) which was calculated for each farm separately (FIG. 1A). Subsequently a stringent heritability analysis was applied to all members of the core microbiome for each breed separately, taking into account farms and dietary components as a confounding effect (farm encompasses other confounding effects such as location and husbandry regime). Moreover, one Holstein farm (UK2) was removed from the analysis as it showed different genetic background (UK2). The present heritability analysis quantifies specifically narrow-sense, unlike twins-based studies where the type of heritability is not strictly defined (14). This is especially true for bovines where twin-rate is low and these individuals are often born unwell, rendering them unfit for such studies. Within the Holstein-Friesian breed (n=650, excluding 166), 39 heritable core microbial OTUs were identified, which were evenly distributed on the rank abundance curve therefore pointing out that low abundance species could also be connected to host genome and suggesting relevance to its requirements.

These mainly belonging to Bacteroidales and Clostridiales orders, but also including representatives from five other bacterial phyla and two fungi, of the genus Neocallimastix (FIG. 1B). Ruminococcus and Fibrobacter are among the core heritable bacteria, consistent with their key role in cellulolysis, as is Succinovibrionaceae, which seems to be a key determinant in between-animal differences in methane emissions (18). These heritable microbial OTUs showed significant heritability estimates ranging from 0.2 to 0.6 (P<0.05 FDR), and revealed a two-fold increase in numbers of microbial heritable species over previous study (15) that included a smaller animal cohort. Furthermore, these highly robust findings also reinforce our previous results in relation to heritable bovine rumen microbes, which are composed of similar taxa. Moreover, based on the genetic relatedness matrix (GRM), the heritability confidence interval lower-limit of all but one microbe was greater than 0.1. Only three bacteria, all with affiliations to Prevotellaceae, were identified as highly heritable within the smaller Nordic Red cohort. In summary, we identified almost ten times more heritable species level microbial OTUs than in a comparable human study (14), further substantiating the deep interaction between the bovine host and its resident rumen microbiome, reflecting presumably the greater dependence of the bovine on its gut microbiome than humans.

Table 1 summarizes all the hereditable bacteria that are associated with traits.

TABLE 1

| | | Correlation | Correlation | SEQ | |
|---|---|---|---|---|---|
| OTU_ID | Host Trait | size | direction | ID NO: | Taxonomy |
| denovo | Rumen | 0.562909203 | Negative | 7 | k__Bacteria; p__Bacteroidetes; c__Bacteroidia; |
| 1359435 | Propionate | | | | o__Bacteroidales; f__RF16; g__; s__ |
| denovo | Rumen | 0.666170664 | Negative | 8 | k__Bacteria; p__Proteobacteria; c__Gammaproteobacteria; |
| 1636556 | Propionate | | | | o__Aeromonadales; f__Succinivibrionaceae; g__; s__ |
| denovo | Rumen | 0.530183154 | Negative | 9 | k__Bacteria; p__Bacteroidetes; c__Bacteroidia; |
| 1690942 | Propionate | | | | o__Bacteroidales; f__Bacteroidaceae; g__BF311; s__ |
| denovo | Rumen | 0.458024186 | Negative | 10 | k__Bacteria; p__Bacteroidetes; c__Bacteroidia; |
| 1708915 | Acetate | | | | o__Bacteroidales; f__; g__; s__ |
| denovo | Milk | 0.302242926 | Negative | 11 | k__Bacteria; p__Lentisphaerae; c__[Lentisphaeria]; |
| 1803355 | lactose | | | | o__Victivallales; f__Victivallaceae; g__; s__ |
| denovo | Milk | 0.294008329 | Negative | 12 | k__Bacteria; p__Lentisphaerae; c__[Lentisphaeria]; |
| 1803355 | yield | | | | o__Victivallales; f__Victivallaceae; g__; s__ |
| denovo | Rumen | 0.520413813 | Negative | 13 | k__Bacteria; p__Lentisphaerae; c__[Lentisphaeria]; |
| 1803355 | Propionate | | | | o__Victivallales; f__Victivallaceae; g__; s__ |
| denovo | Rumen | 0.569716587 | Negative | 14 | k__Bacteria; p__Bacteroidetes; c__Bacteroidia; |
| 2090355 | Propionate | | | | o__Bacteroidales; f__Prevotellaceae; g__Prevotella; s__ |
| denovo | Rumen | 0.506248906 | Negative | 15 | k__Bacteria; p__Verrucomicrobia; c__Verruco-5; |
| 264956 | Propionate | | | | o__WCHB1-41; f__RFP12; g__; s__ |
| denovo | Rumen | 0.560982196 | Positive | 16 | k__Bacteria; p__Bacteroidetes; c__Bacteroidia; |
| 1359435 | Acetate | | | | o__Bacteroidales; f__RF16; g__; s__ |
| denovo | Milk fat | 0.316869663 | Positive | 17 | k__Bacteria; p__Bacteroidetes; c__Bacteroidia; |
| 1690942 | | | | | o__Bacteroidales; f__Bacteroidaceae; g__BF311; s__ |
| denovo | Rumen | 0.521038537 | Positive | 18 | k__Bacteria; p__Bacteroidetes; c__Bacteroidia; |
| 1690942 | Acetate | | | | o__Bacteroidales; f__Bacteroidaceae; g__BF311; s__ |
| denovo | Rumen | 0.283654532 | Positive | 19 | k__Bacteria; p__Bacteroidetes; c__Bacteroidia; |
| 1690942 | pH | | | | o__Bacteroidales; f__Bacteroidaceae; g__BF311; s__ |
| denovo | Plasma | 0.319545607 | Positive | 20 | k__Bacteria; p__Bacteroidetes; c__Bacteroidia; |
| 2090355 | BHB | | | | o__Bacteroidales; f__Prevotellaceae; g__Prevotella; s__ |
| denovo | Rumen | 0.410797419 | Positive | 21 | k__Bacteria; p__Bacteroidetes; c__Bacteroidia; |
| 2090355 | Butyrate | | | | o__Bacteroidales; f__Prevotellaceae; g__Prevotella; s__ |
| denovo | Rumen | 0.328619039 | Positive | 22 | k__Bacteria; p__Fibrobacteres; c__Fibrobacteria; |
| 2090357 | Acetate | | | | o__Fibrobacterales; f__Fibrobacteraceae; g__Fibrobacter; |
| | | | | | s__succinogenes |
| denovo | Rumen | 0.396476088 | Positive | 23 | k__Bacteria; p__Verrucomicrobia; c__Verruco-5; |
| 264956 | Acetate | | | | o__WCHB1-41; f__RFP12; g__; s__ |
| denovo | Rumen | 0.358083607 | Positive | 24 | k__Bacteria; p__Firmicutes; c__Clostridia; o__Clostridiales; |
| 642135 | Butyrate | | | | f__Lachnospiraceae |
| denovo | Rumen | 0.618988642 | Positive | 25 | k__Bacteria; p__Proteobacteria; c__Gammaproteobacteria; |
| 1636556 | Acetate | | | | o__Aeromonadales; f__Succinivibrionaceae; g__; s__ |
| denovo | Rumen | 0.387638669 | Positive | 26 | k__Bacteria; p__Bacteroidetes; c__Bacteroidia; |
| 1708915 | Propionate | | | | o__Bacteroidales; f__; g__; s__ |
| denovo | Rumen | 0.513679373 | Positive | 27 | k__Bacteria; p__Lentisphaerae; c__[Lentisphaeria]; |
| 1803355 | Acetate | | | | o__Victivallales; f__Victivallaceae; g__; s__ |
| denovo | Rumen | 0.371548345 | Positive | 28 | k__Bacteria; p__Bacteroidetes; c__Bacteroidia; |
| 244987 | Butyrate | | | | o__Bacteroidales; f__Prevotellaceae; g__Prevotella; s__ |

Table 2 summarizes all bacteria which correlated with a trait identified in this study.

TABLE 2

| | | | | | SEQ | |
|---|---|---|---|---|---|---|
| | | Correlation | Correlation | | ID | Is |
| OTU_ID | Host Trait | size | direction | Taxonomy | NO: | heritable? |
| deno- | Rumen | 0.562909203 | Negative | k_Bacteria; p_Bacteroidetes; c_Bacteroidia; | 29 | YES |
| vo1359435 | Propionate | | | o_Bacteroidales; f_RF16; g_; s_ | | |
| deno- | Rumen | 0.666170664 | Negative | k_Bacteria; p_Proteobacteria; | 30 | YES |
| vo1636556 | Propionate | | | c_Gammaproteobacteria; o_Aeromonadales; | | |
| | | | | f_Succinivibrionaceae; g_; s_ | | |
| deno- | Rumen | 0.530183154 | Negative | k_Bacteria; p_Bacteroidetes; c_Bacteroidia; | 31 | YES |
| vo1690942 | Propionate | | | o_Bacteroidales; f_Bacteroidaceae; g_BF311; | | |
| | | | | s_ | | |
| deno- | Rumen | 0.458024186 | Negative | k_Bacteria; p_Bacteroidetes; c_Bacteroidia; | 32 | YES |
| vo1708915 | Acetate | | | o_Bacteroidales; f_; g_; s_ | | |
| deno- | Milk | 0.302242926 | Negative | k_Bacteria; p_Lentisphaerae; c_[Lentisphaeria]; | 33 | YES |
| vo1803355 | lactose | | | o_Victivallales; f_Victivallaceae; g_; s_ | | |
| deno- | Milk | 0.294008329 | Negative | k_Bacteria; p_Lentisphaerae; c_[Lentisphaeria]; | 34 | YES |
| vo1803355 | yield | | | o_Victivallales; f_Victivallaceae; g_; s_ | | |
| deno- | Rumen | 0.520413813 | Negative | k_Bacteria; p_Lentisphaerae; c_[Lentisphaeria]; | 35 | YES |
| vo1803355 | Propionate | | | o_Victivallales; f_Victivallaceae; g_; s_ | | |
| deno- | Rumen | 0.569716587 | Negative | k_Bacteria; p_Bacteroidetes; c_Bacteroidia; | 36 | YES |
| vo2090355 | Propionate | | | o_Bacteroidales; f_Prevotellaceae; g_Prevotella; | | |
| | | | | s_ | | |
| deno- | Rumen | 0.506248906 | Negative | k_Bacteria; p_Verrucomicrobia; c_Verruco-5; | 37 | YES |
| vo264956 | Propionate | | | o_WCHB1-41; f_RFP12; g_; s_ | | |
| deno- | Rumen | 0.560982196 | Positive | k_Bacteria; p_Bacteroidetes; c_Bacteroidia; | 38 | YES |
| vo1359435 | Acetate | | | o_Bacteroidales; f_RF16; g_; s_ | | |
| deno- | Milk fat | 0.316869663 | Positive | k_Bacteria; p_Bacteroidetes; c_Bacteroidia; | 39 | YES |
| vo1690942 | | | | o_Bacteroidales; f_Bacteroidaceae; g_BF311; | | |
| | | | | s_ | | |
| deno- | Rumen | 0.521038537 | Positive | k_Bacteria; p_Bacteroidetes; c_Bacteroidia; | 40 | YES |
| vo1690942 | Acetate | | | o_Bacteroidales; f_Bacteroidaceae; g_BF311; | | |
| | | | | s_ | | |
| deno- | Rumen | 0.283654532 | Positive | k_Bacteria; p_Bacteroidetes; c_Bacteroidia; | 41 | YES |
| vo1690942 | pH | | | o_Bacteroidales; f_Bacteroidaceae; g_BF311; | | |
| | | | | s_ | | |
| deno- | Plasma | 0.319545607 | Positive | k_Bacteria; p_Bacteroidetes; c_Bacteroidia; | 42 | YES |
| vo2090355 | BHB | | | o_Bacteroidales; f_Prevotellaceae; g_Prevotella; | | |
| | | | | s_ | | |
| deno- | Rumen | 0.410797419 | Positive | k_Bacteria; p_Bacteroidetes; c_Bacteroidia; | 43 | YES |
| vo2090355 | Butyrate | | | o_Bacteroidales; f_Prevotellaceae; g_Prevotella; | | |
| | | | | s_ | | |
| deno- | Rumen | 0.328619039 | Positive | k_Bacteria; p_Fibrobacteres; c_Fibrobacteria; | 44 | YES |
| vo2090357 | Acetate | | | o_Fibrobacterales; f_Fibrobacteraceae; | | |
| | | | | g_Fibrobacter; s_succinogenes | | |
| deno- | Rumen | 0.396476088 | Positive | k_Bacteria; p_Verrucomicrobia; c_Verruco-5; | 45 | YES |
| vo264956 | Acetate | | | o_WCHB1-41; f_RFP12; g_; s_ | | |
| deno- | Rumen | 0.358083607 | Positive | k_Bacteria; p_Firmicutes; c_Clostridia; | 46 | YES |
| vo642135 | Butyrate | | | o_Clostridiales; f_Lachnospiraceae | | |
| deno- | Rumen | 0.618988642 | Positive | k_Bacteria; p_Proteobacteria; | 47 | YES |
| vo1636556 | Acetate | | | c_Gammaproteobacteria; o_Aeromonadales; | | |
| | | | | f_Succinivibrionaceae; g_; s_ | | |
| deno- | Rumen | 0.387638669 | Positive | k_Bacteria; p_Bacteroidetes; c_Bacteroidia; | 48 | YES |
| vo1708915 | Propionate | | | o_Bacteroidales; f_; g_; s_ | | |
| deno- | Rumen | 0.513679373 | Positive | k_Bacteria; p_Lentisphaerae; c_[Lentisphaeria]; | 49 | YES |
| vo1803355 | Acetate | | | o_Victivallales; f_Victivallaceae; g_; s_ | | |
| deno- | Rumen | 0.371548345 | Positive | k_Bacteria; p_Bacteroidetes; c_Bacteroidia; | 50 | YES |
| vo244987 | Butyrate | | | o_Bacteroidales; f_Prevotellaceae; g_Prevotella; | | |
| | | | | s_ | | |
| deno- | Rumen | 0.292996955 | Negative | k_Bacteria; p_Spirochaetes; c_Spirochaetes; | 51 | YES |
| vo1003904 | Valerate | | | o_Spirochaetales; f_Spirochaetaceae; | | |
| | | | | g_Treponema; s_ | | |
| deno- | Rumen | 0.387235172 | Negative | k_Bacteria; p_Firmicutes; c_Clostridia; | 52 | |
| vo1004279 | Acetate | | | o_Clostridiales; f_Ruminococcaceae; | | |
| | | | | g_Ruminococcus; s_flavefaciens | | |
| deno- | Rumen | 0.536485658 | Negative | k_Bacteria; p_Bacteroidetes; c_Bacteroidia; | 53 | |
| vo1018333 | Acetate | | | o_Bacteroidales; f_; g_; s_ | | |
| deno- | Rumen | 0.345790434 | Negative | k_Bacteria; p_Bacteroidetes; c_Bacteroidia; | 54 | |
| vo101870 | Propionate | | | o_Bacteroidales; f_Prevotellaceae; g_Prevotella; | | |
| | | | | s_ | | |
| deno- | Rumen | 0.578791411 | Negative | k_Bacteria; p_Bacteroidetes; c_Bacteroidia; | 55 | |
| vo1045128 | Acetate | | | o_Bacteroidales; f_Prevotellaceae; g_Prevotella; | | |
| | | | | s_ | | |
| deno- | Rumen | 0.30770895 | Negative | k_Bacteria; p_Bacteroidetes; c_Bacteroidia; | 56 | |
| vo1046267 | Propionate | | | o_Bacteroidales; f_S24-7; g_; s_ | | |
| deno- | Rumen | 0.658373488 | Negative | k_Bacteria; p_Bacteroidetes; c_Bacteroidia; | 57 | |
| vo1065963 | Propionate | | | o_Bacteroidales; f_[Paraprevotellaceae]; | | |
| | | | | g_CF231; s_ | | |
| deno- | Rumen | 0.447040755 | Negative | k_Bacteria; p_Elusimicrobia; c_Endomicrobia; | 58 | |
| vo1070363 | Propionate | | | o_; f_; g_; s_ | | |
| deno- | Rumen | 0.410872244 | Negative | k_Bacteria; p_Bacteroidetes; c_Bacteroidia; | 59 | |
| vo1086049 | Acetate | | | o_Bacteroidales; f_Prevotellaceae; g_Prevotella; | | |
| | | | | s_ | | |
| deno- | Rumen | 0.477090339 | Negative | k_Bacteria; p_Firmicutes; c_Clostridia; | 60 | |
| vo1096469 | Acetate | | | o_Clostridiales; f_Veillonellaceae; g_Dialister; | | |
| | | | | s_ | | |
| deno- | Rumen | 0.296121358 | Negative | k_Bacteria; p_Bacteroidetes; c_Bacteroidia | 61 | |
| vo115455 | Propionate | | | o_Bacteroidales; f_BS11; g_; s_ | | |
| deno- | Rumen | 0.422917201 | Negative | k_Bacteria; p_Verrucomicrobia; c_Verruco-5; | 62 | |
| vo1163072 | Propionate | | | o_WCHB1-41; f_RFP12; g_; s_ | | |
| deno- | Rumen | 0.518874312 | Negative | k_Bacteria; p_Bacteroidetes; c_Bacteroidia; | 63 | |
| vo1178104 | Acetate | | | o_Bacteroidales; f_Prevotellaceae; g_Prevotella; | | |
| | | | | s_ | | |
| deno- | Rumen | 0.571431102 | Negative | k_Bacteria; p_Firmicutes; c_Clostridia; | 64 | |
| vo1209472 | Acetate | | | o_Clostridiales; f_Lachnospiraceae; | | |
| | | | | g_Shuttleworthia; s_ | | |
| deno- | Rumen | 0.539632299 | Negative | k_Bacteria; p_Proteobacteria; | 65 | |
| vo1221142 | Propionate | | | c_Gammaproteobacteria; o_Aeromonadales; | | |
| | | | | f_Succinivibrionaceae; g_; s_ | | |
| deno- | Plasma | 0.305747467 | Negative | k_Bacteria; p_Bacteroidetes; c_Bacteroidia; | 66 | |
| vo1221444 | BHB | | | o_Bacteroidales; f_Prevotellaceae; g_Prevotella; | | |
| | | | | s_ | | |
| deno- | Rumen | 0.574278559 | Negative | k_Bacteria; p_Bacteroidetes; c_Bacteroidia; | 67 | |
| vo1221444 | Acetate | | | o_Bacteroidales; f_Prevotellaceae; g_Prevotella; | | |
| | | | | s_ | | |
| deno- | Rumen | 0.583898459 | Negative | k_Bacteria; p_Proteobacteria; | 68 | |
| vo1229628 | Propionate | | | c_Gammaproteobacteria; o_Aeromonadales; | | |
| | | | | f_Succinivibrionaceae; g_; s_ | | |
| deno- | Rumen | 0.332414705 | Negative | k_Bacteria; p_Bacteroidetes; c_Bacteroidia; | 69 | |
| vo1239670 | Propionate | | | o_Bacteroidales; f_S24-7; g_; s_ | | |
| deno- | Rumen | 0.391739677 | Negative | k_Bacteria; p_Bacteroidetes; c_Bacteroidia; | 70 | |
| vo1240314 | Propionate | | | o_Bacteroidales; f_Prevotellaceae; g_Prevotella; | | |
| | | | | s_ | | |
| deno- | Rumen | 0.526950919 | Negative | k_Bacteria; p_Bacteroidetes; c_Bacteroidia; | 71 | |
| vo1244578 | Acetate | | | o_Bacteroidales; f_Prevotellaceae; g_Prevotella; | | |
| | | | | s_ | | |
| deno- | Rumen | 0.546554782 | Negative | k_Bacteria; p_Proteobacteria; | 72 | |
| vo1256657 | Propionate | | | c_Gammaproteobacteria; o_Aeromonadales; | | |
| | | | | f_Succinivibrionaceae; g_; s_ | | |
| deno- | Rumen | 0.324465923 | Negative | k_Bacteria; p_Bacteroidetes; c_Bacteroidia; | 73 | |
| vo1283388 | Propionate | | | o_Bacteroidales; f_Prevotellaceae; g_Prevotella; | | |
| | | | | s_ | | |
| deno- | Rumen | 0.391932774 | Negative | k_Bacteria; p_Bacteroidetes; c_Bacteroidia; | 74 | |
| vo129818 | Acetate | | | o_Bacteroidales; f_Prevotellaceae; g_Prevotella; | | |
| | | | | s_ | | |
| deno- | Rumen | 0.360530961 | Negative | k_Bacteria; p_Bacteroidetes; c_Bacteroidia; | 75 | |
| vo1308850 | Acetate | | | o_Bacteroidales; f_Prevotellaceae; g_Prevotella; | | |
| | | | | s_ | | |
| deno- | Rumen | 0.552484974 | Negative | k_Bacteria; p_Bacteroidetes; c_Bacteroidia; | 76 | |
| vo131546 | Acetate | | | o_Bacteroidales; f_Prevotellaceae; g_Prevotella; | | |
| | | | | s_ | | |
| deno- | Rumen | 0.328526465 | Negative | k_Bacteria; p_Firmicutes; c_Clostridia; | 77 | |
| vo1322523 | Propionate | | | o_Clostridiales; f_Lachnospiraceae | | |
| deno- | Rumen | 0.493200828 | Negative | k_Bacteria; p_Firmicutes; c_Clostridia; | 78 | |
| vo1325041 | Acetate | | | o_Clostridiales; f_Lachnospiraceae | | |
| deno- | Rumen | 0.385909986 | Negative | k_Bacteria; p_Bacteroidetes; c_Bacteroidia; | 79 | |
| vo1326222 | Propionate | | | o_Bacteroidales; f_Prevotellaceae; g_Prevotella; | | |
| | | | | s_ | | |
| deno- | Milk fat | 0.427566538 | Negative | k_Bacteria; p_Bacteroidetes; c_Bacteroidia; | 80 | |
| vo1329931 | | | | o_Bacteroidales; f_Prevotellaceae; g_Prevotella; | | |
| | | | | s_ | | |
| deno- | Rumen | 0.597323455 | Negative | k_Bacteria; p_Bacteroidetes; c_Bacteroidia; | 81 | |
| vo1329931 | Acetate | | | o_Bacteroidales; f_Prevotellaceae; g_Prevotella; | | |
| | | | | s_ | | |
| deno- | Rumen | 0.566493969 | Negative | k_Bacteria; p_Bacteroidetes; c_Bacteroidia; | 82 | |
| vo1361244 | Acetate | | | o_Bacteroidales; f_Prevotellaceae; g_Prevotella; | | |
| | | | | s_ | | |
| deno- | Rumen | 0.371684952 | Negative | k_Bacteria; p_Bacteroidetes; c_Bacteroidia; | 83 | |
| vo1366510 | Propionate | | | o_Bacteroidales; f_Prevotellaceae; g_Prevotella; | | |
| | | | | s_ | | |
| deno- | Diet | 0.405050837 | Negative | k_Bacteria; p_Bacteroidetes; c_Bacteroidia; | 84 | |
| vo1377006 | starch | | | o_Bacteroidales; f_Prevotellaceae; g_Prevotella; | | |
| | | | | s_ | | |
| deno- | Milk fat | 0.385571225 | Negative | k_Bacteria; p_Bacteroidetes; c_Bacteroidia; | 85 | |
| vo1380399 | | | | o_Bacteroidales; f_Prevotellaceae; g_Prevotella; | | |
| | | | | s_ | | |
| deno- | Plasma | 0.328883904 | Negative | k_Bacteria; p_Bacteroidetes; c_Bacteroidia; | 86 | |
| vo1380399 | BHB | | | o_Bacteroidales; f_Prevotellaceae; g_Prevotella; | | |
| | | | | s_ | | |
| deno- | Rumen | 0.595652117 | Negative | k_Bacteria; p_Bacteroidetes; c_Bacteroidia; | 87 | |
| vo1380399 | Acetate | | | o_Bacteroidales; f_Prevotellaceae; g_Prevotella; | | |
| | | | | s_ | | |
| deno- | Rumen | 0.604178171 | Negative | k_Bacteria; p_Bacteroidetes; c_Bacteroidia; | 88 | |
| vo1385456 | Propionate | | | o_Bacteroidales; f_Prevotellaceae; g_Prevotella; | | |
| | | | | s_ | | |
| deno- | Milk fat | 0.385396271 | Negative | k_Bacteria; p_Bacteroidetes; c_Bacteroidia; | 89 | |
| vo1389131 | | | | o_Bacteroidales; f_Prevotellaceae; g_Prevotella; | | |
| | | | | s_ | | |
| deno- | Rumen | 0.589883672 | Negative | k_Bacteria; p_Bacteroidetes; c_Bacteroidia; | 90 | |
| vo1389131 | Acetate | | | o_Bacteroidales; f_Prevotellaceae; g_Prevotella; | | |
| | | | | s_ | | |
| deno- | Rumen | 0.470131286 | Negative | k_Bacteria; p_Bacteroidetes; c_Bacteroidia; | 91 | |
| vo1410364 | Acetate | | | o_Bacteroidales; f_Prevotellaceae; g_Prevotella; | | |
| | | | | s_ | | |
| deno- | Rumen | 0.493109492 | Negative | k_Bacteria; p_Bacteroidetes; c_Bacteroidia; | 92 | |
| vo1411011 | Propionate | | | o_Bacteroidales; f_Prevotellaceae; g_Prevotella; | | |
| | | | | s_ | | |
| deno- | Rumen | 0.599278408 | Negative | k_Bacteria; p_Bacteroidetes; c_Bacteroidia; | 93 | |
| vo1423479 | Acetate | | | o_Bacteroidales; f_Prevotellaceae; g_Prevotella; | | |
| | | | | s_ | | |
| deno- | Diet | 0.366823421 | Negative | k_Bacteria; p_Firmicutes; c_Clostridia; | 94 | |
| vo1432874 | crude | | | o_Clostridiales; f_Lachnospiraceae; | | |
| | protein | | | g_Butyrivibrio; s_ | | |
| deno- | Rumen | 0.579158536 | Negative | k_Bacteria; p_Bacteroidetes; c_Bacteroidia; | 95 | |
| vo1440570 | Acetate | | | o_Bacteroidales; f_Prevotellaceae; g_Prevotella; | | |
| | | | | s_ | | |
| deno- | Rumen | 0.3728422 | Negative | k_Bacteria; p_Firmicutes; c_Clostridia; | 96 | |
| vo1444540 | Acetate | | | o_Clostridiales; f_Lachnospiraceae; g_; s_ | | |
| deno- | Rumen | 0.275783872 | Negative | k_Bacteria; p_Bacteroidetes; c_Bacteroidia; | 97 | |
| vo1446200 | Acetate | | | o_Bacteroidales; f_Prevotellaceae; g_Prevotella | | |
| deno- | Rumen | 0.436317109 | Negative | k_Bacteria; p_Firmicutes; c_Clostridia; | 98 | |
| vo145213 | Acetate | | | o_Clostridiales; f_; g_; s_ | | |
| deno- | Rumen | 0.513658167 | Negative | k_Bacteria; p_Bacteroidetes; c_Bacteroidia; | 99 | |
| vo1462600 | Propionate | | | o_Bacteroidales; f_[Paraprevotellaceae]; g_; s_ | | |
| deno- | Rumen | 0.433939263 | Negative | k_Bacteria; p_Bacteroidetes; c_Bacteroidia; | 100 | |
| vo1464133 | Acetate | | | o_Bacteroidales; f_Prevotellaceae; g_Prevotella; | | |
| | | | | s_ | | |
| deno- | Rumen | 0.433480186 | Negative | k_Bacteria; p_Bacteroidetes; c_Bacteroidia; | 101 | |
| vo1465009 | Propionate | | | o_Bacteroidales; f_Prevotellaceae; g_Prevotella; | | |
| | | | | s_ | | |
| deno- | Rumen | 0.455541868 | Negative | k_Bacteria; p_Fibrobacteres; c_Fibrobacteria; | 102 | |
| vo1470326 | Propionate | | | o_Fibrobacterales; f_Fibrobacteraceae; | | |
| | | | | g_Fibrobacter; s_succinogenes | | |
| deno- | Rumen | 0.365568791 | Negative | k_Bacteria; p_Firmicutes; c_Clostridia; | 103 | |
| vo1473970 | Propionate | | | o_Clostridiales; f_; g_; s_ | | |
| deno- | CH4 | 0.459205968 | Negative | k_Bacteria; p_Firmicutes; c_Clostridia; | 104 | |
| vo1477974 | g/kg | | | o_Clostridiales; f_Lachnospiraceae; | | |
| | ECM | | | g_Shuttleworthia; s_ | | |
| deno- | Rumen | 0.633050029 | Negative | k_Bacteria; p_Firmicutes; c_Clostridia; | 105 | |
| vo1477974 | Acetate | | | o_Clostridiales; f_Lachnospiraceae; | | |
| | | | | g_Shuttleworthia; s_ | | |
| deno- | Diet | 0.246876495 | Negative | k_Bacteria; p_Firmicutes; c_Clostridia; | 106 | |
| vo1494447 | organic | | | o_Clostridiales; f_; g_; s_ | | |
| | matter | | | | | |
| deno- | Rumen | 0.537025222 | Negative | k_Bacteria; p_Firmicutes; c_Clostridia; | 107 | |
| vo1497746 | Acetate | | | o_Clostridiales; f_Veillonellaceae; g_Dialister; | | |
| | | | | s_ | | |
| deno- | Milk fat | 0.374808564 | Negative | k_Bacteria; p_Bacteroidetes; c_Bacteroidia; | 108 | |
| vo1503183 | | | | o_Bacteroidales; f_Prevotellaceae; g_Prevotella; | | |
| | | | | s_ | | |
| deno- | Rumen | 0.610725696 | Negative | k_Bacteria; p_Bacteroidetes; c_Bacteroidia; | 109 | |
| vo1503183 | Acetate | | | o_Bacteroidales; f_Prevotellaceae; g_Prevotella; | | |
| | | | | s_ | | |
| deno- | Rumen | 0.260691489 | Negative | k_Bacteria; p_Firmicutes; c_Clostridia; | 110 | |
| vo1510345 | Propionate | | | o_Clostridiales; f_Lachnospiraceae; g_; s_ | | |
| deno- | Rumen | 0.480926229 | Negative | k_Bacteria; p_Proteobacteria; | 111 | |
| vo1513549 | Acetate | | | c_Gammaproteobacteria; o_Aeromonadales; | | |
| | | | | f_Succinivibrionaceae; g_; s_ | | |
| deno- | Rumen | 0.381710542 | Negative | k_Bacteria; p_Proteobacteria; | 112 | |
| vo1518048 | Acetate | | | c_Gammaproteobacteria; o_Aeromonadales; | | |
| | | | | f_Succinivibrionaceae; g_; s_ | | |
| deno- | Rumen | 0.402592719 | Negative | k_Bacteria; p_Proteobacteria; | 113 | |
| vo1550126 | Acetate | | | c_Gammaproteobacteria; o_Aeromonadales; | | |
| | | | | f_Succinivibrionaceae; g_; s_ | | |
| deno- | Rumen | 0.367094432 | Negative | k_Bacteria; p_Bacteroidetes; c_Bacteroidia; | 114 | |
| vo1558177 | Propionate | | | o_Bacteroidales; f_Prevotellaceae; g_Prevotella; | | |
| | | | | s_ | | |
| deno- | Rumen | 0.358494508 | Negative | k_Bacteria; p_Bacteroidetes; c_Bacteroidia; | 115 | |
| vo1558873 | Propionate | | | o_Bacteroidales; f_S24-7; g_; s_ | | |
| deno- | Rumen | 0.510525409 | Negative | k_Bacteria; p_Bacteroidetes; c_Bacteroidia; | 116 | |
| vo1559976 | Acetate | | | o_Bacteroidales; f_Prevotellaceae; g_Prevotella; | | |
| | | | | s_ | | |
| deno- | Rumen | 0.545649043 | Negative | k_Bacteria; p_Verrucomicrobia; c_Verruco-5; | 117 | |
| vo156185 | Propionate | | | o_WCHB1-41; f_RFP12; g_; s_ | | |
| deno- | Rumen | 0.343537966 | Negative | k_Bacteria; p_Bacteroidetes; c_Bacteroidia; | 118 | |
| vo1563532 | Propionate | | | o_Bacteroidales; f_[Paraprevotellaceae]; | | |
| | | | | g_YRC22; s_ | | |
| deno- | Rumen | 0.560722816 | Negative | k_Bacteria; p_Proteobacteria; | 119 | |
| vo1566947 | Propionate | | | c_Gammaproteobacteria; o_Aeromonadales; | | |
| | | | | f_Succinivibrionaceae; g_; s_ | | |
| deno- | Rumen | 0.477650459 | Negative | k_Bacteria; p_Firmicutes; c_Clostridia; | 120 | |
| vo1570766 | Propionate | | | o_Clostridiales; f_; g_; s_ | | |
| deno- | Rumen | 0.383700701 | Negative | k_Bacteria; p_Bacteroidetes; c_Bacteroidia; | 121 | |
| vo1582440 | Propionate | | | o_Bacteroidales; f_; g_; s_ | | |
| deno- | Rumen | 0.423375801 | Negative | k_Bacteria; p_Bacteroidetes; c_Bacteroidia; | 122 | |
| vo1603432 | Acetate | | | o_Bacteroidales; f_Prevotellaceae; g_Prevotella; | | |
| | | | | s_ | | |
| deno- | Rumen | 0.660150537 | Negative | k_Bacteria; p_Bacteroidetes; c_Bacteroidia; | 123 | |
| vo1603971 | Propionate | | | o_Bacteroidales; f_Prevotellaceae; g_Prevotella; | | |
| | | | | s_ | | |
| deno- | Rumen | 0.431310878 | Negative | k_Bacteria; p_Verrucomicrobia; c_Verruco-5; | 124 | |
| vo1613585 | Propionate | | | o_WCHB1-41; f_RFP12; g_; s_ | | |
| deno- | Rumen | 0.44276672 | Negative | k_Bacteria; p_Firmicutes; c_Clostridia; | 125 | |
| vo1627012 | Acetate | | | o_Clostridiales; f_Lachnospiraceae; | | |
| | | | | g_ Coprococcus; s_ | | |
| deno- | Diet | 0.341073038 | Negative | k_Bacteria; p_Firmicutes; c_Clostridia; | 126 | |
| vo1637096 | starch | | | o_Clostridiales; f_; g_; s_ | | |
| deno- | Rumen | 0.656242697 | Negative | k_Bacteria; p_Bacteroidetes; c_Bacteroidia; | 127 | |
| vo1641807 | Acetate | | | o_Bacteroidales; f_Prevotellaceae; g_Prevotella; | | |
| | | | | s_ | | |
| deno- | Rumen | 0.513982458 | Negative | k_Bacteria; p_Bacteroidetes; c_Bacteroidia; | 128 | |
| vo1645223 | Propionate | | | o_Bacteroidales; f_[Paraprevotellaceae]; | | |
| | | | | g_CF231; s_ | | |
| deno- | Rumen | 0.34542444 | Negative | k_Bacteria; p_Bacteroidetes; c_Bacteroidia; | 129 | |
| vo1645230 | Propionate | | | o_Bacteroidales; f_[Paraprevotellaceae]; g_; s_ | | |
| deno- | Rumen | 0.341473091 | Negative | k_Bacteria; p_Verrucomicrobia; c_Verruco-5; | 130 | |
| vo1649599 | Propionate | | | o_WCHB1-41; f_RFP12; g_; s_ | | |
| deno- | Rumen | 0.443483302 | Negative | k_Bacteria; p_Bacteroidetes; c_Bacteroidia; | 131 | |
| vo1654182 | Propionate | | | o_Bacteroidales; f_; g_; s_ | | |
| deno- | Rumen | 0.467304701 | Negative | k_Bacteria; p_Proteobacteria; | 132 | |
| vo1665986 | Propionate | | | c_Gammaproteobacteria; o_Aeromonadales; | | |
| | | | | f_Succinivibrionaceae; g_; s_ | | |
| deno- | Rumen | 0.546722709 | Negative | k_Bacteria; p_Bacteroidetes; c_Bacteroidia; | 133 | |
| vo167470 | Propionate | | | o_Bacteroidales; f_[Paraprevotellaceae]; | | |
| | | | | g_YRC22; s_ | | |
| deno- | Rumen | 0.457834467 | Negative | k_Bacteria; p_Firmicutes; c_Clostridia; | 134 | |
| vo1678620 | Acetate | | | o_Clostridiales; f_Veillonellaceae | | |
| deno- | Rumen | 0.453143204 | Negative | k_Bacteria; p_Proteobacteria; | 135 | |
| vo1678621 | Acetate | | | c_Deltaproteobacteria; o_Desulfovibrionales; | | |
| | | | | f_Desulfovibrionaceae; g_Desulfovibrio; s_D168 | | |
| deno- | Rumen | 0.441467417 | Negative | k_Bacteria; p_Firmicutes; c_Clostridia; | 136 | |
| vo1685547 | Propionate | | | o_Clostridiales; f_Ruminococcaceae | | |
| deno- | Rumen | 0.64071958 | Negative | k_Bacteria; p_Bacteroidetes; c_Bacteroidia; | 137 | |
| vo170257 | Propionate | | | o_Bacteroidales; f_Prevotellaceae; g_Prevotella; | | |
| | | | | s_ | | |
| deno- | Rumen | 0.403102807 | Negative | k_Bacteria; p_Verrucomicrobia; c_Verruco-5; | 138 | |
| vo1702990 | Propionate | | | o_WCHB1-41; f_RFP12; g_; s_ | | |
| deno- | Diet | 0.321836219 | Negative | k_Bacteria; p_Actinobacteria; c_Coriobacteriia; | 139 | |
| vo1713211 | crude | | | o_Coriobacteriales; f_Coriobacteriaceae; g_; s_ | | |
| | protein | | | | | |
| deno- | Rumen | 0.309478342 | Negative | k_Bacteria; p_Actinobacteria; c_Coriobacteriia; | 140 | |
| vo1717065 | Acetate | | | o_Coriobacteriales; f_Coriobacteriaceae; g_; s_ | | |
| deno- | Rumen | 0.355576877 | Negative | k_Bacteria; p_Bacteroidetes; c_Bacteroidia; | 141 | |
| vo1722008 | Acetate | | | o_Bacteroidales; f_Prevotellaceae; g_Prevotella; | | |
| | | | | s_ | | |
| deno- | Rumen | 0.43370624 | Negative | k_Bacteria; p_Bacteroidetes; c_Bacteroidia; | 142 | |
| vo172528 | Propionate | | | o_Bacteroidales; f_RF16; g_; s_ | | |
| deno- | Rumen | 0.646547401 | Negative | k_Bacteria; p_Bacteroidetes; c_Bacteroidia; | 143 | |
| vo173062 | Acetate | | | o_Bacteroidales; f_Prevotellaceae; g_Prevotella; | | |
| | | | | s_ | | |
| deno- | Rumen | 0.473424114 | Negative | k_Bacteria; p_Firmicutes; c_Clostridia; | 144 | |
| vo174108 | Propionate | | | o_Clostridiales; f_Ruminococcaceae; | | |
| | | | | g_Ruminococcus; s_ | | |
| deno- | Rumen | 0.387368207 | Negative | k_Bacteria; p_Bacteroidetes; c_Bacteroidia; | 145 | |
| vo1756558 | Acetate | | | o_Bacteroidales; f_Prevotellaceae; g_Prevotella; | | |
| | | | | s_ | | |
| deno- | Rumen | 0.613684022 | Negative | k_Bacteria; p_Firmicutes; c_Clostridia; | 146 | |
| vo1795734 | Acetate | | | o_Clostridiales; f_Veillonellaceae; g_Dialister; | | |
| | | | | s_ | | |
| deno- | Rumen | 0.526643757 | Negative | k_Bacteria; p_Bacteroidetes; c_Bacteroidia; | 147 | |
| vo1801715 | Acetate | | | o_Bacteroidales; f_Prevotellaceae; g_Prevotella; | | |
| | | | | s_ | | |
| deno- | Rumen | 0.543134797 | Negative | k_Bacteria; p_Bacteroidetes; c_Bacteroidia; | 148 | |
| vo1803997 | Propionate | | | o_Bacteroidales; f_Prevotellaceae; g_Prevotella; | | |
| | | | | s_ | | |
| deno- | Rumen | 0.450636906 | Negative | k_Bacteria; p_Proteobacteria; | 149 | |
| vo1806325 | Propionate | | | c_Gammaproteobacteria; o_Aeromonadales; | | |
| | | | | f_Succinivibrionaceae; g_; s_ | | |
| deno- | Rumen | 0.306158893 | Negative | k_Bacteria; p_Firmicutes; c_Clostridia; | 150 | |
| vo18129 | Propionate | | | o_Clostridiales; f_Ruminococcaceae; g_; s_ | | |
| deno- | Rumen | 0.476603738 | Negative | k_Bacteria; p_Bacteroidetes; c_Bacteroidia; | 151 | |
| vo183477 | Propionate | | | o_Bacteroidales; f_BS11; g_; s_ | | |
| deno- | Rumen | 0.316263438 | Negative | k_Bacteria; p_Bacteroidetes; c_Bacteroidia; | 152 | |
| vo1843907 | Propionate | | | o_Bacteroidales; f_Prevotellaceae; g_Prevotella; | | |
| | | | | s_ | | |
| deno- | Rumen | 0.56609256 | Negative | k_Bacteria; p_Proteobacteria; | 153 | |
| vo1845242 | Propionate | | | c_Gammaproteobacteria; o_Aeromonadales; | | |
| | | | | f_Succinivibrionaceae; g_; s_ | | |
| deno- | Rumen | 0.288796896 | Negative | k_Bacteria; p_Bacteroidetes; c_Bacteroidia; | 154 | |
| vo1863743 | Acetate | | | o_Bacteroidales; f_Prevotellaceae; g_Prevotella; | | |
| | | | | s_ | | |
| deno- | Rumen | 0.491340511 | Negative | k_Bacteria; p_Firmicutes; c_Clostridia; | 155 | |
| vo1871583 | Propionate | | | o_Clostridiales; f_Christensenellaceae; g_; s_ | | |
| deno- | Rumen | 0.610285791 | Negative | k_Bacteria; p_Firmicutes; c_Clostridia; | 156 | |
| vo1872170 | Acetate | | | o_Clostridiales; f_Lachnospiraceae; | | |
| | | | | g_Butyrivibrio; s_ | | |
| deno- | Rumen | 0.484306143 | Negative | k_Bacteria; p_Lentisphaerae; c_[Lentisphaeria]; | 157 | |
| vo1875086 | Propionate | | | o_Z20; f_R4-45B; g_; s_ | | |
| deno- | Plasma | 0.3689923 | Negative | k_Bacteria; p_Bacteroidetes; c_Bacteroidia; | 158 | |
| vo1879715 | BHB | | | o_Bacteroidales; f_Prevotellaceae; g_Prevotella; | | |
| | | | | s_ | | |
| deno- | Rumen | 0.567492056 | Negative | k_Bacteria; p_Bacteroidetes; c_Bacteroidia; | 159 | |
| vo1879715 | Acetate | | | o_Bacteroidales; f_Prevotellaceae; g_Prevotella; | | |
| | | | | s_ | | |
| deno- | Rumen | 0.365600767 | Negative | k_Bacteria; p_Bacteroidetes; c_Bacteroidia; | 160 | |
| vo188900 | Propionate | | | o_Bacteroidales; f_[Paraprevotellaceae]; | | |
| | | | | g_YRC22; s_ | | |
| deno- | Rumen | 0.472238606 | Negative | k_Bacteria; p_Proteobacteria; | 161 | |
| vo1891669 | Propionate | | | c_Gammaproteobacteria; o_Aeromonadales; | | |
| | | | | f_Succinivibrionaceae; g_; s_ | | |
| deno- | Rumen | 0.321339869 | Negative | k_Bacteria; p_Spirochaetes; c_Spirochaetes; | 162 | |
| vo1913481 | Propionate | | | o_Spirochaetales; f_Spirochaetaceae; | | |
| | | | | g_Treponema; s_ | | |
| deno- | Rumen | 0.686936954 | Negative | k_Bacteria; p_Proteobacteria; | 163 | |
| vo1937263 | Propionate | | | c_Gammaproteobacteria; o_Aeromonadales; | | |
| | | | | f_Succinivibrionaceae; g_Ruminobacter; s_ | | |
| deno- | Rumen | 0.38597929 | Negative | k_Bacteria; p_Bacteroidetes; c_Bacteroidia; | 164 | |
| vo194317 | Propionate | | | o_Bacteroidales; f_; g_; s_ | | |
| deno- | Rumen | 0.585375043 | Negative | k_Bacteria; p_Bacteroidetes; c_Bacteroidia; | 165 | |
| vo1951663 | Propionate | | | o_Bacteroidales; f_BS11; g_; s_ | | |
| deno- | Rumen | 0.563691443 | Negative | k_Bacteria; p_Bacteroidetes; c_Bacteroidia; | 166 | |
| vo1966905 | Acetate | | | o_Bacteroidales; f_Prevotellaceae; g_Prevotella; | | |
| | | | | s_ | | |
| deno- | Rumen | 0.543337997 | Negative | k_Bacteria; p_Bacteroidetes; c_Bacteroidia; | 167 | |
| vo1988814 | Acetate | | | o_Bacteroidales; f_Prevotellaceae; g_Prevotella; | | |
| | | | | s_ | | |
| deno- | Rumen | 0.333739375 | Negative | k_Bacteria; p_Bacteroidetes; c_Bacteroidia; | 168 | |
| vo1997498 | Propionate | | | o_Bacteroidales; f_RF16; g_; s_ | | |
| deno- | Milk fat | 0.418523841 | Negative | k_Bacteria; p_Bacteroidetes; c_Bacteroidia; | 169 | |
| vo2021807 | | | | o_Bacteroidales; f_Prevotellaceae; g_Prevotella; | | |
| | | | | s_ | | |
| deno- | Rumen | 0.563277483 | Negative | k_Bacteria; p_Bacteroidetes; c_Bacteroidia; | 170 | |
| vo2021807 | Acetate | | | o_Bacteroidales; f_Prevotellaceae; g_Prevotella; | | |
| | | | | s_ | | |
| deno- | Rumen | 0.279381292 | Negative | k_Bacteria; p_Bacteroidetes; c_Bacteroidia; | 171 | |
| vo2047686 | Propionate | | | o_Bacteroidales; f_Prevotellaceae; g_Prevotella; | | |
| | | | | s_ | | |
| deno- | Rumen | 0.552168468 | Negative | k_Bacteria; p_Bacteroidetes; c_Bacteroidia; | 172 | |
| vo206654 | Acetate | | | o_Bacteroidales; f_Prevotellaceae; g_Prevotella; | | |
| | | | | s_ | | |
| deno- | Rumen | 0.323888336 | Negative | k_Bacteria; p_Bacteroidetes; c_Bacteroidia; | 173 | |
| vo2069744 | Acetate | | | o_Bacteroidales; f_Prevotellaceae; g_Prevotella; | | |
| | | | | s_ | | |
| deno- | Rumen | 0.704788685 | Negative | k_Bacteria; p_Bacteroidetes; c_Bacteroidia; | 174 | |
| vo2070846 | Propionate | | | o_Bacteroidales; f_Prevotellaceae; g_; s_ | | |
| deno- | Rumen | 0.437520305 | Negative | k_Bacteria; p_Bacteroidetes; c_Bacteroidia; | 175 | |
| vo2081094 | Acetate | | | o_Bacteroidales; f_; g_; s_ | | |
| deno- | Rumen | 0.410316173 | Negative | k_Bacteria; p_Bacteroidetes; c_Bacteroidia; | 176 | |
| vo2091417 | Propionate | | | o_Bacteroidales; f_; g_; s_ | | |
| deno- | Rumen | 0.328630235 | Negative | k_Bacteria; p_Bacteroidetes; c_Bacteroidia; | 177 | |
| vo2093314 | Acetate | | | o_Bacteroidales; f_Prevotellaceae; g_Prevotella; | | |
| | | | | s_ | | |
| deno- | Rumen | 0.557235664 | Negative | k_Bacteria; p_Bacteroidetes; c_Bacteroidia; | 178 | |
| vo2141299 | Propionate | | | o_Bacteroidales; f_RF16; g_; s_ | | |
| deno- | Rumen | 0.520142205 | Negative | k_Bacteria; p_Proteobacteria; | 179 | |
| vo2141307 | Propionate | | | c_Deltaproteobacteria; o_Desulfovibrionales; | | |
| | | | | f_Desulfovibrionaceae; g_Desulfovibrio; s_D168 | | |
| deno- | Rumen | 0.394654154 | Negative | k_Bacteria; p_Bacteroidetes; c_Bacteroidia; | 180 | |
| vo2162210 | Propionate | | | o_Bacteroidales; f_[Paraprevotellaceae]; | | |
| | | | | g_CF231; s_ | | |
| deno- | Diet | 0.313965691 | Negative | k_Bacteria; p_Bacteroidetes; c_Bacteroidia; | 181 | |
| vo2163819 | starch | | | o_Bacteroidales; f_Prevotellaceae; g_; s_ | | |
| deno- | Rumen | 0.48757474 | Negative | k_Bacteria; p_Bacteroidetes; c_Bacteroidia; | 182 | |
| vo2171865 | Propionate | | | o_Bacteroidales; f_Prevotellaceae; g_Prevotella; | | |
| | | | | s_ | | |
| deno- | Rumen | 0.616742126 | Negative | k_Bacteria; p_Firmicutes; c_Clostridia; | 183 | |
| vo2190261 | Acetate | | | o_Clostridiales; f_Lachnospiraceae | | |
| deno- | Rumen | 0.602375547 | Negative | k_Bacteria; p_Bacteroidetes; c_Bacteroidia; | 184 | |
| vo2199124 | Propionate | | | o_Bacteroidales; f_; g_; s_ | | |
| deno- | Rumen | 0.431163721 | Negative | k_Bacteria; p_Bacteroidetes; c_Bacteroidia; | 185 | |
| vo2222214 | Propionate | | | o_Bacteroidales; f_; g_; s_ | | |
| deno- | Rumen | 0.415075077 | Negative | k_Bacteria; p_Firmicutes; c_Clostridia; | 186 | |
| vo2227499 | Acetate | | | o_Clostridiales; f_Lachnospiraceae; | | |
| | | | | g_Coprococcus; s_ | | |
| deno- | Rumen | 0.336170773 | Negative | k_Bacteria; p_Bacteroidetes; c_Bacteroidia; | 187 | |
| vo2243771 | Acetate | | | o_Bacteroidales; f_S24-7; g_; s_ | | |
| deno- | Rumen | 0.369440664 | Negative | k_Bacteria; p_Firmicutes; c_Clostridia; | 188 | |
| vo2251647 | Propionate | | | o_Clostridiales; f_Ruminococcaceae; | | |
| | | | | g_Ruminococcus; s_ | | |
| deno- | CH4 | 0.433582323 | Negative | k_Bacteria; p_Bacteroidetes; c_Bacteroidia; | 189 | |
| vo2260584 | g/kg | | | o_Bacteroidales; f_Prevotellaceae; g_Prevotella; | | |
| | ECM | | | s_copri | | |
| deno- | Rumen | 0.622291365 | Negative | k_Bacteria; p_Bacteroidetes; c_Bacteroidia; | 190 | |
| vo2260584 | Acetate | | | o_Bacteroidales; f_Prevotellaceae; g_Prevotella; | | |
| | | | | s_copri | | |
| deno- | Rumen | 0.429903504 | Negative | k_Bacteria; p_Bacteroidetes; c_Bacteroidia; | 191 | |
| vo2266377 | Propionate | | | o_Bacteroidales; f_Prevotellaceae; g_Prevotella; | | |
| | | | | s_ | | |
| deno- | Rumen | 0.481702579 | Negative | k_Bacteria; p_Firmicutes; c_Clostridia; | 192 | |
| vo2294592 | Propionate | | | o_Clostridiales; f_Lachnospiraceae; g_Moryella; | | |
| | | | | s_ | | |
| deno- | Rumen | 0.576875105 | Negative | k_Bacteria; p_Firmicutes; c_Clostridia; | 193 | |
| vo2301555 | Acetate | | | o_Clostridiales; f_Lachnospiraceae | | |
| deno- | Rumen | 0.358194853 | Negative | k_Bacteria; p_Proteobacteria; | 194 | |
| vo2308695 | Acetate | | | c_Gammaproteobacteria; o_Aeromonadales; | | |
| | | | | f_Succinivibrionaceae; g_; s_ | | |
| deno- | Rumen | 0.46602001 | Negative | k_Bacteria; p_Spirochaetes; c_Spirochaetes; | 195 | |
| vo2310307 | Propionate | | | o_Spirochaetales; f_Spirochaetaceae; | | |
| | | | | g_Treponema; s_ | | |
| deno- | Rumen | 0.589371688 | Negative | k_Bacteria; p_Bacteroidetes; c_Bacteroidia; | 196 | |
| vo2318873 | Acetate | | | o_Bacteroidales; f_Prevotellaceae; g_Prevotella; | | |
| | | | | s_ | | |
| deno- | Rumen | 0.707206721 | Negative | k_Bacteria; p_Bacteroidetes; c_Bacteroidia; | 197 | |
| vo2323272 | Propionate | | | o_Bacteroidales; f_; g_; s_ | | |
| deno- | Rumen | 0.347403129 | Negative | k_Bacteria; p_Verrucomicrobia; c_Verruco-5; | 198 | |
| vo2345200 | Propionate | | | o_WCHB1-41; f_RFP12; g_; s_ | | |
| deno- | Rumen | 0.412232657 | Negative | k_Bacteria; p_Bacteroidetes; c_Bacteroidia; | 199 | |
| vo2358052 | Acetate | | | o_Bacteroidales; f_Prevotellaceae; g_Prevotella; | | |
| | | | | s_ | | |
| deno- | Rumen | 0.554078933 | Negative | k_Bacteria; p_Bacteroidetes; c_Bacteroidia; | 200 | |
| vo2367108 | Propionate | | | o_Bacteroidales; f_Prevotellaceae; g_Prevotella; | | |
| | | | | s_ | | |
| deno- | Rumen | 0.485296889 | Negative | k_Bacteria; p_Bacteroidetes; c_Bacteroidia; | 201 | |
| vo2367933 | Acetate | | | o_Bacteroidales; f_Prevotellaceae; g_Prevotella; | | |
| | | | | s_ | | |
| deno- | Rumen | 0.332485918 | Negative | k_Bacteria; p_Bacteroidetes; c_Bacteroidia; | 202 | |
| vo252478 | Propionate | | | o_Bacteroidales; f_Prevotellaceae; g_Prevotella; | | |
| | | | | s_ | | |
| deno- | Rumen | 0.505548396 | Negative | k_Bacteria; p_Bacteroidetes; c_Bacteroidia; | 203 | |
| vo278746 | Propionate | | | o_Bacteroidales; f_; g_; s_ | | |
| deno- | Rumen | 0.430582642 | Negative | k_Bacteria; p_Spirochaetes; c_Spirochaetes; | 204 | |
| vo279606 | Propionate | | | o_Spirochaetales; f_Spirochaetaceae; | | |
| | | | | g_Treponema; s_ | | |
| deno- | Rumen | 0.482914884 | Negative | k_Bacteria; p_Firmicutes; c_Clostridia; | 205 | |
| vo279607 | Propionate | | | o_Clostridiales; f_; g_; s_ | | |
| deno- | Rumen | 0.375432167 | Negative | k_Bacteria; p_Proteobacteria; | 206 | |
| vo298878 | Acetate | | | c_Gammaproteobacteria; o_Aeromonadales; | | |
| | | | | f_Succinivibrionaceae; g_; s_ | | |
| deno- | Rumen | 0.304212653 | Negative | k_Bacteria; p_Verrucomicrobia; c_Verruco-5; | 207 | |
| vo308672 | Propionate | | | o_WCHB1-41; f_RFP12; g_; s_ | | |
| deno- | Rumen | 0.455286996 | Negative | k_Bacteria; p_Bacteroidetes; c_Bacteroidia; | 208 | |
| vo314717 | Propionate | | | o_Bacteroidales; f_S24-7; g_; s_ | | |
| deno- | Rumen | 0.442395106 | Negative | k_Bacteria; p_Verrucomicrobia; c_Verruco-5; | 209 | |
| vo318201 | Propionate | | | o_WCHB1-41; f_RFP12; g_; s_ | | |
| deno- | Diet | 0.300984085 | Negative | k_Bacteria; p_Spirochaetes; c_Spirochaetes; | 210 | |
| vo333555 | starch | | | o_Spirochaetales; f_Spirochaetaceae; | | |
| | | | | g_Treponema; s_ | | |
| deno- | Rumen | 0.543649095 | Negative | k_Bacteria; p_Proteobacteria; | 211 | |
| vo33906 | Propionate | | | c_Gammaproteobacteria | | |
| deno- | Rumen | 0.423673986 | Negative | k_Bacteria; p_Bacteroidetes; c_Bacteroidia; | 212 | |
| vo33907 | Propionate | | | o_Bacteroidales; f_Prevotellaceae; g_Prevotella; | | |
| | | | | s_ | | |
| deno- | Rumen | 0.34529075 | Negative | k_Bacteria; p_Bacteroidetes; c_Bacteroidia; | 213 | |
| vo340240 | Acetate | | | o_Bacteroidales; f_Prevotellaceae; g_Prevotella; | | |
| | | | | s_ | | |
| deno- | Rumen | 0.450366255 | Negative | k_Bacteria; p_Bacteroidetes; c_Bacteroidia; | 214 | |
| vo34274 | Acetate | | | o_Bacteroidales; f_Prevotellaceae; g_Prevotella; | | |
| | | | | s_ | | |
| deno- | Rumen | 0.388563116 | Negative | k_Bacteria; p_Bacteroidetes; c_Bacteroidia; | 215 | |
| vo353603 | Propionate | | | o_Bacteroidales; f_Prevotellaceae; g_Prevotella; | | |
| | | | | s_ | | |
| deno- | CH4 | 0.398359404 | Negative | k_Bacteria; p_Proteobacteria; | 216 | |
| vo358994 | g/kg | | | c_Gammaproteobacteria; o_Aeromonadales; | | |
| | DMI | | | f_Succinivibrionaceae; g_; s_ | | |
| deno- | CH4 | 0.497749934 | Negative | k_Bacteria; p_Proteobacteria; | 217 | |
| vo358994 | g/kg | | | c_Gammaproteobacteria; o_Aeromonadales; | | |
| | ECM | | | f_Succinivibrionaceae; g_; s_ | | |
| deno- | Milk fat | 0.356950411 | Negative | k_Bacteria; p_Proteobacteria; | 218 | |
| vo358994 | | | | c_Gammaproteobacteria; o_Aeromonadales; | | |
| | | | | f_Succinivibrionaceae; g_; s_ | | |
| deno- | Plasma | 0.405162312 | Negative | k_Bacteria; p_Proteobacteria; | 219 | |
| vo358994 | BHB | | | c_Gammaproteobacteria; o_Aeromonadales; | | |
| | | | | f_Succinivibrionaceae; g_; s_ | | |
| deno- | Rumen | 0.665319959 | Negative | k_Bacteria; p_Proteobacteria; | 220 | |
| vo358994 | Acetate | | | c_Gammaproteobacteria; o_Aeromonadales; | | |
| | | | | f_Succinivibrionaceae; g_; s_ | | |
| deno- | Rumen | 0.591616637 | Negative | k_Bacteria; p_Proteobacteria; | 221 | |
| vo358994 | Caproate | | | c_Gammaproteobacteria; o_Aeromonadales; | | |
| | | | | f_Succinivibrionaceae; g_; s_ | | |
| deno- | Rumen | 0.511502626 | Negative | k_Bacteria; p_Bacteroidetes; c_Bacteroidia; | 222 | |
| vo370057 | Propionate | | | o_Bacteroidales; f_; g_; s_ | | |
| deno- | Rumen | 0.640016201 | Negative | k_Bacteria; p_Proteobacteria; | 223 | |
| vo384931 | Propionate | | | c_Gammaproteobacteria; o_Aeromonadales; | | |
| | | | | f_Succinivibrionaceae; g_Succinivibrio; s_ | | |
| deno- | Rumen | 0.375580447 | Negative | k_Bacteria; p_Proteobacteria; | 224 | |
| vo384931 | Valerate | | | c_Gammaproteobacteria; o_Aeromonadales; | | |
| | | | | f_Succinivibrionaceae; g_Succinivibrio; s_ | | |
| deno- | Rumen | 0.561274623 | Negative | k_Bacteria; p_Verrucomicrobia; c_Verruco-5; | 225 | |
| vo410508 | Propionate | | | o_WCHB1-41; f_RFP12; g_; s_ | | |
| deno- | Rumen | 0.521852647 | Negative | k_Bacteria; p_Proteobacteria; | 226 | |
| vo433754 | Propionate | | | c_Gammaproteobacteria; o_Aeromonadales; | | |
| | | | | f_Succinivibrionaceae; g_Ruminobacter; s_ | | |
| deno- | Milk fat | 0.512151933 | Negative | k_Bacteria; p_Firmicutes; c_Clostridia; | 227 | |
| vo445030 | | | | o_Clostridiales; f_Veillonellaceae; g_Dialister; | | |
| | | | | s_ | | |
| deno- | Rumen | 0.641452155 | Negative | k_Bacteria; p_Firmicutes; c_Clostridia; | 228 | |
| vo445030 | Acetate | | | o_Clostridiales; f_Veillonellaceae; g_Dialister; | | |
| | | | | s_ | | |
| deno- | Acetate | 0.499354901 | Negative | k_Bacteria; p_Bacteroidetes; c_Bacteroidia; | 229 | |
| vo448814 | Rumen | | | o_Bacteroidales; f_Prevotellaceae; g_Prevotella; | | |
| | | | | s_ | | |
| deno- | Rumen | 0.461721119 | Negative | k_Bacteria; p_Proteobacteria; | 230 | |
| vo454615 | Acetate | | | c_Gammaproteobacteria; o_Aeromonadales; | | |
| | | | | f_Succinivibrionaceae; g_; s_ | | |
| deno- | Plasma | 0.288398481 | Negative | k_Bacteria; p_Bacteroidetes; c_Bacteroidia; | 231 | |
| vo461510 | BHB | | | o_Bacteroidales; f_Prevotellaceae; g_Prevotella; | | |
| | | | | s_ | | |
| deno- | Rumen | 0.376978935 | Negative | k_Bacteria; p_Bacteroidetes; c_Bacteroidia; | 232 | |
| vo461510 | Acetate | | | o_Bacteroidales; f_Prevotellaceae; g_Prevotella; | | |
| | | | | s_ | | |
| deno- | Rumen | 0.393087759 | Negative | k_Bacteria; p_Proteobacteria; | 233 | |
| vo473355 | Acetate | | | c_Gammaproteobacteria; o_Aeromonadales; | | |
| | | | | f_Succinivibrionaceae; g_; s_ | | |
| deno- | Rumen | 0.412943869 | Negative | k_Bacteria; p_Firmicutes; c_Clostridia; | 234 | |
| vo477266 | Acetate | | | o_Clostridiales; f_Lachnospiraceae; | | |
| | | | | g_Butyrivibrio; s_ | | |
| deno- | Rumen | 0.495255744 | Negative | k_Bacteria; p_Proteobacteria; | 235 | |
| vo481551 | Acetate | | | c_Gammaproteobacteria; o_Aeromonadales; | | |
| | | | | f_Succinivibrionaceae; g_; s_ | | |
| deno- | Milk fat | 0.391618207 | Negative | k_Bacteria; p_Bacteroidetes; c_Bacteroidia; | 236 | |
| vo48352 | | | | o_Bacteroidales; f_Prevotellaceae; g_Prevotella; | | |
| | | | | s_ | | |
| deno- | Rumen | 0.522109983 | Negative | k_Bacteria; p_Bacteroidetes; c_Bacteroidia; | 237 | |
| vo48352 | Acetate | | | o_Bacteroidales; f_Prevotellaceae; g_Prevotella; | | |
| | | | | s_ | | |
| deno- | Rumen | 0.372724255 | Negative | k_Bacteria; p_Firmicutes; c_Clostridia; | 238 | |
| vo488679 | Propionate | | | o_Clostridiales; f_Ruminococcaceae; g_; s_ | | |
| deno- | Rumen | 0.488032742 | Negative | k_Bacteria; p_Bacteroidetes; c_Bacteroidia; | 239 | |
| vo506833 | Acetate | | | o_Bacteroidales; f_Prevotellaceae; g_Prevotella; | | |
| | | | | s_ | | |
| deno- | Rumen | 0.425120051 | Negative | k_Bacteria; p_Verrucomicrobia; c_Verruco-5; | 240 | |
| vo510868 | Propionate | | | o_WCHB1-41; f_RFP12; g_; s_ | | |
| deno- | Plasma | 0.32191085 | Negative | k_Bacteria; p_Bacteroidetes; c_Bacteroidia; | 241 | |
| vo514676 | BHB | | | o_Bacteroidales; f_Prevotellaceae; g_Prevotella; | | |
| | | | | s_ | | |
| deno- | Rumen | 0.56089197 | Negative | k_Bacteria; p_Bacteroidetes; c_Bacteroidia; | 242 | |
| vo514676 | Acetate | | | o_Bacteroidales; f_Prevotellaceae; g_Prevotella; | | |
| | | | | s_ | | |
| deno- | Milk fat | 0.366620806 | Negative | k_Bacteria; p_Bacteroidetes; c_Bacteroidia; | 243 | |
| vo521876 | | | | o_Bacteroidales; f_Prevotellaceae; g_Prevotella; | | |
| | | | | s_ | | |
| deno- | Rumen | 0.677037116 | Negative | k_Bacteria; p_Bacteroidetes; c_Bacteroidia; | 244 | |
| vo521876 | Acetate | | | o_Bacteroidales; f_Prevotellaceae; g_Prevotella; | | |
| | | | | s_ | | |
| deno- | Rumen | 0.374482703 | Negative | k_Bacteria; p_Verrucomicrobia; c_Verruco-5; | 245 | |
| vo523957 | Propionate | | | o_WCHB1-41; f_RFP12; g_; s_ | | |
| deno- | Rumen | 0.558045883 | Negative | k_Bacteria; p_Bacteroidetes; c_Bacteroidia; | 246 | |
| vo548248 | Acetate | | | o_Bacteroidales; f_Prevotellaceae; g_Prevotella; | | |
| | | | | s_ | | |
| deno- | Rumen | 0.46748704 | Negative | k_Bacteria; p_Bacteroidetes; c_Bacteroidia; | 247 | |
| vo548248 | Butyrate | | | o_Bacteroidales; f_Prevotellaceae; g_Prevotella; | | |
| | | | | s_ | | |
| deno- | Rumen | 0.653768974 | Negative | k_Bacteria; p_Bacteroidetes; c_Bacteroidia; | 248 | |
| vo554901 | Acetate | | | o_Bacteroidales; f_Prevotellaceae; g_Prevotella; | | |
| | | | | s_ | | |
| deno- | Rumen | 0.591191269 | Negative | k_Bacteria; p_Spirochaetes; c_Spirochaetes; | 249 | |
| vo557568 | Propionate | | | o_Spirochaetales; f_Spirochaetaceae; | | |
| | | | | g_Treponema; s_ | | |
| deno- | Rumen | 0.522971155 | Negative | k_Bacteria; p_Proteobacteria; | 250 | |
| vo560186 | Propionate | | | c_Gammaproteobacteria; o_Aeromonadales; | | |
| | | | | f_Succinivibrionaceae; g_; s_ | | |
| deno- | Milk fat | 0.396155652 | Negative | k_Bacteria; p_Bacteroidetes; c_Bacteroidia; | 251 | |
| vo577780 | | | | o_Bacteroidales; f_Prevotellaceae; g_Prevotella; | | |
| | | | | s_ | | |
| deno- | Plasma | 0.350076414 | Negative | k_Bacteria; p_Bacteroidetes; c_Bacteroidia; | 252 | |
| vo577780 | BHB | | | o_Bacteroidales; f_Prevotellaceae; g_Prevotella; | | |
| | | | | s_ | | |
| deno- | Rumen | 0.5749185 | Negative | k_Bacteria; p_Bacteroidetes; c_Bacteroidia; | 253 | |
| vo577780 | Acetate | | | o_Bacteroidales; f_Prevotellaceae; g_Prevotella; | | |
| | | | | s_ | | |
| deno- | Rumen | 0.415056541 | Negative | k_Bacteria; p_Bacteroidetes; c_Bacteroidia; | 254 | |
| vo582588 | Propionate | | | o_Bacteroidales; f_[Paraprevotellaceae]; | | |
| | | | | g_CF231; s_ | | |
| deno- | Rumen | 0.408928227 | Negative | k_Bacteria; p_Bacteroidetes; c_Bacteroidia; | 255 | |
| vo582825 | Propionate | | | o_Bacteroidales; f_Prevotellaceae; g_; s_ | | |
| deno- | Rumen | 0.412953438 | Negative | k_Bacteria; p_Verrucomicrobia; c_Verruco-5; | 256 | |
| vo582828 | Propionate | | | o_WCHB1-41; f_RFP12; g_; s_ | | |
| deno- | Rumen | 0.463821548 | Negative | k_Bacteria; p_Bacteroidetes; c_Bacteroidia; | 257 | |
| vo585153 | Acetate | | | o_Bacteroidales; f_Prevotellaceae; g_Prevotella; | | |
| | | | | s_ | | |
| deno- | Rumen | 0.585514535 | Negative | k_Bacteria; p_Firmicutes; c_Clostridia; | 258 | |
| vo593859 | Acetate | | | o_Clostridiales; f_Lachnospiraceae; | | |
| | | | | g_Shuttleworthia; s_ | | |
| deno- | Rumen | 0.546728454 | Negative | k_Bacteria; p_Bacteroidetes; c_Bacteroidia; | 259 | |
| vo61024 | Propionate | | | o_Bacteroidales; f_; g_; s_ | | |
| deno- | Rumen | 0.586677066 | Negative | k_Bacteria; p_Firmicutes; c_Clostridia; | 260 | |
| vo612360 | Acetate | | | o_Clostridiales; f_Veillonellaceae; g_Dialister; | | |
| | | | | s_ | | |
| deno- | Rumen | 0.652084467 | Negative | k_Bacteria; p_Bacteroidetes; c_Bacteroidia; | 261 | |
| vo618436 | Propionate | | | o_Bacteroidales; f_[Paraprevotellaceae]; | | |
| | | | | g_CF231; s_ | | |
| deno- | Acetate | 0.422243766 | Negative | k_Bacteria; p_Spirochaetes; c_Spirochaetes; | 262 | |
| vo632834 | Rumen | | | o_Spirochaetales; f_Spirochaetaceae; | | |
| | | | | g_Treponema; s_ | | |
| deno- | Milk fat | 0.40431653 | Negative | k_Bacteria; p_Bacteroidetes; c_Bacteroidia; | 263 | |
| vo63840 | | | | o_Bacteroidales; f_Prevotellaceae; g_Prevotella; | | |
| | | | | s_ | | |
| deno- | Rumen | 0.597741912 | Negative | k_Bacteria; p_Bacteroidetes; c_Bacteroidia; | 264 | |
| vo63840 | Acetate | | | o_Bacteroidales; f_Prevotellaceae; g_Prevotella; | | |
| | | | | s_ | | |
| deno- | Rumen | 0.53239675 | Negative | k_Bacteria; p_Firmicutes; c_Clostridia; | 265 | |
| vo649171 | Acetate | | | o_Clostridiales; f_Lachnospiraceae; | | |
| | | | | g_Butyrivibrio; s_ | | |
| deno- | Rumen | 0.371700142 | Negative | k_Bacteria; p_Bacteroidetes; c_Bacteroidia; | 266 | |
| vo650074 | Acetate | | | o_Bacteroidales; f_Prevotellaceae; g_Prevotella; | | |
| | | | | s_ | | |
| deno- | Rumen | 0.514379445 | Negative | k_Bacteria; p_Bacteroidetes; c_Bacteroidia; | 267 | |
| vo653342 | Propionate | | | o_Bacteroidales; f_; g_; s_ | | |
| deno- | Rumen | 0.346521505 | Negative | k_Bacteria; p_Bacteroidetes; c_Bacteroidia; | 268 | |
| vo671109 | Propionate | | | o_Bacteroidales; f_S24-7; g_; s_ | | |
| deno- | Rumen | 0.254902834 | Negative | k_Bacteria; p_Firmicutes; c_Clostridia; | 269 | |
| vo687413 | Propionate | | | o_Clostridiales; f_Lachnospiraceae; | | |
| | | | | g_Robinsoniella; s_peoriensis | | |
| deno- | Rumen | 0.541326536 | Negative | k_Bacteria; p_Firmicutes; c_Clostridia; | 270 | |
| vo693429 | Propionate | | | o_Clostridiales; f_Clostridiaceae; g_02d06; s_ | | |
| deno- | Rumen | 0.485312522 | Negative | k_Bacteria; p_Bacteroidetes; c_Bacteroidia; | 271 | |
| vo701009 | Acetate | | | o_Bacteroidales; f_[Paraprevotellaceae]; | | |
| | | | | g_CF231; s_ | | |
| deno- | Rumen | 0.551748499 | Negative | k_Bacteria; p_Bacteroidetes; c_Bacteroidia; | 272 | |
| vo701155 | Acetate | | | o_Bacteroidales; f_Prevotellaceae; g_Prevotella; | | |
| | | | | s_ | | |
| deno- | Rumen | 0.345512437 | Negative | k_Bacteria; p_Bacteroidetes; c_Bacteroidia; | 273 | |
| vo745561 | Acetate | | | o_Bacteroidales; f_Prevotellaceae; g_Prevotella; | | |
| | | | | s_ | | |
| deno- | Acetate | 0.453287022 | Negative | k_Bacteria; p_Bacteroidetes; c_Bacteroidia; | 274 | |
| vo775642 | Rumen | | | o_Bacteroidales; f_Prevotellaceae; g_Prevotella; | | |
| | | | | s_ | | |
| deno- | Rumen | 0.513383287 | Negative | k_Bacteria; p_Spirochaetes; c_Spirochaetes; | 275 | |
| vo780633 | Propionate | | | o_Spirochaetales; f_Spirochaetaceae; | | |
| | | | | g_Treponema; s_ | | |
| deno- | Rumen | 0.514550757 | Negative | k_Bacteria; p_Bacteroidetes; c_Bacteroidia; | 276 | |
| vo798795 | Acetate | | | o_Bacteroidales; f_Prevotellaceae; g_Prevotella; | | |
| | | | | s_ | | |
| deno- | Rumen | 0.409641295 | Negative | k_Bacteria; p_Firmicutes; c_Clostridia; | 277 | |
| vo824434 | Acetate | | | o_Clostridiales; f_Lachnospiraceae; | | |
| | | | | g_Shuttleworthia; s_ | | |
| deno- | Rumen | 0.290304066 | Negative | k_Bacteria; p_Bacteroidetes; c_Bacteroidia; | 278 | |
| vo838513 | Propionate | | | o_Bacteroidales; f_Prevotellaceae; g_Prevotella; | | |
| | | | | s_ | | |
| deno- | Plasma | 0.373967032 | Negative | k_Bacteria; p_Bacteroidetes; c_Bacteroidia; | 279 | |
| vo848818 | BHB | | | o_Bacteroidales; f_Prevotellaceae; g_Prevotella; | | |
| | | | | s_ | | |
| deno- | Rumen | 0.631171318 | Negative | k_Bacteria; p_Bacteroidetes; c_Bacteroidia; | 280 | |
| vo848818 | Acetate | | | o_Bacteroidales; f_Prevotellaceae; g_Prevotella; | | |
| | | | | s_ | | |
| deno- | Rumen | 0.596570129 | Negative | k_Bacteria; p_Proteobacteria; | 281 | |
| vo862967 | Propionate | | | c_Gammaproteobacteria; o_Aeromonadales; | | |
| | | | | f_Succinivibrionaceae; g_Ruminobacter; s_ | | |
| deno- | Rumen | 0.352757367 | Negative | k_Bacteria; p_Firmicutes; c_Clostridia; | 282 | |
| vo864695 | Propionate | | | o_Clostridiales; f_Lachnospiraceae; | | |
| | | | | g_Butyrivibrio; s_ | | |
| deno- | Rumen | 0.434873644 | Negative | k_Bacteria; p_Verrucomicrobia; c_Verruco-5; | 283 | |
| vo864696 | Propionate | | | o_WCHB1-41; f_RFP12; g_; s_ | | |
| deno- | Rumen | 0.320733412 | Negative | k_Bacteria; p_Bacteroidetes; c_Bacteroidia; | 284 | |
| vo86669 | Propionate | | | o_Bacteroidales; f_; g_; s_ | | |
| deno- | Rumen | 0.461658072 | Negative | k_Bacteria; p_Bacteroidetes; c_Bacteroidia; | 285 | |
| vo877792 | Acetate | | | o_Bacteroidales; f_Prevotellaceae; g_Prevotella; | | |
| | | | | s_ | | |
| deno- | Rumen | 0.356532674 | Negative | k_Bacteria; p_Bacteroidetes; c_Bacteroidia; | 286 | |
| vo878102 | Acetate | | | o_Bacteroidales; f_Prevotellaceae; g_Prevotella; | | |
| | | | | s_ | | |
| deno- | Milk fat | 0.372358455 | Negative | k_Bacteria; p_Firmicutes; c_Clostridia; | 287 | |
| vo879882 | | | | o_Clostridiales; f_Lachnospiraceae; | | |
| | | | | g_Shuttleworthia; s_ | | |
| deno- | Rumen | 0.559040641 | Negative | k_Bacteria; p_Firmicutes; c_Clostridia; | 288 | |
| vo879882 | Acetate | | | o_Clostridiales; f_Lachnospiraceae; | | |
| | | | | g_Shuttleworthia; s_ | | |
| deno- | Rumen | 0.534352725 | Negative | k_Bacteria; p_Firmicutes; c_Clostridia; | 289 | |
| vo879882 | Butyrate | | | o_Clostridiales; f_Lachnospiraceae; | | |
| | | | | g_Shuttleworthia; s_ | | |
| deno- | Milk fat | 0.392393181 | Negative | k_Bacteria; p_Bacteroidetes; c_Bacteroidia; | 290 | |
| vo882840 | | | | o_Bacteroidales; f_Prevotellaceae; g_Prevotella; | | |
| | | | | s_ | | |
| deno- | Plasma | 0.351298392 | Negative | k_Bacteria; p_Bacteroidetes; c_Bacteroidia; | 291 | |
| vo882840 | BHB | | | o_Bacteroidales; f_Prevotellaceae; g_Prevotella; | | |
| | | | | s_ | | |
| deno- | Rumen | 0.573893107 | Negative | k_Bacteria; p_Bacteroidetes; c_Bacteroidia; | 292 | |
| vo882840 | Acetate | | | o_Bacteroidales; f_Prevotellaceae; g_Prevotella; | | |
| | | | | s_ | | |
| deno- | Rumen | 0.305731561 | Negative | k_Bacteria; p_Firmicutes; c_Clostridia; | 293 | |
| vo886745 | Propionate | | | o_Clostridiales; f_Ruminococcaceae; g_; s_ | | |
| deno- | Rumen | 0.399823549 | Negative | k_Bacteria; p_Bacteroidetes; c_Bacteroidia; | 294 | |
| vo913272 | Propionate | | | o_Bacteroidales; f_Prevotellaceae; g_Prevotella; | | |
| | | | | s_ | | |
| deno- | Rumen | 0.297580584 | Negative | k_Bacteria; p_Bacteroidetes; c_Bacteroidia; | 295 | |
| vo92048 | Propionate | | | o_Bacteroidales; f_Prevotellaceae; g_; s_ | | |
| deno- | Rumen | 0.45601525 | Negative | k_Bacteria; p_Firmicutes; c_Clostridia; | 296 | |
| vo923356 | Propionate | | | o_Clostridiales; f_Ruminococcaceae; | | |
| | | | | g_Ruminococcus; s_ | | |
| deno- | Rumen | 0.631208194 | Negative | k_Bacteria; p_Proteobacteria; | 297 | |
| vo927104 | Propionate | | | c_Gammaproteobacteria; o_Aeromonadales; | | |
| | | | | f_Succinivibrionaceae; g_; s_ | | |
| deno- | Rumen | 0.455798527 | Negative | k_Bacteria; p_Bacteroidetes; c_Bacteroidia; | 298 | |
| vo927921 | Propionate | | | o_Bacteroidales; f_BS11; g_; s_ | | |
| deno- | Rumen | 0.401171823 | Negative | k_Bacteria; p_Bacteroidetes; c_Bacteroidia; | 299 | |
| vo932996 | Propionate | | | o_Bacteroidales; f_[Paraprevotellaceae]; | | |
| | | | | g_YRC22; s_ | | |
| deno- | Rumen | 0.529522744 | Negative | k_Bacteria; p_Firmicutes; c_Clostridia; | 300 | |
| vo938860 | Propionate | | | o_Clostridiales; f_Ruminococcaceae; | | |
| | | | | g_Ruminococcus; s_ | | |
| deno- | Plasma | 0.3660627 | Negative | k_Bacteria; p_Bacteroidetes; c_Bacteroidia; | 301 | |
| vo942112 | BHB | | | o_Bacteroidales; f_Prevotellaceae; g_Prevotella; | | |
| | | | | s_ | | |
| deno- | Rumen | 0.614645075 | Negative | k_Bacteria; p_Bacteroidetes; c_Bacteroidia; | 302 | |
| vo942112 | Acetate | | | o_Bacteroidales; f_Prevotellaceae; g_Prevotella; | | |
| | | | | s_ | | |
| deno- | Rumen | 0.598150527 | Negative | k_Bacteria; p_Bacteroidetes; c_Bacteroidia; | 303 | |
| vo942115 | Acetate | | | o_Bacteroidales; f_Prevotellaceae; g_Prevotella; | | |
| | | | | s_ | | |
| deno- | Rumen | 0.398145697 | Negative | k_Bacteria; p_Verrucomicrobia; c_Verruco-5; | 304 | |
| vo950635 | Propionate | | | o_WCHB1-41; f_RFP12; g_; s_ | | |
| deno- | Rumen | 0.311686056 | Negative | k_Bacteria; p_Verrucomicrobia; c_Verruco-5; | 305 | |
| vo953365 | Propionate | | | o_WCHB1-41; f_RFP12; g_; s_ | | |
| deno- | Rumen | 0.602365866 | Negative | k_Bacteria; p_Bacteroidetes; c_Bacteroidia; | 306 | |
| vo959148 | Acetate | | | o_Bacteroidales; f_Prevotellaceae; g_Prevotella; | | |
| | | | | s_ | | |
| deno- | Rumen | 0.65399403 | Negative | k_Bacteria; p_Bacteroidetes; c_Bacteroidia; | 307 | |
| vo97411 | Acetate | | | o_Bacteroidales; f_Prevotellaceae; g_Prevotella; | | |
| | | | | s_ | | |
| deno- | Rumen | 0.340910992 | Negative | k_Bacteria; p_Firmicutes; c_Clostridia; | 308 | |
| vo991253 | Propionate | | | o_Clostridiales; f_Ruminococcaceae; g_; s_ | | |
| deno- | Milk fat | 0.424935968 | Negative | k_Bacteria; p_Bacteroidetes; c_Bacteroidia; | 309 | |
| vo991831 | | | | o_Bacteroidales; f_Prevotellaceae; g_Prevotella; | | |
| | | | | s_ | | |
| deno- | Rumen | 0.599733351 | Negative | k_Bacteria; p_Bacteroidetes; c_Bacteroidia; | 310 | |
| vo991831 | Acetate | | | o_Bacteroidales; f_Prevotellaceae; g_Prevotella; | | |
| | | | | s_ | | |
| deno- | Rumen | 0.403369849 | Negative | k_Bacteria; p_Bacteroidetes; c_Bacteroidia; | 311 | |
| vo999188 | Acetate | | | o_Bacteroidales; f_Prevotellaceae; g_Prevotella; | | |
| | | | | s_ | | |
| deno- | Rumen | 0.448826211 | Negative | Neocallimastigales; Neocallimastigaceae; | 312 | |
| vo3895 | Acetate | | | Neocallimastix; Neocallimastix 1 | | |
| deno- | Rumen | 0.383491648 | Negative | D_0_Eukaryota; D_1_SAR; D_2_Alveolata; | 313 | |
| vo12500 | Propionate | | | D_3_Ciliophora; D_6_Trichostomatia; | | |
| | | | | D_7_Entodinium; D_8_uncultured rumen | | |
| | | | | protozoa | | |
| deno- | Rumen | 0.31024197 | Positive | k_Bacteria; p_Bacteroidetes; c_Bacteroidia; | 314 | |
| vo1003261 | Propionate | | | o_Bacteroidales; f_Prevotellaceae; g_Prevotella; | | |
| | | | | s_ | | |
| deno- | Rumen | 0.45822073 | Positive | k_Bacteria; p_Firmicutes; c_Clostridia; | 315 | |
| vo1004279 | Propionate | | | o_Clostridiales; f_Ruminococcaceae; | | |
| | | | | g_Ruminococcus; s_flavefaciens | | |
| deno- | Total | 0.351256814 | Positive | k_Bacteria; p_Verrucomicrobia; c_Verruco-5; | 316 | |
| vo1031054 | digestion | | | o_WCHB1-41; f_RFP12; g_; s_ | | |
| | dry | | | | | |
| | matter | | | | | |
| deno- | Milk fat | 0.423028216 | Positive | k_Bacteria; p_Bacteroidetes; c_Bacteroidia; | 317 | |
| vo1035747 | | | | o_Bacteroidales; f_; g_; s_ | | |
| deno- | Rumen | 0.60470706 | Positive | k_Bacteria; p_Bacteroidetes; c_Bacteroidia; | 318 | |
| vo1035747 | Acetate | | | o_Bacteroidales; f_; g_; s_ | | |
| deno- | Rumen | 0.649391594 | Positive | k_Bacteria; p_Bacteroidetes; c_Bacteroidia; | 319 | |
| vo1045128 | Propionate | | | o_Bacteroidales; f_Prevotellaceae; g_Prevotella; | | |
| | | | | s_ | | |
| deno- | Rumen | 0.556977754 | Positive | k_Bacteria; p_Bacteroidetes; c_Bacteroidia; | 320 | |
| vo1065963 | Acetate | | | o_Bacteroidales; f_[Paraprevotellaceae]; | | |
| | | | | g_CF231; s_ | | |
| deno- | Rumen | 0.371014008 | Positive | k_Bacteria; p_Elusimicrobia; c_Endomicrobia; | 321 | |
| vo1070363 | Acetate | | | o_; f_; g_; s_ | | |
| deno- | Rumen | 0.456520588 | Positive | k_Bacteria; p_Bacteroidetes; c_Bacteroidia; | 322 | |
| vo1086049 | Propionate | | | o_Bacteroidales; f_Prevotellaceae; g_Prevotella; | | |
| | | | | s_ | | |
| deno- | Rumen | 0.588685735 | Positive | k_Bacteria; p_Bacteroidetes; c_Bacteroidia; | 323 | |
| vo1107934 | Propionate | | | o_Bacteroidales; f_Prevotellaceae; g_Prevotella; | | |
| | | | | s_ | | |
| deno- | Plasma | 0.3081968 | Positive | k_Bacteria; p_Bacteroidetes; c_Bacteroidia; | 324 | |
| vo1115149 | BHB | | | o_Bacteroidales; f_Prevotellaceae; g_Prevotella; | | |
| | | | | s_ | | |
| deno- | Rumen | 0.317715069 | Positive | k_Bacteria; p_Bacteroidetes; c_Bacteroidia; | 325 | |
| vo1140040 | Butyrate | | | o_Bacteroidales; f_Prevotellaceae; g_; s_ | | |
| deno- | Rumen | 0.324548903 | Positive | k_Bacteria; p_Bacteroidetes; c_Bacteroidia; | 326 | |
| vo115455 | Acetate | | | o_Bacteroidales; f_BS11; g_; s_ | | |
| deno- | Rumen | 0.404232389 | Positive | k_Bacteria; p_Verrucomicrobia; c_Verruco-5; | 327 | |
| vo1163072 | Acetate | | | o_WCHB1-41; f_RFP12; g_; s_ | | |
| deno- | Rumen | 0.317239795 | Positive | k_Bacteria; p_Fibrobacteres; c_Fibrobacteria; | 328 | |
| vo1177927 | Acetate | | | o_Fibrobacterales; f_Fibrobacteraceae; | | |
| | | | | g_Fibrobacter; s_succinogenes | | |
| deno- | Rumen | 0.349143313 | Positive | k_Bacteria; p_Bacteroidetes; c_Bacteroidia; | 329 | |
| vo1189086 | Butyrate | | | o_Bacteroidales; f_Prevotellaceae; g_Prevotella; | | |
| | | | | s_ | | |
| deno- | Rumen | 0.36887195 | Positive | k_Bacteria; p_Bacteroidetes; c_Bacteroidia; | 330 | |
| vo1197961 | Butyrate | | | o_Bacteroidales; f_[Paraprevotellaceae]; | | |
| | | | | g_YRC22; s_ | | |
| deno- | Rumen | 0.481511178 | Positive | k_Bacteria; p_Proteobacteria; | 331 | |
| vo1221142 | Acetate | | | c_Gammaproteobacteria; o_Aeromonadales; | | |
| | | | | f_Succinivibrionaceae; g_; s_ | | |
| deno- | Rumen | 0.44497999 | Positive | k_Bacteria; p_Bacteroidetes; c_Bacteroidia; | 332 | |
| vo1240314 | Acetate | | | o_Bacteroidales; f_Prevotellaceae; g_Prevotella; | | |
| | | | | s_ | | |
| deno- | Rumen | 0.378863391 | Positive | k_Bacteria; p_Spirochaetes; c_Spirochaetes; | 333 | |
| vo1240985 | Acetate | | | o_Spirochaetales; f_Spirochaetaceae; | | |
| | | | | g_Treponema; s_ | | |
| deno- | Rumen | 0.584095721 | Positive | k_Bacteria; p_Bacteroidetes; c_Bacteroidia; | 334 | |
| vo1244578 | Propionate | | | o_Bacteroidales; f_Prevotellaceae; g_Prevotella; | | |
| | | | | s_ | | |
| deno- | Rumen | 0.372631843 | Positive | k_Bacteria; p_Bacteroidetes; c_Bacteroidia; | 335 | |
| vo1247348 | Propionate | | | o_Bacteroidales; f_Prevotellaceae; g_Prevotella; | | |
| | | | | s_ | | |
| deno- | Rumen | 0.517626391 | Positive | k_Bacteria; p_Proteobacteria; | 336 | |
| vo1256657 | Acetate | | | c_Gammaproteobacteria; o_Aeromonadales; | | |
| | | | | f_Succinivibrionaceae; g_; s_ | | |
| deno- | Rumen | 0.450142194 | Positive | k_Bacteria; p_Bacteroidetes; c_Bacteroidia; | 337 | |
| vo129818 | Propionate | | | o_Bacteroidales; f_Prevotellaceae; g_Prevotella; | | |
| | | | | s_ | | |
| deno- | Rumen | 0.424417579 | Positive | k_Bacteria; p_Bacteroidetes; c_Bacteroidia; | 338 | |
| vo1302941 | Acetate | | | o_Bacteroidales; f_; g_; s_ | | |
| deno- | Rumen | 0.351613548 | Positive | k_Bacteria; p_Firmicutes; c_Clostridia; | 339 | |
| vo1306025 | Propionate | | | o_Clostridiales; f_; g_; s_ | | |
| deno- | Rumen | 0.437781305 | Positive | k_Bacteria; p_Bacteroidetes; c_Bacteroidia; | 340 | |
| vo1308850 | Propionate | | | o_Bacteroidales; f_Prevotellaceae; g_Prevotella; | | |
| | | | | s_ | | |
| deno- | Rumen | 0.330646664 | Positive | k_Bacteria; p_Bacteroidetes; c_Bacteroidia; | 341 | |
| vo1309148 | Acetate | | | o_Bacteroidales; f_; g_; s_ | | |
| deno- | Rumen | 0.591716158 | Positive | k_Bacteria; p_Bacteroidetes; c_Bacteroidia; | 342 | |
| vo131546 | Propionate | | | o_Bacteroidales; f_Prevotellaceae; g_Prevotella; | | |
| | | | | s_ | | |
| deno- | Rumen | 0.465095106 | Positive | k_Bacteria; p_Firmicutes; c_Clostridia; | 343 | |
| vo1319394 | Propionate | | | o_Clostridiales; f_; g_; s_ | | |
| deno- | Rumen | 0.540144738 | Positive | k_Bacteria; p_Firmicutes; c_Clostridia; | 344 | |
| vo1325041 | Propionate | | | o_Clostridiales; f_Lachnospiraceae | | |
| deno- | Rumen | 0.302042085 | Positive | k_Bacteria; p_Bacteroidetes; c_Bacteroidia; | 345 | |
| vo1325386 | Butyrate | | | o_Bacteroidales; f_; g_; s_ | | |
| deno- | CH4 g/d | 0.409078292 | Positive | k_Bacteria; p_Bacteroidetes; c_Bacteroidia; | 346 | |
| vo1333663 | | | | o_Bacteroidales; f_Prevotellaceae; g_Prevotella; | | |
| | | | | s_ | | |
| deno- | Milk fat | 0.424975846 | Positive | k_Bacteria; p_Bacteroidetes; c_Bacteroidia; | 347 | |
| vo1333663 | | | | o_Bacteroidales; f_Prevotellaceae; g_Prevotella; | | |
| | | | | s_ | | |
| deno- | Rumen | 0.356581063 | Positive | k_Bacteria; p_Bacteroidetes; c_Bacteroidia; | 348 | |
| vo1369518 | Butyrate | | | o_Bacteroidales; f_Prevotellaceae; g_Prevotella; | | |
| | | | | s_ | | |
| deno- | Milk fat | 0.465756002 | Positive | k_Bacteria; p_Bacteroidetes; c_Bacteroidia; | 349 | |
| vo1385456 | | | | o_Bacteroidales; f_Prevotellaceae; g_Prevotella; | | |
| | | | | s_ | | |
| deno- | Rumen | 0.398447374 | Positive | k_Bacteria; p_Bacteroidetes; c_Bacteroidia; | 350 | |
| vo1385456 | Butyrate | | | o_Bacteroidales; f_Prevotellaceae; g_Prevotella; | | |
| | | | | s_ | | |
| deno- | Rumen | 0.421938073 | Positive | k_Bacteria; p_Bacteroidetes; c_Bacteroidia; | 351 | |
| vo1387720 | Acetate | | | o_Bacteroidales; f_Prevotellaceae; g_Prevotella; | | |
| | | | | s_ | | |
| deno- | Rumen | 0.405112012 | Positive | k_Bacteria; p_Bacteroidetes; c_Bacteroidia; | 352 | |
| vo1387720 | Caproate | | | o_Bacteroidales; f_Prevotellaceae; g_Prevotella; | | |
| | | | | s_ | | |
| deno- | Intake | 0.253821016 | Positive | k_Bacteria; p_Firmicutes; c_Clostridia; | 353 | |
| vo1396891 | Crude | | | o_Clostridiales | | |
| | Protein | | | | | |
| deno- | Intake | 0.263866226 | Positive | k_Bacteria; p_Firmicutes; c_Clostridia; | 354 | |
| vo1396891 | dry | | | o_Clostridiales | | |
| | matter | | | | | |
| deno- | Intake | 0.268139522 | Positive | k_Bacteria; p_Firmicutes; c_Clostridia; | 355 | |
| vo1396891 | NDF | | | o_Clostridiales | | |
| deno- | Intake | 0.262706108 | Positive | k_Bacteria; p_Firmicutes; c_Clostridia; | 356 | |
| vo1396891 | organic | | | o_Clostridiales | | |
| | matter | | | | | |
| deno- | Rumen | 0.298870834 | Positive | k_Bacteria; p_Firmicutes; c_Clostridia; | 357 | |
| vo1398878 | Propionate | | | o_Clostridiales; f_; g_; s_ | | |
| deno- | Rumen | 0.517332574 | Positive | k_Bacteria; p_Bacteroidetes; c_Bacteroidia; | 358 | |
| vo141080 | Propionate | | | o_Bacteroidales; f_Prevotellaceae; g_Prevotella; | | |
| | | | | s_ | | |
| deno- | Rumen | 0.400050911 | Positive | k_Bacteria; p_Fibrobacteres; c_Fibrobacteria; | 359 | |
| vo1419200 | Propionate | | | o_Fibrobacterales; f_Fibrobacteraceae; | | |
| | | | | g_Fibrobacter; s_ | | |
| deno- | Rumen | 0.648274629 | Positive | k_Bacteria; p_Bacteroidetes; c_Bacteroidia; | 360 | |
| vo1423479 | Propionate | | | o_Bacteroidales; f_Prevotellaceae; g_Prevotella; | | |
| | | | | s_ | | |
| deno- | Rumen | 0.66519568 | Positive | k_Bacteria; p_Bacteroidetes; c_Bacteroidia; | 361 | |
| vo1440570 | Propionate | | | o_Bacteroidales; f_Prevotellaceae; g_Prevotella; | | |
| | | | | s_ | | |
| deno- | Rumen | 0.408037685 | Positive | k_Bacteria; p_Firmicutes; c_Clostridia; | 362 | |
| vo1444540 | Propionate | | | o_Clostridiales; f_Lachnospiraceae; g_; s_ | | |
| deno- | Rumen | 0.308484663 | Positive | k_Bacteria; p_Bacteroidetes; c_Bacteroidia; | 363 | |
| vo1446200 | Propionate | | | o_Bacteroidales; f_Prevotellaceae; g_Prevotella | | |
| deno- | Rumen | 0.518888532 | Positive | k_Bacteria; p_Firmicutes; c_Clostridia; | 364 | |
| vo145213 | Propionate | | | o_Clostridiales; f_; g_; s_ | | |
| deno- | Milk Fat | 0.291942886 | Positive | k_Bacteria; p_Bacteroidetes; c_Bacteroidia; | 365 | |
| vo145907 | | | | o_Bacteroidales; f_Prevotellaceae; g_Prevotella; | | |
| | | | | s_ | | |
| deno- | Rumen | 0.457847817 | Positive | k_Bacteria; p_Bacteroidetes; c_Bacteroidia; | 366 | |
| vo1464133 | Propionate | | | o_Bacteroidales; f_Prevotellaceae; g_Prevotella; | | |
| | | | | s_ | | |
| deno- | Rumen | 0.326607017 | Positive | k_Bacteria; p_Bacteroidetes; c_Bacteroidia; | 367 | |
| vo1466475 | Propionate | | | o_Bacteroidales; f_Prevotellaceae; g_Prevotella; | | |
| | | | | s_ | | |
| deno- | Rumen | 0.383804157 | Positive | k_Bacteria; p_Firmicutes; c_Clostridia; | 368 | |
| vo1473970 | Acetate | | | o_Clostridiales; f_; g_; s_ | | |
| deno- | Plasma | 0.383723239 | Positive | k_Bacteria; p_Firmicutes; c_Clostridia; | 369 | |
| vo147816 | BHB | | | o_Clostridiales; f_Ruminococcaceae; | | |
| | | | | g_Ruminococcus; s_bromii | | |
| deno- | Rumen | 0.25170442 | Positive | k_Bacteria; p_Firmicutes; c_Clostridia; | 370 | |
| vo1479708 | Butyrate | | | o_Clostridiales; f_Lachnospiraceae | | |
| deno- | Rumen | 0.390248359 | Positive | k_Bacteria; p_Bacteroidetes; c_Bacteroidia; | 371 | |
| vo1483010 | Ammonia | | | o_Bacteroidales; f_Prevotellaceae; g_Prevotella; | | |
| | | | | s_ | | |
| deno- | Rumen | 0.23751816 | Positive | k_Bacteria; p_Bacteroidetes; c_Bacteroidia; | 372 | |
| vo1494221 | Butyrate | | | o_Bacteroidales; f_Prevotellaceae; g_Prevotella; | | |
| | | | | s_ | | |
| deno- | Rumen | 0.586832286 | Positive | k_Bacteria; p_Firmicutes; c_Clostridia; | 373 | |
| vo1497746 | Propionate | | | o_Clostridiales; f_Veillonellaceae; g_Dialister; | | |
| | | | | s_ | | |
| deno- | Rumen | 0.526567075 | Positive | k_Bacteria; p_Proteobacteria; | 374 | |
| vo1513549 | Propionate | | | c_Gammaproteobacteria; o_Aeromonadales; | | |
| | | | | f_Succinivibrionaceae; g_; s_ | | |
| deno- | Rumen | 0.462527234 | Positive | k_Bacteria; p_Proteobacteria; | 375 | |
| vo1518048 | Propionate | | | c_Gammaproteobacteria; o_Aeromonadales; | | |
| | | | | f_Succinivibrionaceae; g_; s_ | | |
| deno- | Rumen | 0.295893868 | Positive | k_Bacteria; p_Bacteroidetes; c_Bacteroidia; | 376 | |
| vo1528840 | Propionate | | | o_Bacteroidales; f_Prevotellaceae; g_Prevotella; | | |
| | | | | s_ | | |
| deno- | Rumen | 0.342893597 | Positive | k_Bacteria; p_Firmicutes; c_Clostridia; | 377 | |
| vo1544624 | Propionate | | | o_Clostridiales; f_Lachnospiraceae | | |
| deno- | Total | 0.328607403 | Positive | k_Bacteria; p_Verrucomicrobia; c_Verruco-5; | 378 | |
| vo156185 | digestion | | | o_WCHB1-41; f_RFP12; g_; s_ | | |
| | dry | | | | | |
| | matter | | | | | |
| deno- | Rumen | 0.539294375 | Positive | k_Bacteria; p_Proteobacteria; | 379 | |
| vo1566947 | Acetate | | | c_Gammaproteobacteria; o_Aeromonadales; | | |
| | | | | f_Succinivibrionaceae; g_; s_ | | |
| deno- | Rumen | 0.363343548 | Positive | k_Bacteria; p_Bacteroidetes; c_Bacteroidia; | 380 | |
| vo1582440 | Acetate | | | o_Bacteroidales; f_; g_; s_ | | |
| deno- | Rumen | 0.489196747 | Positive | k_Bacteria; p_Bacteroidetes; c_Bacteroidia; | 381 | |
| vo1603432 | Propionate | | | o_Bacteroidales; f_Prevotellaceae; g_Prevotella; | | |
| | | | | s_ | | |
| deno- | Rumen | 0.450223852 | Positive | k_Bacteria; p_Bacteroidetes; c_Bacteroidia; | 382 | |
| vo1603794 | Acetate | | | o_Bacteroidales; f_Prevotellaceae; g_Prevotella; | | |
| | | | | s_ | | |
| deno- | Rumen | 0.456247262 | Positive | k_Bacteria; p_Verrucomicrobia; c_Verruco-5; | 383 | |
| vo1613585 | Acetate | | | o_WCHB1-41; f_RFP12; g_; s_ | | |
| deno- | Rumen | 0.37436513 | Positive | k_Bacteria; p_Bacteroidetes; c_Bacteroidia; | 384 | |
| vo1614905 | Butyrate | | | o_Bacteroidales; f_; g_; s_ | | |
| deno- | Rumen | 0.526770855 | Positive | k_Bacteria; p_Firmicutes; c_Clostridia; | 385 | |
| vo1627012 | Propionate | | | o_Clostridiales; f_Lachnospiraceae; | | |
| | | | | g_Coprococcus; s_ | | |
| deno- | Rumen | 0.319795561 | Positive | k_Bacteria; p_Firmicutes; c_Clostridia; | 386 | |
| vo1627012 | Valerate | | | o_Clostridiales; f_Lachnospiraceae; | | |
| | | | | g_Coprococcus; s_ | | |
| deno- | Rumen | 0.549089985 | Positive | k_Bacteria; p_Bacteroidetes; c_Bacteroidia; | 387 | |
| vo1629621 | Propionate | | | o_Bacteroidales; f_Prevotellaceae; g_Prevotella; | | |
| | | | | s_ | | |
| deno- | Rumen | 0.745898239 | Positive | k_Bacteria; p_Bacteroidetes; c_Bacteroidia; | 388 | |
| vo1641807 | Propionate | | | o_Bacteroidales; f_Prevotellaceae; g_Prevotella; | | |
| | | | | s_ | | |
| deno- | Rumen | 0.570542031 | Positive | k_Bacteria; p_Bacteroidetes; c_Bacteroidia; | 389 | |
| vo1645223 | Acetate | | | o_Bacteroidales; f_[Paraprevotellaceae]; | | |
| | | | | g_CF231; s_ | | |
| deno- | Rumen | 0.440932808 | Positive | k_Bacteria; p_Verrucomicrobia; c_Verruco-5; | 390 | |
| vo1649599 | Acetate | | | o_WCHB1-41; f_RFP12; g_; s_ | | |
| deno- | Milk fat | 0.425341942 | Positive | k_Bacteria; p_Bacteroidetes; c_Bacteroidia; | 391 | |
| vo1651093 | | | | o_Bacteroidales; f_; g_; s_ | | |
| deno- | Rumen | 0.514876686 | Positive | k_Bacteria; p_Bacteroidetes; c_Bacteroidia; | 392 | |
| vo1651093 | Acetate | | | o_Bacteroidales; f_; g_; s_ | | |
| deno- | Rumen | 0.445625932 | Positive | k_Bacteria; p_Bacteroidetes; c_Bacteroidia; | 393 | |
| vo1654182 | Acetate | | | o_Bacteroidales; f_; g_; s_ | | |
| deno- | Rumen | 0.344416424 | Positive | k_Bacteria; p_Firmicutes; c_Clostridia; | 394 | |
| vo1656455 | Acetate | | | o_Clostridiales; f_Ruminococcaceae; | | |
| | | | | g_Ruminococcus; s_ | | |
| deno- | Rumen | 0.309976509 | Positive | k_Bacteria; p_Firmicutes; c_Clostridia; | 395 | |
| vo1656455 | Isobutyrate | | | o_Clostridiales; f_Ruminococcaceae; | | |
| | | | | g_Ruminococcus; s_ | | |
| deno- | Total | 0.315908568 | Positive | k_Bacteria; p_Bacteroidetes; c_Bacteroidia; | 396 | |
| vo1659598 | digestion | | | o_Bacteroidales; f_; g_; s_ | | |
| | dry | | | | | |
| | matter | | | | | |
| deno- | Rumen | 0.432343776 | Positive | k_Bacteria; p_Proteobacteria; | 397 | |
| vo1665986 | Acetate | | | c_Gammaproteobacteria; o_Aeromonadales; | | |
| | | | | f_Succinivibrionaceae; g_; s_ | | |
| deno- | Rumen | 0.462827611 | Positive | k_Bacteria; p_Firmicutes; c_Clostridia; | 398 | |
| vo1678620 | Propionate | | | o_Clostridiales; f_Veillonellaceae | | |
| deno- | Rumen | 0.505615233 | Positive | k_Bacteria; p_Proteobacteria; | 399 | |
| vo1678621 | Propionate | | | c_Deltaproteobacteria; o_Desulfovibrionales; | | |
| | | | | f_Desulfovibrionaceae; g_Desulfovibrio; s_D168 | | |
| deno- | Rumen | 0.432065341 | Positive | k_Bacteria; p_Firmicutes; c_Clostridia; | 400 | |
| vo1685547 | Acetate | | | o_Clostridiales; f_Ruminococcaceae | | |
| deno- | Rumen | 0.471198549 | Positive | k_Bacteria; p_Bacteroidetes; c_Bacteroidia; | 401 | |
| vo168993 | Propionate | | | o_Bacteroidales; f_Prevotellaceae; g_Prevotella; | | |
| | | | | s_ | | |
| deno- | Rumen | 0.404799028 | Positive | k_Bacteria; p_Bacteroidetes; c_Bacteroidia; | 402 | |
| vo170160 | Butyrate | | | o_Bacteroidales; f_Prevotellaceae; g_Prevotella; | | |
| | | | | s_ | | |
| deno- | CH4 | 0.440183538 | Positive | k_Bacteria; p_Bacteroidetes; c_Bacteroidia; | 403 | |
| vo170257 | g/kg | | | o_Bacteroidales; f_Prevotellaceae; g_Prevotella; | | |
| | ECM | | | s_ | | |
| deno- | Rumen | 0.578441063 | Positive | k_Bacteria; p_Bacteroidetes; c_Bacteroidia; | 404 | |
| vo170257 | Acetate | | | o_Bacteroidales; f_Prevotellaceae; g_Prevotella; | | |
| | | | | s_ | | |
| deno- | Rumen | 0.362415744 | Positive | k_Bacteria; p_Verrucomicrobia; c_Verruco-5; | 405 | |
| vo1702990 | Acetate | | | o_WCHB1-41; f_RFP12; g_; s_ | | |
| deno- | Rumen | 0.392118605 | Positive | k_Bacteria; p_Bacteroidetes; c_Bacteroidia; | 406 | |
| vo1716654 | Butyrate | | | o_Bacteroidales; f_Prevotellaceae; g_Prevotella; | | |
| | | | | s_ | | |
| deno- | Rumen | 0.349678626 | Positive | k_Bacteria; p_Actinobacteria; c_Coriobacteriia; | 407 | |
| vo1717065 | Propionate | | | o_Coriobacteriales; f_Coriobacteriaceae; g_; s_ | | |
| deno- | Rumen | 0.39329244 | Positive | k_Bacteria; p_Bacteroidetes; c_Bacteroidia; | 408 | |
| vo1722008 | Propionate | | | o_Bacteroidales; f_Prevotellaceae; g_Prevotella; | | |
| | | | | s_ | | |
| deno- | Rumen | 0.399938247 | Positive | k_Bacteria; p_Bacteroidetes; c_Bacteroidia; | 409 | |
| vo1728005 | Acetate | | | o_Bacteroidales; f_; g_; s_ | | |
| deno- | Rumen | 0.406815731 | Positive | k_Bacteria; p_Bacteroidetes; c_Bacteroidia; | 410 | |
| vo1734495 | Acetate | | | o_Bacteroidales; f_; g_; s_ | | |
| deno- | Rumen | 0.450342348 | Positive | k_Bacteria; p_Bacteroidetes; c_Bacteroidia; | 411 | |
| vo1756558 | Propionate | | | o_Bacteroidales; f_Prevotellaceae; g_Prevotella; | | |
| | | | | s_ | | |
| deno- | Milk fat | 0.280243847 | Positive | k_Bacteria; p_Bacteroidetes; c_Bacteroidia; | 412 | |
| vo1783497 | | | | o_Bacteroidales; f_Prevotellaceae; g_Prevotella; | | |
| | | | | s_ | | |
| deno- | Rumen | 0.307713795 | Positive | k_Bacteria; p_Bacteroidetes; c_Bacteroidia; | 413 | |
| vo1783497 | Acetate | | | o_Bacteroidales; f_Prevotellaceae; g_Prevotella; | | |
| | | | | s_ | | |
| deno- | Rumen | 0.663197373 | Positive | k_Bacteria; p_Firmicutes; c_Clostridia; | 414 | |
| vo1795734 | Propionate | | | o_Clostridiales; f_Veillonellaceae; g_Dialister; | | |
| | | | | s_ | | |
| deno- | Rumen | 0.603317082 | Positive | k_Bacteria; p_Bacteroidetes; c_Bacteroidia; | 415 | |
| vo1801715 | Propionate | | | o_Bacteroidales; f_Prevotellaceae; g_Prevotella; | | |
| | | | | s_ | | |
| deno- | CH4 | 0.445222147 | Positive | k_Bacteria; p_Bacteroidetes; c_Bacteroidia; | 416 | |
| vo1803997 | g/kg | | | o_Bacteroidales; f_Prevotellaceae; g_Prevotella; | | |
| | ECM | | | s_ | | |
| deno- | Rumen | 0.648946495 | Positive | k_Bacteria; p_Bacteroidetes; c_Bacteroidia; | 417 | |
| vo1803997 | Acetate | | | o_Bacteroidales; f_Prevotellaceae; g_Prevotella; | | |
| | | | | s_ | | |
| deno- | Plasma | 0.347544376 | Positive | k_Bacteria; p_Firmicutes; c_Clostridia; | 418 | |
| vo1804005 | BHB | | | o_Clostridiales; f_Ruminococcaceae; | | |
| | | | | g_Ruminococcus; s_ | | |
| deno- | Rumen | 0.42688138 | Positive | k_Bacteria; p Actinobacteria; c_Coriobacteriia; | 419 | |
| vo1858871 | Propionate | | | o_Coriobacteriales; f_Coriobacteriaceae; g_; s_ | | |
| deno- | Rumen | 0.528590935 | Positive | k_Bacteria; p_Firmicutes; c_Clostridia; | 420 | |
| vo1871583 | Acetate | | | o_Clostridiales; f_Christensenellaceae; g_; s_ | | |
| deno- | Rumen | 0.330536348 | Positive | k_Bacteria; p_Bacteroidetes; c_Bacteroidia; | 421 | |
| vo1874224 | Butyrate | | | o_Bacteroidales; f_Prevotellaceae; g_Prevotella; | | |
| | | | | s_ | | |
| deno- | Rumen | 0.477798965 | Positive | k_Bacteria; p_Lentisphaerae; c_[Lentisphaeria]; | 422 | |
| vo1875086 | Acetate | | | o_Z20; f_R4-45B; g_; s_ | | |
| deno- | Plasma | 0.284202587 | Positive | k_Bacteria; p_Bacteroidetes; c_Bacteroidia; | 423 | |
| vo1880747 | BHB | | | o_Bacteroidales; f_Prevotellaceae; g_Prevotella; | | |
| | | | | s_ | | |
| deno- | Rumen | 0.334415115 | Positive | k_Bacteria; p_Bacteroidetes; c_Bacteroidia; | 424 | |
| vo1885363 | Acetate | | | o_Bacteroidales; f_Prevotellaceae; g_Prevotella; | | |
| | | | | s_ | | |
| deno- | Rumen | 0.364886872 | Positive | k_Bacteria; p_Bacteroidetes; c_Bacteroidia; | 425 | |
| vo188900 | Acetate | | | o_Bacteroidales; f_[Paraprevotellaceae]; | | |
| | | | | g_YRC22; s_ | | |
| deno- | Rumen | 0.411224939 | Positive | k_Bacteria; p_Proteobacteria; | 426 | |
| vo1891669 | Acetate | | | c_Gammaproteobacteria; o_Aeromonadales; | | |
| | | | | f_Succinivibrionaceae; g_; s_ | | |
| deno- | Rumen | 0.453892923 | Positive | k_Bacteria; p_Bacteroidetes; c_Bacteroidia; | 427 | |
| vo1934186 | Propionate | | | o_Bacteroidales; f_Prevotellaceae; g_Prevotella; | | |
| | | | | s_ | | |
| deno- | Milk fat | 0.425557249 | Positive | k_Bacteria; p_Proteobacteria; | 428 | |
| vo1937263 | | | | c_Gammaproteobacteria; o_Aeromonadales; | | |
| | | | | f_Succinivibrionaceae; g_Ruminobacter; s_ | | |
| deno- | Rumen | 0.396862716 | Positive | k_Bacteria; p_Bacteroidetes; c_Bacteroidia; | 429 | |
| vo194317 | Acetate | | | o_Bacteroidales; f_; g_; s_ | | |
| deno- | Total | 0.314779367 | Positive | k_Bacteria; p_Bacteroidetes; c_Bacteroidia; | 430 | |
| vo194317 | digestion | | | o_Bacteroidales; f_; g_; s_ | | |
| | dry | | | | | |
| | matter | | | | | |
| deno- | Rumen | 0.3225831 | Positive | k_Bacteria; p_Bacteroidetes; c_Bacteroidia; | 431 | |
| vo1958235 | Butyrate | | | o_Bacteroidales; f_Prevotellaceae; g_Prevotella; | | |
| | | | | s_ | | |
| deno- | Rumen | 0.516081875 | Positive | k_Bacteria; p_Bacteroidetes; c_Bacteroidia; | 432 | |
| vo1966905 | Propionate | | | o_Bacteroidales; f_Prevotellaceae; g_Prevotella; | | |
| | | | | s_ | | |
| deno- | Rumen | 0.587762831 | Positive | k_Bacteria; p_Bacteroidetes; c_Bacteroidia; | 433 | |
| vo1988814 | Propionate | | | o_Bacteroidales; f_Prevotellaceae; g_Prevotella; | | |
| | | | | s_ | | |
| deno- | Rumen | 0.28821759 | Positive | k_Bacteria; p_Bacteroidetes; c_Bacteroidia; | 434 | |
| vo2000236 | Propionate | | | o_Bacteroidales; f_Prevotellaceae; g_Prevotella; | | |
| | | | | s_ | | |
| deno- | Rumen | 0.296981224 | Positive | k_Bacteria; p_Tenericutes; c_Mollicutes; | 435 | |
| vo2047207 | pH | | | o_Anaeroplasmatales; f_Anaeroplasmataceae; | | |
| | | | | g_Anaeroplasma; s_ | | |
| deno- | Rumen | 0.458257833 | Positive | k_Bacteria; p_Firmicutes; c_Clostridia; | 436 | |
| vo2059914 | Propionate | | | o_Clostridiales; f_Lachnospiraceae; | | |
| | | | | g_Coprococcus; s_ | | |
| deno- | Rumen | 0.365985898 | Positive | k_Bacteria; p_Bacteroidetes; c_Bacteroidia; | 437 | |
| vo2069744 | Propionate | | | o_Bacteroidales; f_Prevotellaceae; g_Prevotella; | | |
| | | | | s_ | | |
| deno- | Rumen | 0.41864429 | Positive | k_Bacteria; p_Bacteroidetes; c_Bacteroidia; | 438 | |
| vo2091417 | Acetate | | | o_Bacteroidales; f_; g_; s_ | | |
| deno- | Rumen | 0.402781851 | Positive | k_Bacteria; p_Bacteroidetes; c_Bacteroidia; | 439 | |
| vo2093314 | Propionate | | | o_Bacteroidales; f_Prevotellaceae; g_Prevotella; | | |
| | | | | s_ | | |
| deno- | Rumen | 0.35482363 | Positive | k_Bacteria; p_Bacteroidetes; c_Bacteroidia; | 440 | |
| vo2108360 | Propionate | | | o_Bacteroidales; f_Prevotellaceae; g_Prevotella; | | |
| | | | | s_ | | |
| deno- | Rumen | 0.321584543 | Positive | k_Bacteria; p_Bacteroidetes; c_Bacteroidia; | 441 | |
| vo211105 | Propionate | | | o_Bacteroidales; f_Prevotellaceae; g_Prevotella; | | |
| | | | | s_ | | |
| deno- | Rumen | 0.291938458 | Positive | k_Bacteria; p_Bacteroidetes; c_Bacteroidia; | 442 | |
| vo211107 | Butyrate | | | o_Bacteroidales; f_Prevotellaceae; g_Prevotella; | | |
| | | | | s_ | | |
| deno- | Rumen | 0.413742415 | Positive | k_Bacteria; p_Firmicutes; c_Clostridia; | 443 | |
| vo2114712 | Propionate | | | o_Clostridiales; f_Ruminococcaceae; | | |
| | | | | g_ Ruminococcus | | |
| deno- | Plasma | 0.327704061 | Positive | k_Bacteria; p_Proteobacteria; | 444 | |
| vo2141307 | BHB | | | c_Deltaproteobacteria; o_Desulfovibrionales; | | |
| | | | | f_Desulfovibrionaceae; g_Desulfovibrio; s_D168 | | |
| deno- | Rumen | 0.680386922 | Positive | k_Bacteria; p_Firmicutes; c_Clostridia; | 445 | |
| vo2190261 | Propionate | | | o_Clostridiales; f_Lachnospiraceae | | |
| deno- | CH4 | 0.464116426 | Positive | k_Bacteria; p_Bacteroidetes; c_Bacteroidia; | 446 | |
| vo2199124 | g/d | | | o_Bacteroidales; f_; g_; s_ | | |
| deno- | Rumen | 0.486910444 | Positive | k_Bacteria; p_Bacteroidetes; c_Bacteroidia; | 447 | |
| vo2213203 | Butyrate | | | o_Bacteroidales; f_Prevotellaceae; g_Prevotella; | | |
| | | | | s_ | | |
| deno- | Rumen | 0.370160329 | Positive | k_Bacteria; p_Bacteroidetes; c_Bacteroidia; | 448 | |
| vo2222214 | Acetate | | | o_Bacteroidales; f_; g_; s_ | | |
| deno- | Rumen | 0.47000907 | Positive | k_Bacteria; p_Firmicutes; c_Clostridia; | 449 | |
| vo2227499 | Propionate | | | o_Clostridiales; f_Lachnospiraceae; | | |
| | | | | g_Coprococcus; s_ | | |
| deno- | Rumen | 0.348207567 | Positive | k_Bacteria; p_Proteobacteria; | 450 | |
| vo2236813 | Acetate | | | c_Alphaproteobacteria; o_Rickettsiales; f_; g_; | | |
| | | | | s_ | | |
| deno- | Rumen | 0.290090709 | Positive | k_Bacteria; p_Bacteroidetes; c_Bacteroidia; | 451 | |
| vo2256055 | Butyrate | | | o_Bacteroidales; f_Prevotellaceae; g_Prevotella; | | |
| | | | | s_ | | |
| deno- | Rumen | 0.390835926 | Positive | k_Bacteria; p_Firmicutes; c_Clostridia; | 452 | |
| vo2276897 | Propionate | | | o_Clostridiales; f_ Ruminococcaceae; | | |
| | | | | g_Ruminococcus; s_flavefaciens | | |
| deno- | Rumen | 0.377541377 | Positive | k_Bacteria; p_Firmicutes; c_Clostridia; | 453 | |
| vo2294592 | Butyrate | | | o_Clostridiales; f_Lachnospiraceae; g_Moryella; | | |
| | | | | s_ | | |
| deno- | Rumen | 0.643034351 | Positive | k_Bacteria; p_Firmicutes; c_Clostridia; | 454 | |
| vo2301555 | Propionate | | | o_Clostridiales; f_Lachnospiraceae | | |
| deno- | Rumen | 0.411914834 | Positive | k_Bacteria; p_Proteobacteria; | 455 | |
| vo2308695 | Propionate | | | c_Gammaproteobacteria; o_Aeromonadales; | | |
| | | | | f_Succinivibrionaceae; g_; s_ | | |
| deno- | Rumen | 0.498419555 | Positive | k_Bacteria; p_Spirochaetes; c_Spirochaetes; | 456 | |
| vo2310307 | Acetate | | | o_Spirochaetales; f_Spirochaetaceae; | | |
| | | | | g_Treponema; s_ | | |
| deno- | Rumen | 0.638857596 | Positive | k_Bacteria; p_Bacteroidetes; c_Bacteroidia; | 457 | |
| vo2318873 | Propionate | | | o_Bacteroidales; f_Prevotellaceae; g_Prevotella; | | |
| | | | | s_ | | |
| deno- | Plasma | 0.344077548 | Positive | k_Bacteria; p_Bacteroidetes; c_Bacteroidia; | 458 | |
| vo2323272 | BHB | | | o_Bacteroidales; f_; g_; s_ | | |
| deno- | Rumen | 0.640150946 | Positive | k_Bacteria; p_Bacteroidetes; c_Bacteroidia; | 459 | |
| vo2323272 | Acetate | | | o_Bacteroidales; f_; g_; s_ | | |
| deno- | Rumen | 0.442665202 | Positive | k_Bacteria; p_Bacteroidetes; c_Bacteroidia; | 460 | |
| vo2358052 | Propionate | | | o_Bacteroidales; f_Prevotellaceae; g_Prevotella; | | |
| | | | | s_ | | |
| deno- | Rumen | 0.525371708 | Positive | k_Bacteria; p_Bacteroidetes; c_Bacteroidia; | 461 | |
| vo2364698 | Propionate | | | o_Bacteroidales; f_Prevotellaceae; g_Prevotella; | | |
| | | | | s_ | | |
| deno- | Rumen | 0.412056477 | Positive | k_Bacteria; p_Bacteroidetes; c_Bacteroidia; | 462 | |
| vo2367933 | Propionate | | | o_Bacteroidales; f_Prevotellaceae; g_Prevotella; | | |
| | | | | s_ | | |
| deno- | Rumen | 0.393559932 | Positive | k_Bacteria; p_Bacteroidetes; c_Bacteroidia; | 463 | |
| vo24845 | Propionate | | | o_Bacteroidales; f_RF16; g_; s_ | | |
| deno- | Rumen | 0.364598961 | Positive | k_Bacteria; p_Bacteroidetes; c_Bacteroidia; | 464 | |
| vo248780 | Acetate | | | o_Bacteroidales; f_Prevotellaceae; g_Prevotella; | | |
| | | | | s_ | | |
| deno- | Rumen | 0.364526976 | Positive | k_Bacteria; p_Bacteroidetes; c_Bacteroidia; | 465 | |
| vo252478 | Acetate | | | o_Bacteroidales; f_Prevotellaceae; g_Prevotella; | | |
| | | | | s_ | | |
| deno- | Rumen | 0.51037541 | Positive | k_Bacteria; p_Firmicutes; c_Clostridia; | 466 | |
| vo260384 | Butyrate | | | o_Clostridiales; f_Veillonellaceae; | | |
| | | | | g_Selenomonas; s_ruminantium | | |
| deno- | Rumen | 0.504304326 | Positive | k_Bacteria; p_Bacteroidetes; c_Bacteroidia; | 467 | |
| vo263528 | Propionate | | | o_Bacteroidales; f_Prevotellaceae; g_Prevotella; | | |
| | | | | s_ | | |
| deno- | Rumen | 0.319264669 | Positive | k_Bacteria; p_Firmicutes; c_Clostridia; | 468 | |
| vo265909 | Ammonia | | | o_Clostridiales; f_Lachnospiraceae; | | |
| | | | | g_Pseudobutyrivibrio; s_ | | |
| deno- | Rumen | 0.459437764 | Positive | k_Bacteria; p_Bacteroidetes; c_Bacteroidia; | 469 | |
| vo275229 | Butyrate | | | o_Bacteroidales; f_; g_; s_ | | |
| deno- | Rumen | 0.579715492 | Positive | k_Bacteria; p_Bacteroidetes; c_Bacteroidia; | 470 | |
| vo278746 | Acetate | | | o_Bacteroidales; f_; g_; s_ | | |
| deno- | Rumen | 0.506830229 | Positive | k_Bacteria; p_Spirochaetes; c_Spirochaetes; | 471 | |
| vo279606 | Acetate | | | o_Spirochaetales; f_Spirochaetaceae; | | |
| | | | | g_Treponema; s_ | | |
| deno- | Rumen | 0.386199974 | Positive | k_Bacteria; p_Bacteroidetes; c_Bacteroidia; | 472 | |
| vo29865 | Acetate | | | o_Bacteroidales; f_[Paraprevotellaceae]; | | |
| | | | | g_CF231; s_ | | |
| deno- | Rumen | 0.379951828 | Positive | k_Bacteria; p_Verrucomicrobia; c_Verruco-5; | 473 | |
| vo318201 | Acetate | | | o_WCHB1-41; f_RFP12; g_; s_ | | |
| deno- | Total | 0.336182439 | Positive | k_Bacteria; p_Verrucomicrobia; c_Verruco-5; | 474 | |
| vo318201 | digestion | | | o_WCHB1-41; f_RFP12; g_; s_ | | |
| | dry | | | | | |
| | matter | | | | | |
| deno- | Rumen | 0.410201661 | Positive | k_Bacteria; p_Bacteroidetes; c_Bacteroidia; | 475 | |
| vo340240 | Propionate | | | o_Bacteroidales; f_Prevotellaceae; g_Prevotella; | | |
| | | | | s_ | | |
| deno- | Rumen | 0.512953367 | Positive | k_Bacteria; p_Bacteroidetes; c_Bacteroidia; | 476 | |
| vo34274 | Propionate | | | o_Bacteroidales; f_Prevotellaceae; g_Prevotella; | | |
| | | | | s_ | | |
| deno- | Milk | 0.306037064 | Positive | k_Bacteria; p_Proteobacteria; | 477 | |
| vo358994 | lactose | | | c_Gammaproteobacteria; o_Aeromonadales; | | |
| | | | | f_Succinivibrionaceae; g_; s_ | | |
| deno- | Milk | 0.304875885 | Positive | k_Bacteria; p_Proteobacteria; | 478 | |
| vo358994 | yield | | | c_Gammaproteobacteria; o_Aeromonadales; | | |
| | | | | f_Succinivibrionaceae; g_; s_ | | |
| deno- | Rumen | 0.26132017 | Positive | k_Bacteria; p_Spirochaetes; c_Spirochaetes; | 479 | |
| vo368299 | pH | | | o_Spirochaetales; f_Spirochaetaceae; | | |
| | | | | g_Treponema; s_ | | |
| deno- | Rumen | 0.564184515 | Positive | k_Bacteria; p_Proteobacteria; | 480 | |
| vo384931 | Acetate | | | c_Gammaproteobacteria; o_Aeromonadales; | | |
| | | | | f_Succinivibrionaceae; g_Succinivibrio; s_ | | |
| deno- | Rumen | 0.515550565 | Positive | k_Bacteria; p_Firmicutes; c_Clostridia; | 481 | |
| vo390275 | Propionate | | | o_Clostridiales; f_; g_; s_ | | |
| deno- | Plasma | 0.324877083 | Positive | k_Bacteria; p_Bacteroidetes; c_Bacteroidia; | 482 | |
| vo398343 | BHB | | | o_Bacteroidales; f_Prevotellaceae; g_Prevotella; | | |
| | | | | s_ | | |
| deno- | Rumen | 0.422574508 | Positive | k_Bacteria; p_Bacteroidetes; c_Bacteroidia; | 483 | |
| vo401466 | Acetate | | | o_Bacteroidales_ | | |
| deno- | Rumen | 0.724129621 | Positive | k_Bacteria; p_Firmicutes; c_Clostridia; | 484 | |
| vo445030 | Propionate | | | o_Clostridiales; f_Veillonellaceae; g_Dialister; | | |
| | | | | s_ | | |
| deno- | Rumen | 0.52957203 | Positive | k_Bacteria; p_Proteobacteria; | 485 | |
| vo454615 | Propionate | | | c_Gammaproteobacteria; o_Aeromonadales; | | |
| | | | | f_Succinivibrionaceae; g_; s_ | | |
| deno- | Rumen | 0.486518382 | Positive | k_Bacteria; p_Bacteroidetes; c_Bacteroidia; | 486 | |
| vo461510 | Propionate | | | o_Bacteroidales; f_Prevotellaceae; g_Prevotella; | | |
| | | | | s_ | | |
| deno- | Rumen | 0.374963958 | Positive | k_Bacteria; p_Proteobacteria; | 487 | |
| vo473355 | Propionate | | | c_Gammaproteobacteria; o_Aeromonadales; | | |
| | | | | f_Succinivibrionaceae; g_; s_ | | |
| deno- | Rumen | 0.46829132 | Positive | k_Bacteria; p_Firmicutes; c_Clostridia; | 488 | |
| vo477266 | Propionate | | | o_Clostridiales; f_Lachnospiraceae; | | |
| | | | | g_Butyrivibrio; s_ | | |
| deno- | Rumen | 0.570286521 | Positive | k_Bacteria; p_Proteobacteria; | 489 | |
| vo481551 | Propionate | | | c_Gammaproteobacteria; o_Aeromonadales; | | |
| | | | | f_Succinivibrionaceae; g_; s_ | | |
| deno- | Rumen | 0.579102572 | Positive | k_Bacteria; p_Bacteroidetes; c_Bacteroidia; | 490 | |
| vo48352 | Propionate | | | o_Bacteroidales; f_Prevotellaceae; g_Prevotella; | | |
| | | | | s_ | | |
| deno- | Rumen | 0.39069299 | Positive | k_Bacteria; p_Firmicutes; c_Clostridia; | 491 | |
| vo488679 | Acetate | | | o_Clostridiales; f_Ruminococcaceae; g_; s_ | | |
| deno- | Rumen | 0.380433591 | Positive | k_Bacteria; p_Verrucomicrobia; c_Verruco-5; | 492 | |
| vo510868 | Acetate | | | o_WCHB1-41; f_RFP12; g_; s_ | | |
| deno- | Milk | 0.266432444 | Positive | k_Bacteria; p_Bacteroidetes; c_Bacteroidia; | 493 | |
| vo521876 | lactose | | | o_Bacteroidales; f_Prevotellaceae; g_Prevotella; | | |
| | | | | s_ | | |
| deno- | Milk | 0.261277785 | Positive | k_Bacteria; p_Bacteroidetes; c_Bacteroidia; | 494 | |
| vo521876 | yield | | | o_Bacteroidales; f_Prevotellaceae; g_Prevotella; | | |
| | | | | s_ | | |
| deno- | Fecal | 0.265907983 | Positive | k_Bacteria; p_Verrucomicrobia; c_Verruco-5; | 495 | |
| vo523957 | AIA | | | o_WCHB1-41; f_RFP12; g_; s_ | | |
| deno- | Rumen | 0.326387563 | Positive | k_Bacteria; p_Bacteroidetes; c_Bacteroidia; | 496 | |
| vo539849 | Butyrate | | | o_Bacteroidales; f_Prevotellaceae; g_Prevotella; | | |
| | | | | s_ | | |
| deno- | Rumen | 0.639519141 | Positive | k_Bacteria; p_Bacteroidetes; c_Bacteroidia; | 497 | |
| vo548248 | Propionate | | | o_Bacteroidales; f_Prevotellaceae; g_Prevotella; | | |
| | | | | s_ | | |
| deno- | Rumen | 0.338403312 | Positive | k_Bacteria; p_Bacteroidetes; c_Bacteroidia; | 498 | |
| vo554901 | Valerate | | | o_Bacteroidales; f_Prevotellaceae; g_Prevotella; | | |
| | | | | s_ | | |
| deno- | Rumen | 0.531430969 | Positive | k_Bacteria; p_Proteobacteria; | 499 | |
| vo560186 | Acetate | | | c_Gammaproteobacteria; o_Aeromonadales; | | |
| | | | | f_Succinivibrionaceae; g_; s_ | | |
| deno- | Rumen | 0.314620973 | Positive | k_Bacteria; p_Proteobacteria; | 500 | |
| vo572244 | Propionate | | | c_Gammaproteobacteria; o_Aeromonadales; | | |
| | | | | f_Succinivibrionaceae; g_; s_ | | |
| deno- | Rumen | 0.515046107 | Positive | k_Bacteria; p_Bacteroidetes; c_Bacteroidia; | 501 | |
| vo576104 | Propionate | | | o_Bacteroidales; f_Prevotellaceae; g_Prevotella; | | |
| | | | | s_ | | |
| deno- | Rumen | 0.327902528 | Positive | k_Bacteria; p_Bacteroidetes; c_Bacteroidia; | 502 | |
| vo577780 | Valerate | | | o_Bacteroidales; f_Prevotellaceae; g_Prevotella; | | |
| | | | | s_ | | |
| deno- | Rumen | 0.519759351 | Positive | k_Bacteria; p_Proteobacteria; | 503 | |
| vo578861 | Acetate | | | c_Gammaproteobacteria; o_Aeromonadales; | | |
| | | | | f_Succinivibrionaceae; g_; s_ | | |
| deno- | Rumen | 0.401448048 | Positive | k_Bacteria; p_Bacteroidetes; c_Bacteroidia; | 504 | |
| vo582588 | Acetate | | | o_Bacteroidales; f_[Paraprevotellaceae]; | | |
| | | | | g_CF231; s_ | | |
| deno- | Rumen | 0.361357135 | Positive | k_Bacteria; p_Bacteroidetes; c_Bacteroidia; | 505 | |
| vo582825 | Acetate | | | o_Bacteroidales; f_Prevotellaceae; g_; s_ | | |
| deno- | Rumen | 0.370970999 | Positive | k_Bacteria; p_Verrucomicrobia; c_Verruco-5; | 506 | |
| vo582828 | Acetate | | | o_WCHB1-41; f_RFP12; g_; s_ | | |
| deno- | Rumen | 0.47302605 | Positive | k_Bacteria; p_Bacteroidetes; c_Bacteroidia; | 507 | |
| vo585153 | Propionate | | | o_Bacteroidales; f_Prevotellaceae; g_Prevotella; | | |
| | | | | s_ | | |
| deno- | Rumen | 0.64942237 | Positive | k_Bacteria; p_Firmicutes; c_Clostridia; | 508 | |
| vo612360 | Propionate | | | o_Clostridiales; f_Veillonellaceae; g_Dialister; | | |
| | | | | s_ | | |
| deno- | Plasma | 0.362291131 | Positive | k_Bacteria; p_Bacteroidetes; c_Bacteroidia; | 509 | |
| vo618436 | BHB | | | o_Bacteroidales; f_[Paraprevotellaceae]; | | |
| | | | | g_CF231; s_ | | |
| deno- | Rumen | 0.576533933 | Positive | k_Bacteria; p_Bacteroidetes; c_Bacteroidia; | 510 | |
| vo618436 | Acetate | | | o_Bacteroidales; f_[Paraprevotellaceae]; | | |
| | | | | g_CF231; s_ | | |
| deno- | Rumen | 0.357327061 | Positive | k_Bacteria; p_Bacteroidetes; c_Bacteroidia; | 511 | |
| vo625380 | Acetate | | | o_Bacteroidales; f_Prevotellaceae; g_Prevotella; | | |
| | | | | s_ | | |
| deno- | Rumen | 0.407983645 | Positive | k_Bacteria; p_Bacteroidetes; c_Bacteroidia; | 512 | |
| vo650074 | Propionate | | | o_Bacteroidales; f_Prevotellaceae; g_Prevotella; | | |
| | | | | s_ | | |
| deno- | Rumen | 0.375766807 | Positive | k_Bacteria; p_Bacteroidetes; c_Bacteroidia; | 513 | |
| vo671109 | Acetate | | | o_Bacteroidales; f_S24-7; g_; s_ | | |
| deno- | Rumen | 0.25803144 | Positive | k_Bacteria; p_Firmicutes; c_Clostridia; | 514 | |
| vo687413 | Acetate | | | o_Clostridiales; f_Lachnospiraceae; | | |
| | | | | g_Robinsoniella; s_peoriensis | | |
| deno- | Rumen | 0.587090398 | Positive | k_Bacteria; p_Firmicutes; c_Clostridia; | 515 | |
| vo693429 | Acetate | | | o_Clostridiales; f_Clostridiaceae; g_02d06; s_ | | |
| deno- | Rumen | 0.492077583 | Positive | k_Bacteria; p_Bacteroidetes; c_Bacteroidia; | 516 | |
| vo701009 | Propionate | | | o_Bacteroidales; f_[Paraprevotellaceae]; | | |
| | | | | g_CF231; s_ | | |
| deno- | Rumen | 0.396731248 | Positive | k_Bacteria; p_Bacteroidetes; c_Bacteroidia; | 517 | |
| vo706011 | Propionate | | | o_Bacteroidales; f_Prevotellaceae; g_Prevotella; | | |
| | | | | s_ | | |
| deno- | Rumen | 0.380530847 | Positive | k_Bacteria; p_Bacteroidetes; c_Bacteroidia; | 518 | |
| vo725148 | Acetate | | | o_Bacteroidales; f_RF16; g_; s_ | | |
| deno- | Rumen | 0.400643777 | Positive | k_Bacteria; p_Bacteroidetes; c_Bacteroidia; | 519 | |
| vo73975 | Acetate | | | o_Bacteroidales_ | | |
| deno- | Rumen | 0.408239893 | Positive | k_Bacteria; p_Bacteroidetes; c_Bacteroidia; | 520 | |
| vo745561 | Propionate | | | o_Bacteroidales; f_Prevotellaceae; g_Prevotella; | | |
| | | | | s_ | | |
| deno- | Rumen | 0.497137849 | Positive | k_Bacteria; p_Bacteroidetes; c_Bacteroidia; | 521 | |
| vo775642 | Propionate | | | o_Bacteroidales; f_Prevotellaceae; g_Prevotella; | | |
| | | | | s_ | | |
| deno- | Rumen | 0.413518306 | Positive | k_Bacteria; p_Firmicutes; c_Clostridia; | 522 | |
| vo778208 | Acetate | | | o_Clostridiales; f_Ruminococcaceae; | | |
| | | | | g_Ruminococcus; s_ | | |
| deno- | Plasma | 0.276418274 | Positive | k_Bacteria; p_Bacteroidetes; c_Bacteroidia; | 523 | |
| vo782634 | BHB | | | o_Bacteroidales; f_Prevotellaceae; g_Prevotella; | | |
| | | | | s_ | | |
| deno- | Rumen | 0.588472259 | Positive | k_Bacteria; p_Bacteroidetes; c_Bacteroidia; | 524 | |
| vo798795 | Propionate | | | o_Bacteroidales; f_Prevotellaceae; g_Prevotella; | | |
| | | | | s_ | | |
| deno- | Rumen | 0.511495935 | Positive | k_Bacteria; p_Firmicutes; c_Clostridia; | 525 | |
| vo824434 | Propionate | | | o_Clostridiales; f_Lachnospiraceae; | | |
| | | | | g_Shuttleworthia; s_ | | |
| deno- | Rumen | 0.515949501 | Positive | k_Bacteria; p_Bacteroidetes; c_Bacteroidia; | 526 | |
| vo846056 | Acetate | | | o_Bacteroidales; f_Prevotellaceae; g_Prevotella; | | |
| | | | | s_ | | |
| deno- | Rumen | 0.268880063 | Positive | k_Bacteria; p_Spirochaetes; c_Spirochaetes; | 527 | |
| vo860783 | Acetate | | | o_Spirochaetales; f_Spirochaetaceae; g_; s_ | | |
| deno- | Rumen | 0.5683119 | Positive | k_Bacteria; p_Proteobacteria; | 528 | |
| vo862967 | Acetate | | | c_Gammaproteobacteria; o_Aeromonadales; | | |
| | | | | f_Succinivibrionaceae; g_Ruminobacter; s_ | | |
| deno- | Rumen | 0.350421556 | Positive | k_Bacteria; p_Bacteroidetes; c_Bacteroidia; | 529 | |
| vo86669 | Acetate | | | o_Bacteroidales; f_; g_; s_ | | |
| deno- | Rumen | 0.396606786 | Positive | k_Bacteria; p_Bacteroidetes; c_Bacteroidia; | 530 | |
| vo878102 | Propionate | | | o_Bacteroidales; f_Prevotellaceae; g_Prevotella; | | |
| | | | | s_ | | |
| deno- | Rumen | 0.354018447 | Positive | k_Bacteria; p_Bacteroidetes; c_Bacteroidia; | 531 | |
| vo913272 | Butyrate | | | o_Bacteroidales; f_Prevotellaceae; g_Prevotella; | | |
| | | | | s_ | | |
| deno- | Rumen | 0.480158539 | Positive | k_Bacteria; p_Firmicutes; c_Clostridia; | 532 | |
| vo923356 | Acetate | | | o_Clostridiales; f_Ruminococcaceae; | | |
| | | | | g_Ruminococcus; s_ | | |
| deno- | Rumen | 0.581828353 | Positive | k_Bacteria; p_Proteobacteria; | 533 | |
| vo927104 | Acetate | | | c_Gammaproteobacteria; o_Aeromonadales; | | |
| | | | | f_Succinivibrionaceae; g_; s_ | | |
| deno- | Rumen | 0.501293497 | Positive | k_Bacteria; p_Bacteroidetes; c_Bacteroidia; | 534 | |
| vo927921 | Acetate | | | o_Bacteroidales; f_BS11; g_; s_ | | |
| deno- | Rumen | 0.388836757 | Positive | k_Bacteria; p_Bacteroidetes; c_Bacteroidia; | 535 | |
| vo932996 | Acetate | | | o_Bacteroidales; f_[Paraprevotellaceae]; | | |
| | | | | g_YRC22; s_ | | |
| deno- | Rumen | 0.548715056 | Positive | k_Bacteria; p_Firmicutes; c_Clostridia; | 536 | |
| vo938860 | Acetate | | | o_Clostridiales; f_Ruminococcaceae; | | |
| | | | | g_Ruminococcus; s_ | | |
| deno- | Fecal | 0.343183563 | Positive | k_Bacteria; p_Verrucomicrobia; c_Verruco-5; | 537 | |
| vo950635 | AIA | | | o_WCHB1-41; f_RFP12; g_; s_ | | |
| deno- | Total | 0.362391601 | Positive | k_Bacteria; p_Verrucomicrobia; c_Verruco-5; | 538 | |
| vo950635 | digestion | | | o_WCHB1-41; f_RFP12; g_; s_ | | |
| | dry | | | | | |
| | matter | | | | | |
| deno- | Rumen | 0.383167576 | Positive | k_Bacteria; p_Bacteroidetes; c_Bacteroidia; | 539 | |
| vo955218 | Acetate | | | o_Bacteroidales; f_Prevotellaceae; g_Prevotella; | | |
| | | | | s_ | | |
| deno- | Rumen | 0.652175887 | Positive | k_Bacteria; p_Bacteroidetes; c_Bacteroidia; | 540 | |
| vo959148 | Propionate | | | o_Bacteroidales; f_Prevotellaceae; g_Prevotella; | | |
| | | | | s_ | | |
| deno- | Rumen | 0.72155158 | Positive | k_Bacteria; p_Bacteroidetes; c_Bacteroidia; | 541 | |
| vo97411 | Propionate | | | o_Bacteroidales; f_Prevotellaceae; g_Prevotella; | | |
| | | | | s_ | | |
| deno- | Rumen | 0.348353311 | Positive | k_Bacteria; p_Bacteroidetes; c_Bacteroidia; | 542 | |
| vo991831 | Valerate | | | o_Bacteroidales; f_Prevotellaceae; g_Prevotella; | | |
| | | | | s_ | | |
| deno- | Rumen | 0.41515642 | Positive | k_Bacteria; p_Bacteroidetes; c_Bacteroidia; | 543 | |
| vo999188 | Propionate | | | o_Bacteroidales; f_Prevotellaceae; g_Prevotella; | | |
| | | | | s_ | | |
| deno- | Rumen | 0.446627797 | Positive | Neocallimastigales; Neocallimastigaceae; | 544 | |
| vo10298 | Acetate | | | Caecomyces; Caecomyces 1; JX184808 | | |
| deno- | Rumen | 0.381333061 | Positive | Neocallimastigales; Neocallimastigaceae; | 545 | |
| vo14261 | Acetate | | | Caecomyces; Caecomyces 1; JX184808 | | |
| deno- | Rumen | 0.412526673 | Positive | Neocallimastigales; Neocallimastigaceae; | 546 | |
| vo89488 | Propionate | | | Neocallimastix; Neocallimastix 1 | | |
| deno- | CH4 | 0.291241129 | Positive | D_0_Eukaryota; D_1_SAR; D_2_Alveolata; | 547 | |
| vo60876 | g/kg | | | D_3_Ciliophora; D_6_Trichostomatia | | |
| | ECM | | | | | |
| deno- | Rumen | 0.478640179 | Positive | D_0_Eukaryota; D_1_SAR; D_2_Alveolata; | 548 | |
| vo98946 | Acetate | | | D_3_Ciliophora; D_6_Trichostomatia; | | |
| | | | | D_7_Entodinium; D_8_uncultured rumen | | |
| | | | | protozoa | | |
| deno- | Rumen | 0.504686418 | Positive | k_Bacteria; p_Bacteroidetes; c_Bacteroidia; | 549 | |
| vo1018333 | Propionate | | | o_Bacteroidales; f_; g_; s_ | | |
| deno- | Rumen | 0.42561654 | Positive | k_Bacteria; p_Bacteroidetes; c_Bacteroidia; | 550 | |
| vo1065229 | Propionate | | | o_Bacteroidales; f_Prevotellaceae; g_Prevotella; | | |
| | | | | s_ | | |
| deno- | Rumen | 0.569265437 | Positive | k_Bacteria; p_Bacteroidetes; c_Bacteroidia; | 551 | |
| vo1178104 | Propionate | | | o_Bacteroidales; f_Prevotellaceae; g_Prevotella; | | |
| | | | | s_ | | |
| deno- | Rumen | 0.648477877 | Positive | k_Bacteria; p_Firmicutes; c_Clostridia; | 552 | |
| vo1209472 | Propionate | | | o_Clostridiales; f_Lachnospiraceae; | | |
| | | | | g_Shuttleworthia; s_ | | |
| deno- | Rumen | 0.639598923 | Positive | k_Bacteria; p_Bacteroidetes; c_Bacteroidia; | 553 | |
| vo1221444 | Propionate | | | o_Bacteroidales; f_Prevotellaceae; g_Prevotella; | | |
| | | | | s_ | | |
| deno- | Rumen | 0.524255564 | Positive | k_Bacteria; p_Proteobacteria; | 554 | |
| vo1229628 | Acetate | | | c_Gammaproteobacteria; o_Aeromonadales; | | |
| | | | | f_Succinivibrionaceae; g_; s_ | | |
| deno- | Rumen | 0.676889517 | Positive | k_Bacteria; p_Bacteroidetes; c_Bacteroidia; | 555 | |
| vo1329931 | Propionate | | | o_Bacteroidales; f_Prevotellaceae; g_Prevotella; | | |
| | | | | s_ | | |
| deno- | Rumen | 0.641170176 | Positive | k_Bacteria; p_Bacteroidetes; c_Bacteroidia; | 556 | |
| vo1361244 | Propionate | | | o_Bacteroidales; f_Prevotellaceae; g_Prevotella; | | |
| | | | | s_ | | |
| deno- | Rumen | 0.665617449 | Positive | k_Bacteria; p_Bacteroidetes; c_Bacteroidia; | 557 | |
| vo1380399 | Propionate | | | o_Bacteroidales; f_Prevotellaceae; g_Prevotella; | | |
| | | | | s_ | | |
| deno- | Rumen | 0.633162435 | Positive | k_Bacteria; p_Bacteroidetes; c_Bacteroidia; | 558 | |
| vo1389131 | Propionate | | | o_Bacteroidales; f_Prevotellaceae; g_Prevotella; | | |
| | | | | s_ | | |
| deno- | Rumen | 0.50970428 | Positive | k_Bacteria; p_Bacteroidetes; c_Bacteroidia; | 559 | |
| vo1410364 | Propionate | | | o_Bacteroidales; f_Prevotellaceae; g_Prevotella; | | |
| | | | | s_ | | |
| deno- | Rumen | 0.477463276 | Positive | k_Bacteria; p_Bacteroidetes; c_Bacteroidia; | 560 | |
| vo1465009 | Acetate | | | o_Bacteroidales; f_Prevotellaceae; g_Prevotella; | | |
| | | | | s_ | | |
| deno- | Rumen | 0.670354828 | Positive | k_Bacteria; p_Firmicutes; c_Clostridia; | 561 | |
| vo1477974 | Propionate | | | o_Clostridiales; f_Lachnospiraceae; | | |
| | | | | g_Shuttleworthia; s_ | | |
| deno- | Rumen | 0.662592355 | Positive | k_Bacteria; p_Bacteroidetes; c_Bacteroidia; | 562 | |
| vo1503183 | Propionate | | | o_Bacteroidales; f_Prevotellaceae; g_Prevotella; | | |
| | | | | s_ | | |
| deno- | Rumen | 0.459225544 | Positive | k_Bacteria; p_Proteobacteria; | 563 | |
| vo1550126 | Propionate | | | c_Gammaproteobacteria; o_Aeromonadales; | | |
| | | | | f_Succinivibrionaceae; g_; s_ | | |
| deno- | Rumen | 0.554128968 | Positive | k_Bacteria; p_Bacteroidetes; c_Bacteroidia; | 564 | |
| vo167470 | Acetate | | | o_Bacteroidales; f_[Paraprevotellaceae]; | | |
| | | | | g_YRC22; s_ | | |
| deno- | Rumen | 0.700333998 | Positive | k_Bacteria; p_Bacteroidetes; c_Bacteroidia; | 565 | |
| vo173062 | Propionate | | | o_Bacteroidales; f_Prevotellaceae; g_Prevotella; | | |
| | | | | s_ | | |
| deno- | Rumen | 0.487738355 | Positive | k_Bacteria; p_Firmicutes; c_Clostridia; | 566 | |
| vo174108 | Acetate | | | o_Clostridiales; f_Ruminococcaceae; | | |
| | | | | g_Ruminococcus; s_ | | |
| deno- | Rumen | 0.459970552 | Positive | k_Bacteria; p_Bacteroidetes; c_Bacteroidia; | 567 | |
| vo1765358 | Propionate | | | o_Bacteroidales; f_Prevotellaceae; g_Prevotella; | | |
| | | | | s_ | | |
| deno- | Rumen | 0.532381049 | Positive | k_Bacteria; p_Bacteroidetes; c_Bacteroidia; | 568 | |
| vo183477 | Acetate | | | o_Bacteroidales; f_BS11; g_; s_ | | |
| deno- | Rumen | 0.503273966 | Positive | k_Bacteria; p_Proteobacteria; | 569 | |
| vo1845242 | Acetate | | | c_Gammaproteobacteria; o_Aeromonadales; | | |
| | | | | f_Succinivibrionaceae; g_; s_ | | |
| deno- | Rumen | 0.689041374 | Positive | k_Bacteria; p_Firmicutes; c_Clostridia; | 570 | |
| vo1872170 | Propionate | | | o_Clostridiales; f_Lachnospiraceae; | | |
| | | | | g_Butyrivibrio; s_ | | |
| deno- | Rumen | 0.663997747 | Positive | k_Bacteria; p_Bacteroidetes; c_Bacteroidia; | 571 | |
| vo1879715 | Propionate | | | o_Bacteroidales; f_Prevotellaceae; g_Prevotella; | | |
| | | | | s_ | | |
| deno- | Rumen | 0.386242852 | Positive | k_Bacteria; p_Bacteroidetes; c_Bacteroidia; | 572 | |
| vo1880747 | Butyrate | | | o_Bacteroidales; f_Prevotellaceae; g_Prevotella; | | |
| | | | | s_ | | |
| deno- | Rumen | 0.559567187 | Positive | k_Bacteria; p_Proteobacteria; | 573 | |
| vo1937263 | Acetate | | | c_Gammaproteobacteria; o_Aeromonadales; | | |
| | | | | f_Succinivibrionaceae; g_Ruminobacter; s_ | | |
| deno- | Rumen | 0.61336496 | Positive | k_Bacteria; p_Bacteroidetes; c_Bacteroidia; | 574 | |
| vo1951663 | Acetate | | | o_Bacteroidales; f_BS11; g_; s_ | | |
| deno- | Rumen | 0.620162334 | Positive | k_Bacteria; p_Bacteroidetes; c_Bacteroidia; | 575 | |
| vo2021807 | Propionate | | | o_Bacteroidales; f_Prevotellaceae; g_Prevotella; | | |
| | | | | s_ | | |
| deno- | Rumen | 0.624125572 | Positive | k_Bacteria; p_Bacteroidetes; c_Bacteroidia; | 576 | |
| vo206654 | Propionate | | | o_Bacteroidales; f_Prevotellaceae; g_Prevotella; | | |
| | | | | s_ | | |
| deno- | Rumen | 0.671998586 | Positive | k_Bacteria; p_Bacteroidetes; c_Bacteroidia; | 577 | |
| vo2070846 | Acetate | | | o_Bacteroidales; f_Prevotellaceae; g_; s_ | | |
| deno- | Rumen | 0.459102553 | Positive | k_Bacteria; p_Bacteroidetes; c_Bacteroidia; | 578 | |
| vo2081094 | Propionate | | | o_Bacteroidales; f_; g_; s_ | | |
| deno- | Rumen | 0.560394557 | Positive | k_Bacteria; p_Bacteroidetes; c_Bacteroidia; | 579 | |
| vo2141299 | Acetate | | | o_Bacteroidales; f_RF16; g_; s_ | | |
| deno- | Rumen | 0.336120081 | Positive | k_Bacteria; p_Bacteroidetes; c_Bacteroidia; | 580 | |
| vo2155406 | Butyrate | | | o_Bacteroidales; f_S24-7; g_; s_ | | |
| deno- | Rumen | 0.501053086 | Positive | k_Bacteria; p_Bacteroidetes; c_Bacteroidia; | 581 | |
| vo2171348 | Ammonia | | | o_Bacteroidales; f_Prevotellaceae; g_Prevotella | | |
| deno- | Rumen | 0.555826179 | Positive | k_Bacteria; p_Bacteroidetes; c_Bacteroidia; | 582 | |
| vo2199124 | Acetate | | | o_Bacteroidales; f_; g_; s_ | | |
| deno- | Rumen | 0.468724668 | Positive | k_Bacteria; p_Firmicutes; c_Clostridia; | 583 | |
| vo2219162 | Propionate | | | o_Clostridiales; f_Ruminococcaceae; | | |
| | | | | g_Ruminococcus; s_albus | | |
| deno- | Rumen | 0.578632322 | Positive | k_Bacteria; p_Bacteroidetes; c_Bacteroidia; | 584 | |
| vo2260584 | Propionate | | | o_Bacteroidales; f_Prevotellaceae; g_Prevotella; | | |
| | | | | s_copri | | |
| deno- | Rumen | 0.389107007 | Positive | k_Bacteria; p_Bacteroidetes; c_Bacteroidia; | 585 | |
| vo2323272 | Butyrate | | | o_Bacteroidales; f_; g_; s_ | | |
| deno- | Rumen | 0.587895876 | Positive | k_Bacteria; p_Bacteroidetes; c_Bacteroidia; | 586 | |
| vo2367108 | Acetate | | | o_Bacteroidales; f_Prevotellaceae; g_Prevotella; | | |
| | | | | s_ | | |
| deno- | Rumen | 0.34281236 | Positive | k_Bacteria; p_Bacteroidetes; c_Bacteroidia; | 587 | |
| vo252745 | Butyrate | | | o_Bacteroidales; f_Prevotellaceae; g_Prevotella; | | |
| | | | | s_ | | |
| deno- | Rumen | 0.510661757 | Positive | k_Bacteria; p_Firmicutes; c_Clostridia; | 588 | |
| vo279607 | Acetate | | | o_Clostridiales; f_; g_; s_ | | |
| deno- | Rumen | 0.415987035 | Positive | k_Bacteria; p_Proteobacteria; | 589 | |
| vo298878 | Propionate | | | c_Gammaproteobacteria; o_Aeromonadales; | | |
| | | | | f_Succinivibrionaceae; g_; s_ | | |
| deno- | Rumen | 0.548221513 | Positive | k_Bacteria; p_Proteobacteria; | 590 | |
| vo33906 | Acetate | | | c_Gammaproteobacteria | | |
| deno- | Rumen | 0.746007146 | Positive | k_Bacteria; p_Proteobacteria; | 591 | |
| vo358994 | Propionate | | | c_Gammaproteobacteria; o_Aeromonadales; | | |
| | | | | f_Succinivibrionaceae; g_; s_ | | |
| deno- | Rumen | 0.524159592 | Positive | k_Bacteria; p_Verrucomicrobia; c_Verruco-5; | 592 | |
| vo410508 | Acetate | | | o_WCHB1-41; f_RFP12; g_; s_ | | |
| deno- | Rumen | 0.445931277 | Positive | k_Bacteria; p_Proteobacteria; | 593 | |
| vo433754 | Acetate | | | c_Gammaproteobacteria; o_Aeromonadales; | | |
| | | | | f_Succinivibrionaceae; g_Ruminobacter; s_ | | |
| deno- | Rumen | 0.552274302 | Positive | k_Bacteria; p_Bacteroidetes; c_Bacteroidia; | 594 | |
| vo448814 | Propionate | | | o_Bacteroidales; f_Prevotellaceae; g_Prevotella; | | |
| | | | | s_ | | |
| deno- | Rumen | 0.565011486 | Positive | k_Bacteria; p_Bacteroidetes; c_Bacteroidia; | 595 | |
| vo514676 | Propionate | | | o_Bacteroidales; f_Prevotellaceae; g_Prevotella; | | |
| | | | | s_ | | |
| deno- | Rumen | 0.731554185 | Positive | k_Bacteria; p_Bacteroidetes; c_Bacteroidia; | 596 | |
| vo521876 | Propionate | | | o_Bacteroidales; f_Prevotellaceae; g_Prevotella; | | |
| | | | | s_ | | |
| deno- | Rumen | 0.707943995 | Positive | k_Bacteria; p_Bacteroidetes; c_Bacteroidia; | 597 | |
| vo554901 | Propionate | | | o_Bacteroidales; f_Prevotellaceae; g_Prevotella; | | |
| | | | | s_ | | |
| deno- | Rumen | 0.632992297 | Positive | k_Bacteria; p_Bacteroidetes; c_Bacteroidia; | 598 | |
| vo577780 | Propionate | | | o_Bacteroidales; f_Prevotellaceae; g_Prevotella; | | |
| | | | | s_ | | |
| deno- | Rumen | 0.590793546 | Positive | k_Bacteria; p_Firmicutes; c_Clostridia; | 599 | |
| vo593859 | Propionate | | | o_Clostridiales; f_Lachnospiraceae; | | |
| | | | | g_Shuttleworthia; s_ | | |
| deno- | Rumen | 0.520524849 | Positive | k_Bacteria; p_Bacteroidetes; c_Bacteroidia; | 600 | |
| vo61024 | Acetate | | | o_Bacteroidales; f_; g_; s_ | | |
| deno- | Rumen | 0.444985563 | Positive | k_Bacteria; p_Spirochaetes; c_pirochaetes; | 601 | |
| vo632834 | Propionate | | | o_Spirochaetales; f_Spirochaetaceae; | | |
| | | | | g_Treponema; s_ | | |
| deno- | Rumen | 0.674716416 | Positive | k_Bacteria; p_Bacteroidetes; c_Bacteroidia; | 602 | |
| vo63840 | Propionate | | | o_Bacteroidales; f_Prevotellaceae; g_Prevotella; | | |
| | | | | s_ | | |
| deno- | Rumen | 0.503235417 | Positive | k_Bacteria; p_Bacteroidetes; c_Bacteroidia; | 603 | |
| vo653342 | Acetate | | | o_Bacteroidales; f_; g_; s_ | | |
| deno- | Rumen | 0.604131703 | Positive | k_Bacteria; p_Bacteroidetes; c_Bacteroidia; | 604 | |
| vo701155 | Propionate | | | o_Bacteroidales; f_Prevotellaceae; g_Prevotella; | | |
| | | | | s_ | | |
| deno- | Rumen | 0.718612527 | Positive | k_Bacteria; p_Bacteroidetes; c_Bacteroidia; | 605 | |
| vo848818 | Propionate | | | o_Bacteroidales; f_Prevotellaceae; g_Prevotella; | | |
| | | | | s_ | | |
| deno- | Rumen | 0.516901735 | Positive | k_Bacteria; p_Bacteroidetes; c_Bacteroidia; | 606 | |
| vo877792 | Propionate | | | o_Bacteroidales; f_Prevotellaceae; g_Prevotella; | | |
| | | | | s_ | | |
| deno- | Rumen | 0.643555038 | Positive | k_Bacteria; p_Firmicutes; c_Clostridia; | 607 | |
| vo879882 | Propionate | | | o_Clostridiales; f_Lachnospiraceae; | | |
| | | | | g_Shuttleworthia; s_ | | |
| deno- | Rumen | 0.666185605 | Positive | k_Bacteria; p_Bacteroidetes; c_Bacteroidia; | 608 | |
| vo882840 | Propionate | | | o_Bacteroidales; f_Prevotellaceae; g_Prevotella; | | |
| | | | | s_ | | |
| deno- | Rumen | 0.690761443 | Positive | k_Bacteria; p_Bacteroidetes; c_Bacteroidia; | 609 | |
| vo942112 | Propionate | | | o_Bacteroidales; f_Prevotellaceae; g_Prevotella; | | |
| | | | | s_ | | |
| deno- | Rumen | 0.668321198 | Positive | k_Bacteria; p_Bacteroidetes; c_Bacteroidia; | 610 | |
| vo942115 | Propionate | | | o_Bacteroidales; f_Prevotellaceae; g_Prevotella; | | |
| | | | | s_ | | |
| deno- | Rumen | 0.650983347 | Positive | k_Bacteria; p_Bacteroidetes; c_Bacteroidia; | 611 | |
| vo991831 | Propionate | | | o_Bacteroidales; f_Prevotellaceae; g_Prevotella; | | |
| | | | | s_ | | |
| deno- | Rumen | 0.439449268 | Positive | k_Bacteria; p_Bacteroidetes; c_Bacteroidia; | 612 | |
| vo305923 | Butyrate | | | o_Bacteroidales; f_Prevotellaceae; g_Prevotella; | | |
| | | | | s_ | | |
| deno- | Rumen | 0.538400419 | Positive | k_Bacteria; p_Bacteroidetes; c_Bacteroidia; | 613 | |
| vo370057 | Acetate | | | o_Bacteroidales; f_; g_; s_ | | |
| deno- | Rumen | 0.454645617 | Positive | k_Bacteria; p_Bacteroidetes; c_Bacteroidia; | 614 | |
| vo398343 | Butyrate | | | o_Bacteroidales; f_Prevotellaceae; g_Prevotella; | | |
| | | | | s_ | | |
| deno- | Rumen | 0.513928051 | Positive | k_Bacteria; p_Bacteroidetes; c_Bacteroidia; | 615 | |
| vo506833 | Propionate | | | o_Bacteroidales; f_Prevotellaceae; g_Prevotella; | | |
| | | | | s_ | | |

Table 3 summarizes all hereditable bacteria identified in this study.

TABLE 3

| | | SEQ | Associated |
|---|---|---|---|
| | | ID | host- |
| OUT ID | Taxonomy | NO: | traits |
| denovo1 | Neocallimastigales; Neocallimastigaceae; | 616 | |
| 00870 | Neocallimastix; Neocallimastix 1 | | |
| denovo5 | Neocallimastigales; Neocallimastigaceae; | 617 | |
| 7586 | Neocallimastix; Neocallimastix 1; JX184608 | | |
| denovo1 | k__Bacteria; p__Bacteroidetes; | 618 | |
| 115154 | c__Bacteroidia; o__Bacteroidales; | | |
| | f__Prevotellaceae; g__Prevotella; s__ | | |
| denovo1 | k__Bacteria; p__Firmicutes; | 619 | |
| 201408 | c__Clostridia; o__Clostridiales; | | |
| | f__Ruminococcaceae; g__Ruminococcus; | | |
| | s__flavefaciens | | |
| denovo1 | k__Bacteria; p__Bacteroidetes; | 620 | |
| 23585 | c__Bacteroidia; o__Bacteroidales; | | |
| | f__Prevotellaceae; g__Prevotella; s__ | | |
| denovo1 | k__Bacteria; p__Bacteroidetes; | 621 | |
| 273092 | c__Bacteroidia; o__Bacteroidales; | | |
| | f__S24-7; g__; s__ | | |
| denovo1 | k__Bacteria; p__Bacteroidetes; | 622 | Rumen |
| 359435 | c__Bacteroidia; o__Bacteroidales; | | Acetate, |
| | f__RF16; g__; s__ | | Rumen |
| | | | Propionate |
| denovo1 | k__Bacteria; p__Bacteroidetes; | 623 | |
| 372339 | c__Bacteroidia; o__Bacteroidales; | | |
| | f__Prevotellaceae; g__Prevotella; s__ | | |
| denovo1 | k__Bacteria; p__Bacteroidetes; | 624 | |
| 388751 | c__Bacteroidia; o__Bacteroidales; | | |
| | f__Prevotellaceae; g__Prevotella; s__ | | |
| denovo1 | k__Bacteria; p__Bacteroidetes; | 625 | |
| 394963 | c__Bacteroidia; o__Bacteroidales; f__; | | |
| | g__; s__ | | |
| denovo1 | k__Bacteria; p__Bacteroidetes; | 626 | |
| 432073 | c__Bacteroidia; o__Bacteroidales; | | |
| | f__Prevotellaceae; g__Prevotella; s__ | | |
| denovo1 | k__Bacteria; p__Firmicutes; c__Clostridia; | 627 | |
| 501742 | o__Clostridiales; f__; g__; s__ | | |
| denovo1 | k__Bacteria; p__Bacteroidetes; | 628 | |
| 502997 | c__Bacteroidia; o__Bacteroidales; | | |
| | f__Prevotellaceae; g__Prevotella; s__ | | |
| denovo1 | k__Bacteria; p__Bacteroidetes; | 629 | |
| 542925 | c__Bacteroidia; o__Bacteroidales; | | |
| | f__Prevotellaceae; g__; s__ | | |
| denovo1 | k__Bacteria; p__Proteobacteria; | 630 | Rumen |
| 636556 | c__Gammaproteobacteria; | | Acetate, |
| | o__Aeromonadales; | | Rumen |
| | f__Succinivibrionaceae; g__; s__ | | Propionate |
| denovo1 | k__Bacteria; p__Bacteroidetes; | 631 | Milk |
| 690942 | c__Bacteroidia; o__Bacteroidales; | | fat, |
| | f__Bacteroidaceae; g__BF311; s__ | | Rumen |
| | | | Acetate, |
| | | | Rumen |
| | | | pH, |
| | | | Rumen |
| | | | Propionate |
| denovo1 | k__Bacteria; p__Bacteroidetes; | 632 | Rumen |
| 708915 | c__Bacteroidia; o__Bacteroidales; f__; | | Acetate, |
| | g__; s__ | | Rumen |
| | | | Propionate |
| denovo1 | k__Bacteria; p Firmicutes; | 633 | |
| 763836 | c__Clostridia; o__Clostridiales; | | |
| | f__Lachnospiraceae; g__; s__ | | |
| denovo1 | k__Bacteria; p__Bacteroidetes; | 634 | |
| 791215 | c__Bacteroidia; o__Bacteroidales; | | |
| | f__Prevotellaceae; g__Prevotella; s__ | | |
| denovo1 | k__Bacteria; p__Lentisphaerae; | 635 | Milk |
| 803355 | c__[Lentisphaeria]; o__Victivallales; | | lactose |
| | f__Victivallaceae; g__; s__ | | Milk |
| | | | yield, |
| | | | Rumen |
| | | | Acetate, |
| | | | Rumen |
| | | | Propionate |
| denovo1 | k__Bacteria; p__Firmicutes; | 636 | |
| 869934 | c__Clostridia; o__Clostridiales; | | |
| | f__Ruminococcaceae; g__Ruminococcus | | |
| denovo1 | k__Bacteria; p__Bacteroidetes; | 637 | |
| 988452 | c__Bacteroidia; o__Bacteroidales; | | |
| | f__S24-7; g__; s__ | | |
| denovo2 | k__Bacteria; p__Firmicutes; | 638 | |
| 004134 | c__Clostridia; o__Clostridiales; | | |
| | f__Lachnospiraceae; g__; s__ | | |
| denovo2 | k__Bacteria; p__Bacteroidetes; | 639 | Plasma |
| 090355 | c__Bacteroidia; o__Bacteroidales; | | BHB, |
| | f__Prevotellaceae; g__Prevotella; s__ | | Rumen |
| | | | Butyrate, |
| | | | Rumen |
| | | | Propionate |
| denovo2 | k__Bacteria; p__Fibrobacteres; | 640 | Rumen |
| 090357 | c__Fibrobacteria; o__Fibrobacterales; | | Acetate |
| | f__Fibrobacteraceae; | | |
| | g__Fibrobacter; s__succinogenes | | |
| denovo2 | k__Bacteria; p__Bacteroidetes; | 641 | |
| 230574 | c__Bacteroidia; o__Bacteroidales; | | |
| | f__Prevotellaceae; g__Prevotella; s__ | | |
| denovo2 | k__Bacteria; p__Tenericutes; | 642 | |
| 327084 | c__Mollicutes; o__Anaeroplasmatales; | | |
| | f__Anaeroplasmataceae; | | |
| | g__Anaeroplasma; s__ | | |
| denovo2 | k__Bacteria; p__Bacteroidetes; | 643 | |
| 362621 | c__Bacteroidia; o__Bacteroidales; f__; | | |
| | g__; s__ | | |
| denovo2 | k__Bacteria; p__Bacteroidetes; | 644 | Rumen |
| 44987 | c__Bacteroidia; o__Bacteroidales; | | Butyrate |
| | f__Prevotellaceae; g__Prevotella; s__ | | |
| denovo2 | k__Bacteria; p Verrucomicrobia; | 645 | Rumen |
| 64956 | c__Verruco-5; o__WCHB1-41; | | Acetate, |
| | f__RFP12; g__; | | Rumen |
| | | | Propionate |
| denovo2 | k__Bacteria; p__Bacteroidetes; | 646 | |
| 91726 | c__Bacteroidia; o__Bacteroidales; | | |
| | f__S24-7; g__; s__ | | |
| denovo3 | k__Bacteria; p__Bacteroidetes; | 647 | |
| 09598 | c__Bacteroidia; o__Bacteroidales; | | |
| | f__Prevotellaceae; g__Prevotella; s__ | | |
| denovo4 | k__Bacteria; p__Firmicutes; | 648 | |
| 70677 | c__Clostridia; o__Clostridiales; | | |
| | f__Ruminococcaceae; | | |
| | g__Ruminococcus; s__albus | | |
| denovo6 | k__Bacteria; p__Bacteroidetes; | 649 | |
| 03054 | c__Bacteroidia; o__Bacteroidales; | | |
| | f__Prevotellaceae; g__Prevotella; s__ | | |
| denovo6 | k__Bacteria; p__Firmicutes; | 650 | Rumen |
| 42135 | c__Clostridia; o__Clostridiales; | | Butyrate |
| | f__Lachnospiraceae | | |
| denovo6 | k__Bacteria; p__Firmicutes; | 651 | |
| 70462 | c__Clostridia; o__Clostridiales; | | |
| | f__Lachnospiraceae; g__Butyrivibrio; s__ | | |
| denovo7 | k__Bacteria; p__Bacteroidetes; | 652 | |
| 06524 | c__Bacteroidia; o__Bacteroidales; | | |
| | f__[Paraprevotellaceae]; g__; s__ | | |
| denovo7 | k__Bacteria; p__Fibrobacteres; | 653 | |
| 89865 | c__Fibrobacteria; o__Fibrobacterales; | | |
| | f__Fibrobacteraceae; g__Fibrobacter; | | |
| | s__succinogenes | | |
| denovo8 | k__Bacteria; p__Firmicutes; | 654 | |
| 15036 | c__Clostridia; o__Clostridiales; | | |
| | f__Lachnospiraceae; g__Roseburia; | | |
| | s__faecis | | |

Overall, when microbial co-occurrence networks were inferred within individual farms, it became evident that heritable microbes are significantly more connected than non-heritable microbes, consistent with the central positions of heritable microbes in the rumen co-occurrence networks (FIG. 1C).

The demonstration here of heritable, interacting microbes raises possibilities of breeding animals for particular microbiomes and thus phenotypic and production properties, on condition that the core can be shown to control these properties. Co-occurrence networks were further investigated for the core abundances relation to phenotypic outcomes.

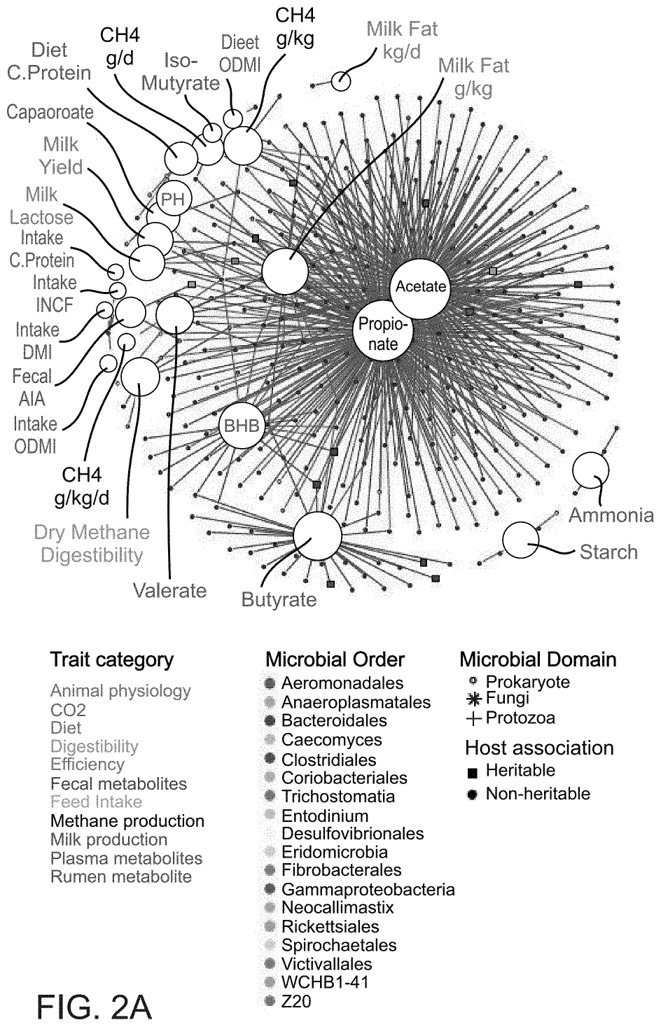

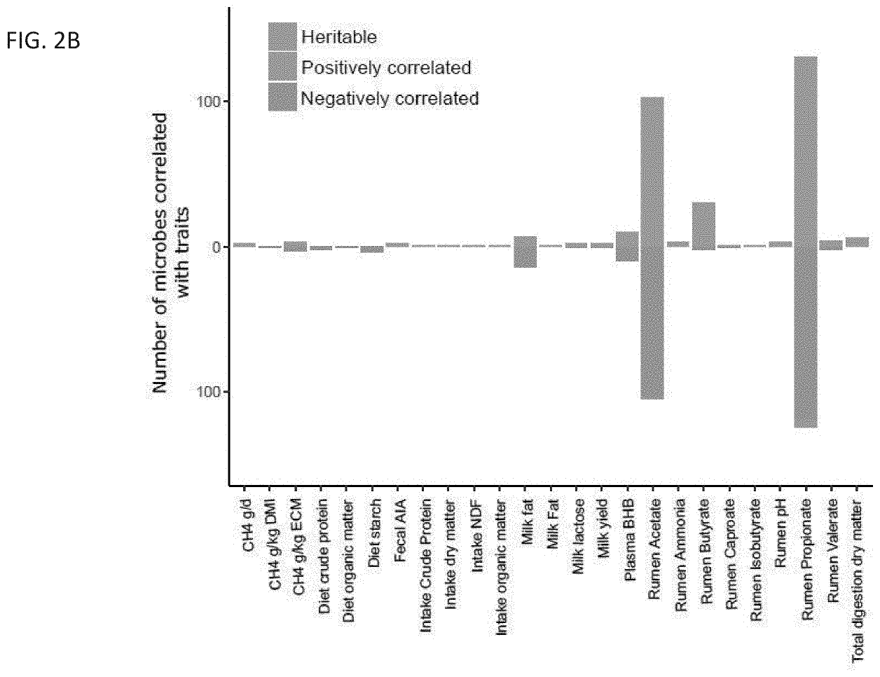

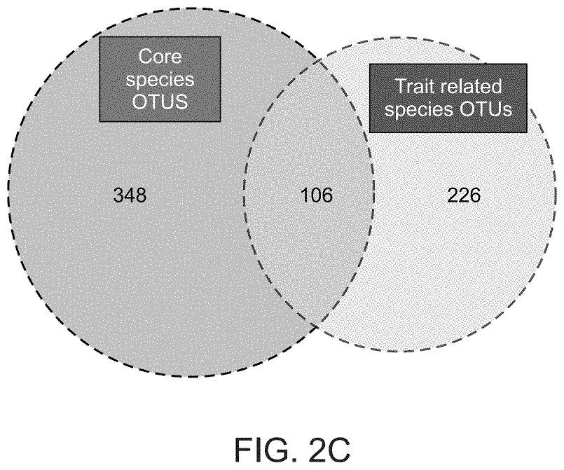

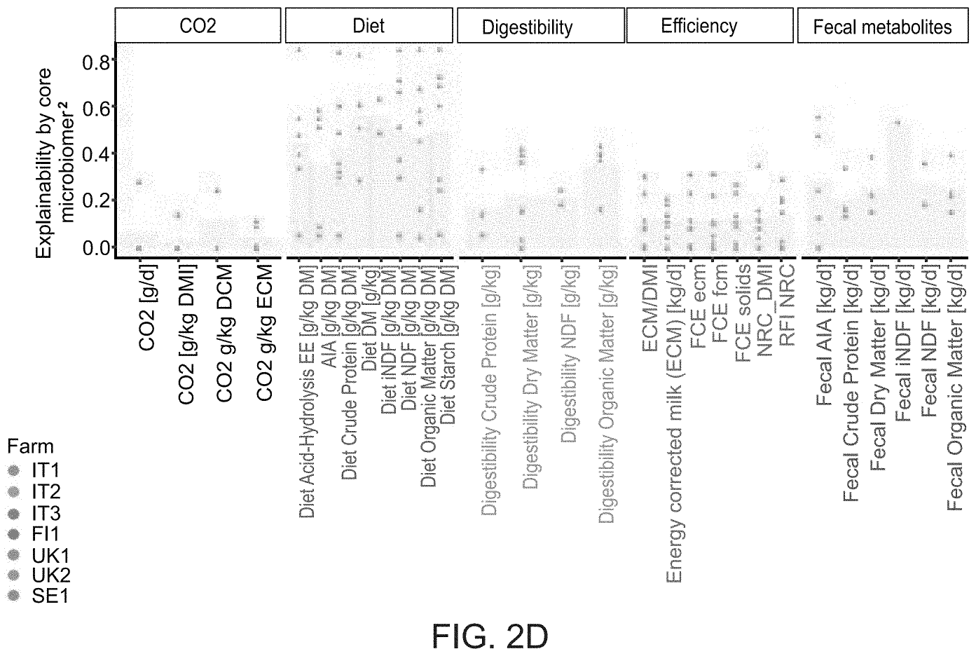

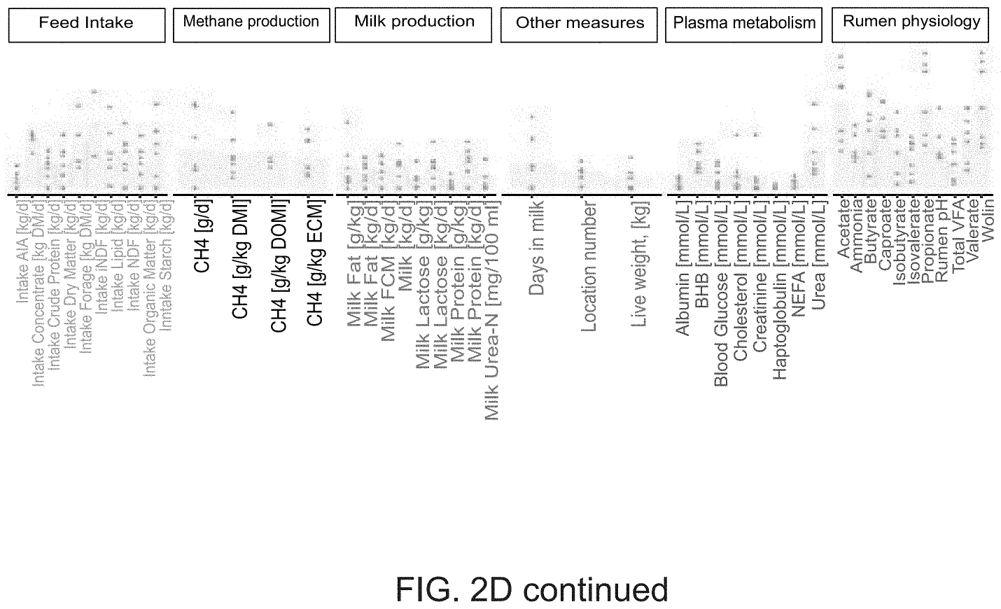

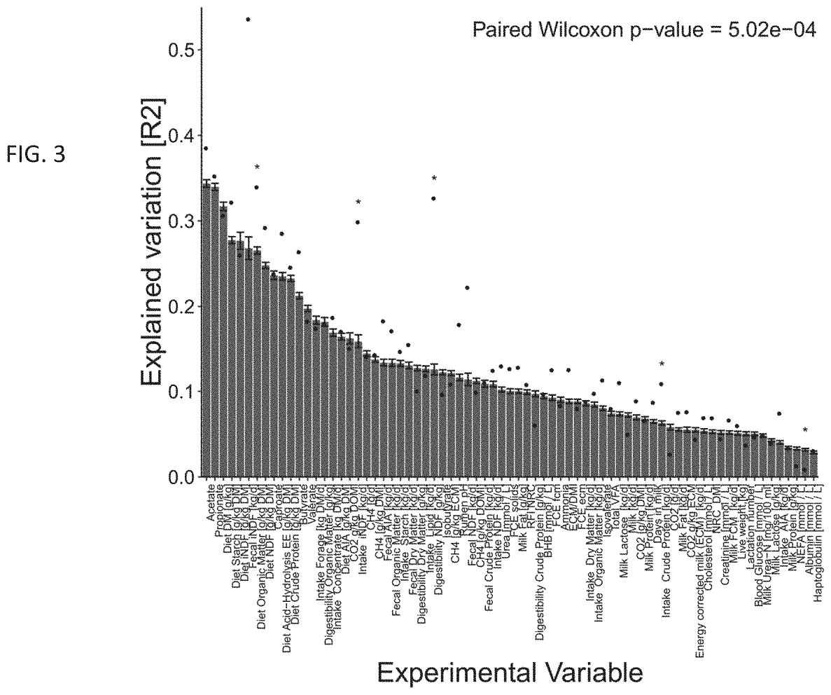

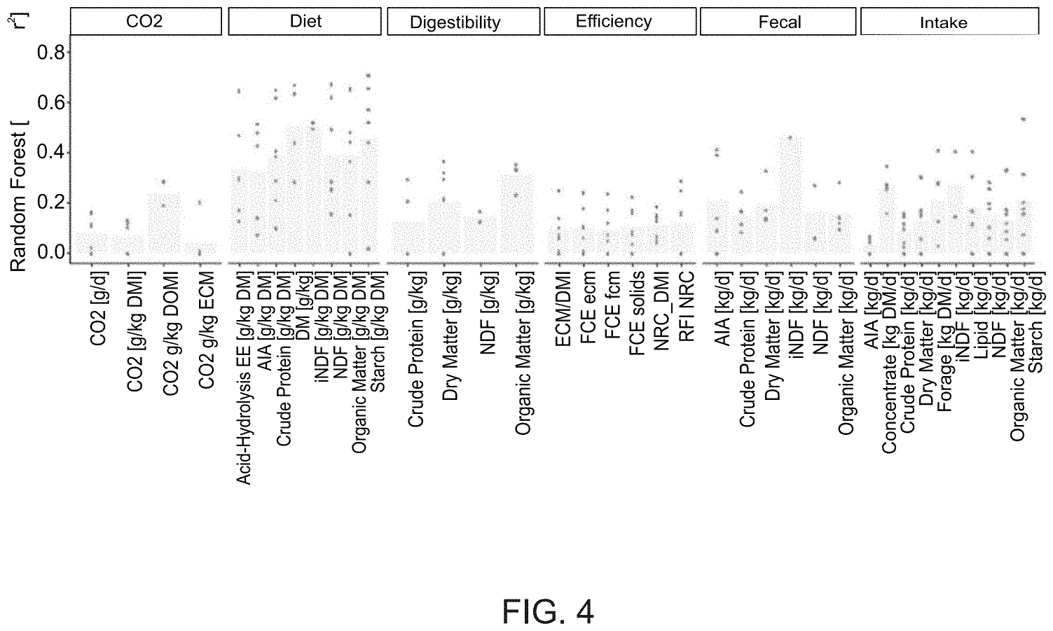

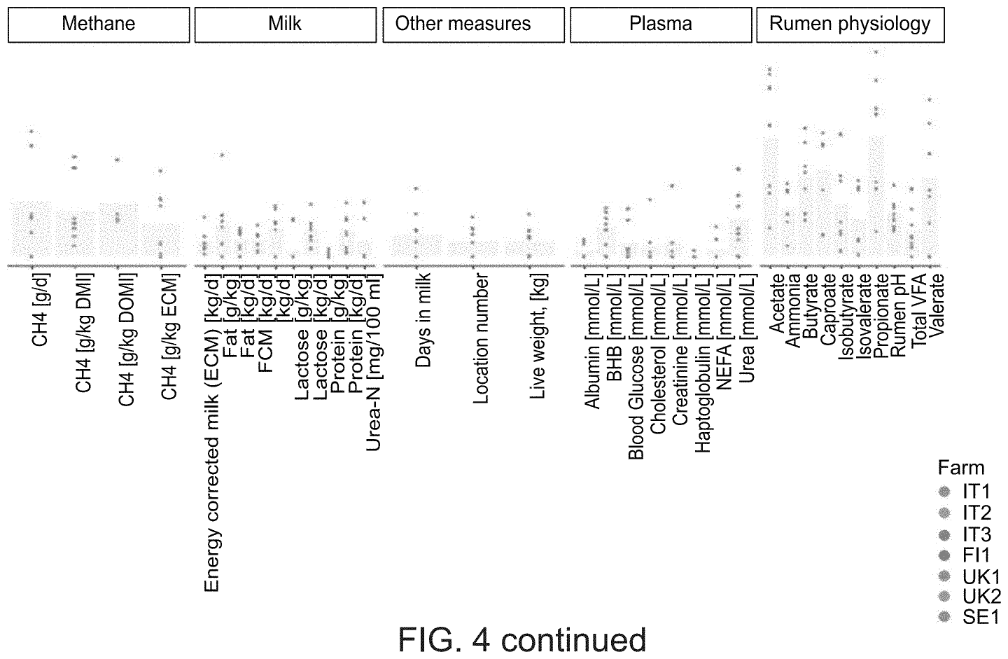

The associations found here are hugely complex (FIG. 2A), with 339 microbes, mostly prokaryotes but also a handful of protozoa and fungi, associated with rumen metabolism and various host phenotypes. The resulting network (FIG. 2A) included only re-occurring significance correlations with same directionality (FDR <0.05) within at least four farms when analysed independently. As would be expected from the nutritional dependence of ruminants on VFA generated by rumen fermentation, large numbers of core microbiome members were found to be associated with traits such as ruminal acetate and propionate concentration, with fewer correlated to production traits like milk production and methane emission (204, 254, 23 and 7, respectively, FIG. 2B). Among those linked to methane emissions are Succinovibrionaceae, confirming what has been found previously in beef cattle (18). Importantly, compared to the overall rumen microbiome, prokaryotic members of the core microbiome are highly enriched with trait-associated microbes (odds-ratio 388 and P<2.2e⁻¹⁶ Fisher Exact between 332 trait-related and 454 prokaryotic core members; FIG. 2C), stressing the importance and central role that the core microbiome plays in host function and microbiome metabolism. Two distinctive machine learning algorithms were applied to predict rumen metabolism diet and host traits, based on core microbiome composition; Ridge regression (19,20) and Random Forest (21,22), using linear regression and decision trees-based approaches respectively. This allowed us to investigate the degree of agreement (r²) between predicted and actual values (FIG. 2D). These tools highlighted the core microbiome as highly explanatory for dietary components and rumen metabolites, with propionate approaching an agreement of r²=0.9 in some farms. Importantly, methane emissions could also be explained, based on rumen microbiome composition, with values reaching r²=0.4 in some farms. Moreover, although having lower explainability, many of the host traits, including host plasma metabolites and milk composition, could be explained to an extent by the core microbiome composition (FIG. 2D). Our findings also show that core microbiome has higher prediction power than host animals' genotype (based on the genomic relationship matrix), as has dietary composition. All in all, in both machine learning algorithms the heritable microbes exhibited on average a significantly higher explanatory power for host phenotypes and other experimental variables compared to other core microbes (FIG. 3, FIG. 4, Wilcoxon paired rank-sum test, P<0.005), further underlining the central role of heritable microbes within rumen microbial ecology and to the host. Importantly, the great majority of these microbes show stability in time and only a small portion of them (39, 3 heritable and one trait-associated) showed seasonality, and of those most do so solely in one of the farms.

### Discussion and Conclusions

The present example shows that a small number of host-determined, heritable microbes make higher contribution to explaining experimental variables and host phenotypes (FIG. 3), and propose microbiome-led breeding/genetic programs to provide a sustainable solution to increase efficiency and lower emissions from ruminant livestock. Based on the genetic determinants of the heritable microbes, it should be possible to optimize their abundance through selective breeding programs. A different, and perhaps more immediate, application of this data could be to modify early-life colonization, a factor that has been shown to drive microbiome composition and activity in later life (23-25). Inoculating key core species associated with feed efficiency or methane emissions as precision probiotics approach could be considered as likely to complement the heritable microbiome towards optimized rumen function.

The present study focused on two bovine dairy breeds, but the results are likely to be applicable to beef animals and other ruminant species. Given the high importance of diet in performance and the composition of the rumen microbiome, such programs should take special cognizance of likely feeding regimes. Within that context, following the overall predictive impact of identified trait-associated heritable microbes on production indices should result in a more efficient and more environmentally friendly ruminant livestock industry.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. H. Steinfeld, P. Gerber, T. Wassenaar, V. Caste, M. Rosales, C. de Haan C, ‘Livestock's Long Shadow.’ (FAO, Rome, 2006).
2. P. R. Myer, T. P. Smith, J. E. Wells, L. A. Kuehn, H. C. I. Freetly, Rumen microbiome from steers differing in feed efficiency. Plos One 10, e0129174 (2015).
3. S. K. Shabat, G. Sasson, A. Doron-Faigenboim, T. Durman, S. Yaacoby, M. E. Berg Miller, B. A. White, N. Shterzer, I. Mizrahi, Specific microbiome-dependent mechanisms underlie the energy harvest efficiency of ruminants. ISME J. 10, 2958-72 (2016).
4. G. Henderson, F. Cox, S. Ganesh, A. Jonker, W. Young, P. H. Janssen. Rumen microbial community composition varies with diet and host, but a core microbiome is found across a wide geographical range. Sci. Rep. 5, 14567 (2015).
5. I. Mizrahi, The Prokaryotes. (Springer Berlin Heidelberg, 2013).
6. A. G. Williams, G. S. Coleman, The rumen microbial ecosystem, (Chapman & Hall, London, 1997).
7. C. J. Newbold, G. de la Fuente, A. Belanche, E. Ramos-Morales, E, N. R. McEwan, The role of ciliate protozoa in the rumen. Front. Microbiol. 6, 1313 (2015).
8. R. J. Gruninger, A. K. Puniya, T. M. Callaghan, J. E. Edwards, N. Youssef, S. S. Dagar, K. Fliegerová, G W. Griffith, R. Forster, A. Tsang, T. McAllister, M. S. Elshahed, Anaerobic fungi (phylum Neocallimastigomycota): advances in understanding their taxonomy, life cycle, ecology, role and biotechnological potential. FEMS Microbiol. Ecol. 90, 1-17 (2014).
9. P. H. Janssen, M. Kirs, Structure of the archaeal community of the rumen. Appl. Environ. Microbiol. 74, 3619-3625 (2008).
10. D. P. Morgavi, E. Rathahao-Paris, M. Popova, J. Boccard, K. F. Nielsen, H. Boudra, Rumen microbial communities influence metabolic phenotypes in lambs. Front. Microbiol. 6, 1060 (2015).
11. B. J. Hayes, K. A. Donoghue, C. M. Reich, B. A. Mason, T. Bird-Gardiner, R. M. Herd, P. F. Arthur, Genomic heritabilities and genomic estimated breeding values for methane traits in Angus cattle. J. Anim. Sci. 94, 902-908 (2016).
12. R. Roehe, R. J. Dewhurst, C. A. Duthie, J. A. Rooke, N. McKain, D. W. Ross, J. J. Hyslop, A. Waterhouse, T. C. Freeman, M. Watson, R. J. Wallace, Bovine host genetic variation influences rumen microbial methane production with best selection criterion for low methane emitting and efficiently feed converting hosts based on metagenomic gene abundance. PLoS Genet. 12, e1005846 (2016).
13. J. A. Rooke, R. J. Wallace, C. A. Duthie, N. McKain, S. M. de Souza, J. J. Hyslop, D. W. Ross, T. Waterhouse, R. Roehe, Hydrogen and methane emissions from beef cattle and their rumen microbial community vary with diet, time after feeding and genotype. Br. J Nutr. 112, 398 (2014).
14. J. K. Goodrich, S. C. Di Rienzi, A. C. Poole, O. Koren, W. A. Walters, J. G. Caporaso, R. Knight, R. E. Ley, Conducting a microbiome study. Cell 158, 250-262 (2014).
15. G. Sasson, S. Kruger Ben-Shabat, E. Seroussi, A. Doron-Faigenboim, N. Shterzer, S. Yaacoby, M. E. Berg Miller, B. A. White, E. Halperin, I. Mizrahi Heritable bovine rumen bacteria are phylogenetically related and correlated with the cow's capacity to harvest energy from its feed. MBio. 8, (2017).
16. A. C. Martiny, K. Treseder, G. Pusch, Phylogenetic conservatism of functional traits in microorganisms. ISME J 7, 830 (2013).
17. J. E. Edwards, N. R. McEwan, A. J. Travis, R. J. Wallace, 16S rDNA library-based analysis of ruminal bacterial diversity. Antonie van Leeuwenhoek 86, 263 (2004).
18. R. J. Wallace, J. A. Rooke, N. McKain, C. A. Duthie, J. J. Hyslop, D. W. Ross, A. Waterhouse, M. Watson, R. Roehe, The rumen microbial metagenome associated with high methane production in cattle. BMC Genomics 16, 839 (2015).
19. D. W. Marquardt, D. S. Ronald “Ridge regression in practice.” The American Statistician 29.1, 3-20 (1975).
20. J. Friedman, T. Hastie, R. Tibshirani. Regularization paths for generalized linear models via coordinate descent. J. Statist. Software 33.1, 1. (2010).
21. A. Liaw, M. Wiener, Classification and regression by randomForest. R News 2, 18-22 (2002).
22. L. Breiman, Random forests. Machine Learning 45, 5-32 (2001).
23. D. R. Yáñez-Ruiz, B. Macias, E. Pinloche, C. J. Newbold. The persistence of bacterial and methanogenic archaeal communities residing in the rumen of young lambs. FEMS Microbiol. Ecol. 72, 272-278. (2010).
24. C. Foditsch, R. V. Pereira, E. K. Ganda, M. S. Gomez, E. C. Marques, T. Santin, R. C. Bicalho, Oral administration of Faecalibacterium prausnitzii decreased the incidence of severe diarrhea and related mortality rate and Increased weight gain in preweaned dairy heifers. Plos One 10, e0145485 (2015).
25. D. R. Yanez-Ruiz, L. Abecia, C. J. Newbold, Manipulating rumen microbiome and fermentation through interventions during early life: a review. Front. Microbiol. 6, 1133 (2015).
26. P. C. Garnsworthy, J. Craigon, J. Hernandez-Medrano, N. Saunders, On-farm methane measurements during milking correlate with total methane production by individual dairy cows. J. Dairy Sci. 95, 3166-3180 (2012).
27. Y. Unal, P. C. Garnsworthy, Estimation of intake and digestibility of forage-based diets in group-fed dairy cows using alkanes as markers. J. Agri. Sci. 133, 419-425 (1999).
28. P. Bani, F. Piccioli Cappelli, A. Minuti, V. Ficuciello, V. Lopreiato, P. C. Garnsworthy and E. Trevisi, Estimation of dry matter intake by n-alkanes in dairy cows fed TMR: effect of dosing technique and faecal collection time. Animal Prod. Sci. 54, 1747-1751 (2014).
29. M. J. Playne, Determination of ethanol, volatile fatty acids, lactic and succinic acids in fermentation liquids by gas chromatography. J. Sci. Food Agric. 36, 638-644 (1985).
30. Z. Yu, M. Morrison, Improved extraction of PCR-quality community DNA from digesta and fecal samples. BioTechniques 36, 808-812 (2004).
31. T. Riaz, W. Shehzad, A. Viari, F. Pompanon, P. Taberlet, E. Coissac, ecoPrimers: inference of new DNA barcode markers from whole genome sequence analysis. Nucl. Acids Res. 39, e145-e145 (2011).
32. F. Boyer, C. Mercier, A. Bonin, B. Y. Le, P. Taberlet, E. Coissac, obitools: a unix-inspired software package for DNA metabarcoding. Mol. Ecol. Resources 16, 176-182 (2016).
33. P. C. Garnsworthy, J. Craigon, J. Hernandez-Medrano, N. Saunders, Variation among individual dairy cows in methane measurements made on farm during milking. J. Dairy Sci. 95, 3181-3189 (2012).
34. P. Huhtanen, E. Cabezas-Garcia, S. Utsumi, S. Zimmerman, Comparison of methods to determine methane emissions from dairy cows in farm conditions. J. Dairy Sci. 98, 3394-3409 (2015).
35. E. Negussie, J. Lehtinen, P. Mantysaari, A. R. Bayat, A. E. Liinamo, E. A. Mantysaari, M. H. Lidauer, Non-invasive individual methane measurement in dairy cows. Animal 11, 890-899 (2017).
36. J. Skinner, R. Brown, L. Roberts, Bovine haptoglobin response in clinically defined field conditions. Veter. Rec. 128, 147-149 (1991).
37. H. Maeda, C. Fujimoto, Y. Haruki, T. Maeda, S. Kokeguchi, M. Petelin, S. Takashiba, Quantitative real-time PCR using TaqMan and SYBR Green for Actinobacillus actinomycetemcomitans, Porphyromonas gingivalis, Prevotella intermedia, tetQ gene and total bacteria. FEMS Immunol. Med. Microbiol. 39, 81-86 (2003).
38. Z. Fuller, P. Louis, A. Mihajlovski, V. Rungapamestry, B. Ratcliffe, A. J. Duncan, Influence of cabbage processing methods and prebiotic manipulation of colonic microflora on glucosinolate breakdown in man. Br. J. Nutr. 98, 364-372 (2007).
39. C. Ramirez-Farias, K. Slezak, Z. Fuller, A. Duncan, G. Holtrop, P. Louis, Effect of inulin on the human gut microbiota: stimulation of Bifidobacterium adolescentis and Faecalibacterium prausnitzii. Br. J. Nutr. 101, 541-550 (2009).
40. S. E. Hook, K. S. Northwood, A. D. G. Wright, B. W. McBride, Long-term monensin supplementation does not significantly affect the quantity or diversity of methanogens in the rumen of the lactating dairy cow. Appl. Environ. Microbiol. 75, 374-380 (2009).
41. J. T. Sylvester, S. K. Karnati, Z. Yu, M. Morrison, J. L. Firkins, Development of an assay to quantify rumen ciliate protozoal biomass in cows using real-time PCR. J. Nutr. 134, 3378-3384 (2004).
42. J. G. Caporaso, J. Kuczynski, J. Stombaugh, K. Bittinger, F. D. Bushman, E. K. Costello, N. Fierer, A. G. Pena, J. K. Goodrich, J. I. Gordon, G. A. Huttley, S. T. Kelley, D. Knights, J. E. Koenig, R. E. Ley, C. A. Lozupone, D. McDonald, B. D. Muegge, M. Pirrung, J. Reeder, J. R. Sevinsky, P. J. Turnbaugh, W. A. Walters, J. Widmann, T. Yatsunenko, J. Zaneveld, R. Knight. QIIME allows analysis of high-throughput community sequencing data. Nature Meth. 7, 335-336 (2010).
43. R. C. Edgar, Search and clustering orders of magnitude faster than BLAST. Bioinformatics 26, 2460-2461 (2010).
44. J. R. Cole, Q. Wang, J. A. Fish, B. Chai, D. M. McGarrell, Y. Sun, C. T. Brown, A. Porras-Alfaro, C. R. Kuske, J. M. Tiedje. Ribosomal Database Project: data and tools for high throughput rRNA analysis. Nucl. Acids Res. 42, D633-D642 (2014).
45. T. Z. DeSantis, P. Hugenholtz, N. Larsen, M. Rojas, E. L. Brodie, K. Keller, T. Huber, D. Dalevi, P. Hu, G. L. Andersen. Greengenes, a chimera-checked 16S rRNA gene database and workbench compatible with ARB. Appl. Environ. Microbiol. 72, 5069-5072 (2006).
46. C. Quast, E. Pruesse, P. Yilmaz, J. Gerken, T. Schweer, P. Yarza, J. Peplies, F. O. Glockner. The SILVA ribosomal RNA gene database project: improved data processing and web-based tools. Nucl. Acids Res. 41, D590-D596 (2012).
47. C. Koetschan, S. Kittelmann, J. Lu, D. Al-Halbouni, G. N. Jarvis, T. Muller, M. Wolf, P. H. Janssen. Internal transcribed spacer 1 secondary structure analysis reveals a common core throughout the anaerobic fungi (Neocallimastigomycota). PloS One 9, e91928 (2014).
48. R Core Team R: A Language and Environment for Statistical Computing. (2015).
49. Y. Benjamini, Y. Hochberg, Controlling the false discovery rate: a practical and powerful approach to multiple testing. J. Royal Stat. Soc. Series B (Methodological) 57, 289-300 (1995).
50. D. V. Zaykin, Optimally weighted Z-test is a powerful method for combining probabilities in meta-analysis. J. Evol. Biol. 24, 1836-1841 (2011).
51. R. Rosenthal, Combining results of independent studies. Psychol. Bull. 85, 185 (1978).
52. Michael Dewey (2018). metap: meta-analysis of significance values. R package version 1.0.
53. Max Kuhn. Contributions from Jed Wing, Steve Weston, Andre Williams, Chris Keefer, Allan Engelhardt, Tony Cooper, Zachary Mayer, Brenton Kenkel, the R Core Team, Michael Benesty, Reynald Lescarbeau, Andrew Ziem, Luca Scrucca, Yuan Tang, Can Candan and Tyler Hunt. (2018). caret: Classification and Regression Training. R package version 6.0-80. www(dot)CRAN.R-project(dot)org/package=caret.
54. S. Purcell, B. Neale, K. Todd-Brown, L. Thomas, M. A. Ferreira, D. Bender, J. Maller, P. Sklar, P. I. de Bakker, M. J. Daly, P. C. Sham. PLINK: a tool set for whole-genome association and population-based linkage analyses. Am. J. Human Gen. 81, 559-575 (2007).
55. X. Zheng, snpgdsGRM: Genetic Relationship Matrix (GRM) for SNP genotype data. In ‘SNPRelate: Parallel Computing Toolset for Relatedness and Principal Component Analysis of SNP Data’ Version 1.14.0.
56. Carter T. Butts (2018). yacca: Yet Another Canonical Correlation Analysis Package. R package version 1.1.1. www(dot)CRAN.R-project(dot)org/package=yacca.
57. J. Yang, S. H. Lee, M. E. Goddard, P. M. Visscher, GCTA: a tool for genome-wide complex trait analysis. Amer. J. Human Genet. 88, 76-82 (2011).
58. J. Yang, B. Benyamin, B. P. McEvoy, S. Gordon, A. K. Henders, D. R. Nyholt, P. A. Madden, A. C. Heath, N. G. Martin, G. W. Montgomery, M. E. Goddard, P. M. Visscher. Common SNPs explain a large proportion of the heritability for human height. Nature Genetics 42, 565-569 (2010).
59. R. Schweiger, E. Fisher, E. Rahmani, L. Shenhav, S. Rosset, E. Halperin Using stochastic approximation techniques to efficiently construct confidence intervals for heritability. J. Comput. Biol. 25, 794-808 (2018).
60. Z. D. Kurtz, C. L. Muller, E. R. Miraldi, D. R. Littman, M. J. Blaser, R. A. Bonneau. Sparse and compositionally robust inference of microbial ecological networks. PLoS Comput. Biol. 11, e1004226 (2015).
61. K. Katoh, K. Misawa, K. I. Kuma, T. Miyata, MAFFT: a novel method for rapid multiple sequence alignment based on fast Fourier transform. Nucl. Acids Res. 30, 3059-3066 (2002).
62. K. Katoh, D. M. Standley, MAFFT multiple sequence alignment software version 7:

improvements in performance and usability. Molec. Biol. Evol. 30, 772-780 (2013).
63. M. N. Price, P. S. Dehal, A. P. Arkin, FastTree: computing large minimum evolution trees with profiles instead of a distance matrix. Molec. Biol. Evol. 26, 1641-1650 (2009).
64. M. N. Price, P. S. Dehal, A. P. Arkin, FastTree 2—approximately maximum-likelihood trees for large alignments. PloS One 5, e9490 (2010).
65. R. J. Wallace, C. A. McPherson. Factors affecting the rate of breakdown of bacterial protein in rumen fluid. Br. J. Nutr. 58, 313-323 (1987).
66. R. A. Leng, J. V. Nolan, Nitrogen metabolism in the rumen. J. Dairy Sci. 67, 1072-1089 (1984).
67. C. J. Newbold, K. Hillman, The effect of ciliate protozoa on the turnover of bacterial and fungal protein in the rumen of sheep. Lett. Appl. Microbiol. 11, 100-102 (1990).
68. I. Tapio, T. J. Snelling, F. Strozzi, R. J. Wallace, The ruminal microbiome associated with methane emissions from ruminant livestock. J. Anim. Sci. Biotechnol. 8, 7 (2017).

What is claimed is:

1. A method of breeding a ruminating animal having a desirable, hereditable trait comprising:
(a) analyzing in the microbiome of the animal for an amount of at least one hereditable bacteria which is associated with said hereditable trait, wherein the amount of said hereditable bacteria is indicative as to whether the animal has a desirable hereditable trait, wherein said hereditable bacteria is of any one of the operational taxonomic units (OTUs) set forth in Table 1, wherein the trait is the corresponding trait to said at least one hereditable bacteria as set forth in Table 1; and
(b) breeding the animal that has the desirable hereditable trait, thereby breeding the ruminating animal having a desirable hereditable trait.

2. A method of managing a herd of ruminating animals comprising:
(a) analyzing in the microbiome of a ruminating animal of the herd for an amount of at least one hereditable bacteria which is associated with said hereditable trait, wherein the amount of said hereditable bacteria is indicative that the animal has a non-desirable hereditable trait, wherein said hereditable bacteria is of any one of the operational taxonomic units (OTUs) set forth in Table 1, wherein the trait is the corresponding trait to said at least one hereditable bacteria as set forth in Table 1; and
(b) removing the animal with said non-desirable trait from the herd.

3. The method of claim 1, wherein said hereditable bacteria is of the family lachnospiraceae or of the genus Prevotella.

4. The method of claim 1, wherein the ruminating animal is a cow.

5. The method of claim 1, further comprising using the selected animal or a progeny thereof for breeding.

6. The method of claim 1, wherein said analyzing an amount is effected by analyzing the expression of at least one gene of the genome of said at least one bacteria.

7. The method of claim 1, wherein said analyzing an amount is effected by sequencing the DNA derived from a sample of said microbiome.

8. The method of claim 1, wherein said microbiome comprises a rumen microbiome or a fecal microbiome.

9. The method of claim 1, wherein when said ruminating animal that has been selected is a female ruminating animal, the method comprises artificially inseminating said female ruminating animal with semen from a male ruminating animal.

10. The method of claim 1, wherein when said ruminating animal that has been selected is a male ruminating animal, the method comprises inseminating a female ruminating animal with semen of said male ruminating animal.

11. A method of increasing the number of ruminating animals having a desirable microbiome comprising breeding a male and female of said ruminating animals, wherein the rumen microbiome of either of said male and/or said female ruminating animals comprises a hereditable microorganism having an OTU as set forth in Table 3 above a predetermined level, thereby increasing the number of ruminating animals having a desirable microbiome.

12. The method of claim 11, wherein said hereditable microorganism is associated with a hereditable trait.

13. A method of altering a trait of a ruminating animal comprising providing a microbial composition to the ruminating animal which comprises at least one microbe having an operational taxonomic unit (OTU) set forth in Table 2 and having a 16S rRNA sequence as set forth in SEQ ID NOs: 38-50 and 314-615, thereby altering the trait of the ruminating animal, wherein the microbial composition does not comprise a microbiome of the ruminating animal, wherein the trait is the corresponding trait to said at least one microbe as set forth in Table 2.

14. The method of claim 13, wherein said microbial composition comprises no more than 50 microbial species.

15. The method of claim 13, wherein said at least one microbe has an OTU set forth in Table 1.

16. A microbial composition comprising at least one microbe having an OTU set forth in Table 2, the microbial composition not being a microbiome.

17. The microbial composition of claim 16, comprising no more than 20 bacterial species.

18. The microbial composition of claim 16, comprising at least two microbes having an OTU as set forth in Table 2.
